(19) United States
(12) Patent Application Publication
(10) Pub. No.: US 2026/0083704 A1
(43) Pub. Date: Mar. 26, 2026

(54) LXR MODULATORS WITH BICYCLIC CORE MOIETY FOR TREATING DYSLIPIDEMIAS

(71) Applicant: OrsoBio, Inc., Menlo Park, CA (US)

(72) Inventors: Archana VIJAYAKUMAR, Foster City, CA (US); Eisuke MURAKAMI, Menlo Park, CA (US); Claus KREMOSER, Heidelberg (DE); Jen-Chieh CHUANG, Winchester, MA (US); Steve WENG, Menlo Park, CA (US); Ryan Steven HUSS, Woodside, CA (US); G. Mani SUBRAMANIAN, Menlo Park, CA (US); Robert P. MYERS, Menlo Park, CA (US)

(21) Appl. No.: 19/196,617

(22) Filed: May 1, 2025

Related U.S. Application Data
(63) Continuation of application No. PCT/US2023/078547, filed on Nov. 2, 2023.
(60) Provisional application No. 63/382,217, filed on Nov. 3, 2022.

(51) Int. Cl. A61K 31/404; A61K 31/4155; A61K 31/416; A61K 31/4178; A61K 31/423; A61K 31/4245; A61K 31/427; A61K 31/437; A61K 31/439; A61K 31/4439; A61K 31/454; A61K 31/4709; A61K 31/496; A61K 31/497; A61P 1/16; A61P 3/06

(52) U.S. Cl. CPC A61K 31/404; A61K 31/4155; A61K 31/416; A61K 31/4178; A61K 31/423; A61K 31/4245; A61K 31/427; A61K 31/437; A61K 31/439; A61K 31/4439; A61K 31/454; A61K 31/4709; A61K 31/496; A61K 31/497; A61P 1/16; A61P 3/06

(57) ABSTRACT

The present disclosure relates to novel compounds which are Liver X Receptor (LXR) modulators and to pharmaceutical compositions containing same. The present disclosure further relates to the use of said compounds in the prophylaxis and/or treatment of diseases which are associated with the modulation of LXR, for example, dyslipidemias. The present disclosure further relates to the use of the present compounds in the prophylaxis and/or treatment of metabolic disorders associated with an impairment in lipid homeostasis.

### CROSS REFERENCE TO RELATED APPLICATIONS

This application claims the benefit of U.S. Provisional Application No. 63/382,217, filed Nov. 3, 2022, the contents of which is incorporated herein by reference in its entirety.

### FIELD

The present disclosure relates to novel compounds which are Liver X Receptor (LXR) modulators and to pharmaceutical compositions containing same. The present disclosure further relates to the use of said compounds in the prophylaxis and/or treatment of diseases which are associated with the modulation of LXR, for example, dyslipidemias. The present disclosure further relates to the use of the present compounds in the prophylaxis and/or treatment of metabolic disorders associated with an impairment in lipid homeostasis.

### BACKGROUND

The Liver X Receptors, LXRα (NR1H3) and LXRβ (NR1H2) are members of the nuclear receptor protein superfamily. Both receptors form heterodimeric complexes with Retinoid X Receptor (RXRα, β or γ) and bind to LXR response elements (e.g. DR4-type elements) located in the promoter regions of LXR responsive genes. Both receptors are transcription factors that are physiologically regulated by binding ligands such as oxysterols or intermediates of the cholesterol biosynthetic pathways such as desmosterol. In the absence of a ligand, the LXR-RXR heterodimer is believed to remain bound to the DR4-type element in complex with co-repressors, such as NCOR1, resulting in repression of the corresponding target genes. Upon binding of an agonist ligand, either an endogenous one such as the oxysterols or steroid intermediates mentioned before or a synthetic, pharmacological ligand, the conformation of the heterodimeric complex is changed, leading to the release of corepressor proteins and to the recruitment of coactivator proteins such as NCOA1 (SRC1), resulting in transcriptional stimulation of the respective target genes. While LXRβ is expressed in most tissues, LXRα is expressed more selectively in cells of the liver, the intestine, adipose tissue and macrophages. The relative expression of LXRα and LXRβ at the mRNA or the protein level may vary between different tissues in the same species or between different species in a given tissue. The LXR's control reverse cholesterol transport, i.e. the mobilization of tissue-bound peripheral cholesterol into HDL and from there into bile and feces, through the transcriptional control of target genes such as ABCA1 and ABCG1 in macrophages and ABCG5 and ABCG8 in liver and intestine. This explains the anti-atherogenic activity of LXR agonists in dietary LDLR-KO mouse models. The LXRs, however, do also control the transcription of genes involved in lipogenesis (e.g. Srebp1c, Scd1, Fasn) which accounts for the liver steatosis observed following prolonged treatment with LXR agonists.

### BRIEF SUMMARY

The present disclosure relates to a method of treating a dyslipidemia or a metabolic disorder associated with an impairment in lipid homeostasis or in a subject in need thereof, comprising administering to the subject in need thereof an effective amount of a compound represented by Formula (I)

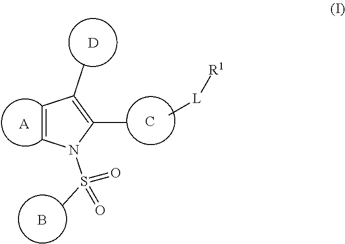

a glycine conjugate, tauro conjugate, enantiomer, diastereomer, tautomer, N-oxide, solvate, prodrug and pharmaceutically acceptable salt thereof, wherein cycle A, B, C, D and residue L and R¹ are described herein.

In some variations, the methods are directed toward the treatment of dyslipidemia. In other variations, the methods are directed toward metabolic disorders, for example, those associated with an impairment in lipid homeostasis. In some variations, the method of treatment is accomplished by administering the compound of Formula (I) or a pharmaceutically acceptable salt thereof.

The compounds of the present disclosure exhibit an advantageous liver/blood-ratio after oral administration so that disruption of the anti-atherosclerotic reverse cholesterol transport governed by LXR in peripheral macrophages can be avoided. The incorporation of an acidic moiety (or a bioisoster thereof) can improve additional parameters, e.g. microsomal stability, solubility and lipophilicity.

Thus, the present disclosure further relates to a pharmaceutical composition comprising a compound according to Formula (I) and at least one pharmaceutically acceptable carrier or excipient.

Accordingly, the present disclosure relates to the prophylaxis and/or treatment of a dyslipidemia, including prevention of clinical sequalae related to dyslipidemia. The present disclosure relates to the prophylaxis and/or treatment of a metabolic disorder associated with an impairment in lipid homeostasis.

### BRIEF DESCRIPTION OF THE DRAWINGS

FIG. 1 shows a representative dose-response curve for relative fluorescence intensity, normalized to dimethylsulfoxide (DMSO) control, in Upcyte hepatocytes treated with a dose range of Compound 21/3.

FIG. 2A shows representative images of lipid accumulation, assessed by fluorescent Bodipy™ staining, and gene expression in normal (HLO) and steatotic (sHLO) human-derived liver organoids. FIG. 2B shows dose-dependent reduction in lipid accumulation in sHLO treated with Compound 21/3 for 3 days. FIG. 2C shows dose-dependent reduction in DNL genes in GCKR WT (CC) and GCKR mutant (TT)-derived sHLOs treated with Compound 21/3 for 3 days. Data in FIG. 2A are obtained from (Ouchi, Togo et al. 2019). Data are shown as mean±SD. n=4 wells per treatment.

FIG. 3A shows plasma exposures in mice dosed with 1 mg/kg of Compound 21/3 or Compound pre-6/3 by oral gavage. FIG. 3B shows liver exposures in mice dosed with 1 mg/kg of Compound 21/3 or Compound pre-6/3 by oral gavage. FIG. 3C shows liver gene expression of LXR target genes 24 hours post a single oral dose of Compound 21/3 or Compound pre-6/3. Data are shown as mean±SD. N=3-8 wells per treatment.

FIG. 4A shows liver target engagement assessed by expression of genes involved in de novo lipogenesis (DNL); FIG. 4B shows liver triglyceride (TG) content; FIG. 4C shows plasma TG levels in diet-induced obese (DIO) mice, high-fat diet (HFD)-fed Zucker diabetic fatty (ZDF) rats, and HFD-fed Sprague-Dawley (SD) rats treated with Compound 21/3 once daily by oral gavage for 14-21 days. FIG. 4D shows ileal Srebp1c expression in DIO mice. FIG. 4E shows intestinal lipid absorption, as measured by the appearance of ³H-triolein in plasma of DIO mice treated with vehicle or Compound pre-6/3 (5 mg/kg by oral gavage). FIG. 4F shows hepatic expression (left) and plasma levels (right) of angiopoietin-line 3 (Angptl3) in HFD-fed ZDF rats treated with vehicle or Compound 21/3 (15 mg/kg, once daily by oral gavage); FIG. 4G shows hepatic gene expression of enzymes involved in circulating TG clearance and/or hepatic TG secretion in HFD-fed ZDF rats treated with vehicle or Compound pre-6/3 (5 mg/kg, once daily by oral gavage). Data are shown as mean±SD. N=5-8 animals per group. *p≤0.05, **p≤0.01, ***p≤0.001, ****p≤0.0001 vs. vehicle, unless otherwise indicated; ^{#}p≤0.05 pioglitazone vs. vehicle Kruskal-Wallis (non-parametric One-Way ANOVA) test with Dunn's multiple comparisons test (FIG. 4A, FIG. 4B, FIG. 4D), repeated measures two-way ANOVA with Dunnett's multiple comparisons test (FIG. 4C), two-way ANOVA with Sidak's multiple comparisons test (FIG. 4E), Mann-Whitney test (FIG. 4F-G).

FIG. 5A shows plasma total cholesterol (TC) levels; FIG. 5B shows liver total cholesterol (TC) levels; FIG. 5C shows plasma alanine aminotransferase (ALT) levels; FIG. 5D shows plasma aspartate aminotransferase (AST) levels in diet-induced obese (DIO) mice, high-fat diet (HFD)-fed Zucker diabetic fatty (ZDF) rats, and HFD-fed Sprague-Dawley (SD) rats treated with Compound 21/3 once daily by oral gavage for 14-21 days as indicated in figure herein. FIG. 5E shows liver, ileum, and buffy coat expression of genes involved in cholesterol synthesis, metabolism, and efflux transport in response to Compound 21/3 in DIO mice. Data are shown as mean±SD. N=5-8 animals per group. *p≤0.05, **p≤0.01, ***p≤0.001, ****p≤0.0001 vs. vehicle, unless otherwise indicated; ^{#}p≤0.05 pioglitazone vs. vehicle repeated measures two-way ANOVA with Dunnett's multiple comparisons test (FIG. 5A, FIG. 5C, FIG. 5D), Kruskal-Wallis (non-parametric one-way ANOVA) test with Dunn's multiple comparisons test (FIG. 5B, FIG. 5E).

FIG. 6A shows liver triglyceride (TG) and total cholesterol content; FIG. 6B shows liver expression of genes involved in lipid synthesis and metabolism in humanized liver chimeric PXB mice treated with Compound 21/3 (1 mg/kg, once daily by oral gavage) for 8 days. Data are shown as mean±SD. N=4-5 animals per group. *p≤0.05, **p≤0.01, ***p≤0.001, ****p≤0.0001 Mann-Whitney test.

FIG. 7A shows time course of glucose infusion rate (GIR) for the duration of the clamp;

FIG. 7B shows steady-state GIR and hepatic glucose production (HGP) during the 1^{st} (8 mU/kg/min) and 2^{nd} (18 mU/kg/min) steps of insulin infusion; FIG. 7C shows tissue glucose uptake measured at the end of the two-step hyperinsulinemic-euglycemic clamp performed in DIO mice treated with vehicle, Compound pre-6/3 (5 mg/kg) or pioglitazone (30 mg/kg) once daily by oral gavage for four weeks. FIG. 7D shows oral glucose tolerance test performed after four weeks of dosing; FIG. 7E shows immunostaining of pancreatic sections with insulin (red, β cells), glucagon (green, α cells) and 4′,6-diamidino-2-phenylindole (DAPI) (blue, nuclei) harvested from HFD-fed ZDF rats treated with vehicle or Compound 21/3 (15 mg/kg) once daily by oral gavage for 5 weeks. Data are shown as mean±SD. N=5-11 animals per group. *p≤0.05, **p≤0.01, ***p≤0.001, ****p≤0.0001 vs. vehicle, unless otherwise indicated; ^{#}p≤0.05 pioglitazone vs. vehicle. Repeated measures Two-way ANOVA with Dunnett's multiple comparisons test (FIG. 7A, FIG. 7D), Two-way ANOVA with Dunnett's multiple comparisons test (FIG. 7B, FIG. 7C). EDL=extensor digitorum longus, GIR=glucose infusion rate, HGP=hepatic glucose production, iWAT=inguinal white adipose tissue.

FIG. 8A shows representative images of hematoxylin and eosin (H&E) or Picrosirus red (PSR)-stained liver sections; FIG. 8B shows liver hydroxyproline, FIG. 8C shows liver collagen, FIG. 8D shows liver triglyceride (TG) content, and FIG. 8E shows terminal liver gene expression from 12-week CDHFD/sodium nitrate-treated rats dosed with vehicle or Compound pre-6/3 (5 mg/kg) once daily by oral gavage for 6 weeks. Data are shown as mean±SD. N=7-8 animals per group. *p≤0.05, **p≤0.01, ***p≤0.001, ****p≤0.0001 Mann-Whitney test.

FIG. 9 shows study schemas for single ascending dose (SAD) cohorts in healthy volunteers in Parts A and C in the overall study design for Phase 1 study.

FIG. 10 shows study schemas for multiple ascending dose (MAD) cohorts in Parts B and C in the overall study design for Phase 1 study.

FIG. 11 shows plasma concentration time profiles on Day 1 in the SAD study.

FIG. 12 shows plasma concentration time profiles on Day 14 in the MAD study.

FIGS. 13A-13H shows relative changes in pre-dose serum lipid parameters during MAD phase of the study. FIG. 13A shows relative (%) change in triglycerides from Day 1 to 14 during the MAD phase of the study. FIG. 13B shows relative (%) change in total cholesterol from Day 1 to 14 during the MAD phase of the study. FIG. 13C shows relative (%) change in HDL-C from Day 1 to 14 during the MAD phase of the study. FIG. 13D shows relative (%) change in TG/HDL-C ratio from Day 1 to 14 during the MAD phase of the study. FIG. 13E shows relative (%) change in LDL-C from Day 1 to 14 during the MAD phase of the study. FIG. 13F shows relative (%) change in LDL particles from Day 1 to 14 during the MAD phase of the study. FIG. 13G shows relative (%) change in small LDL particles from Day 1 to 14 during the MAD phase of the study. FIG. 13H shows relative (%) change in ApoB from Day 1 to 14 during the MAD phase of the study.

FIG. 14A shows levels of ApoC3 in response to Compound 21/3 or placebo treatment from Day 1 to Day 14. FIG. 14B shows levels of ANGPTL3 in response to Compound 21/3 or placebo treatment from Day 1 to Day 14. FIG. 14C shows levels of reverse cholesterol transport (RCT) genes in peripheral blood mononuclear cells (PBMCs) in response to Compound 21/3 and placebo treatment from Day 1 to Day 14.

### DETAILED DESCRIPTION

The present disclosure provides methods for treating or preventing various LXR mediated diseases by administering the compound of Formula (I)

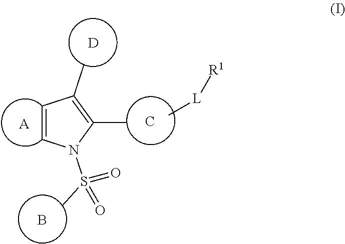

a glycine conjugate, tauro conjugate, enantiomer, diastereomer, tautomer, N-oxide, solvate, prodrug and pharmaceutically acceptable salt thereof, are described herein. In some variations, [symbol] is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
   wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
   wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      wherein \textcircled{D} has a substituent from above in 1,2-orientation regarding to the connection towards

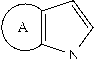

or has an annelated additional cycle in 1,2-orientation;
L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
      R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituent independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR⁷¹) and S(═NR⁷¹)₂;
   n is independently selected from 0 to 2;
   and with the proviso, that the following structures are excluded:

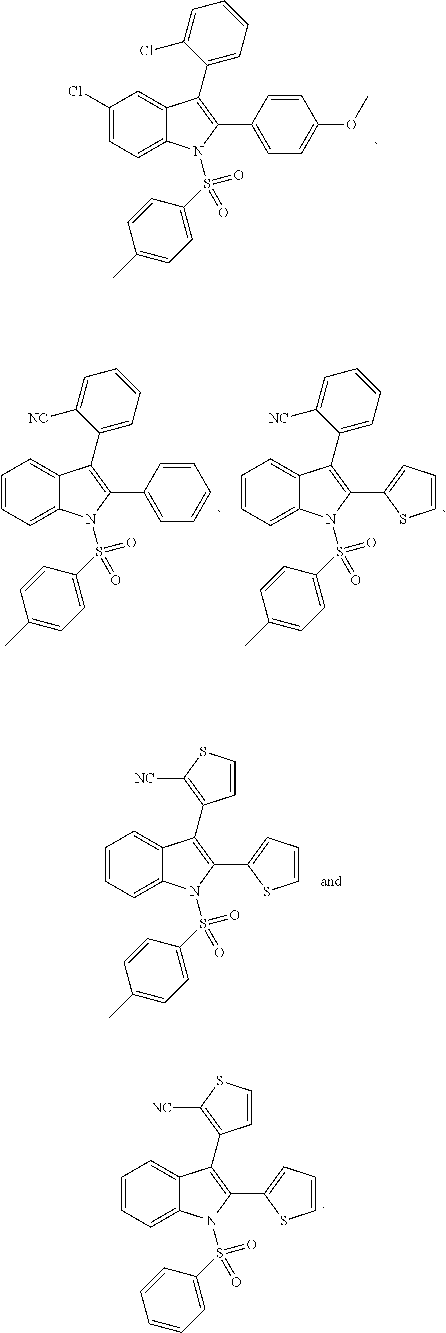

In certain embodiments in combination with any of the above or below embodiments

[symbol]

is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
   wherein optionally two adjacent substituents on the aryl or heteroaryl moiety form a 5- to 8-membered partially unsaturated cycle optionally containing 1 to 3 heteroatoms independently selected from O, S or N, and
   wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl.

In certain embodiments in combination with any of the above or below embodiments

[symbol]

is an annelated phenyl, thiophenyl, thiazolyl, pyridyl, pyrimidinyl, pyridazinyl and pyrazinyl, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl.

In certain embodiments in combination with any of the above or below embodiments

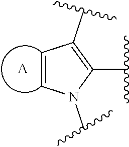

is selected from

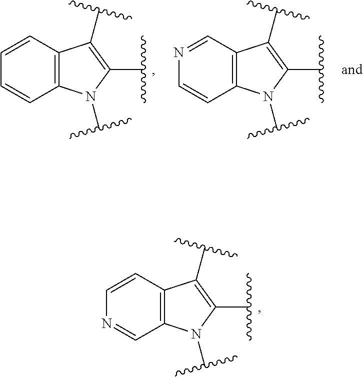

wherein [symbol] is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, NH₂, NHC₁₋₄-alkyl, N(C₁₋₄-alkyl)₂, SO₂—C₁₋₄-alkyl and SO₂-halo-C₁₋₄-alkyl.

In certain embodiments in combination with any of the above or below embodiments

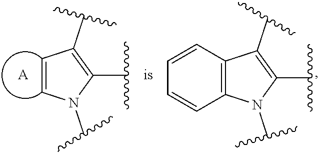

wherein [symbol] is unsubstituted or substituted with 1 to 3 substituents independently selected from the group consisting of F, Cl, Br, CN, Me, Et, CF₃, CHF₂, OH, OMe, OCF₃ and OCHF₃.

In certain embodiments in combination with any of the above or below embodiments

\textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl.

In certain embodiments in combination with any of the above or below embodiments

\textcircled{B} is selected from the group consisting of phenyl, pyridyl and thiophenyl,
wherein phenyl, pyridyl and thiophenyl are substituted with 1 to 4 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the phenyl and pyridyl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl.

In certain embodiments in combination with any of the above or below embodiments

\textcircled{B} is selected from the group consisting of phenyl, naphthyl, pyridyl, pyrimidinyl, thiophenyl, thiazolyl, cyclopentyl, cyclohexyl, bicyclo[1.1.1]pentyl, bicyclo[2.2.2]octyl, bicyclo[2.2.1]heptyl, pentacyclo[4.2.0.0^{2,5}0.0^{3,8}.0^{4,7}]octyl and piperidinyl,
wherein the cycle is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH and halo-C₁₋₄-alkyl-OH; and wherein optionally two adjacent substituents on the phenyl ring form together a —(CH₂)₃—, —(CH₂)₄—, —OCF₂O— and —OCH₂O— group.

In certain embodiments in combination with any of the above or below embodiments

\textcircled{B} is selected from the group consisting of phenyl and pyridyl,
wherein phenyl and pyridyl is substituted with 1 to 2 substituents independently selected from the group consisting of F, Cl, CN, CF₃, CH₂F and CHF₂.

In certain embodiments in combination with any of the above or below embodiments

\textcircled{B} is 4-difluoromethylphenyl.

In certain embodiments in combination with any of the above or below embodiments

\textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
   wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl.

In certain embodiments in combination with any of the above or below embodiments

\textcircled{C} is selected from the group consisting of phenyl, pyridyl and thiophenyl,
wherein phenyl, pyridyl and thiophenyl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
   wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      and wherein residue -L-R¹ is linked in 1,3-orientation regarding the connection towards

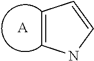

and L is not a bond.

In certain embodiments in combination with any of the above or below embodiments

\textcircled{C} is selected from phenyl, pyridyl and thiophenyl; wherein phenyl, pyridyl and thiophenyl is unsubstituted or substituted with 1 to 3 substituents independently selected from the group consisting of F, Cl, CN, OH, oxo, C₁₋₄-alkyl, halo-C₁₋₄-alkyl, O—C₁₋₄-alkyl and O-halo-C₁₋₄-alkyl; and wherein the residue -L-R¹ is linked in 1,3-orientation regarding the connection towards

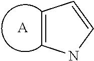

and L is not a bond.

In certain embodiments in combination with any of the above or below embodiments

\textcircled{C} is phenyl, wherein phenyl is unsubstituted or substituted with F, Cl and Me; and wherein the residue -L-R¹ is linked in 1,3-orientation regarding the connection towards

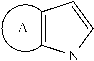

and L is not a bond.

In certain embodiments in combination with any of the above or below embodiments

L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl.

In certain embodiments in combination with any of the above or below embodiments

L is selected from the group consisting of 3- to 10-membered cycloalkylene, 3- to 10-membered heterocycloalkylene containing 1 to 4 heteroatoms independently selected from N, O and S, 6-membered arylene and 5- to 6-membered heteroarylene containing 1 to 2 heteroatoms independently selected from N, O and S,
wherein cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR41R⁴²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl.

In certain embodiments in combination with any of the above or below embodiments

-L-R¹ is selected from

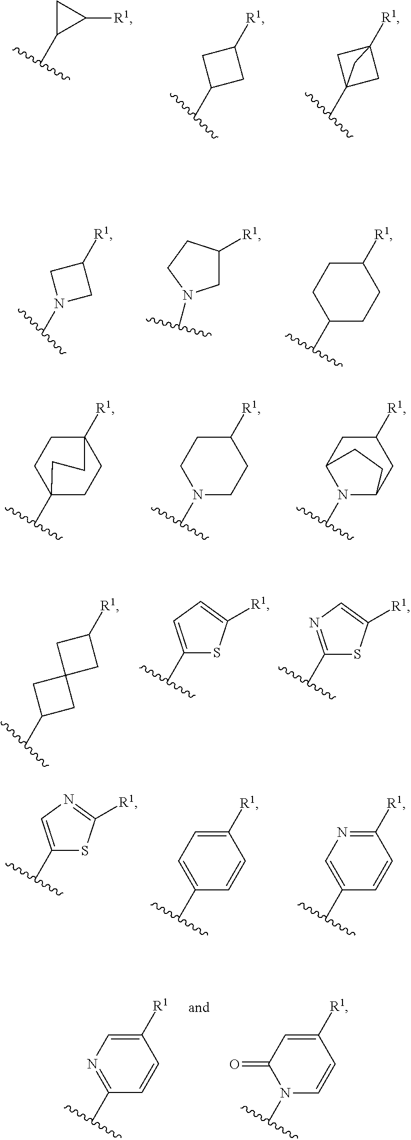

wherein the cycle is unsubstituted or substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

In certain embodiments in combination with any of the above or below embodiments

-L-R¹ is selected from

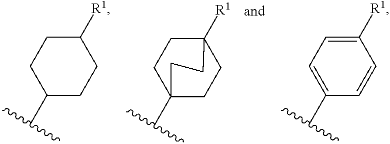

wherein phenyl is unsubstituted or substituted with 1 to 4 substituents independently selected from the group consisting of F, Cl, CN, OH, Me and OMe.

In certain embodiments in combination with any of the above or below embodiments

-L-R¹ is selected from

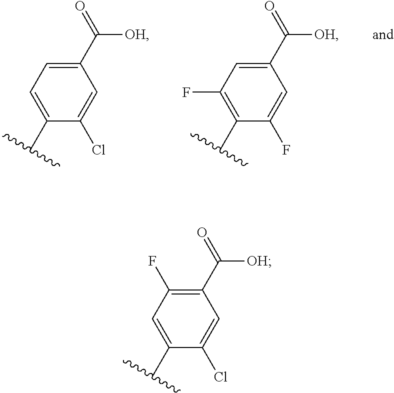 [SMILES: *c1ccc(C(=O)O)cc1Cl ; *c1c(F)cc(C(=O)O)cc1F ; *c1cc(F)c(C(=O)O)cc1Cl]

and optionally the glycine and tauro conjugate thereof.

In certain embodiments in combination with any of the above or below embodiments

R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl.

In certain embodiments in combination with any of the above or below embodiments

R¹ is selected from CO₂H, tetrazole, CH₂CO₂H, OCH₂CO₂H, SO₂CH₂CO₂H, CHMeCO₂H, CMe₂CO₂H, C(OH)MeCO₂H, CONHSO₂Me and CONH(OH); and optionally the glycine and tauro conjugate thereof.

In certain embodiments in combination with any of the above or below embodiments

R¹ is selected from CO₂H and C(OH)MeCO₂H; and optionally the glycine and tauro conjugate thereof.

In certain embodiments in combination with any of the above or below embodiments

\textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      wherein \textcircled{D} has a substituent from above in 1,2-orientation regarding to the connection towards

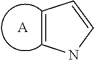

or has an annelated additional cycle in 1,2-orientation.

In certain embodiments in combination with any of the above or below embodiments

\textcircled{D} is selected from the group consisting of 6- or 10-membered aryl and 5- to 10-membered heteroaryl containing 1 to 4 heteroatoms independently selected from N, O and S,
wherein aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹) C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
   wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
   wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
      wherein \textcircled{D} has a substituent from above in 1,2-orientation regarding to the connection towards

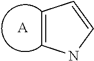

or has an annelated additional cycle in 1,2-orientation.

In certain embodiments in combination with any of the above or below embodiments

\textcircled{D} is selected from the group consisting

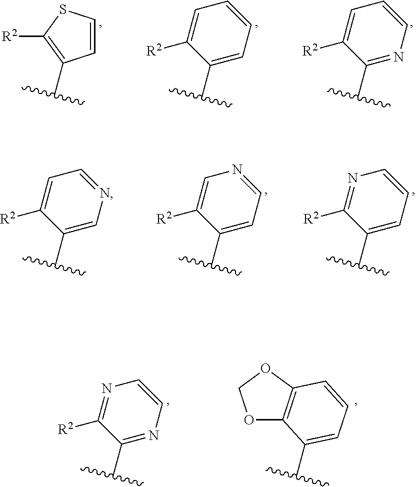

wherein
R² is selected from Me, F, Cl, CN, Me, CHO, CHF₂, CF₃, SO₂Me,

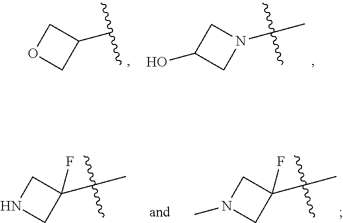

and
wherein \textcircled{D} is not further substituted or further substituted with 1 to 2 substituents selected from the group consisting F, Cl, CN, Me, OMe, CHO, CHF₂ and CF₃.

In certain embodiments in combination with any of the above or below embodiments

\textcircled{D} is selected from the group consisting of

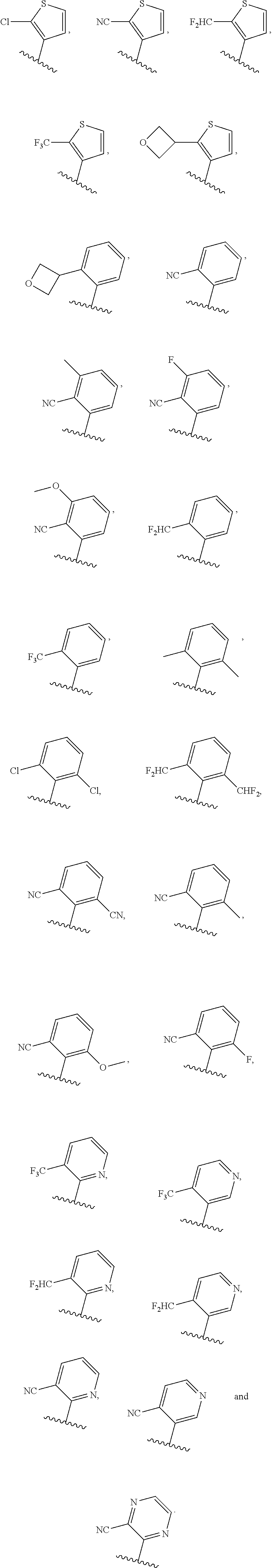

In certain embodiments in combination with any of the above or below embodiments

Formula (I) contains a substituent selected from the group consisting of CO₂H, tetrazole, CONHSO₂Me and CONH(OH); and optionally the glycine and tauro conjugate thereof.

In certain embodiments in combination with any of the above or below embodiments

Formula (I) contains a carboxylic acid moiety and optionally the glycine and tauro conjugate thereof.

In certain embodiments,

### L-R¹ is

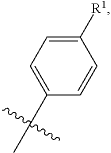

wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

In certain embodiments, R¹ is C₀₋₆-alkylene-CO₂R⁴¹ or C₀₋₆-alkylene-CONR⁴¹R⁴², or a glycine conjugate or tauro conjugate thereof.

In certain embodiments, R¹ is COOH, or a glycine conjugate or tauro conjugate thereof.

In certain embodiments, R¹ is C₀₋₆-alkylene-CONR⁴¹R⁴².

In certain embodiments, R⁴¹ and R⁴² are independently selected from H and C₁₋₄alkyl, wherein C₁₋₄alkyl is unsubstituted or substituted with CO₂H.

In certain embodiments,

### —C-L-R¹ is

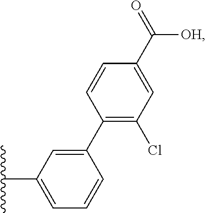 [SMILES: *c1cccc(-c2ccc(C(=O)O)cc2Cl)c1]

or a glycine conjugate or tauro conjugate thereof.

In certain embodiments, L-R¹ is

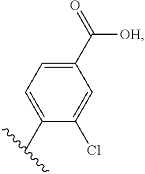 [SMILES: *c1ccc(C(=O)O)cc1Cl]

or a glycine conjugate or tauro conjugate thereof. In certain embodiments, L-R¹ is

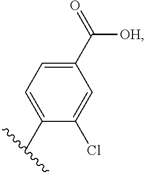 [SMILES: *c1ccc(C(=O)O)cc1Cl]

In certain embodiments, the compound is a glycine conjugate.

In certain embodiments, the compound is

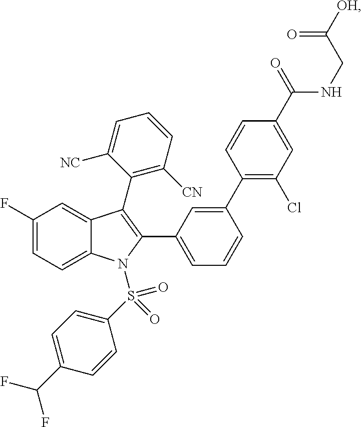 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]

or pharmaceutically acceptable salt thereof. In certain embodiments, the compound is

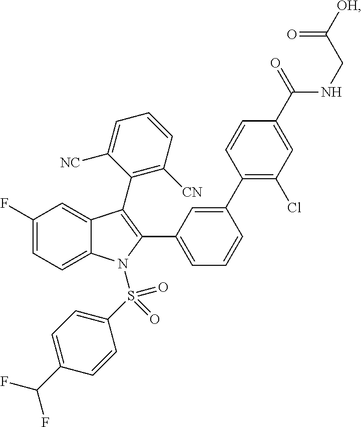 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]

In certain embodiments, the compound is

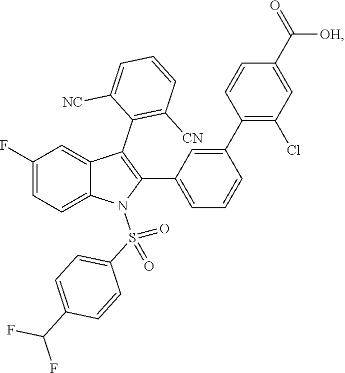 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

or a glycine conjugate thereof, or a pharmaceutically acceptable salt thereof. In certain embodiments, the compound is

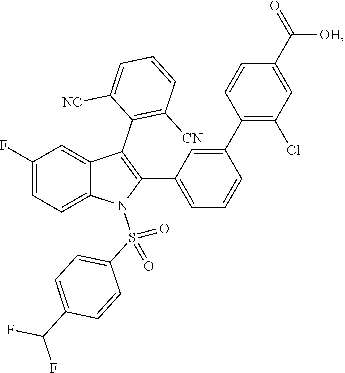 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

or a pharmaceutically acceptable salt thereof. In certain embodiments, the compound is

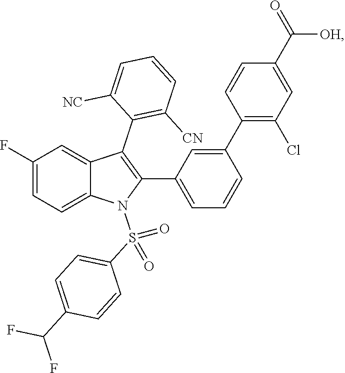 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

The present disclosure also provides the compound of the present disclosure for use as a medicament.

Also provided is the compound of the present disclosure for use in the prophylaxis and/or treatment of diseases amenable for treatment with LXR modulators. LXR controls SREBP1c and other genes such as ACC, FASN, and SCD involved in de novo lipogenesis. LXR regulated apoC3 and ANGPTL3/4/8 genes are involved in triglyceride clearance. LXR also controls genes involved on cholesterol metabolism including IDOL, PCSK9 and CETP.

Also provided is the compound of the present disclosure for use in treating a LXR mediated disease selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

In certain embodiments, the disease is selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome or cardiac steatosis.

In certain embodiments, the disease is cancer.

In certain embodiments, the disease is selected from viral myocarditis, hepatitis C virus infection or its complications.

Also, the present disclosure relates to a method for preventing and/or treating dyslipidemia. The present disclosure relates to a method for preventing and/or treating a metabolic disorder associated with an impairment in lipid homeostasis.

Also provided is the compound of the present disclosure for use in treating dyslipidemia. In certain embodiments, the dyslipidemia includes condition characterized by alterations in triglycerides, cholesterol, low-density lipoprotein cholesterol (LDL-C), high-density lipoprotein (HDL), other lipids, or lipoproteins.

The dyslipidemias can be of any of the following types: Fredrickson phenotype I; Fredrickson phenotype II; Fredrickson phenotype III; Fredrickson phenotype IV; and Fredrickson phenotype V.

In certain embodiments, the dyslipidemia is hypertriglyceridemia. In certain embodiments, the dyslipidemia is severe hypertriglyceridemia.

In certain embodiments, the dyslipidemia is selected from hypertriglyceridemia (HTG), severe hypertriglyceridemia (SHTG), familial hypercholesterolemia, heterozygous familial hypercholesterolemia (HeFH), homozygous familial hypercholesterolemia (HoFH).

In certain embodiments, the dyslipidemia is selected from familial chylomicronemia syndrome and mixed disorder chylomicronemia. In certain embodiments, the dyslipidemia is hypercholesterolemia. In certain embodiments, the dyslipidemia is familial combined hyperlipidemia (e.g., LDL and VLDL elevated). In certain embodiments, the dyslipidemia is familial dysbetalipoproteinemia (e.g., VLDL and chylomicrons elevated). In certain embodiments, the dyslipidemia is hypertrigylceridemia (e.g., VLDL elevated, cholesterol may also be elevated) In certain embodiments, the dyslipidemia is mixed dyslipidemia (e.g., elevated chylomicrons and VLDL, TGs >99% percentile).

In certain embodiments, the dyslipidemia is selected from severe hypertriglyceridemia (SHTG) or hypertriglyceridemia (HTG). In certain embodiments, the compound is for use in treating SHTG characterized by serum triglyceride levels greater than 1000 mg per dL. In certain embodiments, the compound is for use in lowering triglyceride level to decrease a risk of pancreatitis. In certain embodiments, the method decreases a risk of pancreatitis in a subject.

In certain embodiments, In certain embodiments, the compound is for use in lowering triglyceride level to reduce cardiovascular risk. In certain embodiments, the method reduces cardiovascular risk in a subject.

Also provided is the compound of the present disclosure for use in treating clinical sequalae of dyslipidemia selected from hypertriglyceridemia (HTG), severe hypertriglyceridemia (SHTG), familial hypercholesterolemia, heterozygous familial hypercholesterolemia, homozygous familial hypercholesterolemia, familial chylomicronemia syndrome, mixed disorder chylomicronemia, hypercholesterolemia, familial combined hyperlipidemia, familial dysbetalipoproteinemia, and mixed dyslipidemia.

Also provided is the compound of the present disclosure for use in treating a metabolic disorder associated with an impairment in lipid homeostasis, for example, an impairment in de novo lipogenesis.

Also provided is the compound of the present disclosure for use in treating a metabolic disorder associated with an impairment in lipid homeostasis.

Also provided is the compound of the present disclosure for use in treating non-alcoholic fatty liver disease (NAFLD) or nonalcoholic steatohepatitis (NASH) in a subject that has a glucokinase regulatory protein (GCKR) phenotype. In certain embodiments, de novo lipogenesis is elevated in the subject. In certain embodiments, expression of lipogenic genes in the subject and/or lipid accumulation in the subject is reduced by the treatment. In certain embodiments, the method comprises treating a metabolic disorder associated with an impairment in de novo lipogenesis. In certain embodiments, the method comprises treating non-alcoholic fatty liver disease (NAFLD) or nonalcoholic steatohepatitis (NASH) in a subject in need thereof, the subject having a glucokinase regulatory protein (GCKR) phenotype In certain embodiments, de novo lipogenesis is elevated in the subject.

The present disclosure further relates to a method for preventing and/or treating diseases mediated by LXRs, the method comprising administering a compound of the present disclosure in an effective amount to a subject in need thereof.

More specifically, the present disclosure relates to a method for preventing and/or treating diseases selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

Also, the present disclosure relates to a method for preventing and/or treating diseases such as alcoholic liver disease or alcoholic hepatitis. In certain embodiments, alcoholic liver disease or alcoholic hepatitis is also associated with increased DNL (de novo lipogenesis)/steatosis. Types of alcoholic liver disease include fatty liver, alcoholic hepatitis, and alcoholic cirrhosis. Circulating lipids can be associated with alcoholic liver disease. Consumption of alcohol can lead to high triglyceride and cholesterol in the liver. Also, as the body breaks down the alcohol, there can be high triglyceride levels in the blood.

Moreover, the present disclosure also relates to the use of a compound according to the present disclosure in the preparation of a medicament for the prophylaxis and/or treatment of a LXR mediated disease.

More specifically, the present disclosure relates to the use of a compound according to the present disclosure in the preparation of a medicament for the prophylaxis and/or treatment of a LXR mediated disease, wherein the disease is selected from non-alcoholic fatty liver disease, non-alcoholic steatohepatitis, liver inflammation, liver fibrosis, obesity, insulin resistance, type II diabetes, familial hypercholesterolemia, hypercholesterolemia in nephrotic syndrome, metabolic syndrome, cardiac steatosis, cancer, viral myocarditis, hepatitis C virus infection or its complications, and unwanted side-effects of long-term glucocorticoid treatment in diseases such as rheumatoid arthritis, inflammatory bowel disease and asthma.

Also, the present disclosure relates to the use of a compound according to the present disclosure in the preparation of a medicament for the prophylaxis and/or treatment of a LXR mediated disease.

Also, the present disclosure relates to the use of a compound according to the present disclosure in the preparation of a medicament for the prophylaxis and/or treatment of dyslipidemia. The present disclosure relates to the use of a compound according to the present disclosure in the preparation of a medicament for the prophylaxis and/or treatment of a metabolic disorder associated with an impairment in lipid homeostasis. In certain embodiments, expression of lipogenic genes in the subject and/or lipid accumulation in the subject is reduced.

Also provided is a pharmaceutical composition comprising the compound of the present disclosure and a pharmaceutically acceptable carrier or excipient.

In the context of the present disclosure “C₁₋₆-alkyl” means a saturated alkyl chain having 1 to 6 carbon atoms which may be straight chained or branched. Examples thereof include methyl, ethyl, propyl, isopropyl, n-butyl, isobutyl, tert-butyl, n-pentyl, isopentyl, n-hexyl and isohexyl. Similarly, “C₁₋₄-alkyl” means a saturated alkyl chain having 1 to 4 carbon atoms which may be straight chained or branched. Examples thereof include methyl, ethyl, propyl, isopropyl, n-butyl, isobutyl, and tert-butyl.

The term “halo-C₁₋₄-alkyl” means that one or more hydrogen atoms in the alkyl chain are replaced by a halogen. A example thereof is CH₂F, CHF₂ and CF₃.

A “C₀₋₆-alkylene” means that the respective group is divalent and connects the attached residue with the remaining part of the molecule. Moreover, in the context of the present disclosure, “C₀-alkylene” is meant to represent a bond, whereas C₁-alkylene means a methylene linker, C₂-alkylene means a ethylene linker or a methyl-substituted methylene linker and so on. In the context of the present disclosure, a C₀₋₆-alkylene preferably represents a bond, a methylene, a ethylene group or a propylene group.

Similarly, a “C₂₋₆-alkenylene” and a “C₂₋₆-alkinylene” means a divalent alkenyl or alkynyl group which connects two parts of the molecule.

A 3- to 10-membered cycloalkyl group means a saturated or partially unsaturated mono-, bi-, spiro- or multicyclic ring system comprising 3 to 10 carbon atoms. Examples include cyclopropyl, cyclobutyl, cyclopentyl, cyclohexyl, cyclohexenyl, bicyclo[2.2.2]octyl, bicyclo[3.2.1]octanyl, spiro[3.3]heptyl, bicyclo[2.2.1]heptyl, adamantyl and pentacyclo[4.2.0.0^{2,5}0.0^{3,8}.0^{4,7}]octyl. Consequently, a 3- to 6-membered cycloalkyl group means a saturated or partially unsaturated mono-bi-, or spirocyclic ring system comprising 3 to 6 carbon atoms whereas a 5- to 8-membered cycloalkyl group means a saturated or partially unsaturated mono-, bi-, or spirocyclic ring system comprising 5 to 8 carbon atoms.

A 3- to 10-membered heterocycloalkyl group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O, S, SO and SO₂. Examples thereof include epoxidyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom. An example for a S-linked heterocycloalkyl is the cyclic sulfonimidamide

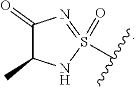 [SMILES: *S1(=O)=NC(=O)[C@H](C)N1]

A 5- to 14-membered mono-, bi- or tricyclic heteroaromatic ring system (within the application also referred to as heteroaryl) means an aromatic ring system containing up to 6 heteroatoms independently selected from N, O, S, SO and SO₂. Examples of monocyclic heteroaromatic rings include pyrrolyl, imidazolyl, furanyl, thiophenyl (thienyl), pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzofuranyl, benzoxazolyl, indolyl, indolizinyl 1,5-naphthyridinyl, 1,7-naphthyridinyl and pyrazolo[1,5-a]pyrimidinyl. Examples of tricyclic heteroaromatic rings include acridinyl, benzo[b][1,5]naphthyridinyl and pyrido[3,2-b][1,5]naphthyridinyl.

The nitrogen or sulphur atom of the heteroaryl system may also be optionally oxidized to the corresponding N-oxide, S-oxide or S,S-dioxide.

If not stated otherwise, the heteroaryl system can be connected via a carbon or nitrogen atom. Examples for N-linked heterocycles are

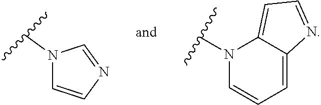 [SMILES: *n1ccnc1 ; "and" ; *n1cccc2nccc1-2]

A 6- to 14-membered mono-, bi- or tricyclic aromatic ring system (within the application also referred to as aryl) means an aromatic carbon cycle such as phenyl, naphthyl, anthracenyl or phenanthrenyl.

The term “N-oxide” denotes compounds, where the nitrogen in the heteroaromatic system (preferably pyridinyl) is oxidized. Such compounds can be obtained in a known manner by reacting a compound of the present disclosure (such as in a pyridinyl group) with H₂O₂ or a peracid in an inert solvent.

Halogen is selected from fluorine, chlorine, bromine and iodine, more preferably fluorine or chlorine and most preferably fluorine.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the compounds of the present disclosure are partly subject to tautomerism. For example, if a heteroaromatic group containing a nitrogen atom in the ring is substituted with a hydroxy group on the carbon atom adjacent to the nitrogen atom, the following tautomerism can appear:

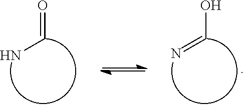

A cycloalkyl or heterocycloalkyl group can be connected straight or spirocyclic, e.g. when cyclohexane is substituted with the heterocycloalkyl group oxetane, the following structures are possible:

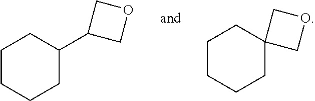 [SMILES: C1CCC(C2COC2)CC1 ; C1CCC2(CC1)COC2]

The term “1,3-orientation” means that on a ring the substituents have at least one possibility, where 3 atoms are between the two substituents attached to the ring system, e.g.

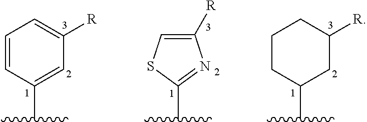

The term “1,2-orientation” (ortho) means that on a ring the substituents have one possibility, where 2 atoms are between the two substituents attached to the ring system, e.g.

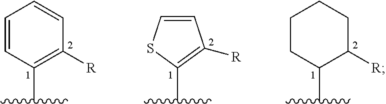

alternatively the residue R can be incorporated in an annelated additional cycle, e.g.

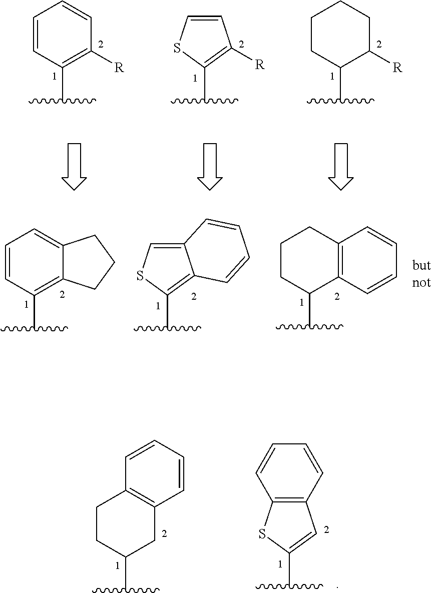

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

The compounds of the present disclosure can be in the form of a prodrug compound. “Prodrug compound” means a derivative that is converted into a compound according to the present disclosure by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present disclosure is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present disclosure according to well-known methods. Other examples of the prodrug are compounds (referred to as “ester prodrug” in the application, wherein the carboxylate in a compound of the present disclosure is, for example, converted into an alkyl-, aryl-, arylalkylene-, amino-, choline-, acyloxyalkyl-, 1-((alkoxycarbonyl)oxy)-2-alkyl, or linolenoyl-ester. Exemplary structures for prodrugs of carboxylic acids are

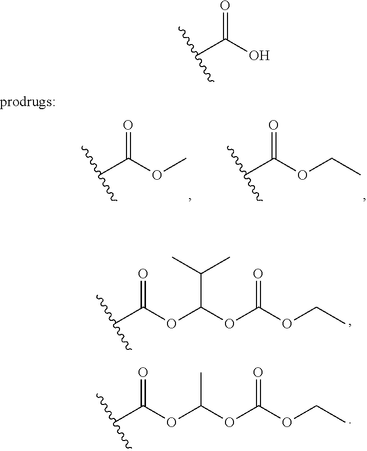

A ester prodrug can also be formed, when a carboxylic acid forms a lactone with a hydroxy group from the molecule. An exemplary example is

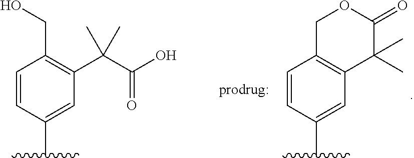 [SMILES: *c1ccc(CO)c(C(C)(C)C(=O)O)c1 ; *c1ccc2c(c1)C(C)(C)C(=O)OC2]

The term “—CO₂H or an ester thereof” means that the carboxylic acid and the alkyl esters are intended, e.g.

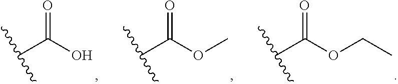

The term “glycine conjugate or tauro conjugate thereof” means, that the carboxylic acid moiety in the molecule is connected with glycine or taurine, respectively, to form the conjugate (and potentially a prodrug, solvate or pharmaceutically acceptable salt thereof):

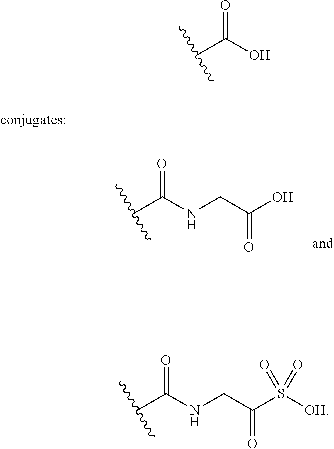

Metabolites of compounds of the present disclosure are also within the scope of the present disclosure.

Where tautomerism, like e.g. keto-enol tautomerism, of compounds of the present disclosure or their prodrugs may occur, the individual forms, like e.g. the keto and enol form, are each within the scope of the present disclosure as well as their mixtures in any ratio. Same applies for stereoisomers, like e.g. enantiomers, cis/trans-isomers, atropisomers, conformers and the like.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present disclosure may be obtained from stereoselective synthesis using optically pure starting materials. Another way to obtain pure enantiomers from racemic mixtures would use enantioselective crystallization with chiral counterions.

The compounds of the present disclosure can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the present disclosure also comprises their corresponding pharmaceutically or toxicologically acceptable salts, in particular their pharmaceutically utilizable salts. Thus, the compounds of the present disclosure which contain acidic groups can be present on these groups and can be used according to the present disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present disclosure which contain one or more basic groups, i.e. groups which can be protonated, can be present and can be used according to the present disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present disclosure simultaneously contain acidic and basic groups in the molecule, the present disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Further the compounds of the present disclosure may be present in the form of solvates, such as those which include as solvate water, or pharmaceutically acceptable solvates, such as alcohols, in particular ethanol.

Furthermore, the present disclosure provides pharmaceutical compositions comprising at least one compound of the present disclosure, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing at least one compound of the present disclosure and a pharmaceutically acceptable carrier.

The pharmaceutical composition of the present disclosure may additionally comprise one or more other compounds as active ingredients like a prodrug compound or other nuclear receptor modulators.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

The compounds of the present disclosure act as LXR modulators.

Ligands to nuclear receptors including LXR ligands can either act as agonists, antagonists or inverse agonists. An agonist in this context means a small molecule ligand that binds to the receptor and stimulates its transcriptional activity as determined by e.g. an increase of mRNAs or proteins that are transcribed under control of an LXR response element. Transcriptional activity can also be determined in biochemical or cellular in vitro assays that employ just the ligand binding domain of LXRα or LXRβ but use the interaction with a cofactor (i.e. a corepressor or a coactivator), potentially in conjunction with a generic DNA-binding element such as the Gal4 domain, to monitor agonistic, antagonistic or inverse agonistic activity.

Whereas an agonist by this definition stimulates LXR- or LXR-Gal4-driven transcriptional activity, an antagonist is defined as a small molecule that binds to LXRs and thereby inhibits transcriptional activation that would otherwise occur through an endogenous LXR ligand.

An inverse agonist differs from an antagonist in that it not only binds to LXRs and inhibits transcriptional activity but in that it actively shuts down transcription directed by LXR, even in the absence of an endogenous agonist. Whereas it is difficult to differentiate between LXR antagonistic and inverse agonistic activity in vivo, given that there are always some levels of endogenous LXR agonist present, biochemical or cellular reporter assays can more clearly distinguish between the two activities. At a molecular level an inverse agonist does not allow for the recruitment of a coactivator protein or active parts thereof whereas it should lead to an active recruitment of corepressor proteins are active parts thereof. An LXR antagonist in this context would be defined as an LXR ligand that neither leads to coactivator nor to corepressor recruitment but acts just through displacing LXR agonists. Therefore, the use of assays such as the Gal4-mammalian-two-hybrid assay is mandatory in order to differentiate between coactivator or corepressor-recruiting LXR compounds (Kremoser et al., Drug Discov. Today 2007; 12:860; Gronemeyer et al., Nat. Rev. Drug Discov. 2004; 3:950).

Since the boundaries between LXR agonists, LXR antagonists and LXR inverse agonists are not sharp but fluent, the term “LXR modulator” was coined to encompass all compounds which are not clean LXR agonists but show a certain degree of corepressor recruitment in conjunction with a reduced LXR transcriptional activity. LXR modulators therefore encompass LXR antagonists and LXR inverse agonists and it should be noted that even a weak LXR agonist can act as an LXR antagonist if it prevents a full agonist from full transcriptional activation.

The compounds are useful for the prophylaxis and/or treatment of diseases which are mediated by LXRs. Certain of these diseases are disorders associated with steatosis, i.e. tissue fat accumulation. Such diseases encompass the full spectrum of non-alcoholic fatty liver disease including non-alcoholic steatohepatitis, liver inflammation and liver fibrosis, furthermore insulin resistance, metabolic syndrome and cardiac steatosis. An LXR modulator based medicine might also be useful for the treatment of hepatitis C virus infection or its complications and for the prevention of unwanted side-effects of long-term glucocorticoid treatment in diseases such as rheumatoid arthritis, inflammatory bowel disease and asthma.

A different set of applications for LXR modulators might be in the treatment of cancer. LXR antagonists or inverse agonists might useful to counteract the so-called Warburg effect which is associated with a transition from normal differentiated cells towards cancer cells (see Liberti et al., Trends Biochem. Sci. 2016; 41:211; Ward & Thompson, Cancer Cell 2012; 21:297-308). Furthermore, LXR is known to modulate various components of the innate and adaptive immune system. Oxysterols, which are known as endogenous LXR agonists were identified as mediators of an LXR-dependent immunosuppressive effect found in the tumor micro-environment (Traversari et al., Eur. J. Immunol. 2014; 44:1896). Therefore, it is reasonable to assume that LXR antagonists or inverse agonists might be capable of stimulating the immune system and antigen-presenting cells, in particular, to elicit an anti-tumor immune response. The latter effects of LXR antagonists or inverse agonists might be used for a treatment of late stage cancer, in general, and in particular for those types of cancerous solid tumors that show a poor immune response and highly elevated signs of Warburg metabolism.

In more detail, anti-cancer activity of the LXR inverse agonist SR9243 was shown to be mediated by interfering with the Warburg effect and lipogenesis in different tumor cells in vitro and SW620 colon tumor cells in athymic mice in vivo (see Flaveny et al. Cancer Cell. 2015; 28:42; Steffensen, Cancer Cell 2015; 28:3).

Therefore, LXR modulators (preferably LXR inverse agonists) may by useful for the treatment of Warburg-dependent cancers.

LXR modulators (preferably LXR inverse agonists) may counteract the diabetogenic effects of glucocorticoids without compromising the anti-inflammatory effects of glucocorticoids and could therefore be used to prevent unwanted side-effects of long-term glucocorticoid treatment in diseases such as rheumatoid arthritis, inflammatory bowel disease and asthma (Patel et al. Endocrinology 2017:158:1034).

LXR modulators (preferably LXR inverse agonists) may be useful for the treatment of hepatitis C virus mediated liver steatosis (see Garcia-Mediavilla et al. Lab. Invest. 2012; 92:1191).

LXR modulators (preferably LXR inverse agonists) may be useful for the treatment of viral myocarditis (see Papageorgiou et al. Cardiovasc. Res. 2015; 107:78).

LXR modulators (preferably LXR inverse agonists) may be useful for the treatment of insulin resistance (see Zheng et al. PLoS One 2014; 9:e101269).

LXR modulators (preferably LXR inverse agonists) may be useful for the treatment of familial hypercholesterolemia (see Zhou et al. J. Biol. Chem. 2008; 283:2129).

LXR modulators (preferably LXR inverse agonists) may be useful for the treatment of hypercholesterolemia in nephrotic syndrome (see Liu & Vazizi in Nephrol. Dial. Transplant. 2014; 29:538).

The present disclosure relates to a method of treating dyslipidemia in a subject in need thereof, comprising administering to the subject in need thereof an effective amount of a compound of the present disclosure. The claimed compounds are effective in lowering the amounts of lipids, such as triglycerides, in patients. Elevated triglyceride levels in a patient is an unhealthy or risky condition in itself and is implicated in many disorders. High triglycerides can contribute to hardening of the arteries or thickening of the artery walls (atherosclerosis), which can increase the risk of stroke, heart attack, and heart disease. Extremely high triglycerides can also cause acute inflammation of the pancreas (pancreatitis).

Disorders of lipid metabolism, or dyslipidemias, include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and IDL). The term “dyslipidemia” is a disorder of lipoprotein metabolism, including lipoprotein overproduction or deficiency. Dyslipidemias may be manifested by elevation of the total cholesterol, low-density lipoprotein (LDL) cholesterol and/or triglyceride concentrations in the blood, and/or a decrease in high-density lipoprotein (HDL) cholesterol concentration in the blood.

In certain embodiments, the dyslipidemia is characterized by abnormal concentrations of one or more lipids and/or apolipoproteins. In certain embodiments, the dyslipidemia is characterized by an elevated concentration of total cholesterol, LDL cholesterol, triglycerides (TG), or any combination of the foregoing. In certain embodiments, the dyslipidemia is characterized by a decreased concentration of HDL cholesterol.

In the context of the present disclosure dyslipidemia includes severe hypertriglyceridemia (SHTG), familial hypercholesterolemia, and Fredrickson hyperlipoproteinemia phenotypes. Familial hypercholesterolemia includes, for example, heterozygous familial hypercholesterolemia (HeFH) and homozygous familial hypercholesterolemia (HoFH). Fredrickson hyperlipoproteinemia phenotypes include, for example, Fredrickson phenotype I, Fredrickson phenotype II, Fredrickson phenotype II,b Fredrickson phenotype III, Fredrickson phenotype IV, and Fredrickson phenotype V. Fredrickson phenotype I includes familial chylomicronemia syndrome and mixed disorder chylomicronemia syndrome. Fredrickson phenotype II includes hypercholesterolemia. Fredrickson phenotype IIb includes familial combined hyperlipidemia that is characterized by various conditions including elevated concentrations of LDL and/or VLDL. Fredrickson phenotype III includes familial dysbetalipoproteinemia that is characterized by various conditions including elevated concentrations of VLDL and/or chylomicrons. Fredrickson phenotype IV includes hypertriglyceridemia (HTG) that is characterized by various conditions including elevated concentrations of VLDL and/or cholesterol. Fredrickson phenotype V includes mixed dyslipidemia that is characterized by various conditions including elevated concentrations of chylomicrons and/or VLDL, as well as concentrations of triglycerides (TG) greater than >99%.

The present disclosure relates to a method of treating hypertriglyceridemia in a subject in need thereof, comprising administering to the subject in need thereof an effective amount of a compound of the present disclosure. The term “hypertriglyceridemia” refers to high (hyper-) blood levels (-emia) of triglycerides. Elevated levels of triglycerides are associated with atherosclerosis, even in the absence of hypercholesterolemia (high cholesterol levels), and predispose to cardiovascular disease (e.g., contributes to increasing cardiovascular risk). Generally, serum triglyceride levels in the range of 150 to 199 mg per dL [1.70 to 2.25 mmol per L] are considered borderline-high serum triglyceride levels; serum triglyceride levels in the range of 200 to 499 mg per dL [2.26 to 5.64 mmol per L] are considered high serum triglyceride levels; and serum triglyceride levels in the range of 500 mg per dL [5.65 mmol per L] or higher are considered very high triglyceride levels. In certain aspects, these general guidelines may be adjusted based on updated clinical guidelines for clinicians. In certain embodiments, hypertriglyceridemia is severe hypertriglyceridemia, which is a triglyceride level of 500 mg/dL or higher.

The present disclosure relates to a method of treating a metabolic disorder associated with an impairment in lipid homeostasis in a subject in need thereof, comprising administering to the subject in need thereof an effective amount of a compound of the present disclosure.

Glucokinase regulatory protein (GCKR) is a gene variant that can interfere with glucose and lipid homeostasis by regulating glucose storage/disposal and by providing substrates for de novo lipogenesis via inhibition of glucokinase. GCKR may be contribute to the occurrence and severity of non-alcoholic fatty liver disease (NAFLD) and nonalcoholic steatohepatitis (NASH).

### Combinations

In certain embodiments, the method comprises administering at least one or more second agents. In a further embodiment, the one or more second agents is a therapeutic agent. In certain embodiments, a composition may further comprise a pharmaceutically active antihyperlipidemic agent, or a dietary supplement that can also have an impact on lipid content. For example, suitable agents include statins, fish oil, FGF21 agonists, THRβ agonists, and PCSK9 inhibitors.

### Dosing

The present disclosure relates to administration of a pharmaceutical composition comprising the compound of formula (I), a glycine conjugate, tauro conjugate, enantiomer, diastereomer, tautomer, N-oxide, solvate, prodrug and pharmaceutically acceptable salt thereof, to a subject in need thereof. Reference to compound of formula (I) herein can include a glycine conjugate, tauro conjugate, enantiomer, diastereomer, tautomer, N-oxide, solvate, prodrug or a pharmaceutically acceptable salt thereof.

The present disclosure provides for administration of a pharmaceutical composition at low doses with efficacy. The pharmacokinetic profile of the compounds of the present disclosure provides for transient exposures and lower sustained exposures with the metabolites.

The present disclosure provides a method of treating a disorder in a subject in need thereof, comprising administering daily 0.1 to 25 mg of a compound of formula (I), a glycine conjugate, tauro conjugate, enantiomer, diastereomer, tautomer, N-oxide, solvate, prodrug and pharmaceutically acceptable salt thereof.

In certain embodiments, the compound is compound 21/3 or pre 6/3. In certain embodiments, the compound is compound 21/3. In certain embodiments, the compound is compound pre 6/3.

In some embodiments, the pharmaceutical composition comprises about 0.1 mg to about 25 mg of the compound of formula (I), a glycine conjugate, tauro conjugate, enantiomer, diastereomer, tautomer, N-oxide, solvate, prodrug and pharmaceutically acceptable salt thereof, per administration. In some embodiments, the pharmaceutical composition comprises about 0.1 mg to about 20 mg of the compound of formula (I), a glycine conjugate, tauro conjugate, enantiomer, diastereomer, tautomer, N-oxide, solvate, prodrug and pharmaceutically acceptable salt thereof, per administration. In some embodiments, the pharmaceutical composition comprises about 0.5 mg to about 25 mg of the compound of formula (I), a glycine conjugate, tauro conjugate, enantiomer, diastereomer, tautomer, N-oxide, solvate, prodrug and pharmaceutically acceptable salt thereof, per administration. In some embodiments, the pharmaceutical composition comprises about 0.5 mg to about 20 mg of the compound of formula (I), a glycine conjugate, tauro conjugate, enantiomer, diastereomer, tautomer, N-oxide, solvate, prodrug and pharmaceutically acceptable salt thereof, per administration.

In certain embodiments, the pharmaceutical composition is administered: a) in an amount of 0.1 to 0.5 mg the compound of formula (I), a glycine conjugate, tauro conjugate, enantiomer, diastereomer, tautomer, N-oxide, solvate, prodrug and pharmaceutically acceptable salt thereof; b) in an amount of 0.5-1 mg the compound of formula (I), a glycine conjugate, tauro conjugate, enantiomer, diastereomer, tautomer, N-oxide, solvate, prodrug and pharmaceutically acceptable salt thereof; c) in an amount of 1-5 mg the compound of formula (I), a glycine conjugate, tauro conjugate, enantiomer, diastereomer, tautomer, N-oxide, solvate, prodrug and pharmaceutically acceptable salt thereof; d) in an amount of 5-10 mg the compound of formula (I), a glycine conjugate, tauro conjugate, enantiomer, diastereomer, tautomer, N-oxide, solvate, prodrug and pharmaceutically acceptable salt thereof; e) in an amount of 10-20 mg the compound of formula (I), a glycine conjugate, tauro conjugate, enantiomer, diastereomer, tautomer, N-oxide, solvate, prodrug and pharmaceutically acceptable salt thereof; or f) in an amount of 20-25 mg the compound of formula (I), a glycine conjugate, tauro conjugate, enantiomer, diastereomer, tautomer, N-oxide, solvate, prodrug and pharmaceutically acceptable salt thereof.

In certain embodiments, the pharmaceutical composition is administered: a) in an amount of 5-15 mg the compound of formula (I), a glycine conjugate, tauro conjugate, enantiomer, diastereomer, tautomer, N-oxide, solvate, prodrug and pharmaceutically acceptable salt thereof; b) in an amount of 5-10 mg the compound of formula (I), a glycine conjugate, tauro conjugate, enantiomer, diastereomer, tautomer, N-oxide, solvate, prodrug and pharmaceutically acceptable salt thereof; or c) in an amount of 10-15 mg the compound of formula (I), a glycine conjugate, tauro conjugate, enantiomer, diastereomer, tautomer, N-oxide, solvate, prodrug and pharmaceutically acceptable salt thereof.

In certain embodiments, the pharmaceutical composition is administered in an amount of 0.5, 2, 6, 12, or 20 mg the compound of formula (I), a glycine conjugate, tauro conjugate, enantiomer, diastereomer, tautomer, N-oxide, solvate, prodrug and pharmaceutically acceptable salt thereof. In certain embodiments, the compound is compound 21/3 or pre 6/3. In certain embodiments, the compound is compound 21/3. In certain embodiments, the compound is compound pre 6/3.

### EXEMPLARY EMBODIMENTS

Embodiment I-1. A method of treating dyslipidemia or a metabolic disorder associated with an impairment in lipid homeostasis in a subject in need thereof, comprising administering to the subject in need thereof an effective amount of a compound represented by Formula (I)

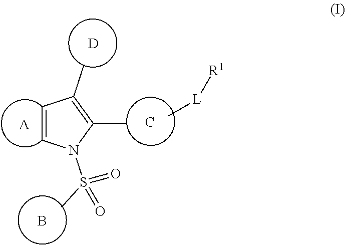

a glycine conjugate, tauro conjugate, enantiomer, diastereomer, tautomer, N-oxide, solvate, prodrug and pharmaceutically acceptable salt thereof,
wherein
\textcircled{A} is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
\textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
\textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
\textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards

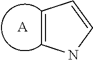

or has an annelated additional cycle in 1,2-orientation;
L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
X is independently selected from O, NR⁵¹, S(O)ₙ, S(═NR⁷¹), S(O)(═NR⁷¹) and S(═NR⁷¹)₂;
Y is independently selected from a bond, O, NR⁵¹, S(O)ₙ, S(═NR⁷¹), S(O)(═NR⁷¹) and S(═NR⁷¹)₂;
n is independently selected from 0 to 2;
and with the proviso, that the following structures are excluded:

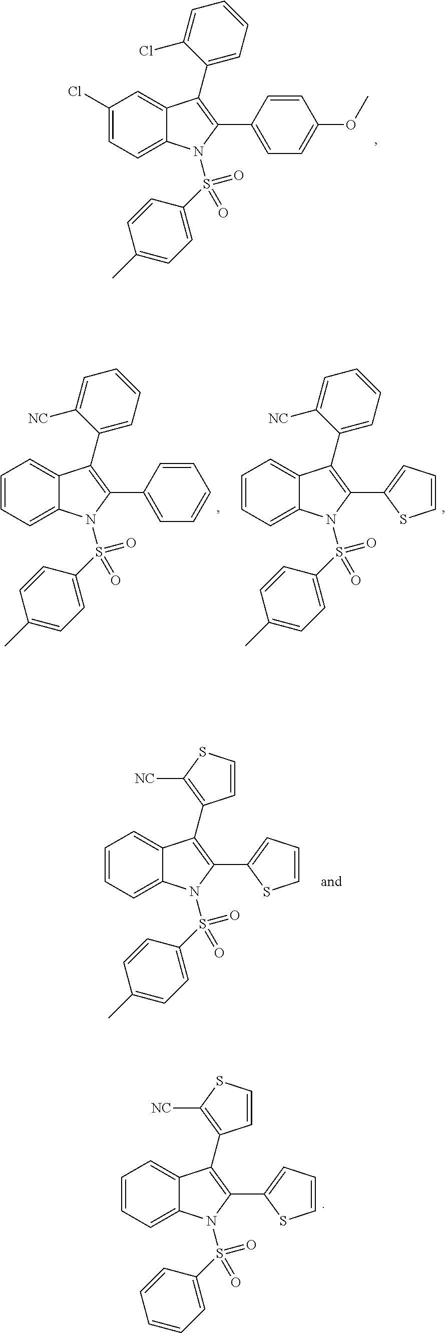

Embodiment I-2. The method of Embodiment I-1, wherein

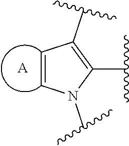

is selected from

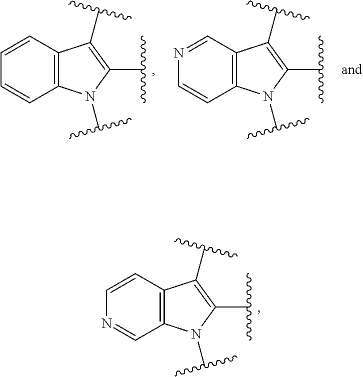

wherein [symbol] is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, NH₂, NHC₁₋₄-alkyl, N(C₁₋₄-alkyl)₂, SO₂—C₁₋₄-alkyl and SO₂-halo-C₁₋₄-alkyl.

Embodiment I-3. The method of Embodiment I-1 or I-2, wherein

\textcircled{B} is selected from the group consisting of phenyl, naphthyl, pyridyl, pyrimidinyl, thiophenyl, thiazolyl, cyclopentyl, cyclohexyl, bicyclo[1.1.1]pentyl, bicyclo[2.2.2]octyl, bicyclo[2.2.1]heptyl, pentacyclo[4.2.0.0^{2,5}.0^{3,8}.0^{4,7}]octyl and piperidinyl,
wherein the cycle is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH and halo-C₁₋₄-alkyl-OH; and wherein optionally two adjacent substituents on the phenyl ring form together a —(CH₂)₃—, —(CH₂)₄—, —OCF₂O— and —OCH₂O— group.

Embodiment I-4. The method of any one of Embodiments I-1 to I-3, wherein

\textcircled{C} is selected from phenyl, pyridyl and thiophenyl; wherein phenyl, pyridyl and thiophenyl is unsubstituted or substituted with 1 to 3 substituents independently selected from the group consisting of F, Cl, CN, OH, oxo, C₁₋₄-alkyl, halo-C₁₋₄-alkyl, O—C₁₋₄-alkyl and O-halo-C₁₋₄-alkyl; and wherein residue -L-R¹ is linked in 1,3-orientation regarding the connection towards

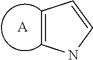

and L is not a bond.

Embodiment I-5. The method of any one of Embodiments I-1 to I-4, wherein

-L-R¹ is selected from

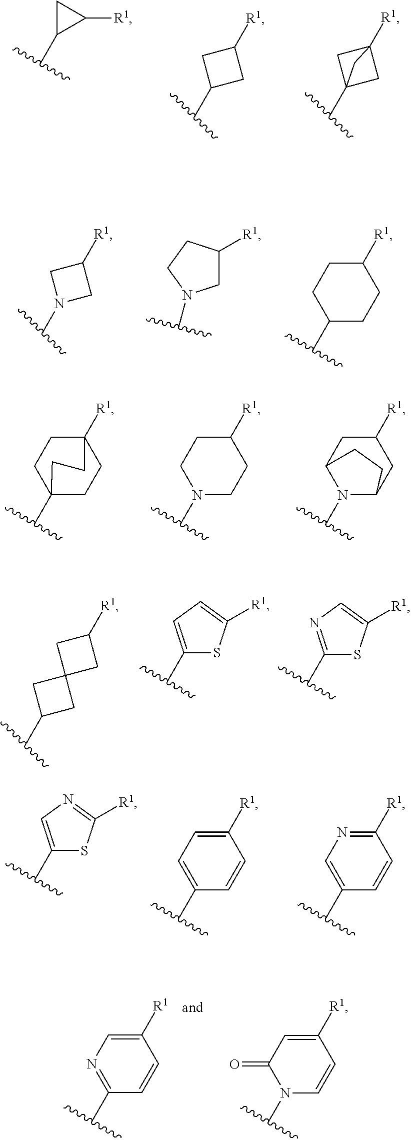

wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

Embodiment I-6. The method of any one of Embodiments I-1 to I-5, wherein

R¹ is selected from CO₂H, tetrazole, CH₂CO₂H, OCH₂CO₂H, SO₂CH₂CO₂H, CHMeCO₂H, CMe₂CO₂H, C(OH)MeCO₂H, CONHSO₂Me and CONH(OH); and optionally the glycine and tauro conjugate thereof.

Embodiment I-7. The method of any one of Embodiments I-1 to I-6, wherein

-L-R¹ is selected from

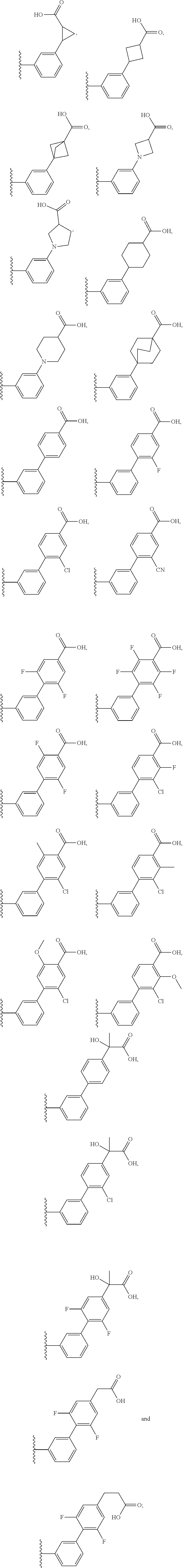

and optionally the glycine and tauro conjugate thereof.

Embodiment I-8. The method of any one of Embodiments I-1 to I-7, wherein

\textcircled{D} is selected from the group consisting

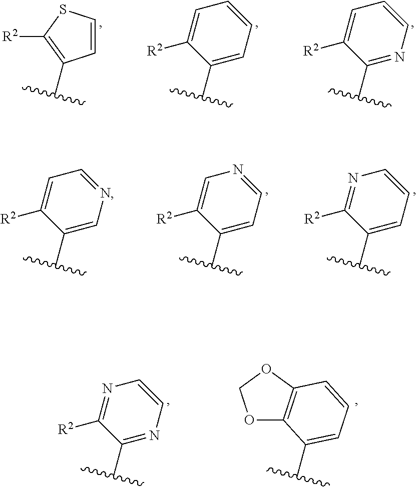

wherein
R² is selected from Me, F, Cl, CN, Me, CHO, CHF₂, CF₃, SO₂Me,

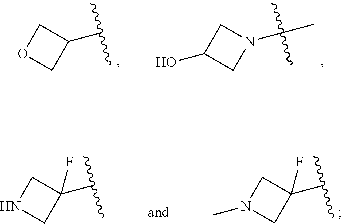

and
wherein \textcircled{D} is optionally further substituted with 1 to 2 substituents selected from the group consisting F, Cl, CN, Me, OMe, CHO, CHF₂ and CF₃.

Embodiment I-9. The method of any one of Embodiments I-1 to I-8, wherein

\textcircled{D} is selected from the group consisting of

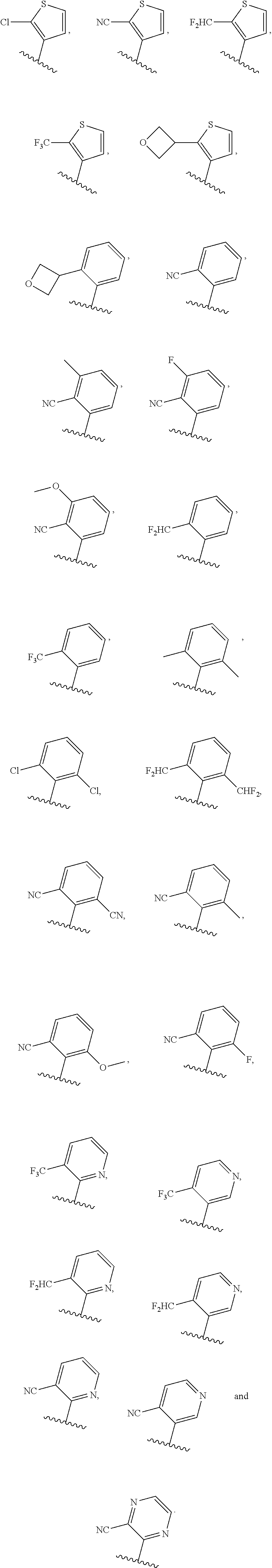

Embodiment I-10. The method of any one of Embodiments I-1 to I-9, wherein Formula (I) contains a substituent selected from the group consisting of CO₂H, tetrazole, CONHSO₂Me and CONH(OH); and optionally the glycine and tauro conjugate thereof.

Embodiment I-11. The method of any one of Embodiments I-1 to I-10, wherein

### L-R¹ is

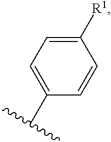

wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

Embodiment I-12. The method of any one of Embodiments I-1 to I-11, wherein

R¹ is C₀₋₆-alkylene-CO₂R⁴¹ or C₀₋₆-alkylene-CONR⁴¹R⁴², or a glycine conjugate or tauro conjugate thereof.

Embodiment I-13. The method of any one of Embodiments I-1 to I-12, wherein

R¹ is COOH, or a glycine conjugate or tauro conjugate thereof.

Embodiment I-14. The method of any one of Embodiments I-1 to I-12, wherein

R¹ is C₀₋₆-alkylene-CONR⁴¹R⁴²

Embodiment I-15. The method of Embodiment I-14, wherein

R⁴¹ and R⁴² are independently selected from H and C₁₋₄alkyl, wherein C₁₋₄alkyl is unsubstituted or substituted with CO₂H.

Embodiment I-16. The method of any one of Embodiments I-1 to I-11, wherein

### L-R¹ is

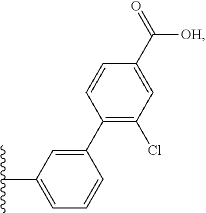 [SMILES: *c1cccc(-c2ccc(C(=O)O)cc2Cl)c1]

or a glycine conjugate or tauro conjugate thereof.

Embodiment I-17. The method of any one of Embodiments I-1 to I-16, where the compound is a glycine conjugate.

Embodiment I-18. The method of any one of Embodiments I-1 to I-17, that is

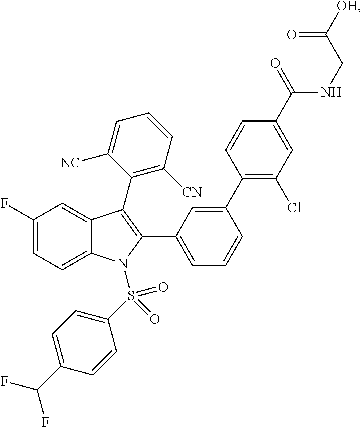 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]

Embodiment I-19. The method of any one of Embodiments I-1 to I-17, that is

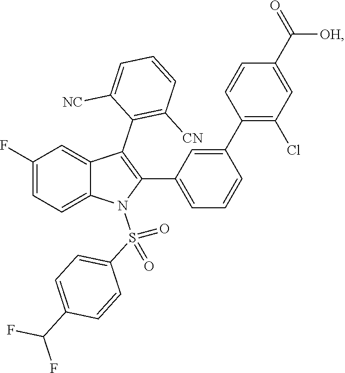 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

or a glycine conjugate thereof.

Embodiment I-20. The method of any one of Embodiments I-1 to I-17, selected from

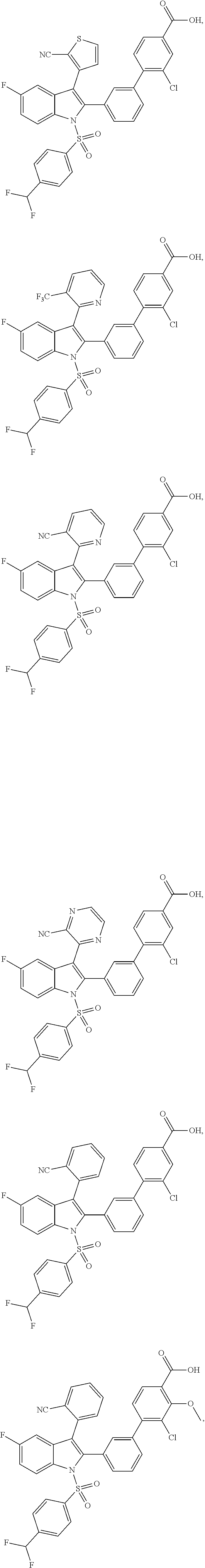

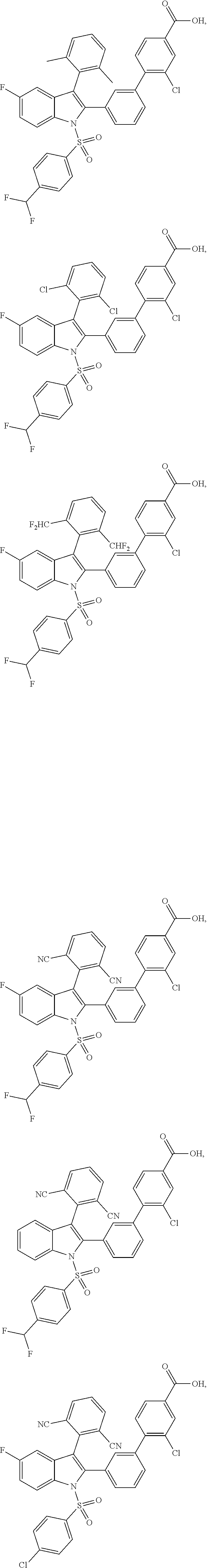

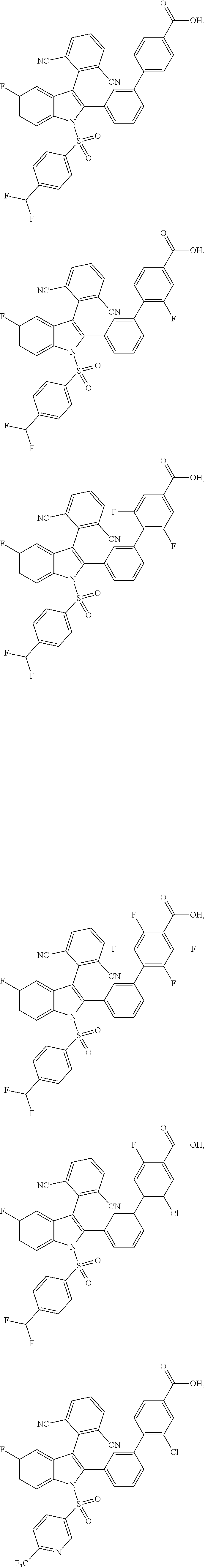

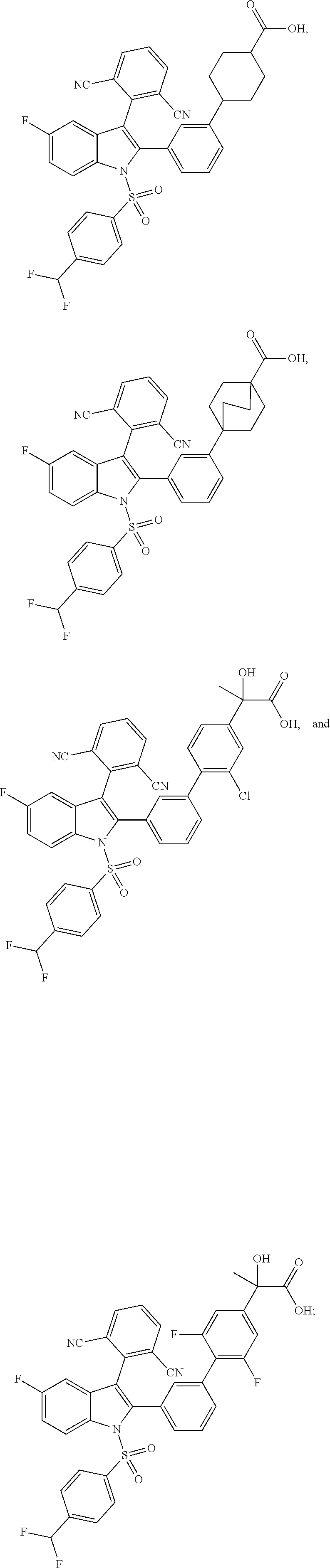 [SMILES: N#Cc1cccc(C#N)c1-c1c(-c2cccc(C3CCC(C(=O)O)CC3)c2)n(S(=O)(=O)c2ccc(C(F)F)cc2)c2ccc(F)cc12 ; N#Cc1cccc(C#N)c1-c1c(-c2cccc(C34CCC(C(=O)O)(CC3)CC4)c2)n(S(=O)(=O)c2ccc(C(F)F)cc2)c2ccc(F)cc12 ; CC(O)(C(=O)O)c1ccc(-c2cccc(-c3c(-c4c(C#N)cccc4C#N)c4cc(F)ccc4n3S(=O)(=O)c3ccc(C(F)F)cc3)c2)c(Cl)c1 ; CC(O)(C(=O)O)c1cc(F)c(-c2cccc(-c3c(-c4c(C#N)cccc4C#N)c4cc(F)ccc4n3S(=O)(=O)c3ccc(C(F)F)cc3)c2)c(F)c1]

or a glycine conjugate or tauro conjugate thereof; and
an enantiomer, diastereomer, tautomer, N-oxide, solvate, prodrug and pharmaceutically acceptable salt thereof.

Embodiment I-21. The method of any one of Embodiments I-1 to I-20, wherein the method is for treating dyslipidemia.

Embodiment I-22. The method of any one of Embodiments I-1 to I-21, wherein the dyslipidemia is hypertriglyceridemia (HTG), severe hypertriglyceridemia (SHTG), familial hypercholesterolemia, heterozygous familial hypercholesterolemia, homozygous familial hypercholesterolemia, familial chylomicronemia syndrome, mixed disorder chylomicronemia, hypercholesterolemia, familial combined hyperlipidemia, familial dysbetalipoproteinemia, and mixed dyslipidemia.

Embodiment I-23. The method of any one of Embodiments I-1 to I-21, wherein the dyslipidemia is severe hypertriglyceridemia (SHTG).

Embodiment I-24. The method of any one of Embodiments I-1 to I-23, wherein the dyslipidemia is characterized by abnormal concentrations of one or more lipids and/or apolipoproteins.

Embodiment I-25. The method of any one of Embodiments I-1 to I-24, wherein the dyslipidemia is characterized by an elevated concentration of total cholesterol, LDL cholesterol, triglycerides (TG), or any combination of the foregoing.

Embodiment I-26. The method of any one of Embodiments I-1 to I-25, wherein the dyslipidemia is characterized by a decreased concentration of HDL cholesterol.

Embodiment I-27. The method of any one of Embodiments I-1 to I-26, wherein the method decreases a risk of pancreatitis in the subject.

Embodiment I-28. The method of any one of Embodiments I-1 to I-20, wherein the method is for treating a metabolic disorder associated with an impairment in lipid homeostasis.

Embodiment I-29. The method of any one of Embodiments I-1 to I-20 and I-28, wherein the method comprises treating a metabolic disorder associated with an impairment in de novo lipogenesis.

Embodiment I-30. The method of any one of Embodiments I-1 to I-20 and I-28 to I-29,

wherein the method comprises treating non-alcoholic fatty liver disease (NAFLD) or nonalcoholic steatohepatitis (NASH) in a subject in need thereof, the subject having a glucokinase regulatory protein (GCKR) phenotype.

Embodiment I-31. The method of Embodiment I-30, wherein de novo lipogenesis is elevated in the subject.

Embodiment I-33. The method of Embodiments I-30 or I-31, wherein expression of lipogenic genes in the subject and/or lipid accumulation in the subject is reduced.

Embodiment II-1. A method of treating dyslipidemia or a metabolic disorder associated with an impairment in lipid homeostasis in a subject in need thereof, comprising administering to the subject in need thereof an effective amount of a compound represented by Formula (I)

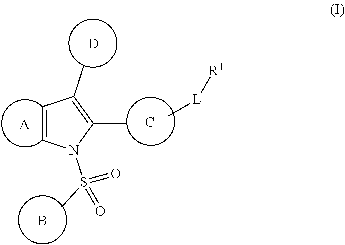

a glycine conjugate, tauro conjugate, enantiomer, diastereomer, tautomer, N-oxide, solvate, prodrug and pharmaceutically acceptable salt thereof,
wherein [symbol]
is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards

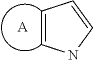

or has an annelated additional cycle in 1,2-orientation;
   L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
   R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR⁷¹) and S(═NR⁷¹)₂;
   n is independently selected from 0 to 2;
   and with the proviso, that the following structures are excluded:

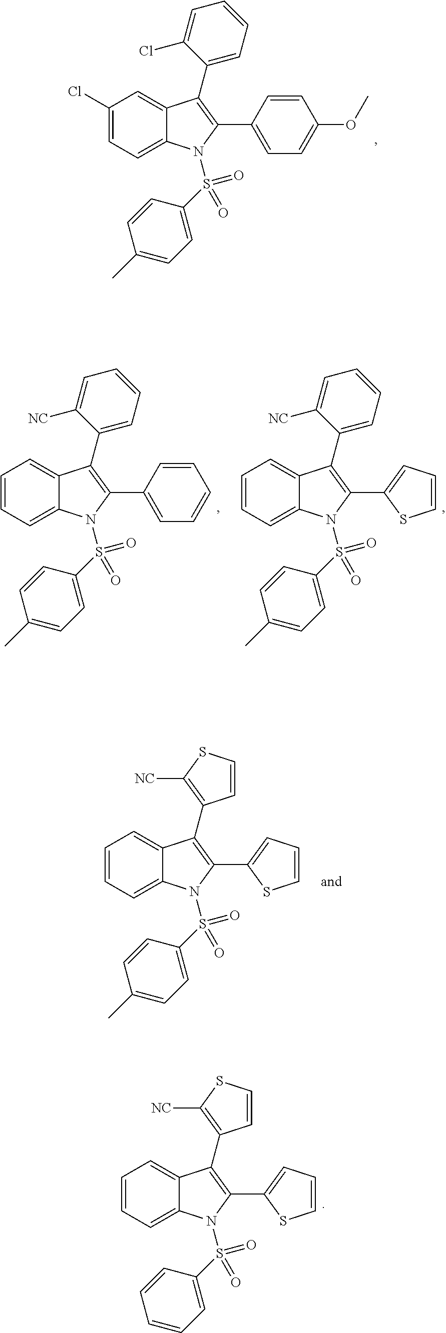

Embodiment II-2. Use of a compound in treating dyslipidemia or a metabolic disorder associated with an impairment in lipid homeostasis, wherein the compound is represented by Formula (I)

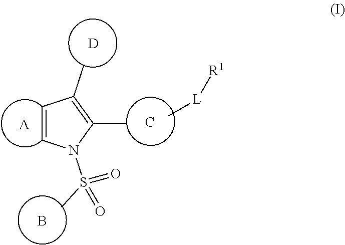

a glycine conjugate, tauro conjugate, enantiomer, diastereomer, tautomer, N-oxide, solvate, prodrug and pharmaceutically acceptable salt thereof,
wherein [symbol]
is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, 0-C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards

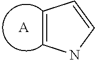

or has an annelated additional cycle in 1,2-orientation;
   L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
   R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR⁷¹) and S(═NR⁷¹)₂;
   n is independently selected from 0 to 2;
   and with the proviso, that the following structures are excluded:

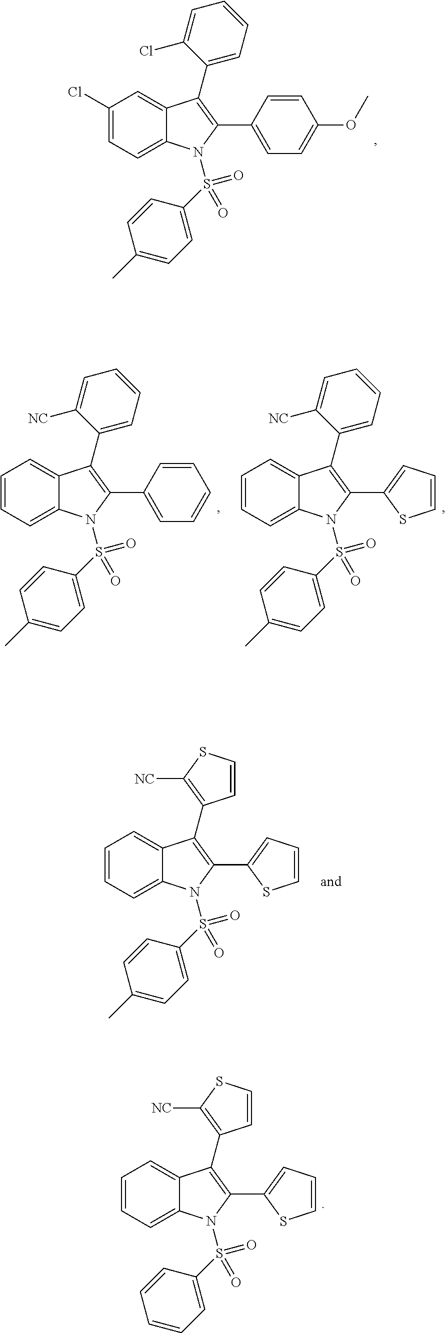

Embodiment II-3. Use of a compound in the manufacture of medicament for treating dyslipidemia or a metabolic disorder associated with an impairment in lipid homeostasis, wherein the compound is represented by Formula (I)

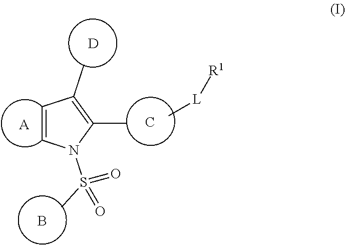

a glycine conjugate, tauro conjugate, enantiomer, diastereomer, tautomer, N-oxide, solvate, prodrug and pharmaceutically acceptable salt thereof,
wherein [symbol]
an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, 0-C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards

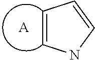

or has an annelated additional cycle in 1,2-orientation;
   L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
   R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR⁷¹) and S(═NR⁷¹)₂;
   n is independently selected from 0 to 2;
   and with the proviso, that the following structures are excluded:

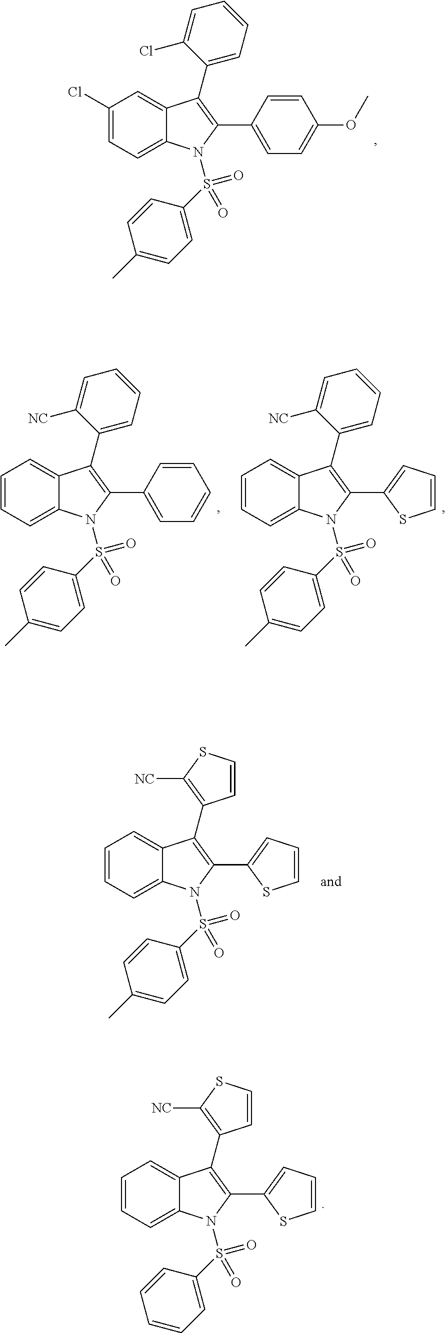

Embodiment II-4. A compound for use in treating dyslipidemia or a metabolic disorder associated with an impairment in lipid homeostasis, wherein the compound is represented by Formula (I)

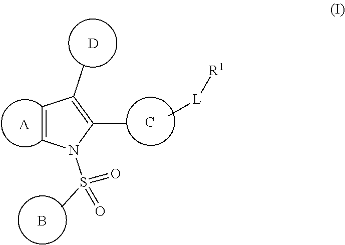

a glycine conjugate, tauro conjugate, enantiomer, diastereomer, tautomer, N-oxide, solvate, prodrug and pharmaceutically acceptable salt thereof,
wherein [symbol]
is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, 0-C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection
   towards

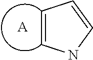

or has an annelated additional cycle in 1,2-orientation;
   L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
   R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR^{7′}) and S(═NR⁷¹)₂;
   n is independently selected from 0 to 2;
   and with the proviso, that the following structures are excluded:

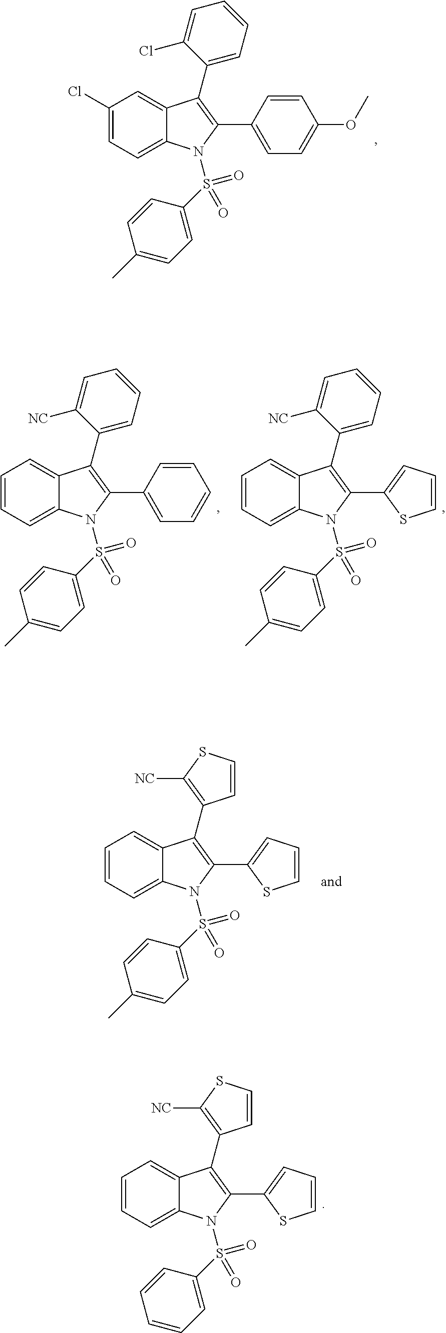

Embodiment II-5. A medicament for treating dyslipidemia or a metabolic disorder associated with an impairment in lipid homeostasis, the medicament comprising a compound represented by Formula (I)

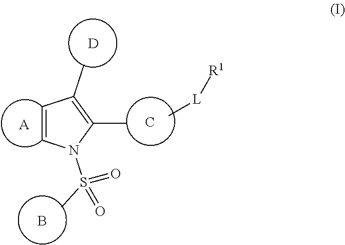

a glycine conjugate, tauro conjugate, enantiomer, diastereomer, tautomer, N-oxide, solvate, prodrug and pharmaceutically acceptable salt thereof,
wherein [symbol]
is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
   \textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   \textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, 0-C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards

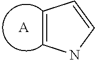

or has an annelated additional cycle in 1,2-orientation;
   L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
   R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
   R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
   R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
   X is independently selected from O, NR⁵¹, S(O)ₙ, S(═NR⁷¹), S(O)(═NR⁷¹) and S(═NR⁷¹)₂;
   Y is independently selected from a bond, O, NR⁵¹, S(O)ₙ, S(═NR⁷¹), S(O)(═NR⁷¹) and S(═NR⁷¹)₂;
   n is independently selected from 0 to 2;
   and with the proviso, that the following structures are excluded:

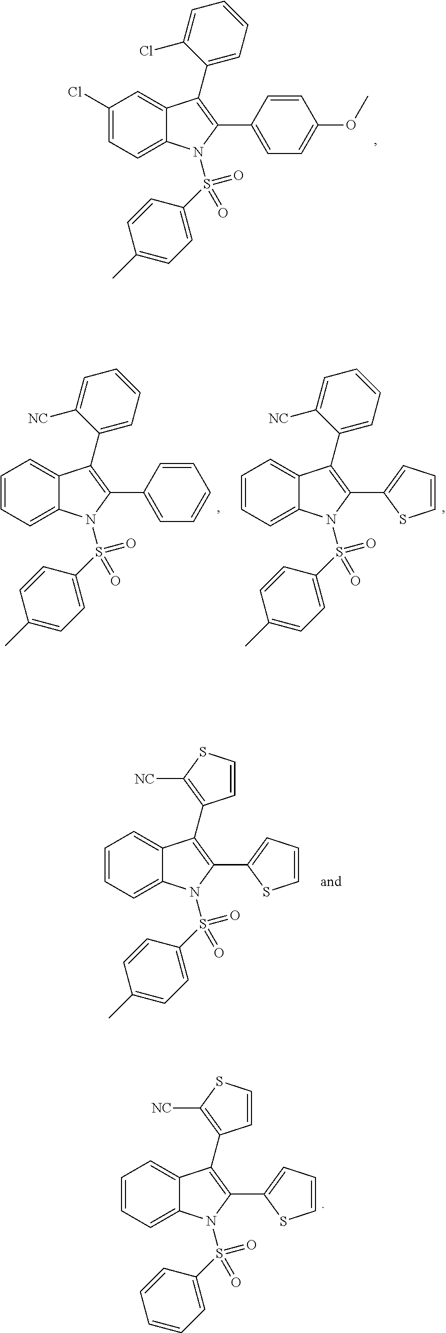

Embodiment II-6. The method, use, compound, or medicament according to any one of Embodiments II-1 to II-5, wherein

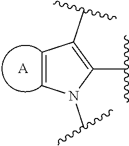

is selected from

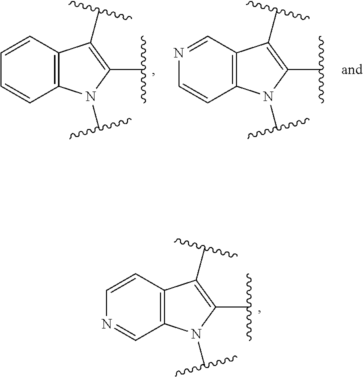

wherein [symbol] is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, NH₂, NHC₁₋₄-alkyl, N(C₁₋₄-alkyl)₂, SO₂—C₁₋₄-alkyl and SO₂-halo-C₁₋₄-alkyl.

Embodiment II-7. The method, use, compound, or medicament according to any one of Embodiments II-1 to II-6, wherein

\textcircled{B} is selected from the group consisting of phenyl, naphthyl, pyridyl, pyrimidinyl, thiophenyl, thiazolyl, cyclopentyl, cyclohexyl, bicyclo[1.1.1]pentyl, bicyclo[2.2.2]octyl, bicyclo[2.2.1]heptyl, pentacyclo[4.2.0.0^{2,5}0.0^{3,8}.0^{4,7}]octyl and piperidinyl,
wherein the cycle is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH and halo-C₁₋₄-alkyl-OH; and wherein optionally two adjacent substituents on the phenyl ring form together a —(CH₂)₃—, —(CH₂)₄—, —OCF₂O— and —OCH₂O— group.

Embodiment II-8. The method, use, compound, or medicament according to any one of Embodiments II-1 to II-7, wherein

\textcircled{C} is selected from phenyl, pyridyl and thiophenyl; wherein phenyl, pyridyl and thiophenyl is unsubstituted or substituted with 1 to 3 substituents independently selected from the group consisting of F, Cl, CN, OH, oxo, C₁₋₄-alkyl, halo-C₁₋₄-alkyl, O—C₁₋₄-alkyl and O-halo-C₁₋₄-alkyl;
and wherein residue -L-R¹ is linked in 1,3-orientation regarding the connection towards

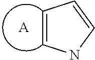

and L is not a bond.

Embodiment II-9. The method, use, compound, or medicament according to any one of Embodiments II-1 to II-8, wherein

-L-R¹ is selected from

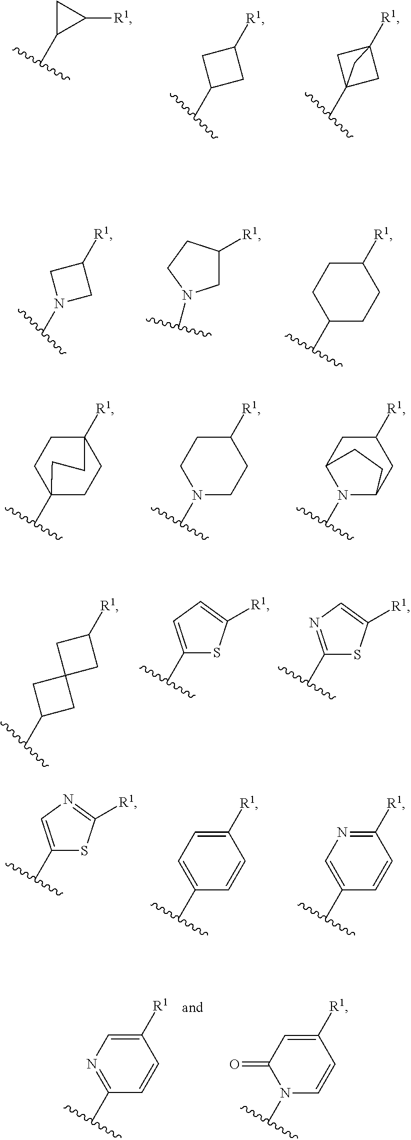

wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

Embodiment II-10. The method, use, compound, or medicament according to any one of Embodiments II-1 to II-9, wherein R¹ is selected from CO₂H, tetrazole, CH₂CO₂H, OCH₂CO₂H, SO₂CH₂CO₂H, CHMeCO₂H, CMe₂CO₂H, C(OH)MeCO₂H, CONHSO₂Me and CONH(OH); and optionally the glycine and tauro conjugate thereof.

Embodiment II-11. The method, use, compound, or medicament according to any one of Embodiments II-1 to II-10, wherein

\textcircled{D} is selected from the group consisting

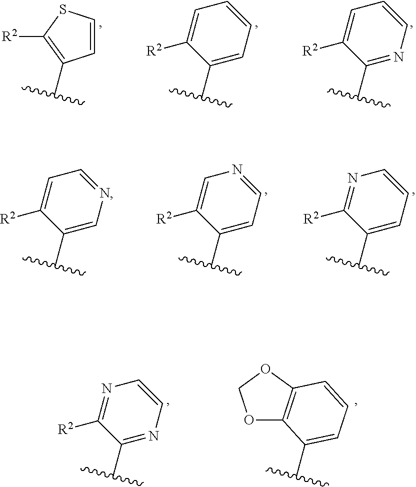

wherein
R² is selected from Me, F, Cl, CN, Me, CHO, CHF₂, CF₃, SO₂Me,

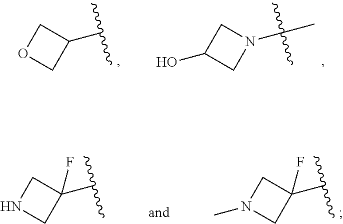

and
wherein \textcircled{D} is optionally further substituted with 1 to 2 substituents selected from the group consisting F, Cl, CN, Me, OMe, CHO, CHF₂ and CF₃.

Embodiment II-12. The method, use, compound, or medicament according to any one of Embodiments II-1 to II-11, wherein

\textcircled{D} is selected from the group consisting of

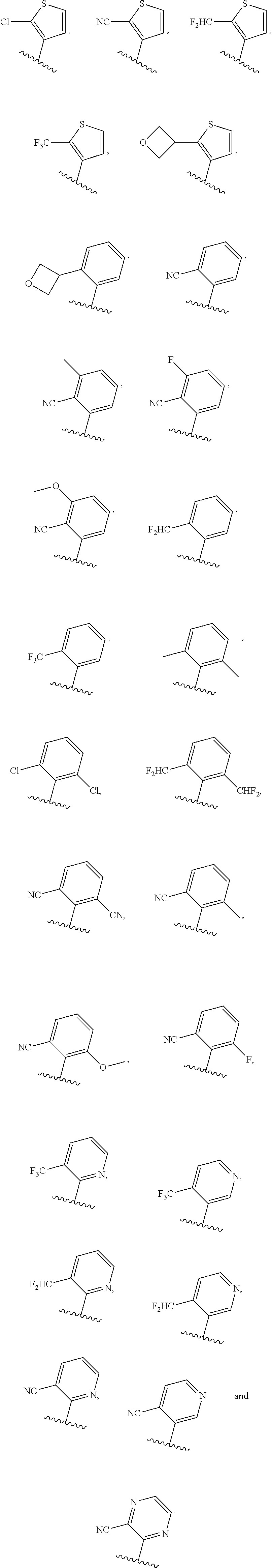

Embodiment II-13. The method, use, compound, or medicament according to any one of Embodiments II-1 to II-12, wherein Formula (I) contains a substituent selected from the group consisting of CO₂H, tetrazole, CONHSO₂Me and CONH(OH); and optionally the glycine and tauro conjugate thereof.

Embodiment II-14. The method, use, compound, or medicament according to any one of Embodiments II-1 to II-13, wherein

### L-R¹ is

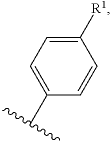

wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

Embodiment II-15. The method, use, compound, or medicament according to any one of Embodiments II-1 to II-104, wherein R¹ is C₀₋₆-alkylene-CO₂R⁴¹ or C₀₋₆-alkylene-CONR⁴¹R⁴², or a glycine conjugate or tauro conjugate thereof.

Embodiment II-16. The method, use, compound, or medicament according to any one of Embodiments II-1 to II-15, wherein R¹ is COOH, or a glycine conjugate or tauro conjugate thereof.

Embodiment II-17. The method, use, compound, or medicament according to any one of Embodiments II-1 to 15, wherein R¹ is C₀₋₆-alkylene-CONR⁴¹R⁴².

Embodiment II-18. The method, use, compound, or medicament according to Embodiment II-17, wherein R⁴¹ and R⁴² are independently selected from H and C₁₋₄alkyl, wherein C₁₋₄alkyl is unsubstituted or substituted with CO₂H.

Embodiment II-19. The method, use, compound, or medicament according to any one of Embodiments II-1 to II-1, wherein —C-L-R¹ is

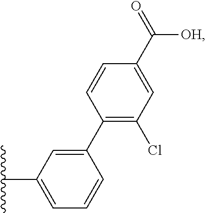 [SMILES: *c1cccc(-c2ccc(C(=O)O)cc2Cl)c1]

or a glycine conjugate or tauro conjugate thereof.

Embodiment II-20. The method, use, compound, or medicament according to any one of Embodiments II-1 to I-19, wherein the compound is a glycine conjugate.

Embodiment II-21. The method, use, compound, or medicament according to any one of Embodiments II-1 to II-20, wherein the compound is

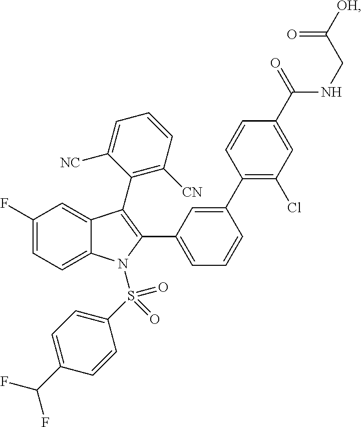 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]

or a pharmaceutically acceptable salt thereof.

Embodiment II-22. The method, use, compound, or medicament according to any one of Embodiments II-1 to II-21, wherein the compound is

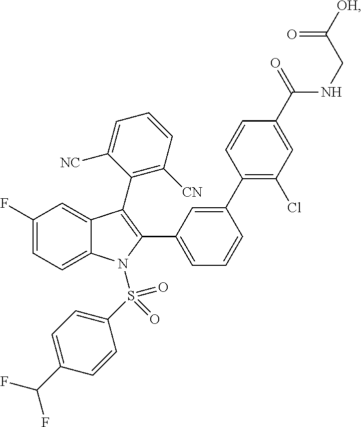 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]

Embodiment II-23. The method, use, compound, or medicament according to any one of Embodiments II-1 to II-20, wherein the compound is

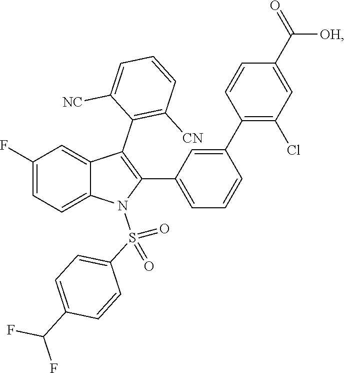 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

or a glycine conjugate thereof.

Embodiment II-24. The method, use, compound, or medicament according to any one of Embodiments II-1 to II-23, wherein the compound is

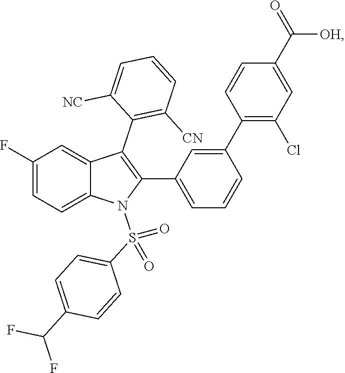 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

or a pharmaceutically acceptable salt thereof.

Embodiment II-25. The method, use, compound, or medicament according to any one of Embodiments II-1 to II-24, wherein the compound is

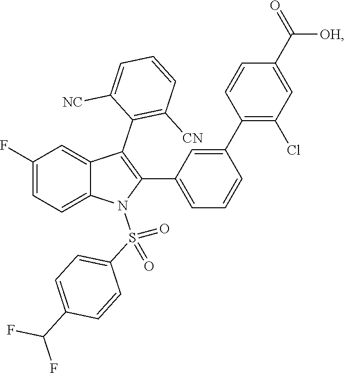 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

Embodiment II-26. The method, use, compound, or medicament according to any one of Embodiments II-1 to II-25, wherein the method is for treating dyslipidemia.

Embodiment II-27. The method, use, compound, or medicament according to any one of Embodiments II-1 to II-256, wherein the dyslipidemia is hypertriglyceridemia (HTG), severe hypertriglyceridemia (SHTG), familial hypercholesterolemia, heterozygous familial hypercholesterolemia, homozygous familial hypercholesterolemia, familial chylomicronemia syndrome, mixed disorder chylomicronemia, hypercholesterolemia, familial combined hyperlipidemia, familial dysbetalipoproteinemia, or mixed dyslipidemia.

Embodiment II-28. The method, use, compound, or medicament according to any one of Embodiments II-1 to II-27, wherein the dyslipidemia is severe hypertriglyceridemia (SHTG).

Embodiment II-29. The method, use, compound, or medicament according to any one of Embodiments II-1 to II-28, wherein the dyslipidemia is characterized by abnormal concentrations of one or more lipids and/or apolipoproteins.

Embodiment II-30. The method, use, compound, or medicament according to any one of Embodiments II-1 to II-29, wherein the dyslipidemia is characterized by an elevated concentration of total cholesterol, LDL cholesterol, triglycerides (TG), or any combination of the foregoing.

Embodiment II-31. The method, use, compound, or medicament according to any one of Embodiments II-1 to II-30, wherein the dyslipidemia is characterized by a decreased concentration of HDL cholesterol.

Embodiment II-32. The method, use, compound, or medicament according to any one of Embodiments II-1 to II-31, wherein the method decreases a risk of pancreatitis in the subject.

Embodiment II-33. The method, use, compound, or medicament according to any one of Embodiments II-1 to II-25, wherein the method is for treating a metabolic disorder associated with an impairment in lipid homeostasis.

Embodiment II-34. The method, use, compound, or medicament according to any one of Embodiments II-1 to II-25 and II-33, wherein the method comprises treating a metabolic disorder associated with an impairment in de novo lipogenesis.

Embodiment II-35. The method, use, compound, or medicament according to any one of Embodiments II-1 to II-25 and II-33 to II-34, wherein the method comprises treating non-alcoholic fatty liver disease (NAFLD) or nonalcoholic steatohepatitis (NASH) in a subject in need thereof, the subject having a glucokinase regulatory protein (GCKR) phenotype.

Embodiment II-36. The method, use, compound, or medicament according to Embodiment II-34, wherein de novo lipogenesis is elevated in the subject.

Embodiment II-37. The method, use, compound, or medicament according to Embodiment II-34 or Embodiment II-35, wherein expression of lipogenic genes in the subject and/or lipid accumulation in the subject is reduced.

Embodiment II-38. The method, use, compound, or medicament according to any one of Embodiments II-1 to II-37, wherein the subject is administered 0.1 to 25 mg per day of the compound of formula (I), or a glycine conjugate, tauro conjugate, enantiomer, diastereomer, tautomer, N-oxide, solvate, prodrug and pharmaceutically acceptable salt thereof.

Embodiment II-39. The method, use, compound, or medicament of Embodiment II-38, wherein 5 to 15 mg of the compound is administered.

### EXPERIMENTAL SECTION

As described in WO 2020/002611, the compounds of the present disclosure can be prepared by a combination of methods known in the art including the procedures described in Schemes I to V below.

The synthetic route depicted in Scheme I starts with the preparation of alkynes I-c by Sonogashira couplings. Subsequently, the free amino group of I-c is reacted with sulfonyl chlorides I-d in the presence of an appropriate base and appropriate solvent to afford alkynesulfonamides I-e. I-e undergoes cyclization and concomitant reaction with aromatic halides I-f in the presence of appropriate catalyst (e.g. Pd-catalysts), appropriate solvent and temperature to afford compounds of the present disclosure (I). Further manipulation of functional groups present in R¹ by standard methods, known to persons skilled in the art (e.g. ester hydrolysis, amide bond formation), can give rise to further compounds of the present disclosure. Alternatively, alkyneamine I-c can be transformed into alkynetrifluoroacetamides I-g which can also undergo aforementioned cyclization and concomitant reaction with aromatic halides I-f to afford intermediates I-h with an unsubstituted NH. Reaction with sulfonyl chlorides I-d in the presence of an appropriate base and appropriate solvent also affords compounds of Formula (I).

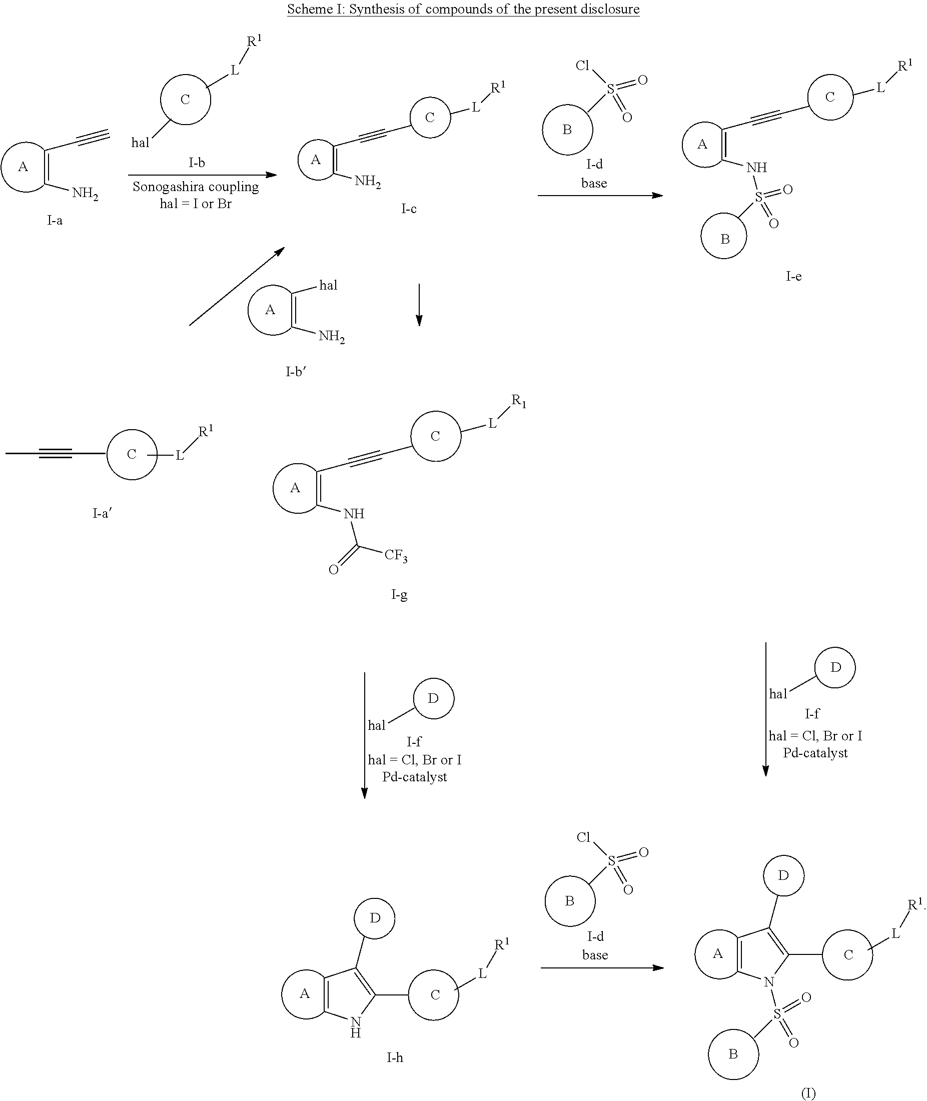

A variation of the routes shown in Scheme I is shown in Scheme II. Alkynesulfonamide I-e is reacted in the presence of NIS to afford iodinated intermediates II-b which can be substrates for Suzuki couplings to afford compounds (I). Alternatively, cyclization of alkynesulfonamide I-e in the presence of appropriate catalyst (e.g. Pd-catalysts), appropriate solvent and temperature but without the presence of halides I-f afford 3-unsubstituted intermediates II-d. Reactions with NBS afford brominated intermediates II-e which are likewise substrates for Suzuki coupling reactions to afford compounds of Formula (I).

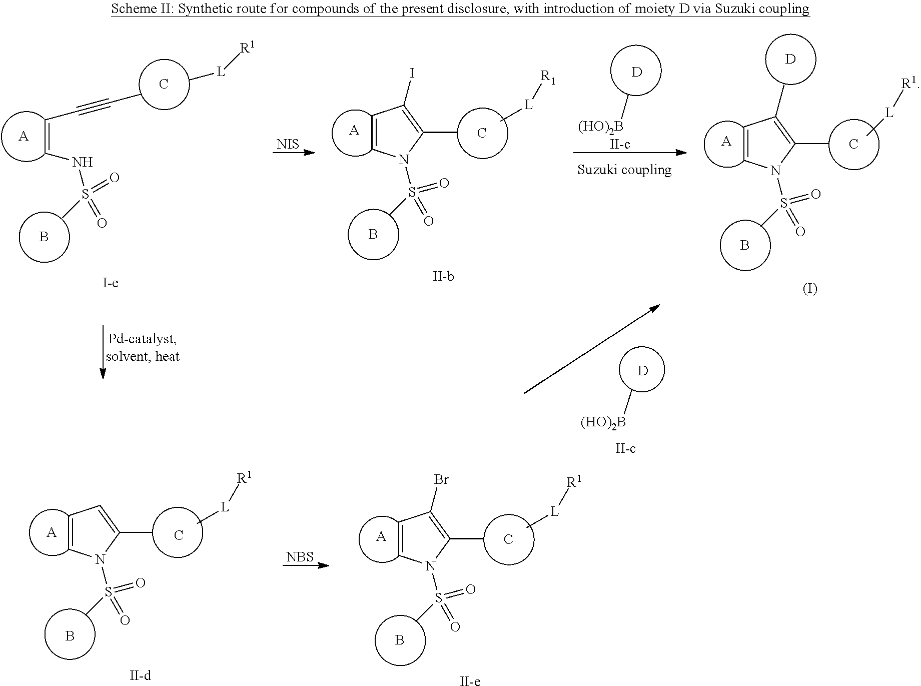

A further variation of the synthetic route depicted in Schemes I and II is shown in Scheme III. In the presence of B₂Pin₂, appropriate catalyst (e.g. Pd-catalysts), appropriate solvent, additives and temperature, intermediates I-e can undergo cyclization and concomitant formation of 3-pinacolyl boronic esters III-b. These can be substrates for Suzuki coupling reactions to afford compounds of the present disclosure with Formula (I).

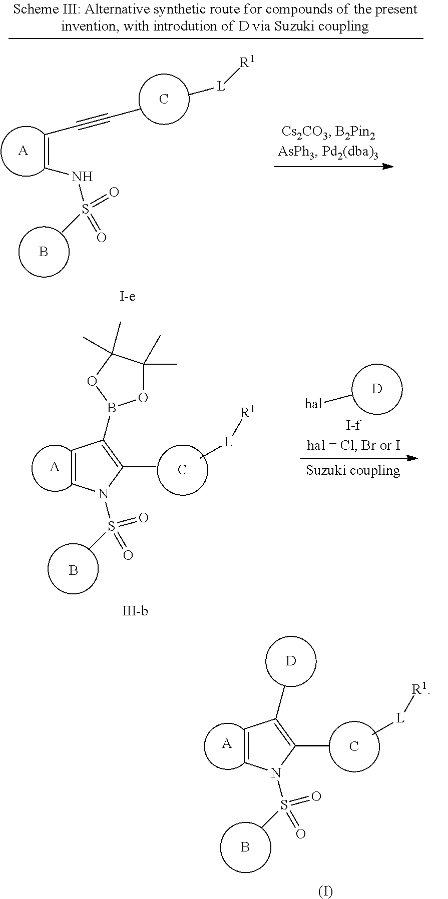

In Scheme IV is depicted a synthetic route for the late stage introduction of the right hand side moieties -L-R¹ to the compounds of the present disclosure. Sonogashira coupling of I-a with bromo-iodo-aromatics IV-a afford bromo-alkyneamines IV-b which can be transformed to sulfonamides IV-c. These can undergo cyclization and concomitant reaction with aromatic bromides IV-d in the presence of appropriate catalysts (e.g. Pd-catalysts), appropriate solvent and temperature to afford advanced intermediates IV-e with a bromo substituent on ring C. Finally, intermediates IV-e can be used as substrates for Suzuki couplings to afford compounds of Formula (I).

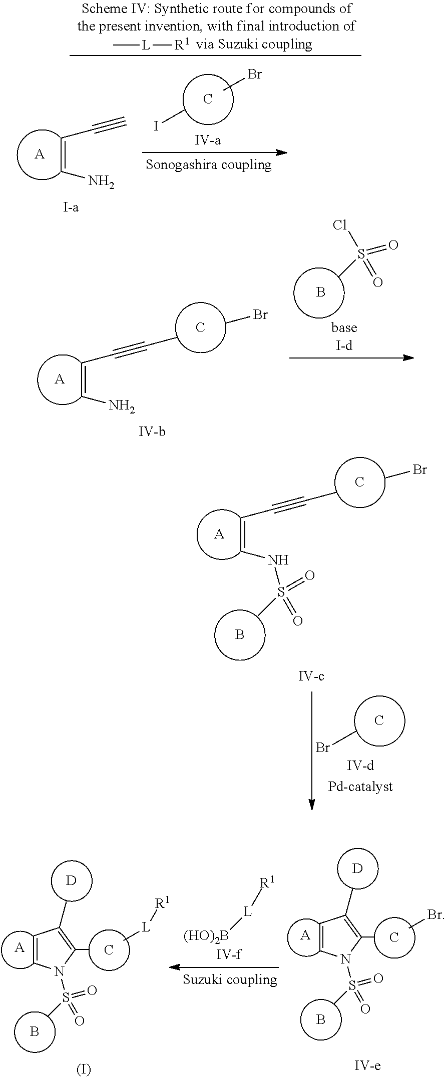

In Scheme V are summarized the synthetic routes for the preparation of the compounds of the present disclosure starting from the preformed central pyrrolo-annelated bicyclic aromatic. N-protected 2-pinacolyl boronic esters V-a can undergo Suzuki coupling with halides V-b to afford intermediates V-c. After bromination with NBS the 3-bromo intermediates V-d are obtained, which, after a second Suzuki coupling, are converted to N-protected advanced intermediates V-e. When starting with N-protected 3-pinacolyl boronic esters V-a, first Suzuki coupling and then bromination of the 2-position and subsequent second Suzuki coupling affords likewise intermediates V-e. After deprotection and reaction of the free NH with sulfonyl chlorides I-d, in the presence of an appropriate base and solvent, compounds (I) are obtained.

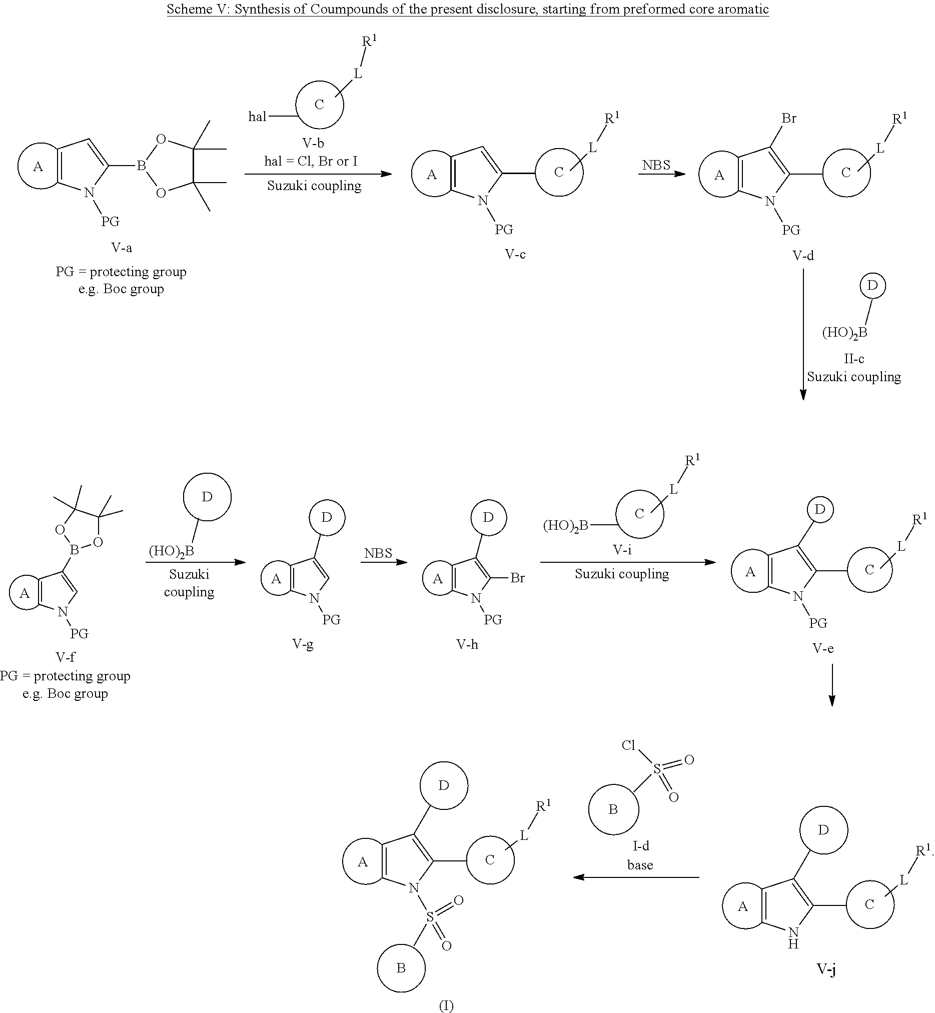

### Example Compounds Set 1

Example compounds are shown below.

| # | building block(s) | structure | analytical data |
|---|---|---|---|
| 1/23 | 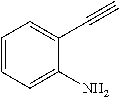 | 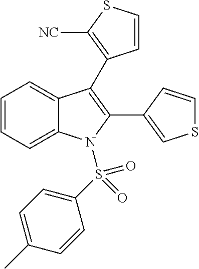 [SMILES: Cc1ccc(S(=O)(=O)n2c(-c3ccsc3)c(-c3ccsc3C#N)c3ccccc32)cc1] | ¹H-NMR (500 MHz, DMSO- d₆) δ: 8.25 (d, J = 8.5 Hz, 1H), 8.02 (d, J = 5.5 Hz, 1H), 7.57 (dd, J = 3.0, 5.0 Hz, 1H), 7.49 (dd, J = 4.5, 8.5 Hz, 1H), 7.45-7.43 (m, 3H), 7.36 (d, J = 4.0 Hz, 2H), 7.32 (d, J = 8.5 Hz, 2H), 7.11 (dd, J = 1.3, 4.8 Hz, 1H), 6.98 (d, J = 5.0 Hz, 1H), 2.31 (s, 3H); MS: 482.7 (M + Na)⁺. |
| | 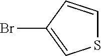 | | |
| 1/26 | 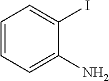 | 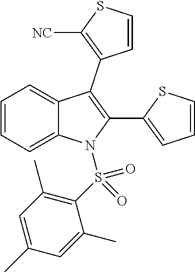 [SMILES: Cc1cc(C)c(S(=O)(=O)n2c(-c3cccs3)c(-c3ccsc3C#N)c3ccccc32)c(C)c1] | ¹H-NMR (500 MHz, DMSO- d₆) δ: 8.21 (d, J = 8.5 Hz, 1H), 7.99 (d, J = 5.0 Hz, 1H), 7.55-7.51 (m, 2H), 7.46 (d, J = 7.5 Hz 1H), 7.40 (t, J = 7.5 Hz, 1H), 6.99 (d, J = 5.0 Hz, 1H), 6.94-6.89 (m, 4H), 2.23 (s, 3H), 2.06 (s, 6H); MS: 510.8 (M + Na)⁺. |
| | 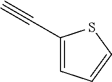 | | |
| | 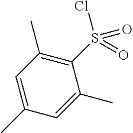 | | |
| 1/27 | 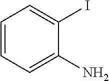 | 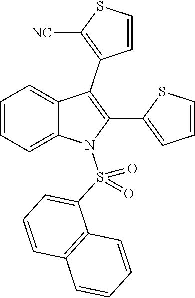 [SMILES: N#Cc1sccc1-c1c(-c2cccs2)n(S(=O)(=O)c2cccc3ccccc23)c2ccccc12] | ¹H-NMR (500 MHz, DMSO- d₆) δ: 8.33 (d, J = 8.0 Hz, 1H), 8.24 (d, J = 8.0 Hz, 1H), 8.08 (d, J = 8.0 Hz, 1H), 8.02 (d, J = 8.0 Hz, 1H), 7.98 (d, J = 5.0 Hz, 1H), 7.70 (d, J = 7.5 Hz, 1H), 7.63-7.43 (m, 7H), 7.05-6.95 (m, 3H); MS: 519.3 (M + Na)⁺. |
| | 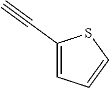 | | |
| | 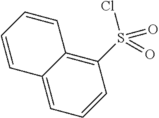 [SMILES: O=S(=O)(Cl)c1cccc2ccccc12] | | |
| 1/28 | 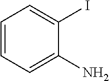 | 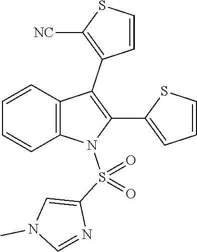 [SMILES: Cn1cnc(S(=O)(=O)n2c(-c3cccs3)c(-c3ccsc3C#N)c3ccccc32)c1] | ¹H-NMR (500 MHz, DMSO- d₆) δ: 8.20 (d, J = 10.5 Hz, 1H), 8.02 (d, J = 6.5 Hz, 1H), 7.81 (s, 1H), 7.78 (s, 1H), 7.67-7.66 (m, 1H), 7.50-7.30 (m, 4H), 7.09 (dd, J = 4.5, 6.5 Hz, 1H), 7.04 (d, J = 6.0 Hz, 1H), 3.63 (s, 3H); MS: 450.8 (M + 1)⁺. |
| | 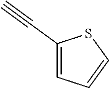 | | |
| | 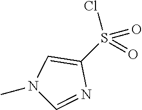 | | |
| 1/39 | 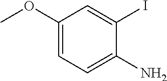 | 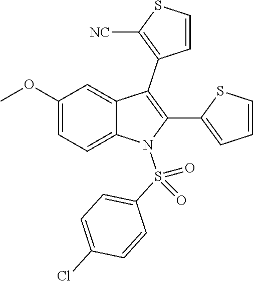 [SMILES: COc1ccc2c(c1)c(-c1ccsc1C#N)c(-c1cccs1)n2S(=O)(=O)c1ccc(Cl)cc1] | ¹H-NMR (500 MHz, DMSO- d₆) δ: 8.16 (d, J = 9.5 Hz, 1H), 8.04 (d, J = 5.0 Hz, 1H), 7.74 (d, J = 4.5 Hz, 1H), 7.60 (d, J = 8.5 Hz, 2H), 7.49 (d, J = 8.5 Hz, 2H), 7.22-7.21 (m, 1H), 7.16-7.12 (m, 2H), 7.03 (d, J = 5.5 Hz, 1H), 6.78 (s, 1H), 3.76 (s, 3H); MS: 510.8 (M + 1)⁺. |
| | 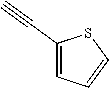 | | |
| | 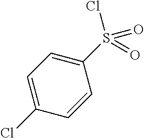 [SMILES: O=S(=O)(Cl)c1ccc(Cl)cc1] | | |
| 1/40 | 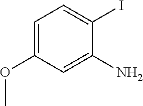 | 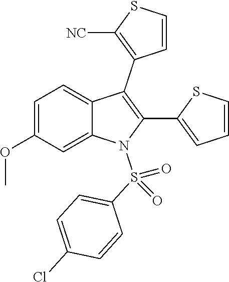 [SMILES: COc1ccc2c(-c3ccsc3C#N)c(-c3cccs3)n(S(=O)(=O)c3ccc(Cl)cc3)c2c1] | ¹H-NMR (500 MHz, DMSO- d₆) δ: 8.03 (d, J = 5.0 Hz, 1H), 7.76 (d, J = 1.5 Hz, 1H), 7.72 (d, J = 5.0 Hz, 1H), 7.61 (d, J = 8.5 Hz, 2H), 7.55 (d, J = 8.5 Hz, 2H), 7.27 (d, J = 9.0 Hz, 1H), 7.19-7.17 (m, 1H), 7.11-7.09 (m, 1H), 7.05 (dd, J = 2.5, 8.5 Hz, 1H), 7.01 (d, J = 5.5 Hz, 1H), 3.91 (s, 3H); MS: 510.8 (M + 1)⁺. |
| | 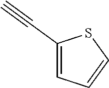 | | |
| | 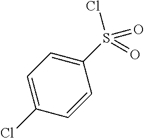 [SMILES: O=S(=O)(Cl)c1ccc(Cl)cc1] | | |
| 1/41 | 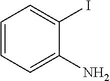 | 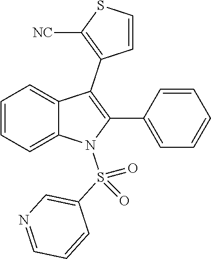 [SMILES: N#Cc1sccc1-c1c(-c2ccccc2)n(S(=O)(=O)c2cccnc2)c2ccccc12] | ¹H-NMR (500 MHz, DMSO- d₆) δ: 8.82 (d, 4.0 Hz, 1H), 8.62 (s, 1H), 8.30 (d, J = 8.0 Hz, 1H), 8.01 (d, J = 4.5 Hz, 1H), 7.87 (d, J = 8.5 Hz, 1H), 7.58-7.53 (m, 2H), 7.46-7.35 (m, 5H), 7.30 (d, J = 7.0 Hz, 2H), 6.97 (d, J = 4.5 Hz, 1H); MS: 441.9 (M + 1)⁺. |
| | 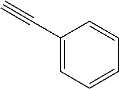 | | |
| | 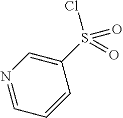 | | |
| 1/42 | 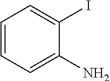 | 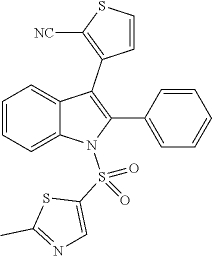 [SMILES: Cc1ncc(S(=O)(=O)n2c(-c3ccccc3)c(-c3ccsc3C#N)c3ccccc32)s1] | ¹H-NMR (500 MHz, DMSO- d₆) δ: 8.19 (d, J = 8.5 Hz, 1H), 8.05-8.02 (m, 2H), 7.57-7.54 (m, 1H), 7.46-7.33 (m, 7H), 6.97 (d, J = 5.0 Hz, 1H), 2.63 (s, 3H); MS: 462.1 (M + 1)⁺. |
| | 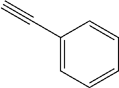 | | |
| | 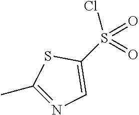 | | |
| 1/ 122 | 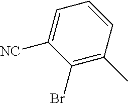 | 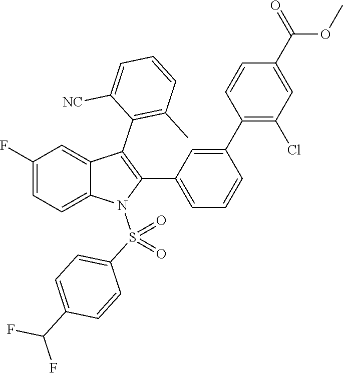 [SMILES: COC(=O)c1ccc(-c2cccc(-c3c(-c4c(C)cccc4C#N)c4cc(F)ccc4n3S(=O)(=O)c3ccc(C(F)F)cc3)c2)c(Cl)c1] | ¹H-NMR (400 MHz, CD₃OD) δ: 8.41 (dd, J = 9.2, 4.4 Hz, 1H), 8.09 (d, J = 1.6 Hz, 1H), 8.00 (dd, J = 8.0, 1.6 Hz, 1H), 7.60-7.15 (m, 13H), 6.73 (dd, J = 2.4, 8.4 Hz, 1H), 6.71 (t, J = 55.6 Hz, 1H), 3.96 (s, 3H), 1.88 (s, 3H). |
| | 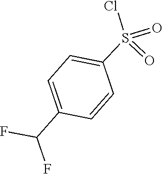 [SMILES: O=S(=O)(Cl)c1ccc(C(F)F)cc1] | | |
| | 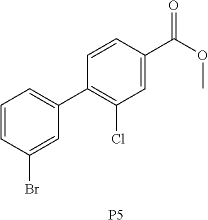 | | |
| 1/ 139 | 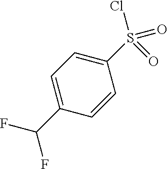 [SMILES: O=S(=O)(Cl)c1ccc(C(F)F)cc1] | 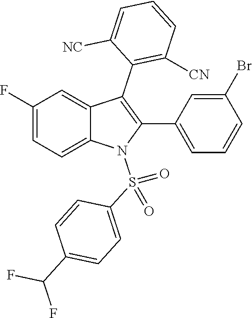 [SMILES: N#Cc1cccc(C#N)c1-c1c(-c2cccc(Br)c2)n(S(=O)(=O)c2ccc(C(F)F)cc2)c2ccc(F)cc12] | ¹H-NMR (500 MHz, DMSO- d₆) δ: 8.32 (dd, J = 9.3, 4.3 Hz, 1H), 8.24 (d, J = 8.5 Hz, 2H), 7.80 (t, J = 8.0 Hz, 1H), 7.72-7.59 (m, 5H), 7.45 (dt, J = 2.5, 9.0 Hz, 1H), 7.35-7.24 (m, 2H), 7.23 (d, J = 2.5 Hz, 1H), 7.25 (d, J = 8.5 Hz, 1H), 7.08 (t, J = 55.0 Hz, 1H). |
| | 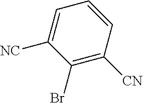 [SMILES: N#Cc1cccc(C#N)c1Br] | | |

### Example Compounds Set 2

Example compounds are shown below.

| # | building block(s) | structure | analytical data |
|---|---|---|---|
| 2 | | 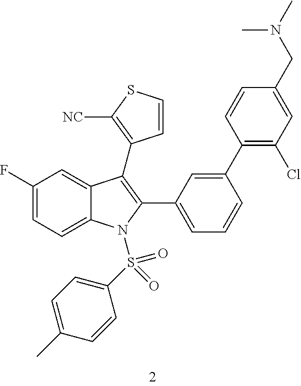 [SMILES: Cc1ccc(S(=O)(=O)n2c(-c3cccc(-c4ccc(CN(C)C)cc4Cl)c3)c(-c3ccsc3C#N)c3cc(F)ccc32)cc1] | |
| 2/1 | 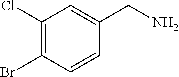 | 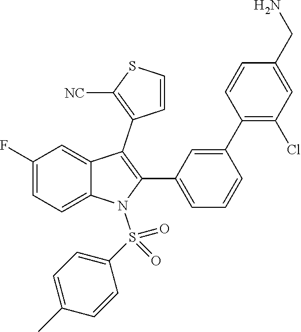 [SMILES: Cc1ccc(S(=O)(=O)n2c(-c3cccc(-c4ccc(CN)cc4Cl)c3)c(-c3ccsc3C#N)c3cc(F)ccc32)cc1] | ¹H-NMR (500 MHz,CD₃OD) δ: 8.41 (dd, J = 9.0, 4.5 Hz, 1H), 7.83 (d, J = 5.5 Hz, 1H), 7.65 (d, J = 1.5 Hz, 1H), 7.50-7.43 (m, 4H), 7.35-7.26 (m, 4H), 7.20-7.15 (m, 3H), 7.05 (dd, J = 8.5, 2.5 Hz, 1H), 6.96 (d, J = 5.0 Hz, 1H), 4.19 (s, 2H), 2.29 (s, 3H); MS: 611.8 (M + 1)⁺. |
| 2/2 | 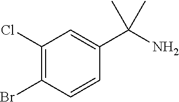 | 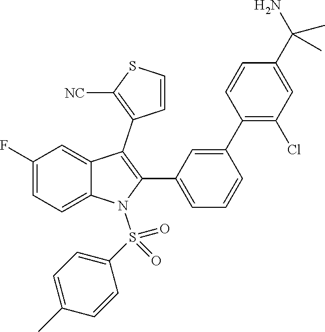 [SMILES: Cc1ccc(S(=O)(=O)n2c(-c3cccc(-c4ccc(C(C)(C)N)cc4Cl)c3)c(-c3ccsc3C#N)c3cc(F)ccc32)cc1] | ¹H-NMR (500 MHz, CD₃OD) δ: 8.41 (dd, J = 9.0, 4.5 Hz, 1H), 7.81 (d, J = 5.0 Hz, 1H), 7.64 (d, J = 2.0 Hz, 1H), 7.52- 7.41 (m, 4H), 7.29-7.25 (m, 4H), 7.16-7.14 (m, 2H), 7.06 (dd, J = 8.5, 3.0 Hz, 1H), 6.97 (s, 1H), 6.93 (d, J = 5.0 Hz, 1H), 2.25 (s, 3H), 1.54 (s, 6H); MS: 639.0 (M + 1)⁺. |
| 2/16 | 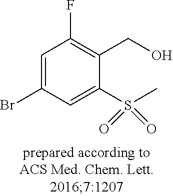 | 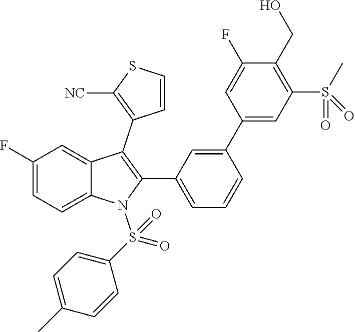 [SMILES: Cc1ccc(S(=O)(=O)n2c(-c3cccc(-c4cc(F)c(CO)c(S(C)(=O)=O)c4)c3)c(-c3ccsc3C#N)c3cc(F)ccc32)cc1] | ¹H-NMR (500 MHz, DMSO- d₆) δ: 8.34-8.29 (m, 1H), 8.01 (d, J = 5.0 Hz, 1H), 7.96 (d, J = 1.5 Hz, 1H), 7.86-7.84 (m, 2H), 7.61 (s, 1H), 7.53 (t, J = 7.5 Hz, 1H), 7.41-7.28 (m, 6H), 7.21- 7.18 (m, 1H), 7.08 (d, J = 5.5 Hz, 1H), 5.59-5.55 (m, 1H), 4.96-4.94 (m, 2H), 3.45 (s, 3H), 2.27 (s, 3H); MS: 691.8 (M + 18)⁺. |
| 2/18 | 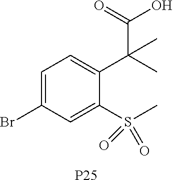 | 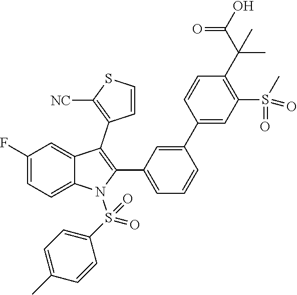 [SMILES: Cc1ccc(S(=O)(=O)n2c(-c3cccc(-c4ccc(C(C)(C)C(=O)O)c(S(C)(=O)=O)c4)c3)c(-c3ccsc3C#N)c3cc(F)ccc32)cc1] | ¹H-NMR (500 MHz, CD₃OD) δ: 8.44-8.41 (m, 1H), 8.16 (s, 1H), 7.85-7.76 (m, 4H), 7.51- 7.48 (m, 1H), 7.39-7.26 (m, 5H), 7.19-7.17 (m, 2H), 7.06- 7.01 (m, 2H), 3.23 (s, 3H), 2.26 (s, 3H), 1.77 (s, 6H); MS: 730.1 (M + 18)⁺. |

### Example Compounds Set 3

Example compounds are shown below.

| # | starting material | structure | analytical data |
|---|---|---|---|
| 3 | | 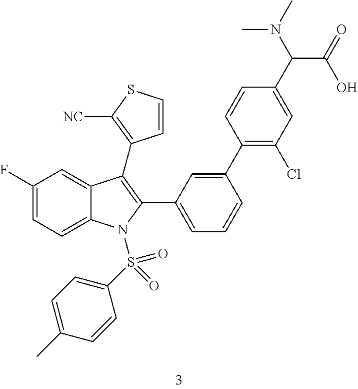 [SMILES: Cc1ccc(S(=O)(=O)n2c(-c3cccc(-c4ccc(C(C(=O)O)N(C)C)cc4Cl)c3)c(-c3ccsc3C#N)c3cc(F)ccc32)cc1] | |
| 3/1 | 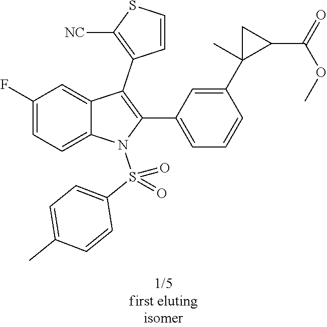 | 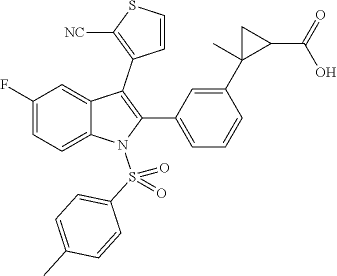 [SMILES: Cc1ccc(S(=O)(=O)n2c(-c3cccc(C4(C)CC4C(=O)O)c3)c(-c3ccsc3C#N)c3cc(F)ccc32)cc1] | ¹H-NMR (500 MHz, CD₃OD) δ: 8.42 (dd, J = 4.5, 9.0 Hz, 1H), 7.80 (d, J = 5.0 Hz, 1H), 7.40 (d, J = 8.0 Hz, 1H), 7.32- 7.17 (m, 7H), 7.04 (dd, J = 2.8, 8.8 Hz, 1H), 6.99 (s, 1H), 6.88 (d, J = 5.5 Hz, 1H), 2.36 (s, 3H), 1.83-1.80 (m, 1H), 1.43 (s, 3H), 1.31-1.29 (m, 1H), 1.22-1.19 (m, 1H); MS: 568.8 (M − 1)⁻. |
| 3/2 | 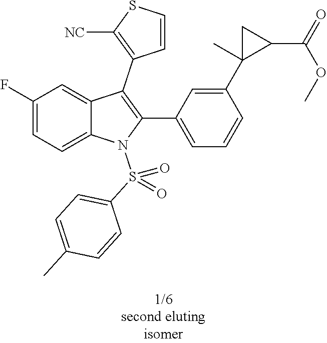 | 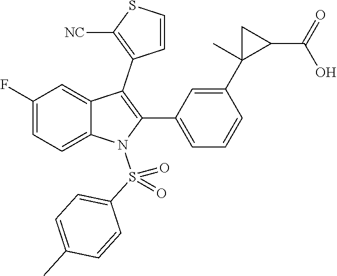 [SMILES: Cc1ccc(S(=O)(=O)n2c(-c3cccc(C4(C)CC4C(=O)O)c3)c(-c3ccsc3C#N)c3cc(F)ccc32)cc1] | ¹H-NMR (500 MHz, CD₃OD) δ: 8.43 (dd, J = 4.3, 8.8 Hz, 1H), 7.74 (d, J = 5.0 Hz, 1H), 7.36-7.21 (m, 8H), 7.13-7.04 (m, 2H), 6.75 (d, J = 5.0 Hz, 1H), 2.35 (s, 3H), 1.94-1.92 (m, 1H), 1.63-1.62 (m, 1H), 1.38 (s, 3H), 1.20-1.17 (m, 1H); MS: 568.8 (M − 1)⁻. |
| 3/3 | 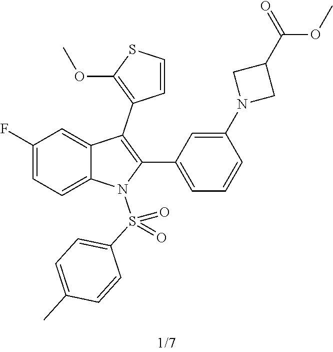 | 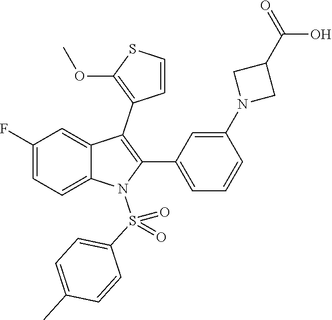 [SMILES: COc1sccc1-c1c(-c2cccc(N3CC(C(=O)O)C3)c2)n(S(=O)(=O)c2ccc(C)cc2)c2ccc(F)cc12] | ¹H-NMR (500 MHz, DMSO-d₆) δ: 8.21 (dd, J = 4.5, 9.0 Hz, 1H), 6.68 (d, J = 9.0 Hz, 2H), 7.31-7.26 (m, 3H), 7.17 (t, J = 8.0 Hz, 1H), 6.97 (dd, J = 2.5, 9.0 Hz, 1H), 6.75 (d, J = 6.0 Hz, 1H), 6.64 (d, J = 8.0 Hz, 1H), 6.48 (dd, J = 2.0, 8.0 Hz, 1H), 6.39 (d, J = 6.0 Hz, 1H), 6.26 (s, 1H), 3.93 (t, J = 8.0 Hz, 2H), 3.79 (t, J = 6.5 Hz, 2H), 3.60 (s, 3H), 3.54-3.47 (m, 1H), 2.32 (s, 3H); MS: 577.1 (M + 1)⁺. |
| 3/4 | 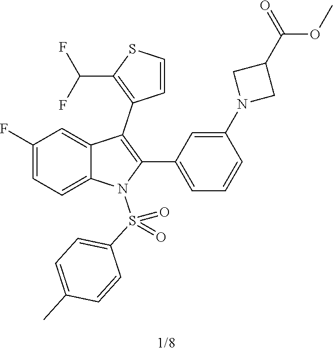 | 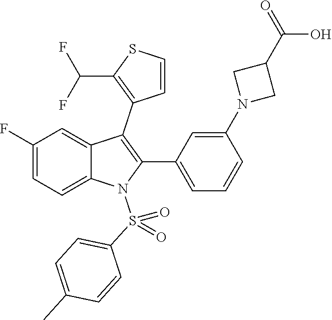 [SMILES: Cc1ccc(S(=O)(=O)n2c(-c3cccc(N4CC(C(=O)O)C4)c3)c(-c3ccsc3C(F)F)c3cc(F)ccc32)cc1] | ¹H-NMR (500 MHz, CD₃OD) δ: 8.36 (dd, J = 4.3, 8.8 Hz, 1H), 7.60 (d, J = 5.5 Hz, 1H), 7.30 (d, J = 8.5 Hz, 2H), 7.25-7.17 (m, 4H), 6.84 (dd, J = 2.5, 8.5 Hz, 1H), 6.75-6.70 (m, 2H), 6.55 (dd, J = 1.8, 8.3 Hz, 1H), 6.19 (t, J = 55.0 Hz, 1H), 6.16 (s, 1H), 3.99-3.82 (m, 4H), 3.58-3.52 (m, 1H), 2.38 (s, 3H); MS: 597.1 (M + 1)⁺. |
| 3/5 | 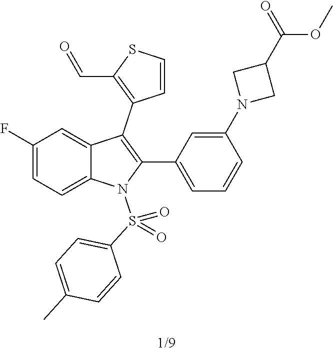 | 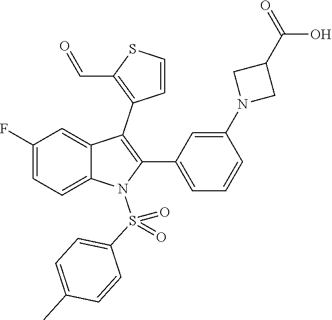 [SMILES: Cc1ccc(S(=O)(=O)n2c(-c3cccc(N4CC(C(=O)O)C4)c3)c(-c3ccsc3C=O)c3cc(F)ccc32)cc1] | ¹H-NMR (500 MHz, DMSO-d₆) δ: 12.64 (br s, 1H), 9.31 (d, J = 1.0 Hz, 1H), 8.26 (dd, J = 4.0, 9.0 Hz, 1H), 8.10 (dd, J = 1.3, 4.8 Hz, 1H), 7.40-7.32 (m, 5H), 7.14-7.06 (m, 3H), 6.60 (br s, 1H), 6.46 (dd, J = 1.8, 8.3 Hz, 1H), 6.20 (br s, 1H), 3.87 (t, J = 8.0 Hz, 2H), 3.74 (t, J = 6.5 Hz, 2H), 3.54-3.48 (m, 1H), 2.33 (s, 3H); MS: 575.0 (M + 1)⁺. |
| 3/6 | 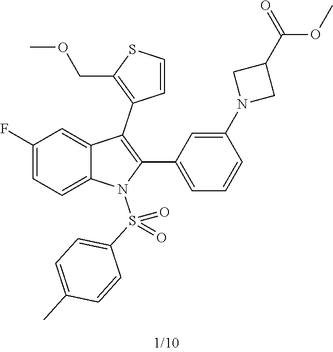 | 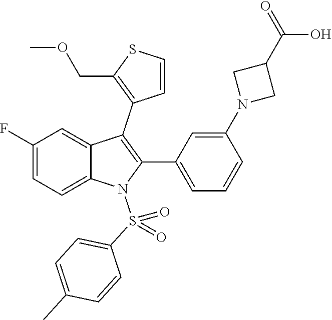 [SMILES: COCc1sccc1-c1c(-c2cccc(N3CC(C(=O)O)C3)c2)n(S(=O)(=O)c2ccc(C)cc2)c2ccc(F)cc12] | ¹H-NMR (500 MHz, DMSO-d₆) δ: 8.23 (dd, J = 4.5, 9.0 Hz, 1H), 7.46 (d, J = 5.0 Hz, 1H), 7.33-7.27 (m, 5H), 7.14 (t, J = 7.8 Hz, 1H), 6.87 (dd, J = 2.5, 8.5 Hz, 1H), 6.63-6.60 (m, 2H), 6.45 (dd, J = 2.0, 8.0 Hz, 1H), 6.18 (s, 1H), 4.14-4.09 (m, 1H), 3.89-3.73 (m, 5H), 3.54-3.48 (m, 1H), 2.99 (s, 3H), 2.31 (s, 3H); MS: 591.1 (M + 1)⁺. |
| 3/7 | 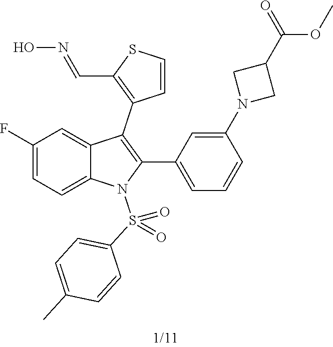 | 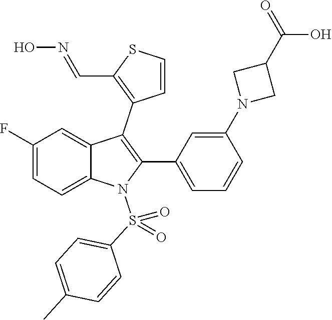 [SMILES: Cc1ccc(S(=O)(=O)n2c(-c3cccc(N4CC(C(=O)O)C4)c3)c(-c3ccsc3/C=N/O)c3cc(F)ccc32)cc1] | ¹H-NMR (500 MHz, DMSO-d₆) δ: 12.69 (br s, 1H), 11.24 (s, 1H), 8.25 (dd, J = 4.3, 9.3 Hz, 1H), 7.49 (d, J = 5.0 Hz, 1H), 7.38-7.29 (m, 6H), 7.13 (d, J = 7.8 Hz, 1H), 6.83 (dd, J = 2.5, 8.5 Hz, 1H), 6.72 (d, J = 5.5 Hz, 1H), 6.60-6.57 (m, 1H), 6.45 (dd, J = 2.0, 8.0 Hz, 1H), 6.19 (s, 1H), 3.91-3.87 (m, 2H), 3.79-3.75 (m, 2H), 3.54-3.50 (m, 1H), 2.32 (m, 3H); MS: 590.0 (M + 1)⁺. |
| 3/8 | 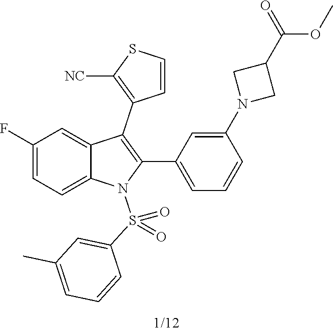 | 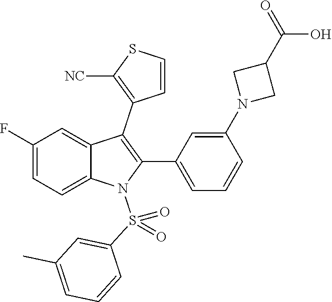 [SMILES: Cc1cccc(S(=O)(=O)n2c(-c3cccc(N4CC(C(=O)O)C4)c3)c(-c3ccsc3C#N)c3cc(F)ccc32)c1] | ¹H-NMR (500 MHz, DMSO-d₆) δ: 8.28 (dd, J = 4.8, 9.3 Hz, 1H), 8.24 (d, J = 5.0 Hz, 1H), 7.49 (d, J = 7.5 Hz, 1H), 7.42-7.33 (m, 3H), 7.23 (s, 1H), 7.15- 7.11 (m, 2H), 6.94 (d, J = 5.0 Hz, 1H), 6.52 (d, J = 7.5 Hz, 1H), 6.47 (dd, J = 1.3, 8.3 Hz, 1H), 6.17 (s, 1H), 3.86 (t, J = 8.0 Hz, 2H), 3.75 (t, J = 6.5 Hz, 2H), 3.45- 3.40 (m, 1H), 2.28 (s, 3H); MS: 569.8 (M − 1)⁻. |
| 3/9 | 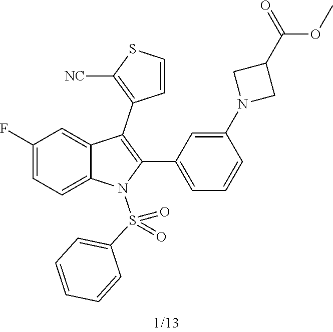 | 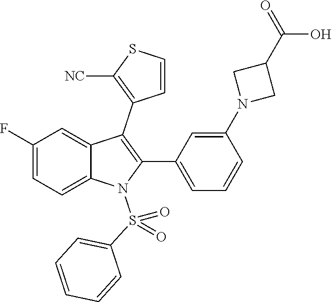 [SMILES: N#Cc1sccc1-c1c(-c2cccc(N3CC(C(=O)O)C3)c2)n(S(=O)(=O)c2ccccc2)c2ccc(F)cc12] | ¹H-NMR (500 MHz, DMSO-d₆) δ: 8.28 (dd, J = 4.5, 9.5 Hz, 1H), 7.98 (d, J = 4.5 Hz, 1H), 7.71-7.67 (m, 1H), 7.54- 7.50 (m, 4H), 7.37- 7.33 (m, 1H), 7.14-7.11 (m, 2H), 6.93 (d, J = 5.0 Hz, 1H), 6.53 (d, J = 7.5 Hz, 1H), 6.45 (dd, J = 1.8, 8.3 Hz, 1H), 6.18 (s, 1H), 3.83 (t, J = 7.8 Hz, 2H), 3.74 (t, J = 6.3 Hz, 2H), 3.34-3.30 (m, 1H); MS: 557.9 (M + 1)⁺. |
| 3/ 10 | 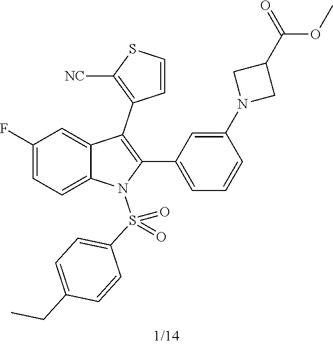 | 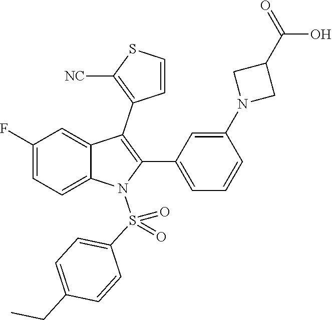 [SMILES: CCc1ccc(S(=O)(=O)n2c(-c3cccc(N4CC(C(=O)O)C4)c3)c(-c3ccsc3C#N)c3cc(F)ccc32)cc1] | ¹H-NMR (500 MHz, DMSO-d₆) δ: 8.28 (dd, J = 4.0, 9.0 Hz, 1H), 7.98 (d, J = 5.0 Hz, 1H), 7.44 (d, J = 8.0 Hz, 2H), 7.37- 7.33 (m, 3H), 7.16-7.11 (m, 2H), 6.94 (d, J = 5.0 Hz, 1H), 6.55 (d, J = 7.5 Hz, 1H), 6.48 (dd, J = 1.5, 8.0 Hz, 1H), 6.21 (s, 1H), 3.89 (t, J = 7.5 Hz, 2H), 3.77 (t, J = 6.3 Hz, 2H), 3.49-3.44 (m, 1H), 2.63 (q, J = 7.5 Hz, 2H), 1.12 (t, J = 7.5 Hz, 3H); MS: 586.8 (M + 1)⁺. |
| 3/ 11 | 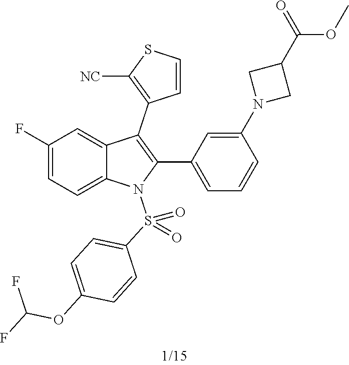 | 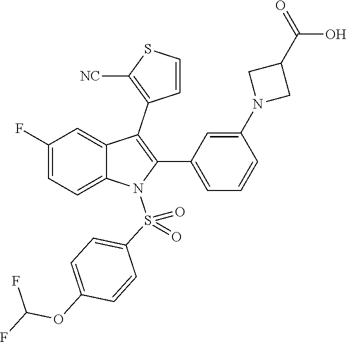 [SMILES: N#Cc1sccc1-c1c(-c2cccc(N3CC(C(=O)O)C3)c2)n(S(=O)(=O)c2ccc(OC(F)F)cc2)c2ccc(F)cc12] | ¹H-NMR (500 MHz, DMSO-d₆) δ: 8.28 (dd, J = 4.3, 9.3 Hz, 1H), 7.99 (d, J = 5.0 Hz, 1H), 7.58 (d, J = 9.0 Hz, 2H), 7.52-7.23 (m, 4H), 7.15-7.12 (m, 2H), 6.95 (d, J = 5.0 Hz, 1H), 6.53 (d, J = 7.5 Hz, 1H), 6.49-6.48 (m, 1H), 6.27 (s, 1H), 3.90 (t, J = 8.0 Hz, 2H), 3.79 (t, J = 6.5 Hz, 2H), 3.46- 3.41 (m, 1H); MS: 623.7 (M + 1)⁺. |
| 3/ 12 | 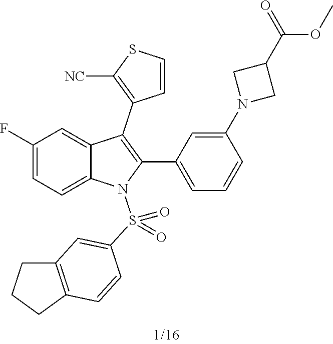 | 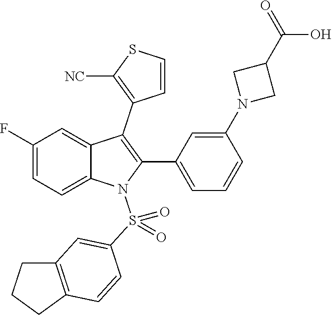 [SMILES: N#Cc1sccc1-c1c(-c2cccc(N3CC(C(=O)O)C3)c2)n(S(=O)(=O)c2ccc3c(c2)CCC3)c2ccc(F)cc12] | ¹H-NMR (500 MHz, DMSO-d₆) δ: 8.28 (dd, J = 4.5, 9.0 Hz, 1H), 7.97 (d, J = 5.0 Hz, 1H), 7.37-7.30 (m, 3H), 7.27 (s, 1H), 7.15-7.10 (m, 2H), 6.94 (d, J = 5.5 Hz, 1H), 6.54 (d, J = 7.5 Hz, 1H), 6.46 (dd, J = 1.8, 8.3 Hz, 1H), 6.14 (s, 1H), 3.84 (t, J = 8.0 Hz, 2H), 3.74 (t, J = 6.5 Hz, 2H), 3.41-3.35 (m, 1H), 2.88-2.80 (m, 4H), 2.02-1.97 (m, 2H); MS: 598.2 (M + 1)⁺. |
| 3/ 13 | 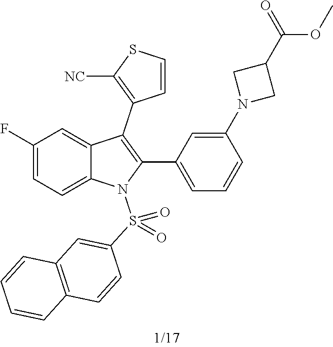 | 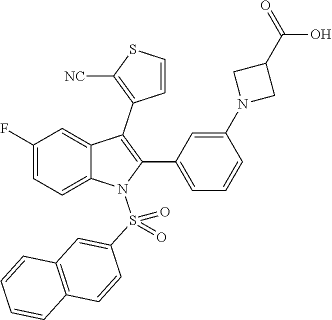 [SMILES: N#Cc1sccc1-c1c(-c2cccc(N3CC(C(=O)O)C3)c2)n(S(=O)(=O)c2ccc3ccccc3c2)c2ccc(F)cc12] | ¹H-NMR (500 MHz, DMSO-d₆) δ: 8.38 (dd, J = 4.0, 9.5 Hz, 1H), 8.18 (s, 1H), 8.06- 7.96 (m, 4H), 7.76-7.68 (m, 2H), 7.46 (d, J = 9.0 Hz, 1H), 7.39-7.36 (m, 1H), 7.15- 7.12 (m, 2H), 6.95 (d, J = 5.0 Hz, 1H), 6.59 (d, J = 7.0 Hz, 1H), 6.45 (d, J = 7.5 Hz, 1H), 6.08 (s, 1H), 3.69-3.59 (m, 4H), 3.42-3.32 (m, 1H); MS: 608.2 (M + 1)⁺. |
| 3/ 14 | 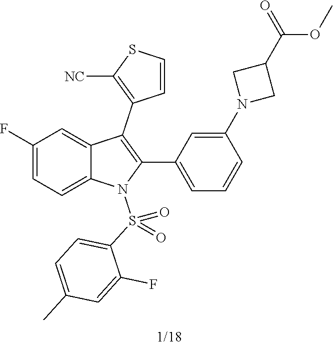 | 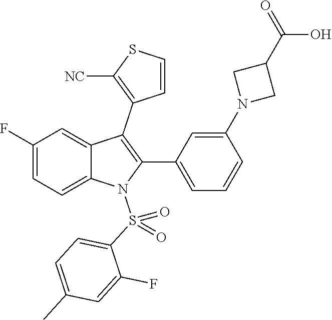 [SMILES: Cc1ccc(S(=O)(=O)n2c(-c3cccc(N4CC(C(=O)O)C4)c3)c(-c3ccsc3C#N)c3cc(F)ccc32)c(F)c1] | ¹H-NMR (500 MHz, DMSO-d₆) δ: 8.16 (dd, J = 4.5, 9.0 Hz, 1H), 7.98 (d, J = 5.5 Hz, 1H), 7.36-7.17 (m, 4H), 7.05 (t, J = 7.5 Hz, 2H), 6.99 (d, J = 5.0 Hz, 1H), 6.45-6.40 (m, 2H), 5.97 (s, 1H), 3.79 (t, J = 8.0 Hz, 2H), 3.68 (t, J = 6.3 Hz, 2H), 3.45-3.40 (m, 1H), 2.36 (s, 3H); MS: 589.9 (M + 1)⁺. |
| 3/ 15 | 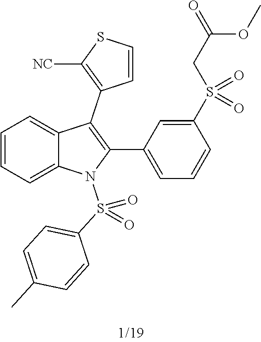 | 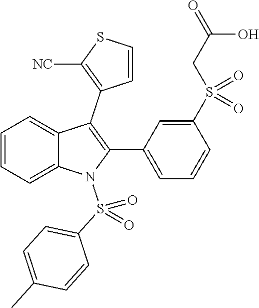 [SMILES: Cc1ccc(S(=O)(=O)n2c(-c3cccc(S(=O)(=O)CC(=O)O)c3)c(-c3ccsc3C#N)c3ccccc32)cc1] | ¹H-NMR (500 MHz, DMSO-d₆) δ: 8.42 (d, J = 8.5 Hz, 1H), 8.03- 8.01 (m, 1H), 7.83 (d, J = 5.0 Hz, 1H), 7.79 (s, 1H), 7.67-7.62 (m, 2H), 7.56-7.53 (m, 1H), 7.42-7.39 (m, 2H), 7.34 (d, J = 8.0 Hz, 2H), 7.24 (d, J = 8.5 Hz, 2H), 6.97 (d, J = 5.0 Hz, 1H), 4.26 (s, 2H), 2.35 (s, 3H); MS: 576.7 (M + 1)⁺. |
| 3/ 16 | 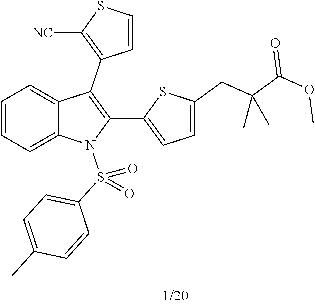 | 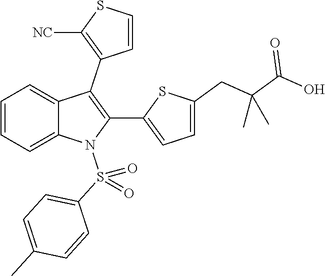 [SMILES: Cc1ccc(S(=O)(=O)n2c(-c3ccc(CC(C)(C)C(=O)O)s3)c(-c3ccsc3C#N)c3ccccc32)cc1] | ¹H-NMR (500 MHz, DMSO-d₆) δ: 12.07 (br s, 1H), 8.28 (d, J = 8.0 Hz, 1H), 8.02 (d, J = 5.0 Hz, 1H), 7.53-7.50 (m, 1H), 7.44 (d, J = 8.0 Hz, 2H), 7.38 (d, J = 4.0 Hz, 2H), 7.31 (d, J = 8.5 Hz, 2H), 7.05 (d, J = 3.5 Hz, 1H), 6.99 (d, J = 5.5 Hz, 1H), 6.83 (d, J = 4.0 Hz, 1H), 2.97 (s, 2H), 2.31 (s, 3H), 1.09 (s, 6H); MS: 559.0 (M − 1)⁻. |
| 3/ 17 | 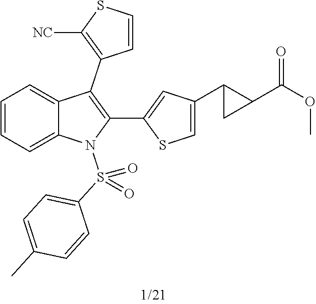 | 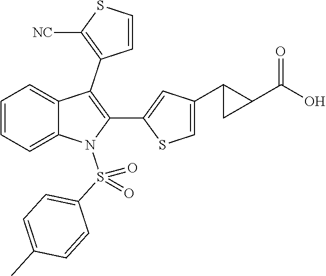 [SMILES: Cc1ccc(S(=O)(=O)n2c(-c3cc(C4CC4C(=O)O)cs3)c(-c3ccsc3C#N)c3ccccc32)cc1] | ¹H-NMR (500 MHz, DMSO-d₆) δ: 8.26 (d, J = 11.0 Hz, 1H), 8.05 (d, J = 6.0 Hz, 1H), 7.54-7.32 (m, 8H), 7.04 (d, J = 6.0 Hz, 1H), 6.91 (d, J = 2.0 Hz, 1H), 2.39-2.34 (m, 1H), 2.32 (s, 3H), 1.75-1.70 (m, 1H), 1.39-1.35 (m, 1H), 1.20-1.15 (m, 1H); MS: 562.1 (M + 18)⁺. |
| 3/ 18 | 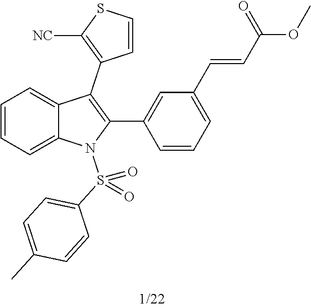 | 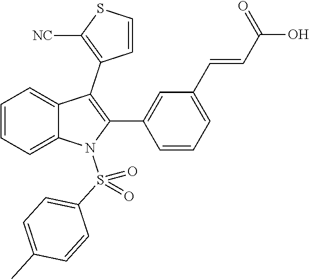 [SMILES: Cc1ccc(S(=O)(=O)n2c(-c3cccc(/C=C/C(=O)O)c3)c(-c3ccsc3C#N)c3ccccc32)cc1] | ¹H-NMR (500 MHz, CD₃OD) δ: 8.41 (d, J = 10.0 Hz, 1H), 7.80 (d, J = 6.5 Hz, 1H), 7.65-7.49 (m, 3H), 7.43-7.21 (m, 9H), 6.96 (d, J = 6.5 Hz, 1H), 6.35 (d, J = 20.0 Hz, 1H), 2.36 (s, 3H); MS: 524.6 (M + 1)⁺. |
| 3/ 19 | 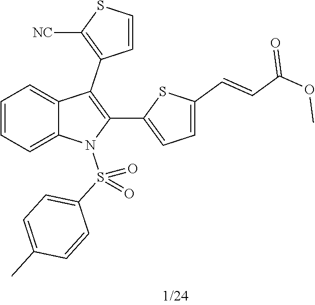 | 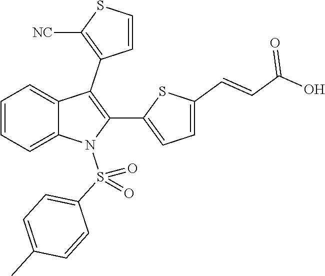 [SMILES: Cc1ccc(S(=O)(=O)n2c(-c3ccc(/C=C/C(=O)O)s3)c(-c3ccsc3C#N)c3ccccc32)cc1] | ¹H-NMR (500 MHz, DMSO-d₆) δ: 12.47 (br s, 1H), 8.25 (d, J = 10.5 Hz, 1H), 8.07 (d, J = 6.5 Hz, 1H), 7.70 (d, J = 20.0 Hz, 1H), 7.56-7.51 (m, 4H), 7.42-7.32 (m, 4H), 7.23 (d, J = 4.5 Hz, 1H), 7.09 (d, J = 6.5 Hz, 1H), 6.18 (d, J = 20.0 Hz, 1H), 2.31 (s, 3H); MS: 530.7 (M + 1)⁺. |
| 3/ 20 | 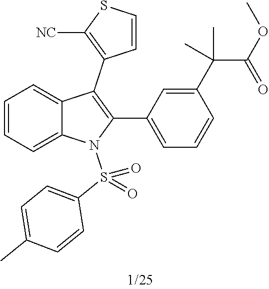 | 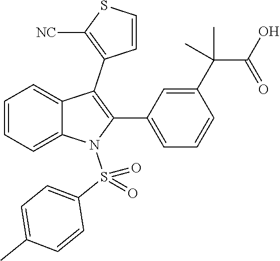 [SMILES: Cc1ccc(S(=O)(=O)n2c(-c3cccc(C(C)(C)C(=O)O)c3)c(-c3ccsc3C#N)c3ccccc32)cc1] | ¹H-NMR (500 MHz, DMSO-d₆) δ: 12.39 (br s, 1H), 8.29 (d, J = 8.5 Hz, 1H), 7.98 (d, J = 5.0 Hz, 1H), 7.51 (t, J = 7.5 Hz, 1H), 7.40-7.28 (m, 9H), 7.06 (s, 1H), 6.92 (d, J = 4.0 Hz, 1H), 2.30 (s, 3H), 1.36 (s, 6H); MS: 563.1 (M + Na)⁺. |
| 3/ 21 | 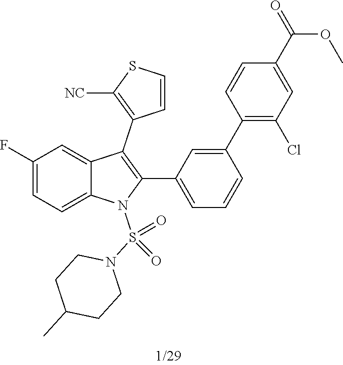 | 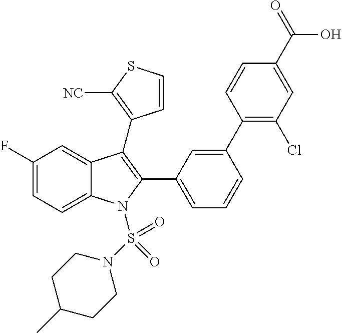 [SMILES: CC1CCN(S(=O)(=O)n2c(-c3cccc(-c4ccc(C(=O)O)cc4Cl)c3)c(-c3ccsc3C#N)c3cc(F)ccc32)CC1] | ¹H-NMR (500 MHz, CD₃OD) δ: 8.26 (dd, J = 4.5, 9.0 Hz, 1H), 8.10 (d, J = 1.0 Hz, 1H), 7.99 (dd, J = 1.8, 7.8 Hz, 1H), 7.86 (d, J = 5.5 Hz, 1H), 7.56- 7.51 (m, 3H), 7.44 (s, 1H), 7.40 (d, J = 8.0 Hz, 1H), 7.25-7.21 (m, 1H), 7.15 (dd, J = 2.8, 8.8 Hz, 1H), 6.98 (d, J = 4.5 Hz, 1H), 3.48 (d, J = 12.5 Hz, 2H), 2.53-2.48 (m, 2H), 1.47 (d, J = 12.5 Hz, 2H), 1.33-1.28 (m, 1H), 0.86-0.77 (m, 5H); MS: 633.9 (M + 1)⁺. |
| 3/ 22 | 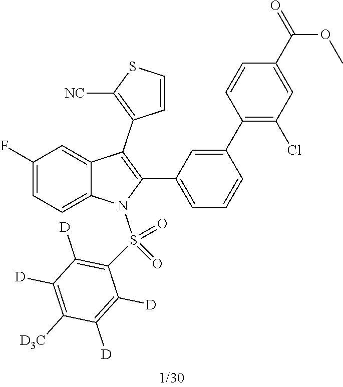 | 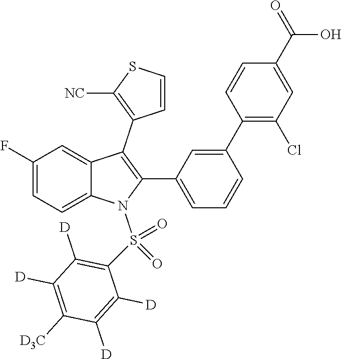 [SMILES: [2H]c1c([2H])c(S(=O)(=O)n2c(-c3cccc(-c4ccc(C(=O)O)cc4Cl)c3)c(-c3ccsc3C#N)c3cc(F)ccc32)c([2H])c([2H])c1C([2H])([2H])[2H]] | ¹H-NMR (500 MHz, CD₃OD) δ: 8.44 (dd, J = 4.5, 9.0 Hz, 1H), 8.09 (d, J = 2.0 Hz, 1H), 8.00 (dd, J = 1.5, 7.5 Hz, 1H), 7.82 (d, J = 5.0 Hz, 1H), 7.52- 7.44 (m, 3H), 7.39 (d, J = 8.0 Hz, 1H), 7.29-6.95 (m, 4H); MS: 632.0 (M − 1)⁻. |
| 3/ 23 | 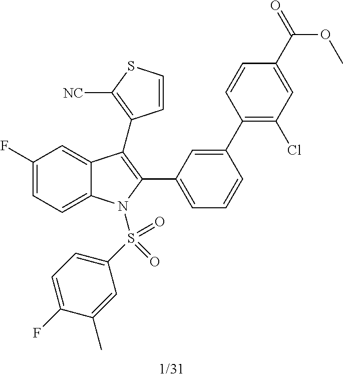 | 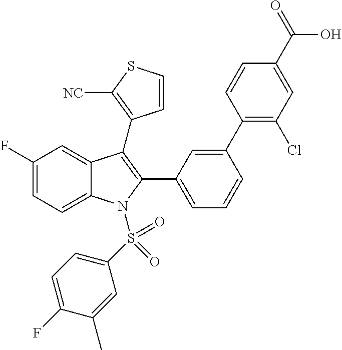 [SMILES: Cc1cc(S(=O)(=O)n2c(-c3cccc(-c4ccc(C(=O)O)cc4Cl)c3)c(-c3ccsc3C#N)c3cc(F)ccc32)ccc1F] | ¹H-NMR (500 MHz, DMSO-d₆) δ: 13.41 (br s, 1H), 8.31 (dd, J = 4.5, 9.0 Hz, 1H), 8.04-7.95 (m, 3H), 7.56-7.48 (m, 3H), 7.42-7.34 (m, 4H), 7.26- 7.18 (m, 3H), 7.03 (d, J = 5.0 Hz, 1H), 2.08 (s, 3H); MS: 642.9 (M − 1)⁻. |
| 3/ 24 | 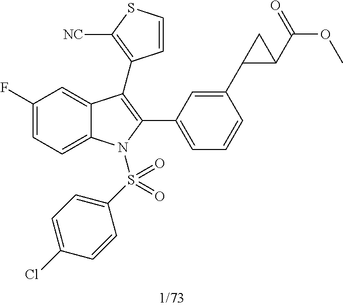 | 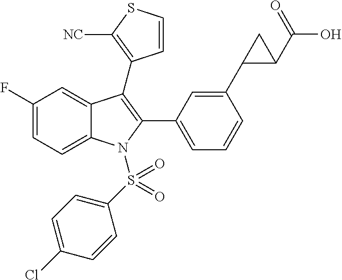 [SMILES: N#Cc1sccc1-c1c(-c2cccc(C3CC3C(=O)O)c2)n(S(=O)(=O)c2ccc(Cl)cc2)c2ccc(F)cc12] | ¹H-NMR (500 MHz, DMSO-d₆) δ: 8.29-8.26 (m, 1H), 8.01 (d, J = 5.5 Hz, 1H), 7.65- 7.55 (m, 2H), 7.45-7.35 (m, 3H), 7.26 (d, J = 5.0 Hz, 1H), 7.18-7.14 (m, 1H), 7.06- 7.03 (m, 1H), 6.97 (d, J = 5.0 Hz, 1H), 6.91 (s, 1H), 2.35-2.30 (m, 1H), 1.70 (s, 1H), 1.41-1.37 (m, 1H), 1.17 (s, 1H); MS: 575.0 (M − 1)⁻. |
| 3/ 25 | 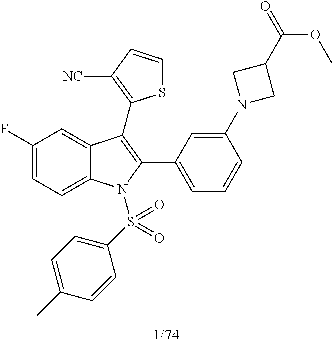 | 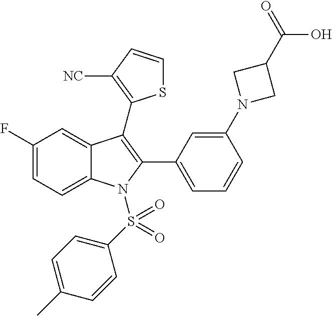 [SMILES: Cc1ccc(S(=O)(=O)n2c(-c3cccc(N4CC(C(=O)O)C4)c3)c(-c3sccc3C#N)c3cc(F)ccc32)cc1] | ¹H-NMR (500 MHz, DMSO-d₆) δ: 12.66 (br s, 1H), 8.31-8.26 (m, 1H), 7.76 (d, J = 5.5 Hz, 1H), 7.46-7.33 (m, 6H), 7.20-7.15 (m, 2H), 6.60 (d, J = 7.5 Hz, 1H), 6.53-6.49 (m, 1H), 6.23 (s, 1H), 3.92 (t, J = 8.0 Hz, 2H), 3.79 (t, J = 6.5 Hz, 2H), 3.54-3.50 (m, 1H), 2.34 (s, 3H); MS: 572.1 (M + 1)⁺. |
| 3/ 26 | 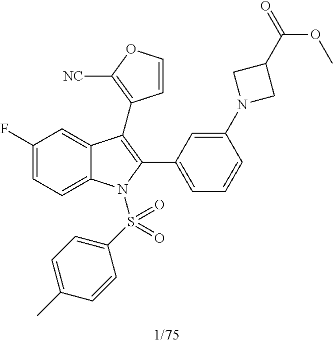 | 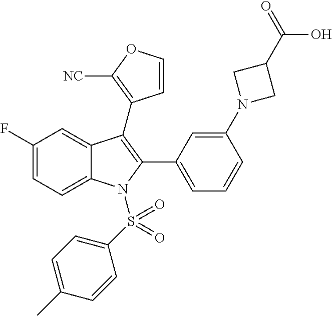 [SMILES: Cc1ccc(S(=O)(=O)n2c(-c3cccc(N4CC(C(=O)O)C4)c3)c(-c3ccoc3C#N)c3cc(F)ccc32)cc1] | ¹H-NMR (500 MHz, CD₃OD) δ: 8.42-8.39 (m, 1H), 7.72 (d, J = 1.5 Hz, 1H), 7.36 (d, J = 8.5 Hz, 2H), 7.28-7.14 (m, 5H), 6.65 (d, J = 8.0 Hz, 1H), 6.66-6.64 (m, 1H), 6.60- 6.57 (m, 1H), 6.35 (d, J = 2.0 Hz, 1H), 6.20 (s, 1H), 4.01 (t, J = 7.5 Hz, 2H), 3.91 (t, J = 6.0 Hz, 2H), 3.54-3.50 (m, 1H), 2.38 (s, 3H); MS: 556.2 (M + 1)⁺. |
| 3/ 27 | 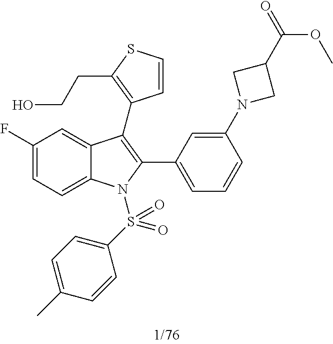 | 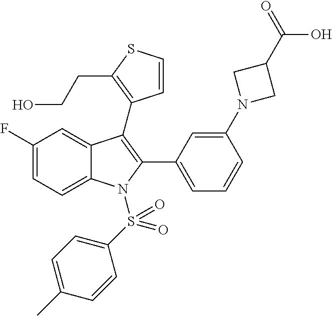 [SMILES: Cc1ccc(S(=O)(=O)n2c(-c3cccc(N4CC(C(=O)O)C4)c3)c(-c3ccsc3CCO)c3cc(F)ccc32)cc1] | ¹H-NMR (500 MHz, DMSO-d₆) δ: 8.23-8.19 (m, 1H), 7.32-7.26 (m, 6H), 7.14 (t, J = 7.5 Hz, 1H), 6.84-6.81 (m, 1H), 6.66-6.62 (m, 1H), 6.55 (d, J = 5.0 Hz, 1H), 6.45-6.42 (m 1H), 6.16 (s, 3H), 3.90-3.85 (m, 2H), 3.77-3.73 (m, 2H), 3.52-3.49 (m, 1H), 3.25-3.19 (m, 2H), 2.48-2.33 (m, 2H), 2.31 (s, 1H); MS: 591.0 (M + 1)⁺. |
| 3/ 28 | 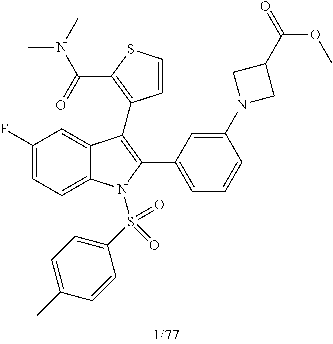 | 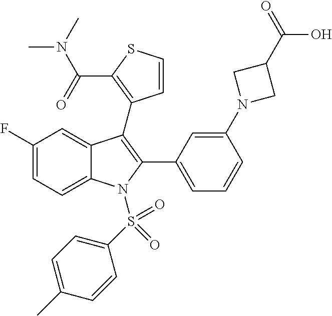 [SMILES: Cc1ccc(S(=O)(=O)n2c(-c3cccc(N4CC(C(=O)O)C4)c3)c(-c3ccsc3C(=O)N(C)C)c3cc(F)ccc32)cc1] | ¹H-NMR (500 MHz, CD₃OD) δ: 8.40-8.37 (m, 1H), 7.54 (d, J = 5.5 Hz, 1H), 7.34 (d, J = 8.0 Hz, 2H), 7.25-7.19 (m, 4H), 7.05-7.03 (m, 1H), 6.75 (d, J = 5.0 Hz, 1H), 6.67-6.59 (m, 2H), 6.26 (s, 1H), 4.05-4.01 (m, 2H), 3.95-3.92 (m, 2H), 3.60-3.57 (m, 1H), 2.65 (s, 3H), 2.38 (s, 3H), 2.33 (s, 3H); MS: 618.0 (M + 1)⁺. |
| C3/ 29 | 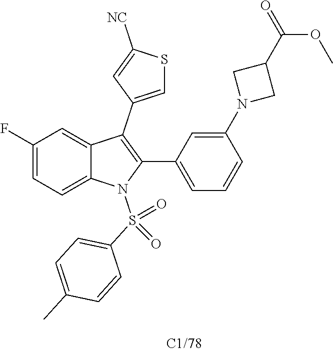 | 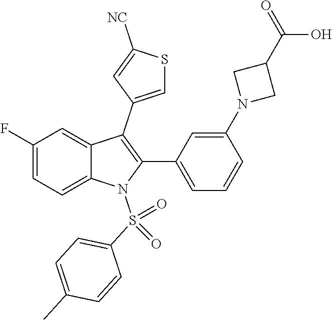 [SMILES: Cc1ccc(S(=O)(=O)n2c(-c3cccc(N4CC(C(=O)O)C4)c3)c(-c3csc(C#N)c3)c3cc(F)ccc32)cc1] | ¹H-NMR (500 MHz, CD₃OD) δ: 8.41-8.38 (m, 1H), 7.63 (d, J = 1.5 Hz, 1H), 7.40-7.35 (m, 3H), 7.30- 7.21 (m, 5H), 6.68- 6.61 (m, 2H), 6.18 (d, J = 1.5 Hz, 1H), 4.03-3.99 (m, 2H), 3.93-3.89 (m, 2H), 3.56- 3.53 (m, 1H), 2.37 (s, 3H); MS: 572.1 (M + 1)⁺. |
| 3/ 30 | 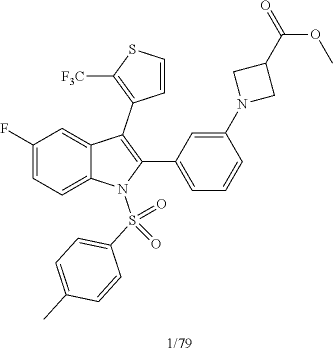 | 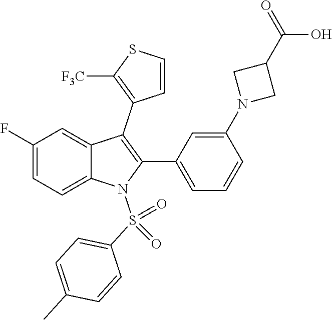 [SMILES: Cc1ccc(S(=O)(=O)n2c(-c3cccc(N4CC(C(=O)O)C4)c3)c(-c3ccsc3C(F)(F)F)c3cc(F)ccc32)cc1] | ¹H-NMR (500 MHz, CD₃OD) δ: 8.35-8.32 (m, 1H), 7.62 (d, J = 5.0 Hz, 1H), 7.31-7.15 (m, 7H), 6.78- 6.75 (m, 1H), 6.66 (br s, 2H), 6.51-6.48 (m, 1H), 3.98-3.79 (m, 4H), 3.55-3.50 (m, 1H), 2.37 (s, 3H); MS: 615.1 (M + 1)⁺. |
| 3/ 31 | 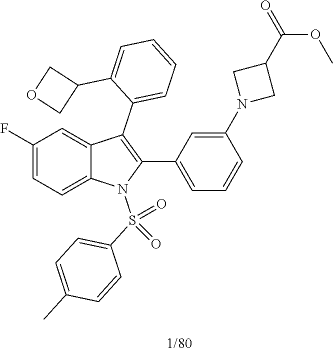 | 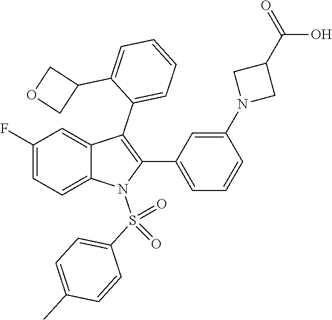 [SMILES: Cc1ccc(S(=O)(=O)n2c(-c3cccc(N4CC(C(=O)O)C4)c3)c(-c3ccccc3C3COC3)c3cc(F)ccc32)cc1] | ¹H-NMR (500 MHz, DMSO-d₆) δ: 8.27-8.23 (m, 1H), 7.62 (d, J = 7.0 Hz, 1H), 7.41 (t, J = 8.0 Hz, 1H), 7.35-7.28 (m, 5H), 7.20 (t, J = 7.0 Hz, 1H), 7.09 (t, J = 7.0 Hz, 1H), 6.87 (d, J = 7.5 Hz, 1H), 6.63-6.56 (m, 2H), 6.38 (d, J = 6.5 Hz, 1H), 6.05-6.01 (m, 1H), 4.41- 4.13 (m, 4H), 3.82- 3.79 (m, 2H), 3.70-3.66 (m, 2H), 3.59-3.55 (m, 1H), 3.43-3.39 (m, 1H), 2.33 (s, 3H); MS: 597.2 (M + 1)⁺. |
| 3/ 32 | 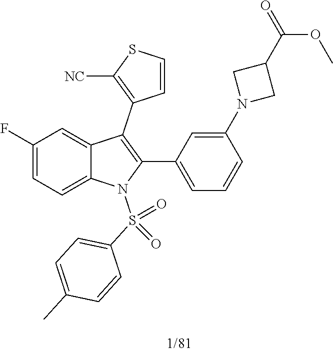 | 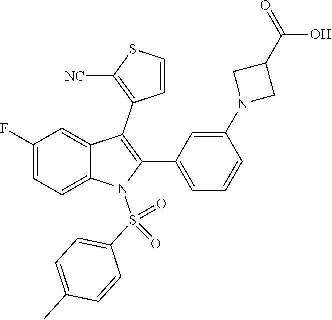 [SMILES: Cc1ccc(S(=O)(=O)n2c(-c3cccc(N4CC(C(=O)O)C4)c3)c(-c3ccsc3C#N)c3cc(F)ccc32)cc1] | ¹H-NMR (500 MHz, DMSO-d₆) δ: 8.29-8.26 (m, 1H), 7.98 (d, J = 5.1 Hz, 1H), 7.41 (d, J = 8.4 Hz, 2H), 7.37-7.31 (m 3H), 7.17-7.09 (m, 2H), 6.94 (d, J = 5.1 Hz, 1H), 6.56 (d, J = 7.6 Hz, 1H), 6.48 (dd, J = 8.1, 1.8 Hz, 1H), 6.20 (s, 1H), 3.88 (t, J = 7.8 Hz, 2H), 3.77 (t, J = 6.5 Hz, 2H), 3.47-3.43 (m, 1H), 2.33 (s, 3H); MS: 569.7 (M − 1)⁻. |
| 3/ 33 | 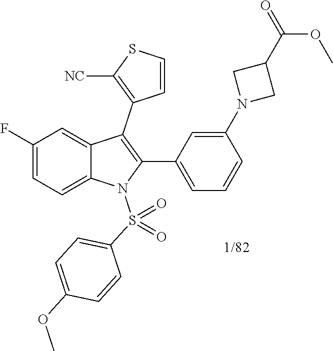 | 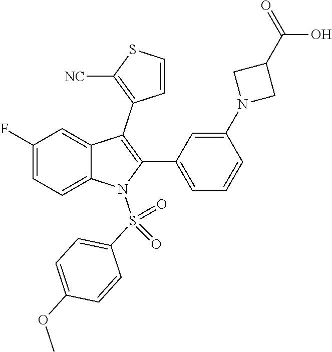 | ¹H-NMR (500 MHz, DMSO-d₆) δ: 8.30-8.26 (m, 1H), 7.98 (d, J = 5.1 Hz, 1H), 7.46 (d, J = 8.9 Hz, 2H), 7.35 (td, J = 9.2, 2.5 Hz, 1H), 7.16-7.10 (m, 2H), 7.02 (d, J = 9.0 Hz, 2H), 6.94 (d, J = 5.1 Hz, 1H), 6.56 (d, J = 7.5 Hz, 1H), 6.48 (d, J = 8.1 Hz, 1H), 6.23 (s, 1H), 3.90 (t, J = 7.9 Hz, 2H), 3.77-3.80 (m, 5H), 3.48-3.44 (m, 1H); MS: 578.8 (M + 1)⁺. |
| 3/ 34 | 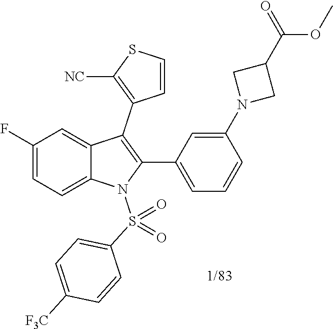 | 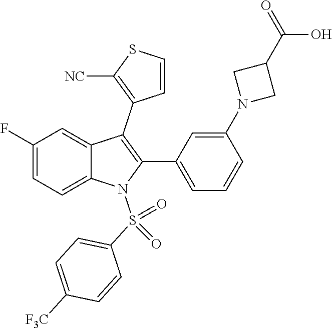 [SMILES: N#Cc1sccc1-c1c(-c2cccc(N3CC(C(=O)O)C3)c2)n(S(=O)(=O)c2ccc(C(F)(F)F)cc2)c2ccc(F)cc12] | ¹H-NMR (500 MHz, DMSO-d₆) δ: 8.31-8.28 (m, 1H), 7.99 (d, J = 5.1 Hz, 1H), 7.92 (d, J = 8.4 Hz, 2H), 7.70 (d, J = 8.3 Hz, 2H), 7.39 (td, J = 9.1, 2.5 Hz, 1H), 7.15-7.11 (m, 2H), 6.94 (d, J = 5.1 Hz, 1H), 6.54-6.47 (m, 2H), 6.24 (s, 1H), 3.89 (t, J = 7.9 Hz, 2H), 3.78 (t, J = 6.4 Hz, 2H), 3.44-3.47 (m, 1H); MS: 625.8 (M + 1)⁺. |
| 3/ 35 | 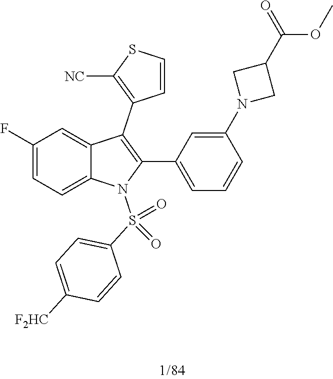 | 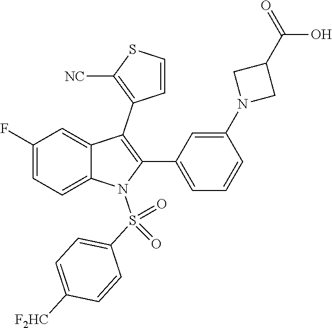 [SMILES: N#Cc1sccc1-c1c(-c2cccc(N3CC(C(=O)O)C3)c2)n(S(=O)(=O)c2ccc(C(F)F)cc2)c2ccc(F)cc12] | ¹H-NMR (500 MHz, DMSO-d₆) δ: 8.30-8.27 (m, 1H), 7.99 (d, J = 5.1 Hz, 1H), 7.73 (d, J = 8.3 Hz, 2H), 7.65 (d, J = 8.3 Hz, 2H), 7.40-7.35 (m, 1H), 7.15-7.12 (m, 2H), 7.09 (t, J = 55.5 Hz, 1H), 6.96 (d, J = 5.1 Hz, 1H), 6.55 (d, J = 7.5 Hz, 1H), 6.48 (d, J = 8.0 Hz, 1H), 6.24 (s, 1H), 3.88 (t, J = 7.9 Hz, 2H), 3.77 (t, J = 6.5 Hz, 2H), 3.42-3.48 (m, 1H); MS: 607.8 (M + 1)⁺. |
| 3/ 36 | 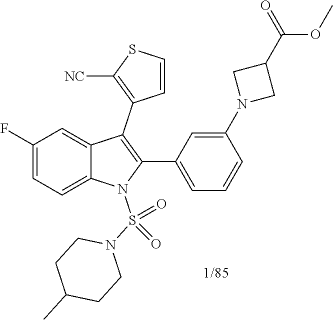 [SMILES: COC(=O)C1CN(c2cccc(-c3c(-c4ccsc4C#N)c4cc(F)ccc4n3S(=O)(=O)N3CCC(C)CC3)c2)C1] | 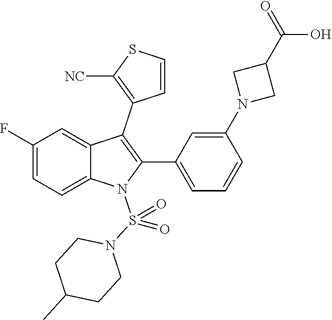 [SMILES: CC1CCN(S(=O)(=O)n2c(-c3cccc(N4CC(C(=O)O)C4)c3)c(-c3ccsc3C#N)c3cc(F)ccc32)CC1] | ¹H-NMR (500 MHz, CD₃OD) δ: 8.24-8.21 (m, 1H), 7.79 (d, J = 5.1 Hz, 1H), 7.23-7.15 (m, 2H), 7.11 (dd, J = 8.7, 2. Hz, 1H), 6.86 (d, J = 5.1 Hz, 1H), 6.77 (d, J = 7.6 Hz, 1H), 6.56-6.53 (m, 2H), 4.05- 4.01 (m, 2H), 3.94-3.90 (m, 2H), 3.50-3.56 (m, 1H), 3.47 (d, J = 12.9 Hz, 2H), 2.48 (t, J = 11.7 Hz, 2H), 1.49 (dd, J = 13.0, 2.1 Hz, |
| | | | 2H), 1.39-1.26 (m, |
| | | | 1H), 0.90-0.76 |
| | | | (m, 5H); MS: |
| | | | 579.0 (M + 1)⁺. |
| 3/ 37 | 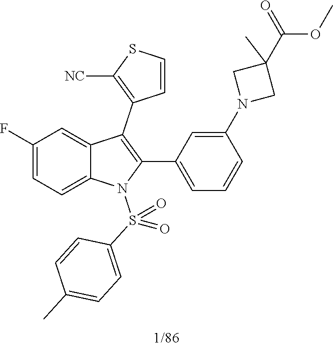 | 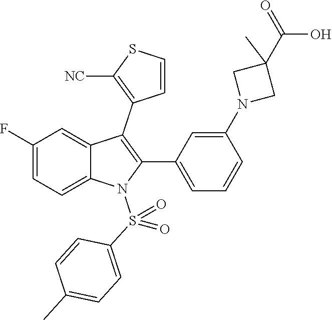 [SMILES: Cc1ccc(S(=O)(=O)n2c(-c3cccc(N4CC(C)(C(=O)O)C4)c3)c(-c3ccsc3C#N)c3cc(F)ccc32)cc1] | ¹H-NMR (500 MHz, DMSO-d₆) δ: 12.77 (s, 1H), 8.27 (dd, J = 9.2, 4.4 Hz, 1H), 7.99 (d, J = 5.1 Hz, 1H), 7.45-7.28 (m, 5H), 7.19-7.08 (m, 2H), 6.95 (d, J = 5.1 Hz, 1H), 6.59-6.57 (m, 1H), 6.51-6.47 (m, 1H), 6.21 (s, 1H), 3.92 (d, J = 7.2 Hz, 2H), 3.54 (d, J = 7.2 Hz, 2H), 2.33 (s, 3H), 1.52 (s, 3H); MS: 586.2 (M + 1)⁺. |
| 3/ 38 | 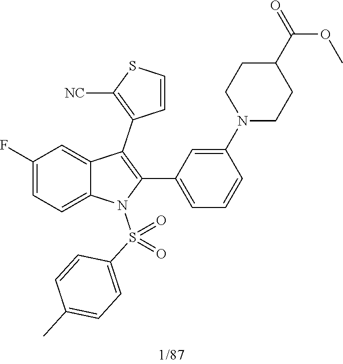 | 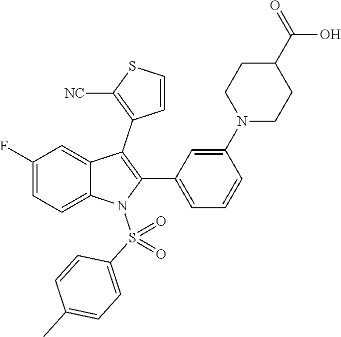 [SMILES: Cc1ccc(S(=O)(=O)n2c(-c3cccc(N4CCC(C(=O)O)CC4)c3)c(-c3ccsc3C#N)c3cc(F)ccc32)cc1] | ¹H-NMR (400 MHz, CD₃OD) δ: 8.40 (dd, J = 9.2, 4.4 Hz, 1H), 7.88 (d, J = 5.1 Hz, 1H), 7.41-7.57 (m, 3H), 7.35-7.20 (m, 5H), 7.10- 6.98 (m, 3H), 3.73- 3.69 (m, 2H), 3.47-3.41 (m, 2H), 2.80-2.67 (m, 1H), 2.36 (s, 3H), 2.26 (d, J = 14.1 Hz, 2H), 2.13-1.98 (m, 2H); MS: 600.2 (M + 1)⁺. |
| 3/ 39 | 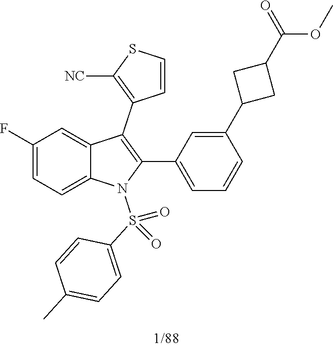 | 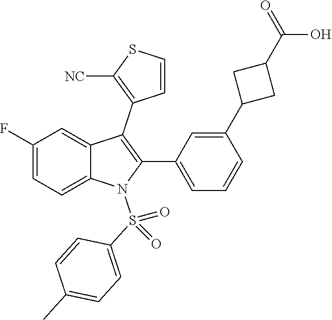 [SMILES: Cc1ccc(S(=O)(=O)n2c(-c3cccc(C4CC(C(=O)O)C4)c3)c(-c3ccsc3C#N)c3cc(F)ccc32)cc1] | ¹H-NMR (500 MHz, DMSO-d₆) δ: 12.14 (s, 1H), 8.30 (dd, J = 9.2, 4.4 Hz, 1H), 8.00- 7.97 (m, 1H), 7.43-7.20 (m, 7H), 7.17-7.08 (m, 2H), 7.06-6.91 (m, 2H), 3.62-3.29 (m, 1H), 3.10-2.92 (m, 1H), 2.50- 2.39 (m, 2H), 2.32 (s, 3H), 2.24- 2.02 (m, 2H); MS: 568.7 (M − 1)⁻. |
| 3/ 40 | 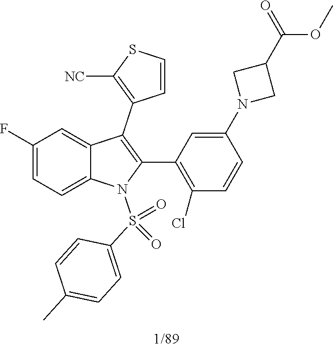 | 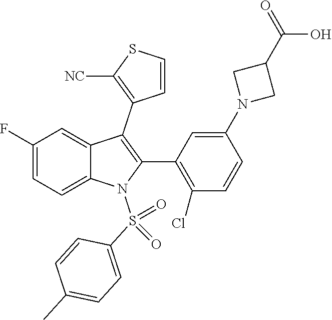 [SMILES: Cc1ccc(S(=O)(=O)n2c(-c3cc(N4CC(C(=O)O)C4)ccc3Cl)c(-c3ccsc3C#N)c3cc(F)ccc32)cc1] | ¹H-NMR (500 MHz, DMSO-d₆) δ: 12.68 (s, 1H), 8.28 (dd, J = 9.2, 4.4 Hz, 1H), 7.98 (d, J = 5.1 Hz, 1H), 7.59 (d, J = 8.4 Hz, 2H), 7.44-7.32 (m, 3H), 7.28-7.17 (m, 2H), 6.94 (d, J = 5.1 Hz, 1H), 6.54 (dd, J = 8.7, 2.8 Hz, 1H), 6.40 (d, J = 2.3 Hz, 1H), 4.02-3.93 (m, 2H), 3.90-3.77 (m, 2H), 3.59-3.48 (m, 1H), 2.36 (s, 3H); MS: 606.1 (M + 1)⁺. |
| 3/ 41 | 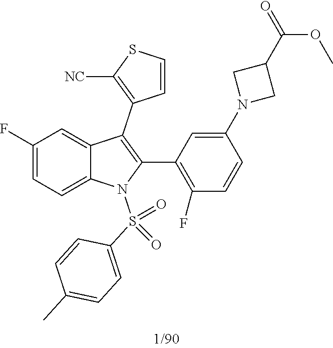 | 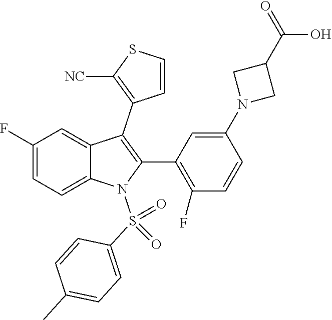 [SMILES: Cc1ccc(S(=O)(=O)n2c(-c3cc(N4CC(C(=O)O)C4)ccc3F)c(-c3ccsc3C#N)c3cc(F)ccc32)cc1] | ¹H-NMR (500 MHz, CDCl₃) δ: 8.30 (dd, J = 9.2, 4.4 Hz, 1H), 7.49 (d, J = 5.1 Hz, 1H), 7.42 (d, J = 8.3 Hz, 2H), 7.20- 7.12 (m, 3H), 7.05 (dd, J = 8.4, 2.4 Hz, 1H), 6.90 (t, J = 8.9 Hz, 1H), 6.83 (d, J = 5.1 Hz, 1H), 6.56-6.49 (m, 1H), 6.39-6.35 (m, 1H), 4.12-3.98 (m, 4H), 3.63-3.55 (m, 1H), 2.35 (s, 3H); MS: 590.1 (M + 1)⁺. |
| 3/ 42 | 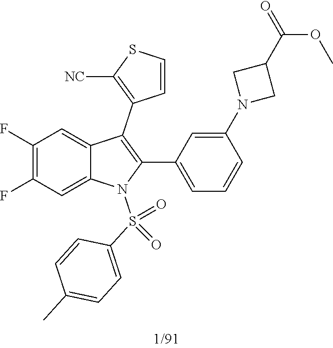 | 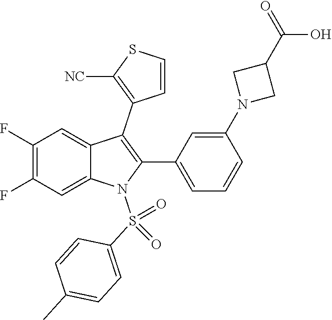 [SMILES: Cc1ccc(S(=O)(=O)n2c(-c3cccc(N4CC(C(=O)O)C4)c3)c(-c3ccsc3C#N)c3cc(F)c(F)cc32)cc1] | ¹H-NMR (500 MHz, CD₃OD) δ: 8.34-8.29 (m, 1H), 7.78 (d, J = 5.0 Hz, 1H), 7.34- 7.15 (m, 6H), 6.87 (d, J = 5.0 Hz, 1H), 6.62 (d, J = 7.5 Hz, 1H), 6.57 (dd, J = 1.5 Hz, 1.0 Hz, 1H), 6.18 (s, 1H), 4.00 (t, J = 8.0 Hz, 2H), 3.89 (t, J = 7.5 Hz, 2H), 3.58- 3.54 (m, 1H), 2.38 (s, 3H); MS: 589.7 (M + 1)⁺. |
| 3/ 43 | 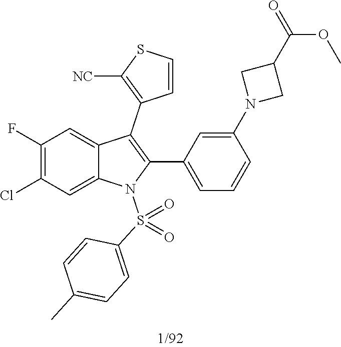 | 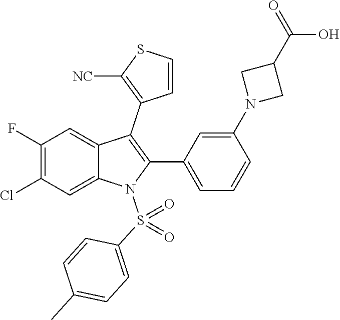 [SMILES: Cc1ccc(S(=O)(=O)n2c(-c3cccc(N4CC(C(=O)O)C4)c3)c(-c3ccsc3C#N)c3cc(F)c(Cl)cc32)cc1] | ¹H-NMR (500 MHz, DMSO-d₆) δ: 12.69 (br s, 1H), 8.40 (d, J = 8.0 Hz, 1H), 7.99 (d, J = 6.5 Hz, 1H), 7.45-7.32 (m, 5H), 7.14 (t, J = 9.5 Hz, 1H), 6.95 (d, J = 6.0 Hz, 1H), 6.55-6.48 (m, 2H), 6.15 (s, 1H), 3.89 (t, J = 9.5 Hz, 2H), 3.74 (t, J = 8.5 Hz, 2H), 3.54-3.49 (m, 1H), 2.34 (s, 3H); MS: 606.1 (M + 1)⁺. |
| 3/ 44 | 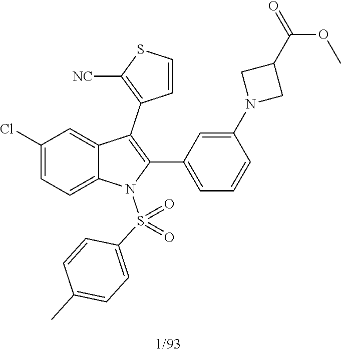 | 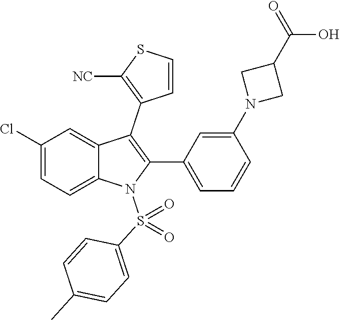 [SMILES: Cc1ccc(S(=O)(=O)n2c(-c3cccc(N4CC(C(=O)O)C4)c3)c(-c3ccsc3C#N)c3cc(Cl)ccc32)cc1] | ¹H-NMR (500 MHz, DMSO-d₆) δ: 8.28 (d, J = 9.0 Hz, 1H), 7.99 (d, J = 5.0 Hz, 1H), 7.52 (dd, J = 2.5, 2.0 Hz, 1H), 7.42 (d, J = 8.5 Hz, 2H), 7.35- 7.31 (m, 3H), 7.13 (t, J = 7.5 Hz, 1H), 6.97 (d, J = 5.0 Hz, 1H), 6.55 (d, J = 7.5 Hz, 1H), 6.50-6.46 (m, 1H), 6.19 (s, 1H), 3.89-3.86 (m, 2H), 3.78-3.74 (m, 2H), 3.45-3.40 (m, 1H), 2.33 (s, 3H); MS: 587.8 (M + 1)⁺. |
| 3/ 45 | 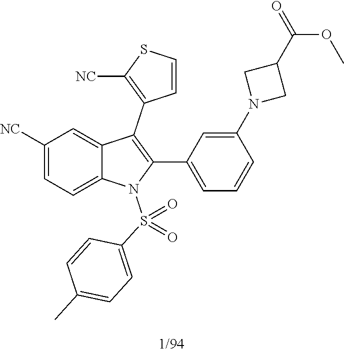 | 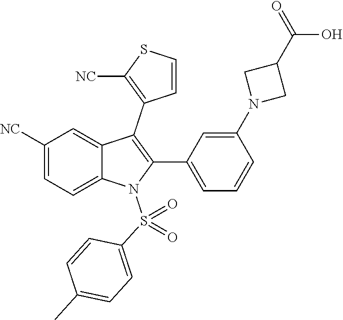 [SMILES: Cc1ccc(S(=O)(=O)n2c(-c3cccc(N4CC(C(=O)O)C4)c3)c(-c3ccsc3C#N)c3cc(C#N)ccc32)cc1] | ¹H-NMR (500 MHz, DMSO-d⁶) δ: 8.46 (d, J = 8.5 Hz, 1H), 8.00 (d, J = 5.0 Hz, 1H), 7.95-7.88 (m, 2H), 7.46 (d, J = 8.5 Hz, 2H), 7.35 (d, J = 8.5 Hz, 2H), 7.16 (t, J = 8.0 Hz, 1H), 7.01 (d, J = 5.0 Hz, 1H), 6.56 (d, J = 7.5 Hz, 1H), 6.50 (dd, J = 1.5, 2.0 Hz, 1H), 6.20 (s, 1H), 3.93-3.87 (m, 2H), 3.80-3.75 (m, 2H), 3.54-3.50 (m, 1H), 2.35 (s, 3H); MS: 579.1 (M + 1)⁺. |
| 3/ 46 | 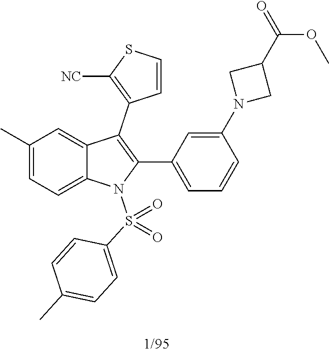 | 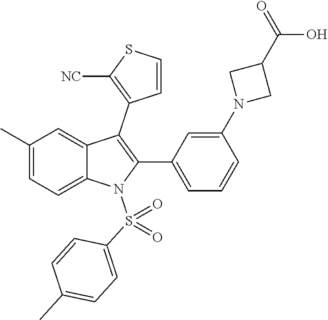 [SMILES: Cc1ccc(S(=O)(=O)n2c(-c3cccc(N4CC(C(=O)O)C4)c3)c(-c3ccsc3C#N)c3cc(C)ccc32)cc1] | ¹H-NMR (500 MHz, DMSO-d₆) δ: 8.12 (d, J = 9.0 Hz, 1H), 7.98 (d, J = 5.0 Hz, 1H), 7.42-7.28 (m, 5H), 7.13 (t, J = 7.5 Hz, 1H), 7.07 (s, 1H), 6.96 (d, J = 5.0 Hz, 1H), 6.57 (d, J = 7.5 Hz, 1H), 6.46 (dd, J = 1.5, 2.0 Hz, 1H), 6.22 (s, 1H), 3.90-3.86 (m, 2H), 3.78-3.74 (m, 2H), 3.45-3.41 (m, 1H), 2.36 (s, 3H), 2.31 (s, 3H); MS: 567.8 (M + 1)⁺. |
| 3/ 47 | 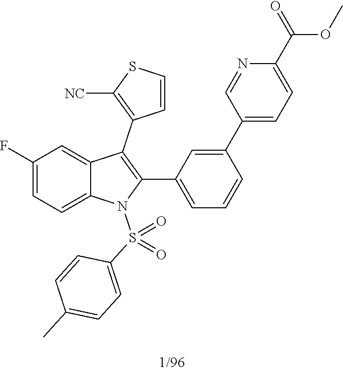 | 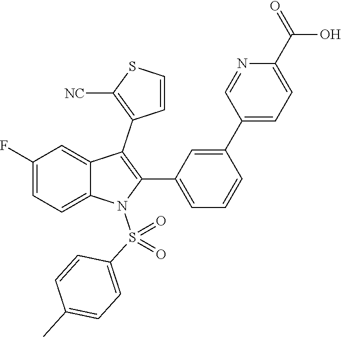 [SMILES: Cc1ccc(S(=O)(=O)n2c(-c3cccc(-c4ccc(C(=O)O)nc4)c3)c(-c3ccsc3C#N)c3cc(F)ccc32)cc1] | ¹H-NMR (500 MHz, DMSO-d₆) δ: 8.81 (s, 1H), 8.33-8.29 (m, 1H), 8.10-8.03 (m, 3H), 7.85 (d, J = 10.0 Hz, 1H), 7.58- 7.52 (m, 2H), 7.42-7.32 (m, 4H), 7.27 (d, J = 10.0 Hz, 2H), 7.21 (dd, J = 3.0, 3.5 Hz, 1H), 7.08 (d, J = 6.0 Hz, 1H), 2.25 (s, 3H); MS: 594.1 (M + 1)⁺. |
| 3/ 48 | 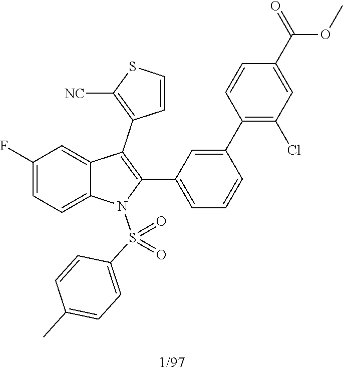 | 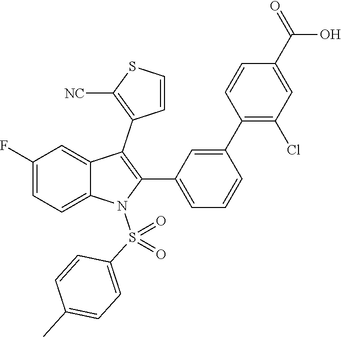 [SMILES: Cc1ccc(S(=O)(=O)n2c(-c3cccc(-c4ccc(C(=O)O)cc4Cl)c3)c(-c3ccsc3C#N)c3cc(F)ccc32)cc1] | ¹H-NMR (500 MHz, CD₃OD) δ: 8.43-8.40 (m, 1H), 8.08 (d, J = 2.0 Hz, 1H), 7.99 (dd, J = 10.0, 2.0 Hz, 1H), 7.83 (d, J = 6.5 Hz, 1H), 7.52-7.44 (m, 3H), 7.38 (d, J = 10.0 Hz, 1H), 7.30-7.25 (m, 3H), 7.15 (d, J = 10.0 Hz, 2H), 7.08- 6.96 (m, 3H), 2.24 (s, 3H); MS: 624.7 (M − 1)⁻. |
| 3/ 49 | 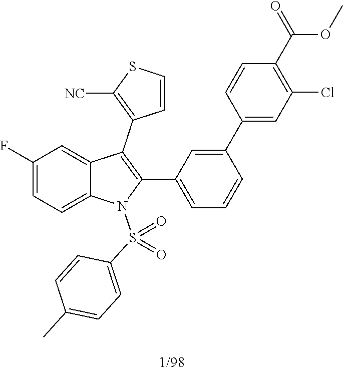 | 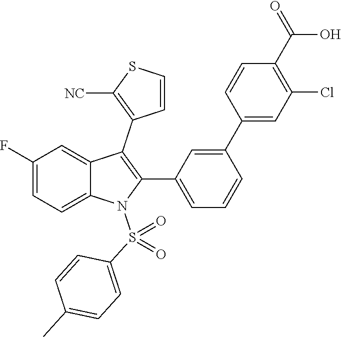 [SMILES: Cc1ccc(S(=O)(=O)n2c(-c3cccc(-c4ccc(C(=O)O)c(Cl)c4)c3)c(-c3ccsc3C#N)c3cc(F)ccc32)cc1] | ¹H-NMR (500 MHz, CD₃OD) δ: 8.44-8.40 (m, 1H), 7.88-7.83 (m, 2H), 7.72 (dd, J = 8.5, 1.5 Hz, 1H), 7.53 (d, J = 2.0 Hz, 1H), 7.51-7.45 (m, 2H), 7.34-7.27 (m, 5H), 7.18 (d, J = 8.5 Hz, 2H), 7.07 (dd, J = 8.5, 2.5 Hz, 1H), 7.00 (d, J = 5.0 Hz, 1H), 2.30 (s, 3H); MS: 624.7 (M − 1)⁻. |
| 3/ 50 | 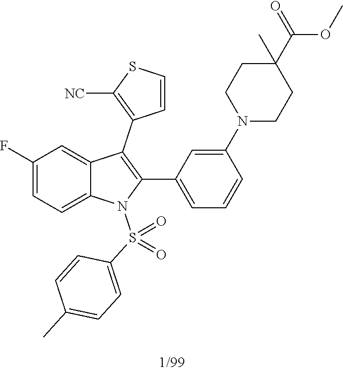 | 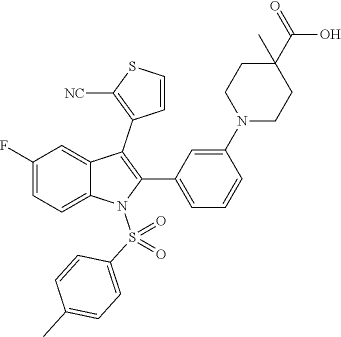 [SMILES: Cc1ccc(S(=O)(=O)n2c(-c3cccc(N4CCC(C)(C(=O)O)CC4)c3)c(-c3ccsc3C#N)c3cc(F)ccc32)cc1] | ¹H-NMR (500 MHz, DMSO-d₆) δ: 12.41 (br s, 1H), 8.29-8.25 (m, 1H), 7.98 (d, J = 5.0 Hz, 1H), 7.39-7.29 (m, 5H), 7.20-7.12 (m, 2H), 7.02-6.95 (m, 2H), 6.72-6.65 (m, 2H), 3.33- 3.30 (m, 2H), 2.83- 2.79 (m, 2H), 2.32 (s, 3H), 2.01- 1.99 (m, 2H), 1.48- 1.43 (m, 2H), 1.16 (s, 3H); MS: 614.0 (M + 1)⁺. |
| 3/ 51 | 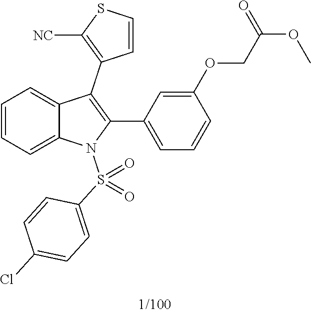 | 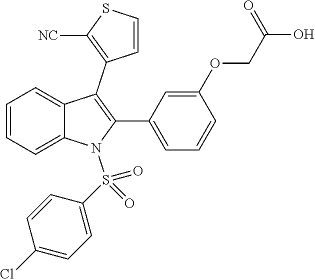 [SMILES: N#Cc1sccc1-c1c(-c2cccc(OCC(=O)O)c2)n(S(=O)(=O)c2ccc(Cl)cc2)c2ccccc12] | ¹H-NMR (500 MHz, DMSO-d₆) δ 13.03 (s, 1H), 8.26 (d, J = 8.5 Hz, 1H), 8.00 (d, J = 5.5 Hz, 1H), 7.61-7.58 (m, 2H), 7.53-7.50 (m, 3H), 7.41-7.34 (m, 2H), 7.28 (t, J = 8.0 Hz, 1H), 7.00- 6.96 (m, 2H), 6.87 (d, J = 7.5 Hz, 1H), 6.82 (s, 1H), 4.63 (s, 2H); MS: 549.0 (M + 1)⁺. |
| 3/ 52 | 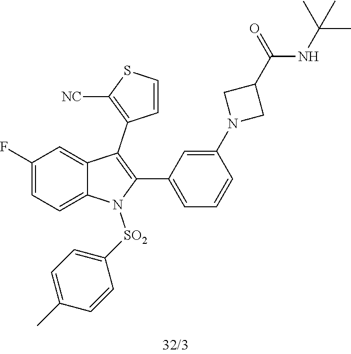 | 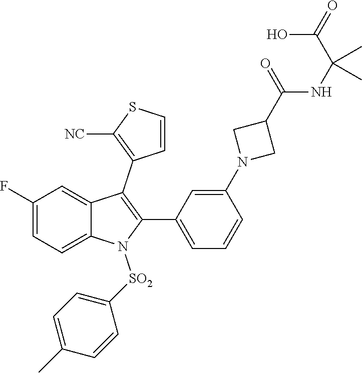 [SMILES: Cc1ccc(S(=O)(=O)n2c(-c3cccc(N4CC(C(=O)NC(C)(C)C(=O)O)C4)c3)c(-c3ccsc3C#N)c3cc(F)ccc32)cc1] | ¹H-NMR (500 MHz, DMSO-d₆) δ: 12.24 (s, 1H), 8.28 (dd, J = 9.2, 4.4 Hz, 1H), 8.19 (s, 1H), 7.98 (d, J = 5.1 Hz, 1H), 7.42 (d, J = 8.4 Hz, 2H), 7.37-7.29 (m, 3H), 7.17- 7.10 (m, 2H), 6.93 (d, J = 5.1 Hz, 1H), 6.57-6.55 (m, 1H), 6.48-6.45 (m, 1H), 6.20 (s, 1H), 3.87-3.83 (m, 2H), 3.73-3.70 (m, 2H), 3.52- 3.39 (m, 1H), 2.33 (s, 3H), 1.36 (s, 6H); MS: 657.0 (M + 1)⁺. |
| 3/ 53 | 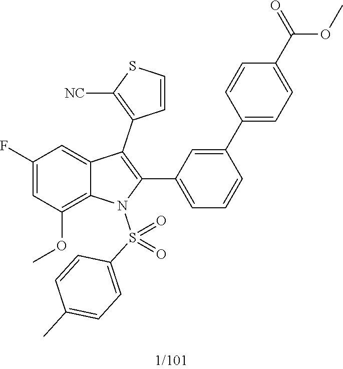 | 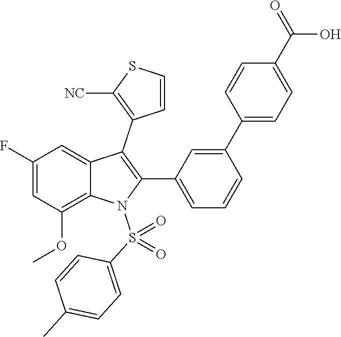 [SMILES: COc1cc(F)cc2c(-c3ccsc3C#N)c(-c3cccc(-c4ccc(C(=O)O)cc4)c3)n(S(=O)(=O)c3ccc(C)cc3)c12] | ¹H-NMR (500 MHz, DMSO-d₆) δ: 13.00 (s, 1H), 8.08 (d, J = 5.1 Hz, 1H), 8.00 (d, J = 8.3 Hz, 2H), 7.73 (d, J = 7.8 Hz, 1H), 7.61-7.56 (m, 4H), 7.50-7.45 (m, 2H), 7.33- 7.28 (m, 3H), 7.08 (d, J = 5.0 Hz, 1H), 7.00 (dd, J = 11.4, 2.1 Hz, 1H), 6.69 (dd, J = 8.0, 2.2 Hz, 1H), 3.80 (s, 1H), 2.33 (s, 3H); MS: 623.0 (M + 1)⁺. |
| 3/ 54 | 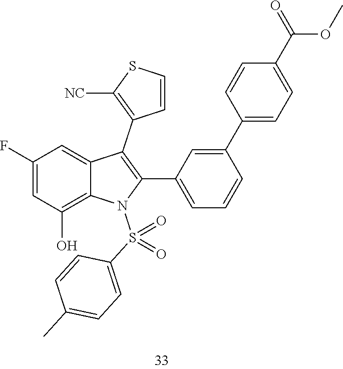 | 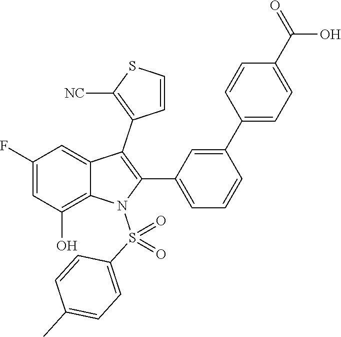 [SMILES: Cc1ccc(S(=O)(=O)n2c(-c3cccc(-c4ccc(C(=O)O)cc4)c3)c(-c3ccsc3C#N)c3cc(F)cc(O)c32)cc1] | ¹H-NMR (500 MHz, DMSO-d₆) δ: 13.02 (s, 1H), 10.68 (s, 1H), 8.08 (d, J = 5.1 Hz, 1H), 8.00 (d, J = 8.4 Hz, 2H), 7.73 (d, J = 8.0 Hz, 1H), 7.60-7.38 (m, 6H), 7.30-7.21 (m, 3H), 7.02 (d, J = 5.1 Hz, 1H), 6.67 (dd, J = 10.9, 2.3 Hz, 1H), 6.51 (dd, J = 8.1, 2.4 Hz, 1H), 2.29 (s, 3H); MS: 609.2(M + 1)⁺. |
| 3/ 55 | 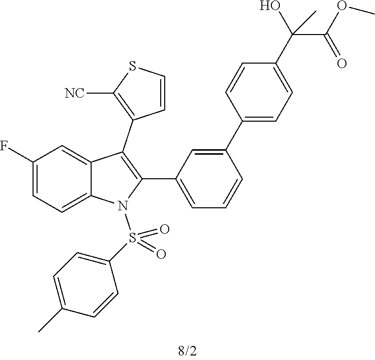 | 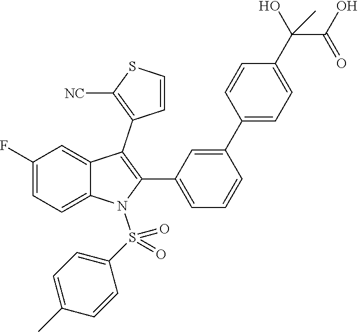 [SMILES: Cc1ccc(S(=O)(=O)n2c(-c3cccc(-c4ccc(C(C)(O)C(=O)O)cc4)c3)c(-c3ccsc3C#N)c3cc(F)ccc32)cc1] | ¹H-NMR (500 MHz, DMSO-d₆) δ: 12.74 (br s, 1H), 8.30 (dd, J = 9.0, 5.0 Hz, 1H), 8.01 (d, J = 5.0 Hz, 1H), 7.72 (d, J = 8.0 Hz, 1H), 7.59 (d, J = 8.5 Hz, 2H), 7.49-7.34 (m, 7H), 7.27- 7.16 (m, 4H), 7.05 (d, J = 5.0 Hz, 1H), 5.83 (br s, 1H), 2.24 (s, 3H), 1.65 (s, 3H); MS: 634.8 (M − 1)⁻. |
| 3/ 56 | 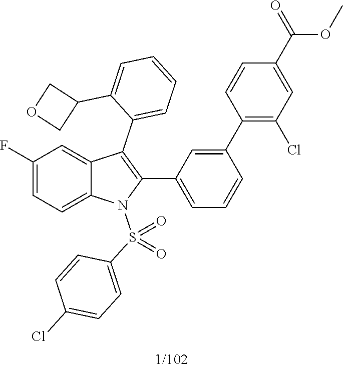 | 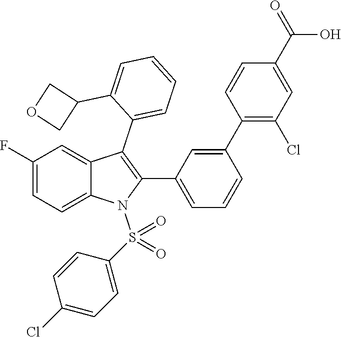 [SMILES: O=C(O)c1ccc(-c2cccc(-c3c(-c4ccccc4C4COC4)c4cc(F)ccc4n3S(=O)(=O)c3ccc(Cl)cc3)c2)c(Cl)c1] | ¹H-NMR (500 MHz, DMSO-d₆) δ: 13.39 (s, 1H), 8.27-8.24 (m, 1H), 7.97 (s, 1H), 7.93 (d, J = 7.0 Hz, 1H), 7.64-7.25 (m, 12H), 7.11 (s, 1H), 7.01 (br s, 1H), 6.75-6.73 (m, 1H), 4.42-4.37 (m, 2H), 4.17- 4.13 (m, 2H), 3.67- 3.63 (m, 1H); MS: 670.0 (M − 1)⁻. |
| 3/ 57 | 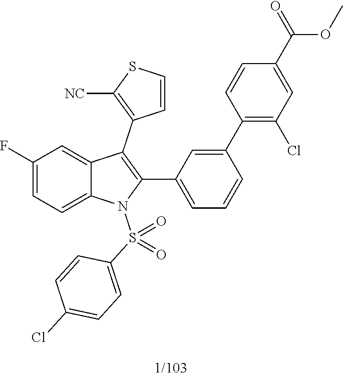 | 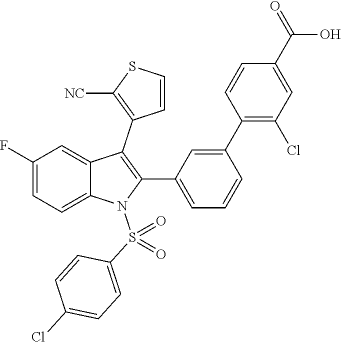 [SMILES: N#Cc1sccc1-c1c(-c2cccc(-c3ccc(C(=O)O)cc3Cl)c2)n(S(=O)(=O)c2ccc(Cl)cc2)c2ccc(F)cc12] | ¹H-NMR (500 MHz, DMSO-d₆) δ: 8.30-8.27 (m, 1H), 8.04 (d, J = 5.0 Hz, 1H), 7.96 (s, 1H), 7.93 (d, J = 8.0 Hz, 1H), 7.55-7.51 (m, 4H), 7.45-7.38 (m, 5H), 7.21-7.19 (m, 1H), 7.09 (s, 1H), 7.04 (d, J = 5.0 Hz, 1H); MS: 644.9 (M − 1)⁻. |
| 3/ 58 | 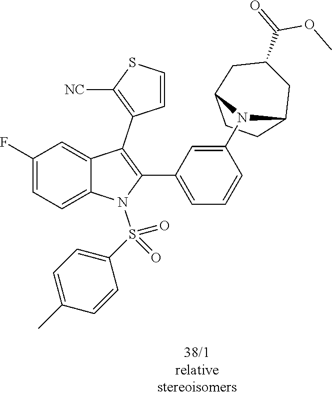 | 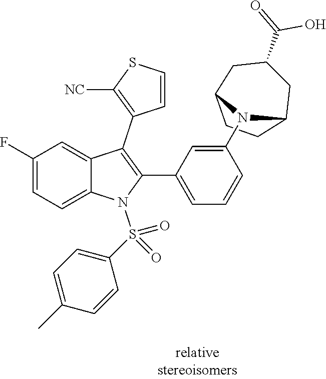 [SMILES: Cc1ccc(S(=O)(=O)n2c(-c3cccc(N4[C@@H]5CC[C@H]4C[C@@H](C(=O)O)C5)c3)c(-c3ccsc3C#N)c3cc(F)ccc32)cc1] | ¹H-NMR (500 MHz, CD₃OD) δ: 8.40 (dd, J = 9.5, 4.0 Hz, 1H), 7.71 (d, J = 5.0 Hz, 1H), 7.33 (d, J = 8.5 Hz, 2H), 7.27- 7.16 (m, 4H), 7.04-7.02 (m, 1H), 6.91-6.89 (m, 1H), 6.80 (d, J = 5.5 Hz, 1H), 6.61 (d, J = 8.0 Hz, 1H), 6.46 (s, 1H), 4.16-4.13 (m, 2H), 2.94-2.90 (m, 1H), 2.35 (s, 3H), 2.11-2.07 (m, 2H), 1.89-1.81 (m, 4H), 1.57-1.54 (m, 2H); MS: 626.2 (M + 1)⁺. |
| 3/ 59 | 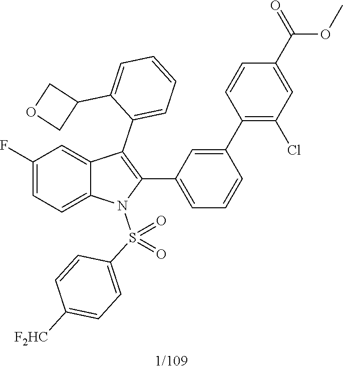 | 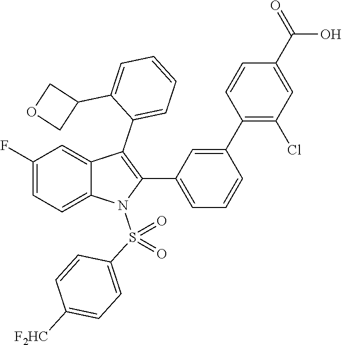 [SMILES: O=C(O)c1ccc(-c2cccc(-c3c(-c4ccccc4C4COC4)c4cc(F)ccc4n3S(=O)(=O)c3ccc(C(F)F)cc3)c2)c(Cl)c1] | ¹H-NMR (500 MHz, CD₃OD) δ: 8.41-8.38 (m, 1H), 8.07 (s, 1H), 7.97 (d, J = 7.5 Hz, 1H), 7.69 (d, J = 8.0 Hz, 1H), 7.60-7.32 (m, 8H), 7.31-7.24 (m, 3H), 7.15-6.97 (m, 2H), 6.77 (t, J = 55.5 Hz, 1H), 6.69-6.66 (m, 1H), 4.53-4.43 (m, 2H), 4.35-4.29 (m, 2H), 3.72-3.64 (m, 1H); MS: 686.0 (M − 1)⁻. |
| 3/ 60 | 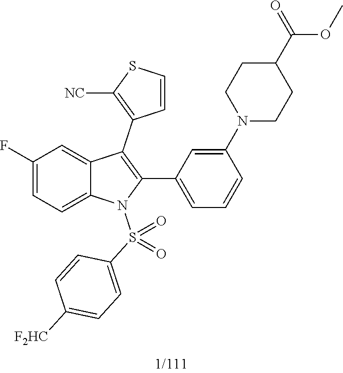 | 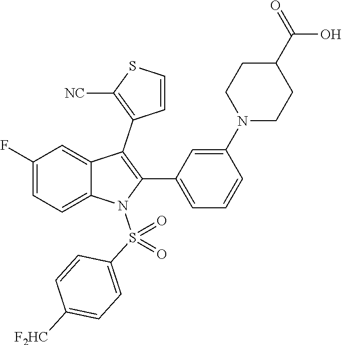 [SMILES: N#Cc1sccc1-c1c(-c2cccc(N3CCC(C(=O)O)CC3)c2)n(S(=O)(=O)c2ccc(C(F)F)cc2)c2ccc(F)cc12] | ¹H-NMR (500 MHz, CD₃OD) δ: 8.41 (dd, J = 9.2, 4.4 Hz, 1H), 7.79 (d, J = 5.1 Hz, 1H), 7.60 (d, J = 8.3 Hz, 2H), 7.53 (d, J = 8.4 Hz, 2H), 7.32-7.15 (m, 2H), 7.08-7.01 (m, 2H), 6.88 (d, J = 5.0 Hz, 1H), 6.81 (t, J = 55.0 Hz, 1H), 6.73- 6.67 (m, 2H), 3.53- 3.50 (m, 2H), 2.76-2.70 (m, 2H), 2.49-2.32 (m, 1H), 1.98-1.96 (m, 2H), 1.81-1.73 (m, 2H); MS: 634.0 (M − 1)⁻. |
| 3/ 61 | 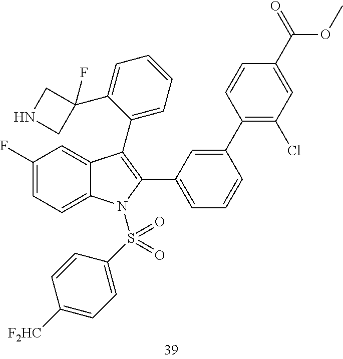 | 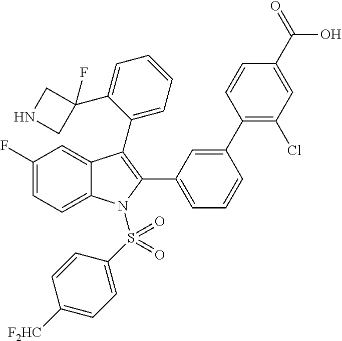 [SMILES: O=C(O)c1ccc(-c2cccc(-c3c(-c4ccccc4C4(F)CNC4)c4cc(F)ccc4n3S(=O)(=O)c3ccc(C(F)F)cc3)c2)c(Cl)c1] | ¹H-NMR (400 MHz, CD₃COCD₃) δ: 8.38-8.34 (m, 1H), 8.07 (s, 1H), 8.00 (d, J = 7.9 Hz, 1H), 7.71 (s, 4H), 7.63-7.23 (m, 9H), 7.14-6.72 (m, 3H), 3.61-3.54 (m, 2H), 3.52- 2.98 (m, 3H); MS: 705.0 (M + 1)⁺. |
| 3/ 62 | 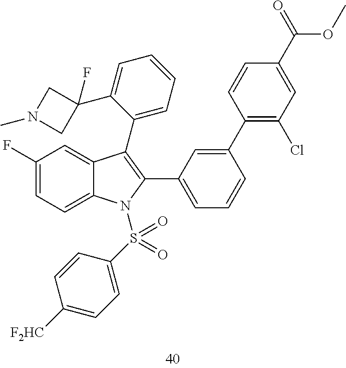 | 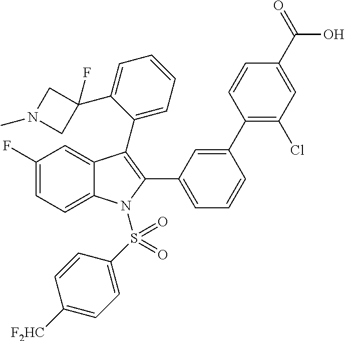 [SMILES: CN1CC(F)(c2ccccc2-c2c(-c3cccc(-c4ccc(C(=O)O)cc4Cl)c3)n(S(=O)(=O)c3ccc(C(F)F)cc3)c3ccc(F)cc23)C1] | ¹H-NMR (500 MHz, CD₃OD) δ: 8.43 (dd, J = 9.2, 4.3 Hz, 1H), 8.09 (s, 1H), 8.00 (br s, 1H), 7.76- 7.14 (m, 14H), 6.93- 6.63 (m, 2H), 4.61-3.78 (m, 4H), 2.81 (s, 3H); MS: 719.0 (M + 1)⁺. |
| 3/ 63 | 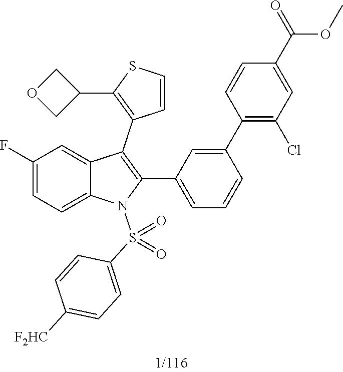 | 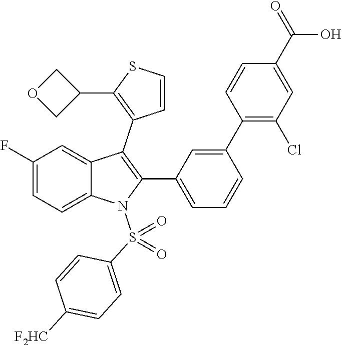 [SMILES: O=C(O)c1ccc(-c2cccc(-c3c(-c4ccsc4C4COC4)c4cc(F)ccc4n3S(=O)(=O)c3ccc(C(F)F)cc3)c2)c(Cl)c1] | ¹H-NMR (400 MHz, CD₃OD) δ: 8.40-8.36 (m, 1H), 8.10 (s, 1H), 8.00 (d, J = 5.5 Hz, 1H), 7.58-7.23 (m, 11H), 6.85-6.80 (m, 2H), 6.75 (t, J = 55.5 Hz, 1H), 4.76-4.73 (m, 1H), 4.58-4.54 (m, 1H), 4.25-4.12 (m, 1H), 4.10-4.09 (m, 1H), 3.89- 3.86 (m, 1H); MS: 691.9 (M − 1)⁻. |
| 3/ 64 | 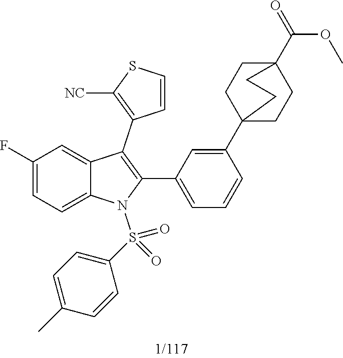 | 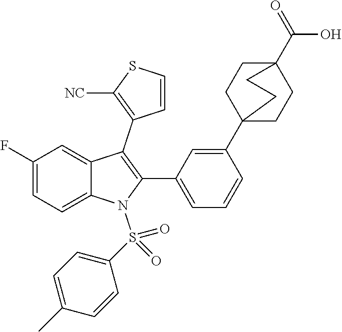 [SMILES: Cc1ccc(S(=O)(=O)n2c(-c3cccc(C45CCC(C(=O)O)(CC4)CC5)c3)c(-c3ccsc3C#N)c3cc(F)ccc32)cc1] | ¹H-NMR (500 MHz, DMSO-d₆) δ: 11.11 (br s, 1H), 8.30 (dd, J = 4.5, 9.5 Hz, 1H), 7.99 (d, J = 5.5 Hz, 1H), 7.39-6.94 (m, 11H), 2.13 (s, 3H), 1.79-1.76 (m, 6H), 1.65- 1.62 (m, 6H); MS: 622.8 (M − 1)⁻. |
| 3/ 65 | 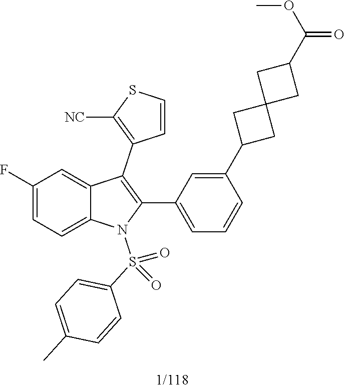 | 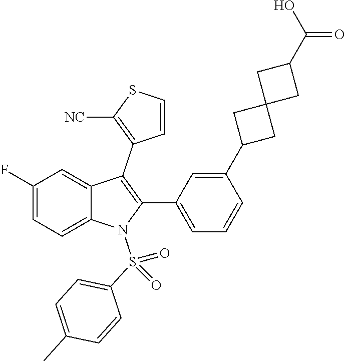 [SMILES: Cc1ccc(S(=O)(=O)n2c(-c3cccc(C4CC5(CC(C(=O)O)C5)C4)c3)c(-c3ccsc3C#N)c3cc(F)ccc32)cc1] | ¹H-NMR (500 MHz, DMSO-d₆) δ: 8.29 (dd, J = 4.3, 9.3 Hz, 1H), 7.99 (d, J = 5.0 Hz, 1H), 7.39-6.91 (m, 11H), 3.33- 3.26 (m, 1H), 2.95-2.90 (m, 1H), 2.41-1.81 (m, 11H); MS: 608.8 (M − 1)⁻. |
| 3/ 66 | 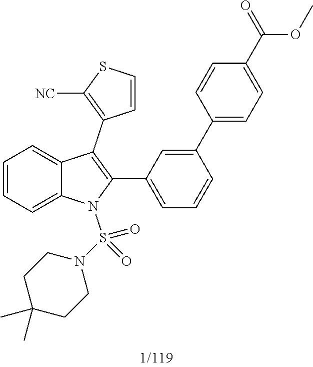 | 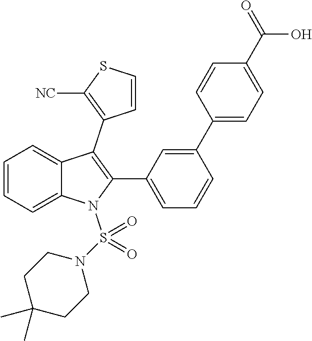 [SMILES: CC1(C)CCN(S(=O)(=O)n2c(-c3cccc(-c4ccc(C(=O)O)cc4)c3)c(-c3ccsc3C#N)c3ccccc32)CC1] | ¹H-NMR (400 MHz, CDCl₃) δ: 8.28-8.14 (m, 3H), 7.72-7.37 (m, 11H), 6.79-6.77 (m, 1H), 2.96- 2.93 (m, 4H), 1.16- 1.10 (m, 4H), 0.75 (s, 6H); MS: 593.7 (M − 1)⁻. |
| 3/ 67 | 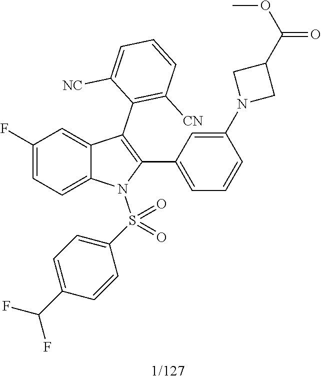 | 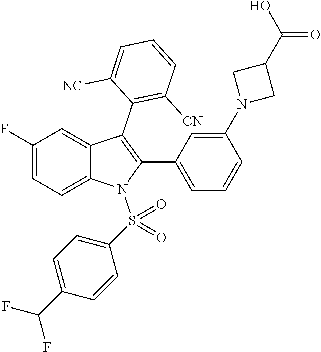 [SMILES: N#Cc1cccc(C#N)c1-c1c(-c2cccc(N3CC(C(=O)O)C3)c2)n(S(=O)(=O)c2ccc(C(F)F)cc2)c2ccc(F)cc12] | ¹H-NMR (500 MHz, CD₃OD) δ: 8.43 (dd, J = 9.3, 4.8 Hz, 1H), 8.04 (d, J = 7.5 Hz, 2H), 7.70 (t, J = 8.0 Hz, 1H), 7.61 (d, J = 9.0 Hz, 2H), 7.54 (d, J = 8.0 Hz, 2H), 7.32 (dt, J = 2.5, 9.3 Hz, 1H), 7.12 (t, J = 8.0 Hz, 1H), 6.90 (dd, J = 2.5, 8.5 Hz, 1H), 6.80 (t, J = 55.8 Hz, 1H), 6.32 (d, J = 7.5 Hz, 1H), 6.53 (dd, J = 1.5, 8.0 Hz, 1H), 6.21 (t, J = 1.8 Hz, 1H), 3.96 (br s, 2H), 3.85 (br s, 2H), 3.57-3.51 (m, 1H); MS: 727.2 (M + 1)⁺. |
| 3/ 68 | 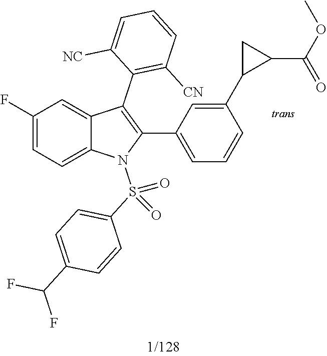 | 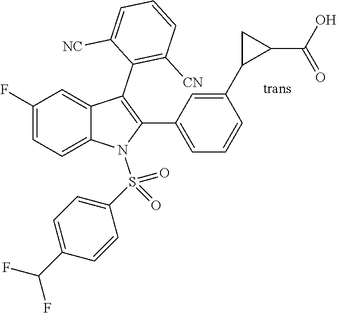 [SMILES: N#Cc1cccc(C#N)c1-c1c(-c2cccc(C3CC3C(=O)O)c2)n(S(=O)(=O)c2ccc(C(F)F)cc2)c2ccc(F)cc12] | ¹H-NMR (500 MHz, CD₃OD) δ: 8.44 (dd, J = 9.4, 4.4 Hz, 1H), 8.06-8.03 (m, 2H), 7.71 (t, J = 8.3 Hz, 1H), 7.61 (d, J = 8.0 Hz, 2H), 7.49 (d, J = 8.0 Hz, 2H), 7.35-7.21 (m, 3H), 7.05-6.84 (m, 4H), 2.42- 2.34 (m, 1H), 1.70- 1.19 (m, 3H); MS: 610.0 (M − 1)⁻. |
| 3/ 69 | 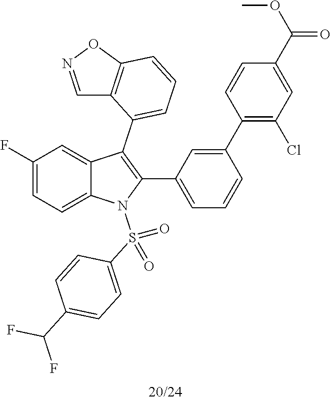 | 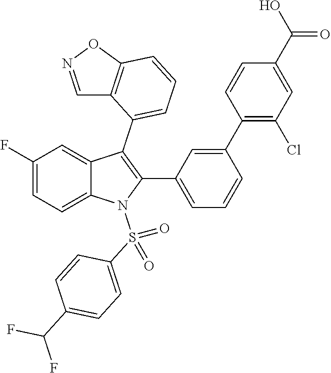 [SMILES: O=C(O)c1ccc(-c2cccc(-c3c(-c4cccc5oncc45)c4cc(F)ccc4n3S(=O)(=O)c3ccc(C(F)F)cc3)c2)c(Cl)c1] | ¹H-NMR (500 MHz, CD₃OD) δ: 8.38 (dd, J = 9.3, 4.3 Hz, 1H), 8.07 (d, J = 1.5 Hz, 1H), 7.97 (dd, J = 8.0, 1.5 Hz, 1H), 7.55-7.47 (m, 8H), 7.39 (t, J = 8.0 Hz, 1H), 7.33 (d, J = 8.0 Hz, 1H), 7.27 (dd, J = 2.5, 9.2 Hz, 1H), 7.26 (br s, 1H), 7.10-6.59 (m, 4H); MS: 670.9 (M − 1)⁻. |
| 3/ 70 | 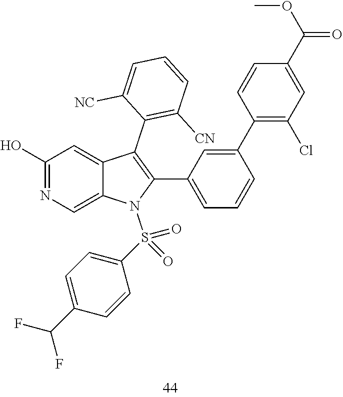 | 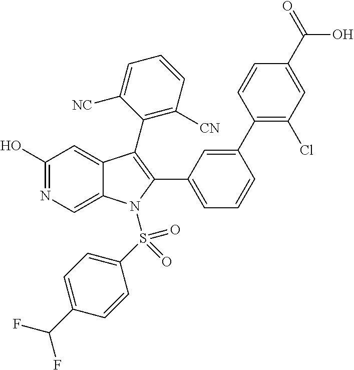 [SMILES: N#Cc1cccc(C#N)c1-c1c(-c2cccc(-c3ccc(C(=O)O)cc3Cl)c2)n(S(=O)(=O)c2ccc(C(F)F)cc2)c2cnc(O)cc12] | ¹H-NMR (500 MHz, CD₃OD) δ: 8.57 (s, 1H), 8.09- 8.07 (m, 3H), 8.00 (dd, J = 8.3, 1.8 Hz, 1H), 7.74 (t, J = 7.8 Hz, 1H), 7.61-7.43 (m, 7H), 7.36 (d, J = 8.0 Hz, 1H), 7.02 (s, 1H), 6.70 (t, J = 55.5 Hz, 1H), 6.14 (d, J = 1.0 Hz, 1H); MS: 681.1 (M + 1)⁺. |
| 3/ 71 | 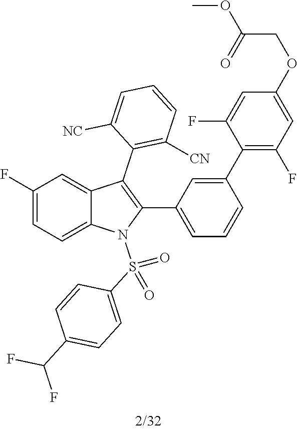 | 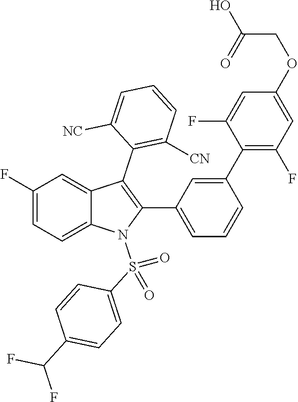 [SMILES: N#Cc1cccc(C#N)c1-c1c(-c2cccc(-c3c(F)cc(OCC(=O)O)cc3F)c2)n(S(=O)(=O)c2ccc(C(F)F)cc2)c2ccc(F)cc12] | ¹H-NMR (500 MHz, CD₃OD) δ: 8.44 (dd, J = 4.3, 9.3 Hz, 1H), 8.07-7.97 (m, 2H), 7.70 (t, J = 8.0 Hz, 1H), 7.53-7.44 (m, 7H), 7.34 (dt, J = 2.5, 9.0 Hz, 1H), 6.94-6.91 (m, 2H), 6.70-6.65 (m, 2H), 6.68 (t, J = 55.5 Hz, 1H), 4.71 (s, 2H); MS: 712.0 (M − H)⁻. |
| 3/ 72 | 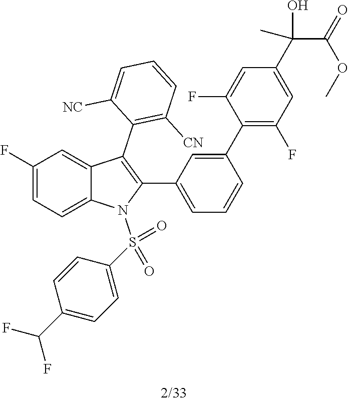 | 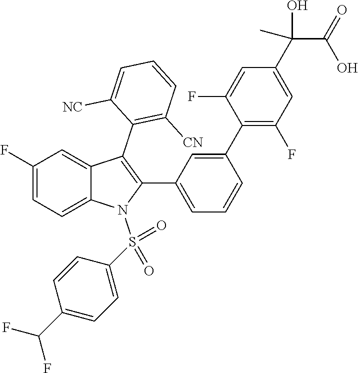 [SMILES: CC(O)(C(=O)O)c1cc(F)c(-c2cccc(-c3c(-c4c(C#N)cccc4C#N)c4cc(F)ccc4n3S(=O)(=O)c3ccc(C(F)F)cc3)c2)c(F)c1] | ¹H-NMR (500 MHz, CD₃OD) δ: 8.45 (dd, J = 9.0, 4.5 Hz, 1H), 8.17-7.92 (m, 2H), 7.71 (t, J = 8.0 Hz, 1H), 7.57-7.48 (m, 7H), 7.36- 7.29 (m, 3H), 6.95- 6.92 (m, 2H), 6.65 (t, J = 55.8 Hz, 1H), 1.75 (s, 3H); MS: 726.0 (M − H)⁻. |
| 3/ 73 | 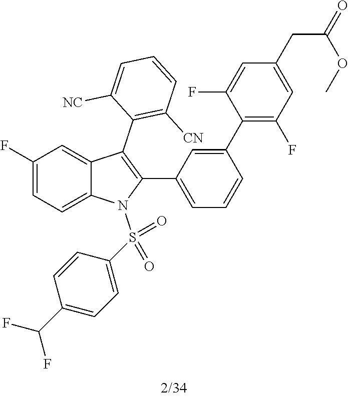 | 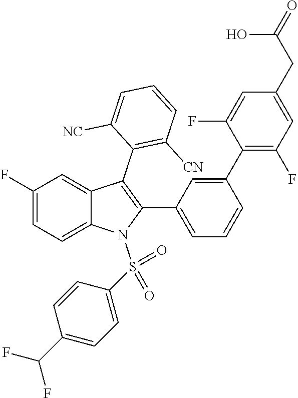 [SMILES: N#Cc1cccc(C#N)c1-c1c(-c2cccc(-c3c(F)cc(CC(=O)O)cc3F)c2)n(S(=O)(=O)c2ccc(C(F)F)cc2)c2ccc(F)cc12] | ¹H-NMR (500 MHz, CD₃OD) δ: 8.45 (dd, J = 4.3, 9.3 Hz, 1H), 8.07-8.02 (m, 2H), 7.71 (t, J = 8.0 Hz, 1H), 7.56-7.48 (m, 7H), 7.34 (dt, J = 2.5, 9.5 Hz, 1H), 7.02 (d, J = 8.5 Hz, 2H), 7.01- 6.93 (m, 2H), 6.66 (t, J = 55.5 Hz, 1H), 3.68 (s, 2H); MS: 652.0 (M − CO₂H)⁻. |

### Example Compounds Set 4

Example compounds are shown below.

| # | starting material | structure | analytical data |
|---|---|---|---|
| 4 | | 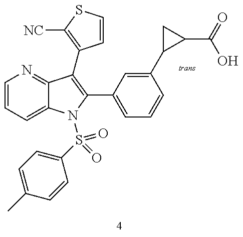 [SMILES: Cc1ccc(S(=O)(=O)n2c(-c3cccc(C4CC4C(=O)O)c3)c(-c3ccsc3C#N)c3ncccc32)cc1] | |
| 4/1 | 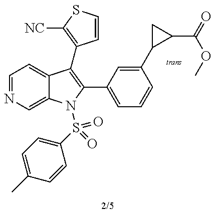 | 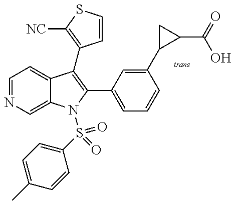 [SMILES: Cc1ccc(S(=O)(=O)n2c(-c3cccc(C4CC4C(=O)O)c3)c(-c3ccsc3C#N)c3ccncc32)cc1] | ¹H-NMR (500 MHz, CD₃OD) δ: 9.84 (s, 1H), 8.65 (d, J = 6.0 Hz, 1H), 8.03 (d, J = 6.0 Hz, 1H), 7.87 (d, J = 5.0 Hz, 1H), 7.34-7.28 (m, 6H), 7.12 (d, J = 6.0 Hz, 1H), 6.99 (d, J = 5.0 Hz, 1H), 6.71 (s, 1H), 2.42-2.36 (m, 4H), 1.76-1.73 (m, 1H), 1.54-1.51 (m, 1H), 1.23 (br s, 1H); MS: 540.1 (M + 1)⁺. |
| 4/2 | 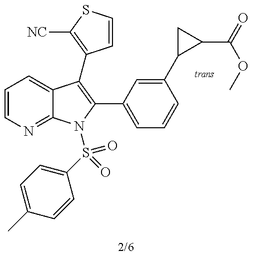 | 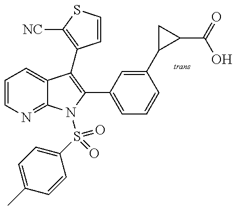 [SMILES: Cc1ccc(S(=O)(=O)n2c(-c3cccc(C4CC4C(=O)O)c3)c(-c3ccsc3C#N)c3cccnc32)cc1] | ¹H-NMR (500 MHz, CD₃OD) δ: 8.51 (dd, J = 1.5, 5.0 Hz, 1H), 7.87 (dd, J = 1.5, 8.0 Hz, 1H), 7.79 (d, J = 8.5 Hz, 1H), 7.68 (d, J = 8.5 Hz, 2H), 7.41 (dd, J = 5.3, 7.7 Hz, 1H), 7.34-7.26 (m, 4H), 7.21 (dd, J = 7.5, 1.5 Hz, 1H), 7.00 (s, 1H), 6.89 (d, J = 5.0 Hz, 1H), 2.47-2.44 (m, 1H), 2.40 (s, 3H), 1.80-1.78 (m, 1H), 1.56-1.52 (m, 1H), 1.30-1.29 (m, 1H); MS: 540.0 (M + 1)⁺. |
| 4/3 | 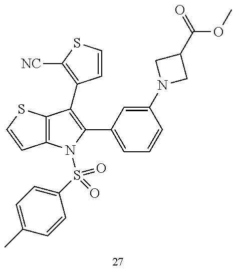 | 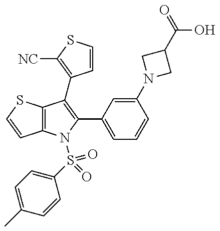 [SMILES: Cc1ccc(S(=O)(=O)n2c(-c3cccc(N4CC(C(=O)O)C4)c3)c(-c3ccsc3C#N)c3sccc32)cc1] | ¹H-NMR (500 MHz, DMSO-d₆) δ: 7.91 (d, J = 5.5 Hz, 1H), 7.68 (d, J = 5.5 Hz, 1H), 7.61-7.60 (m, 1H), 7.38-7.32 (m, 4H), 7.15 (t, J = 7.8 Hz, 1H), 6.81 (d, J = 4.5 Hz, 1H), 6.53-6.50 (m, 2H), 6.13 (d, J = 1.5 Hz, 1H), 3.90 (t, J = 7.8 Hz, 2H), 3.76 (t, J = 6.3 Hz, 2H), 3.53-3.49 (m, 1H), 2.35 (s, 3H); MS: 560.0 (M + 1)⁺. |

### Example Compounds Set 5

Example compounds are shown below.

| # | starting material | structure | analytical data |
|---|---|---|---|
| 5 | | 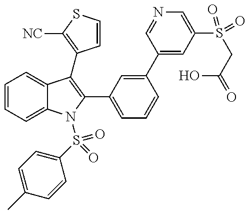 [SMILES: Cc1ccc(S(=O)(=O)n2c(-c3cccc(-c4cncc(S(=O)(=O)CC(=O)O)c4)c3)c(-c3ccsc3C#N)c3ccccc32)cc1] | |
| 5/1 | 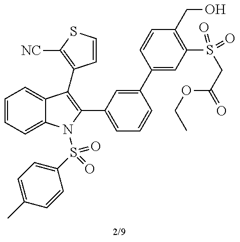 | 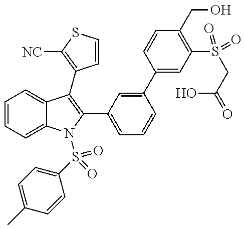 [SMILES: Cc1ccc(S(=O)(=O)n2c(-c3cccc(-c4ccc(CO)c(S(=O)(=O)CC(=O)O)c4)c3)c(-c3ccsc3C#N)c3ccccc32)cc1] | ¹H-NMR (500 MHz, DMSO-d₆) δ: 13.30 (br s, 1H), 8.30 (d, J = 8.5 Hz, 1H), 8.05-8.00 (m, 2H), 7.92- 7.86 (m, 2H), 7.77 (d, J = 8.0 Hz, 1H), 7.55-7.51 (m, 3H), 7.42-7.28 (m, 7H), 7.08 (d, J = 5.5 Hz, 1H), 4.96 (s, 2H), 4.61 (s, 2H), 2.26 (s, 3H); MS: 699.8 (M + 18)⁺. |
| 5/2 | 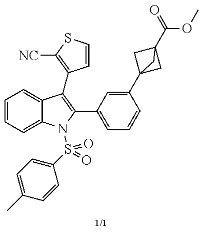 | 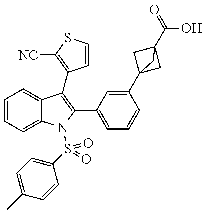 [SMILES: Cc1ccc(S(=O)(=O)n2c(-c3cccc(C45CC(C(=O)O)(C4)C5)c3)c(-c3ccsc3C#N)c3ccccc32)cc1] | ¹H-NMR (500 MHz, DMSO-d₆) δ: 12.43 (s, 1H), 8.27 (d, J = 8.5 Hz, 1H), 8.01 (d, J = 5.0 Hz, 1H), 7.53- 7.49 (m, 1H), 7.38-7.22 (m, 9H), 6.98-6.95 (m, 2H), 2.32 (s, 3H), 2.14 (s, 6H); MS: 562.8 (M − 1)⁻. |
| 5/3 | 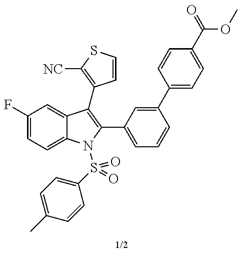 | 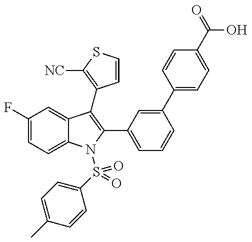 [SMILES: Cc1ccc(S(=O)(=O)n2c(-c3cccc(-c4ccc(C(=O)O)cc4)c3)c(-c3ccsc3C#N)c3cc(F)ccc32)cc1] | ¹H-NMR (500 MHz, DMSO-d₆) δ: 13.05 (s, 1H), 8.31 (dd, J = 9.2, 4.4 Hz, 1H), 8.04-8.02 (m, 3H), 7.80 (d, J = 7.7 Hz, 1H), 7.68 (d, J = 8.3 Hz, 2H), 7.56-7.47 (m, 2H), 7.44- 7.33 (m, 3H), 7.30-7.26 (m, 3H), 7.19 (dd, J = 8.6, 2.5 Hz, 1H), 7.07 (d, J = 5.1 Hz, 1H), 2.25 (s, 3H); MS: m/z 590.6 (M − 1)⁻. |
| 5/4 | 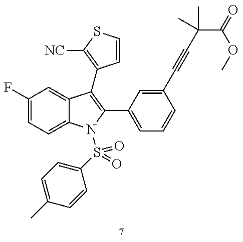 | 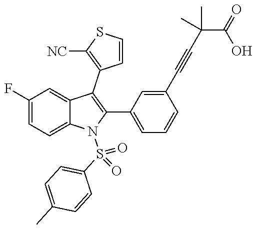 [SMILES: Cc1ccc(S(=O)(=O)n2c(-c3cccc(C#CC(C)(C)C(=O)O)c3)c(-c3ccsc3C#N)c3cc(F)ccc32)cc1] | ¹H-NMR (500 MHz, DMSO-d₆) δ: 12.91 (br s, 1H), 8.27 (dd, J = 9.3, 4.8 Hz, 1H), 8.00 (d, J = 5.0 Hz, 1H), 7.45-7.33 (m, 7H), 7.22-7.17 (m, 3H), 6.97 (d, J = 5.0 Hz, 1H), 2.33 (s, 3H), 1.48 (s, 6H); MS: 583.1 (M + 1)⁺. |
| 5/5 | 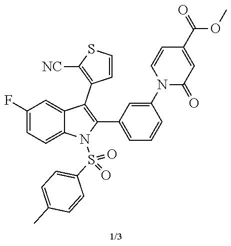 | 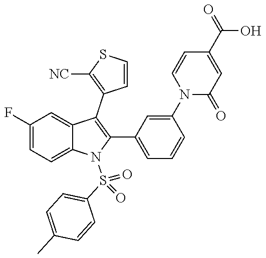 [SMILES: Cc1ccc(S(=O)(=O)n2c(-c3cccc(-n4ccc(C(=O)O)cc4=O)c3)c(-c3ccsc3C#N)c3cc(F)ccc32)cc1] | ¹H-NMR (500 MHz, CD₃OD) δ: 8.41 (dd, J = 9.0, 4.5 Hz, 1H), 7.85 (d, J = 5.0 Hz, 1H), 7.64 (d, J = 7.5 Hz, 1H), 7.57-7.50 (m, 2H), 7.39 (d, J = 8.0 Hz, 2H), 7.32-7.23 (m, 6H), 7.07 (dd, J = 8.5, 2.5 Hz, 1H), 7.02 (d, J = 5.0 Hz, 1H), 6.90 (dd, J = 7.3, 1.8 Hz, 1H), 2.33 (s, 3H); MS: 609.9 (M + 1)⁺. |
| 5/6 | 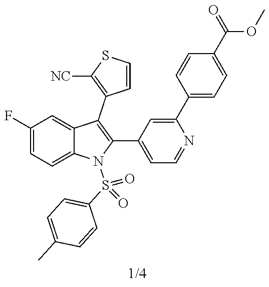 | 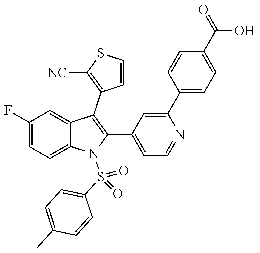 [SMILES: Cc1ccc(S(=O)(=O)n2c(-c3ccnc(-c4ccc(C(=O)O)cc4)c3)c(-c3ccsc3C#N)c3cc(F)ccc32)cc1] | ¹H-NMR (500 MHz, DMSO-d₆) δ: 8.73 (d, J = 5.5 Hz, 1H), 8.30 (dd, J = 9.3, 4.3 Hz, 1H), 8.08-8.04 (m, 5H), 7.92 (s, 1H), 7.48-7.40 (m, 3H), 7.33-7.30 (m, 3H), 7.23 (dd, J = 8.5, 2.0 Hz, 1H), 7.12 (d, J = 5.0 Hz, 1H), 2.29 (s, 3H); MS: 594.1 (M + 1)⁺. |
| 5/7 | 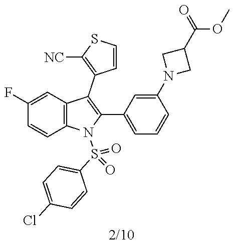 | 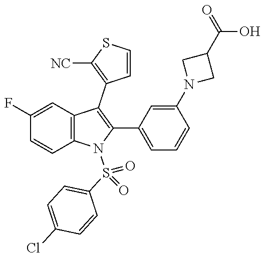 [SMILES: N#Cc1sccc1-c1c(-c2cccc(N3CC(C(=O)O)C3)c2)n(S(=O)(=O)c2ccc(Cl)cc2)c2ccc(F)cc12] | ¹H-NMR (500 MHz, DMSO-d₆) δ: 8.28 (dd, J = 9.0, 4.5 Hz, 1H), 7.99 (d, J = 4.5 Hz, 1H), 7.61 (dd, J = 6.8, 2.3 Hz, 2H), 7.50 (dd, J = 6.8, 1.8 Hz, 2H), 7.39-7.35 (m, 1H), 7.16-7.12 (m, 2H), 6.95 (d, J = 5.0 Hz, 1H), 6.54 (d, J = 7.5 Hz, 1H), 6.48 (dd, J = 8.3, 1.8 Hz, 1H), 6.23 (s, 1H), 3.89 (t, J = 7.8 Hz, 2H), 3.79 (t, J = 6.3 Hz, 2H), 3.48-3.43 (m, 1H); MS: 589.6 (M − 1)⁻. |
| 5/8 | 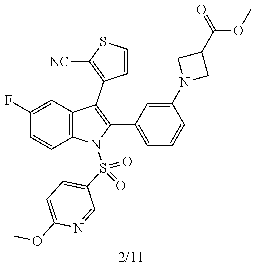 | 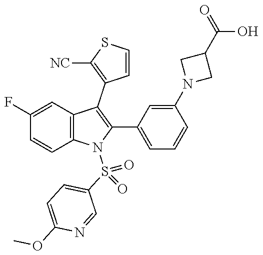 [SMILES: COc1ccc(S(=O)(=O)n2c(-c3cccc(N4CC(C(=O)O)C4)c3)c(-c3ccsc3C#N)c3cc(F)ccc32)cn1] | ¹H-NMR (500 MHz, DMSO-d₆) δ: 8.32-8.27 (m, 2H), 7.99 (d, J = 5.0 Hz, 1H), 7.68 (dd, J = 8.8, 2.8 Hz, 1H), 7.39-7.35 (m, 1H), 7.17-7.13 (m, 2H), 6.96-6.91 (m, 2H), 6.55 (d, J = 7.5 Hz, 1H), 6.48 (dd, J = 8.0, 2.0 Hz, 1H), 6.23 (s, 1H), 3.90- 3.87 (m, 5H), 3.79 (t, J = 6.5 Hz, 2H), 3.46-3.43 (m, 1H); MS: 586.7 (M − 1)⁻. |
| 5/9 | 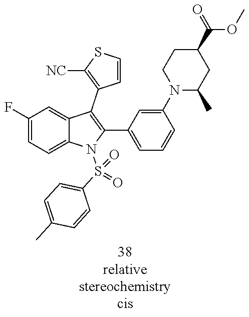 | 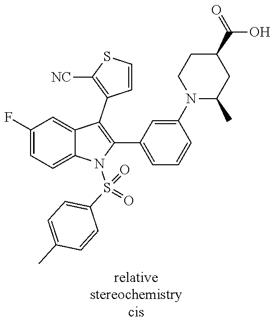 [SMILES: Cc1ccc(S(=O)(=O)n2c(-c3cccc(N4CC[C@@H](C(=O)O)C[C@H]4C)c3)c(-c3ccsc3C#N)c3cc(F)ccc32)cc1] | ¹H-NMR (500 MHz, CD₃OD) δ: 8.41-8.38 (m, 1H), 7.80 (d, J = 5.0 Hz, 1H) 7.34-7.21 (m, 7H), 7.09 (d, J = 7.0 Hz, 1H), 7.04-7.02 (m, 1H), 6.97 (s, 1H), 6.90 (d, J = 5.0 Hz, 1H), 3.11-3.07 (m, 1H), 2.96-2.94 (m, 1H), 2.79-2.74 (m, 1H), 2.50- 2.44 (m, 1H), 2.36 (s, 3H), 2.08- 1.98 (m, 2H), 1.87-1.82 (m, 1H), 1.59-1.51 (m, 1H), 0.86 (d, J = 6.0 Hz, 3H); MS: 614.0 (M + 1)⁺. |
| 5/10 | 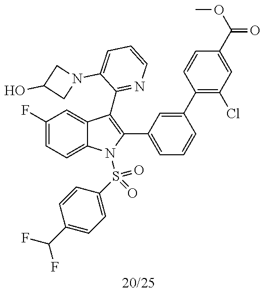 | 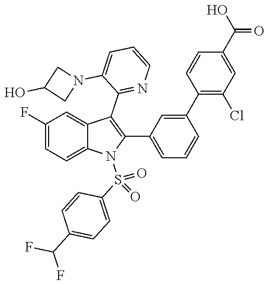 [SMILES: O=C(O)c1ccc(-c2cccc(-c3c(-c4ncccc4N4CC(O)C4)c4cc(F)ccc4n3S(=O)(=O)c3ccc(C(F)F)cc3)c2)c(Cl)c1] | ¹H-NMR (500 MHz, CD₃OD) δ: 8.53 (dd, J = 9.2, 4.3 Hz, 1H), 8.12 (s, 1H), 8.06-8.04 (m, 1H), 7.99- 7.97 (m, 1H), 7.70-7.14 (m, 13H), 6.77 (t, J = 55.5 Hz, 1H), 4.37-4.27 (m, 1H), 3.78-3.36 (m, 3H), 3.24- 3.20 (m, 1H); MS: 704.1 (M + 1)⁺. |

### Example Compounds Set 6

Example compounds are shown below.

| # | starting material | structure | analytical data |
|---|---|---|---|
| 6 | | 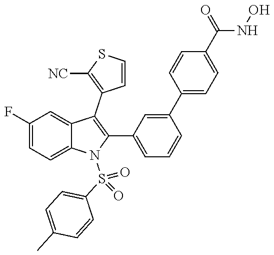 [SMILES: Cc1ccc(S(=O)(=O)n2c(-c3cccc(-c4ccc(C(=O)NO)cc4)c3)c(-c3ccsc3C#N)c3cc(F)ccc32)cc1] | |
| 6/1 | 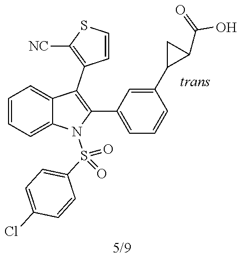 [SMILES: N#Cc1sccc1-c1c(-c2cccc(C3CC3C(=O)O)c2)n(S(=O)(=O)c2ccc(Cl)cc2)c2ccccc12] | 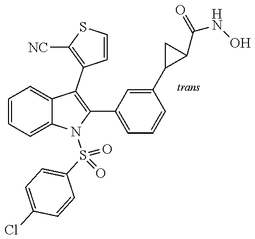 [SMILES: N#Cc1sccc1-c1c(-c2cccc(C3CC3C(=O)NO)c2)n(S(=O)(=O)c2ccc(Cl)cc2)c2ccccc12] | ¹H-NMR (500 MHz, DMSO-d₆) δ: 8.39 (d, J = 8.5 Hz, 1H), 7.82 (d, J = 5.5 Hz, 1H), 7.53-7.23 (m, 9H), 7.04-7.01 (m, 2H), 6.49 (d, J = 5.0 Hz, 1H), 2.43-2.38 (m, 1H), 1.76-1.72 (m, 1H), 1.54-1.50 (m, 1H), 1.25-1.20 (m, 1H); MS: 574.1 (M + 1)⁺. |
| 6/2 | 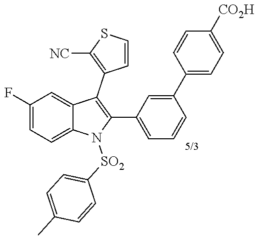 | 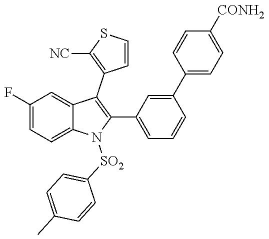 [SMILES: Cc1ccc(S(=O)(=O)n2c(-c3cccc(-c4ccc(C(N)=O)cc4)c3)c(-c3ccsc3C#N)c3cc(F)ccc32)cc1] | ¹H-NMR (500 MHz, DMSO-d₆) δ: 8.29 (dd, J = 9.0, 4.0 Hz, 1H), 8.04-7.96 (m, 4H), 7.78 (d, J = 8.5 Hz, 1H), 7.61 (d, J = 8.5 Hz, 2H), 7.50-7.36 (m, 6H), 7.26 (d, J = 8.0 Hz, 2H), 7.17 (dd, J = 8.0, 2.5 Hz, 1H), 7.06 (d, J = 5.0 Hz, 1H), 2.25 (s, 3 H); MS: 589.8 (M − 1)⁻. |
| 6/3 | 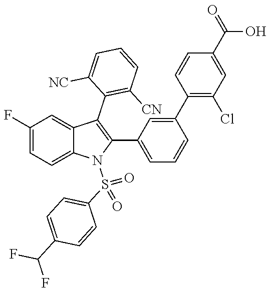 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12] pre-6/3 | 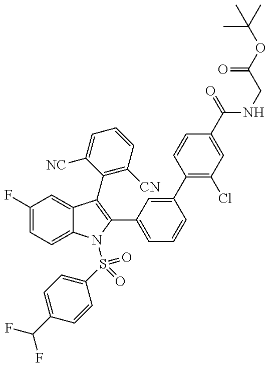 [SMILES: CC(C)(C)OC(=O)CNC(=O)c1ccc(-c2cccc(-c3c(-c4c(C#N)cccc4C#N)c4cc(F)ccc4n3S(=O)(=O)c3ccc(C(F)F)cc3)c2)c(Cl)c1] | ¹H-NMR (400 MHz, CD₃OD) δ: 8.43 (dd, J = 5.5, 9.2 Hz, 1H), 8.05 (d, J = 7.6 Hz, 2H), 7.96 (d, J = 1.2 Hz, 1H), 7.84 (dd, J = 1.8, 7.8 Hz, 1H), 7.71 (t, J = 8.0 Hz, 1H), 7.54-7.30 (m, 9H), 7.09 (s, 1H), 6.94 (dd, J = 2.4, 8.4 Hz, 1H), 6.67 (t, J = 55.4 Hz, 1H), 4.03 (s, 2H), 1.51 (s, 9H); MS: 793.1 (M − 1)⁻. |

### Example Compounds Set 8

Example compounds are shown below.

| # | starting material(s) | | structure | analytical data |
|---|---|---|---|---|
| 8 | | | 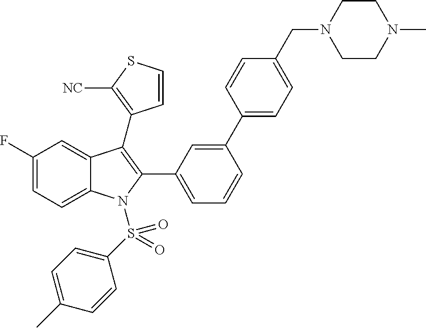 [SMILES: Cc1ccc(S(=O)(=O)n2c(-c3cccc(-c4ccc(CN5CCN(C)CC5)cc4)c3)c(-c3ccsc3C#N)c3cc(F)ccc32)cc1] | |
| 8/1 | 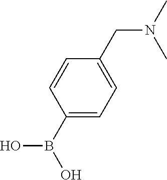 | | 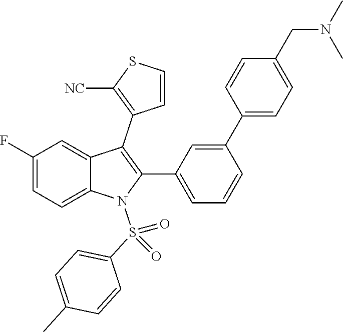 [SMILES: Cc1ccc(S(=O)(=O)n2c(-c3cccc(-c4ccc(CN(C)C)cc4)c3)c(-c3ccsc3C#N)c3cc(F)ccc32)cc1] | ¹H-NMR (500 MHz, DMSO-d₆) δ: 8.31 (dd, J = 4.5, 9.0 Hz, 1H), 8.02 (d, J = 5.0 Hz, 1H), 7.72 (d, J = 8.0 Hz, 1H), 7.49-7.35 (m, 9H), 7.27-7.17 (m, 4H), 7.06 (d, J = 5.0 Hz, 1H), 3.43 (s, 2H), 2.25 (s, 3H), 2.17 (s, 6H); MS: 606.0 (M + 1)⁺. |
| 8/3 | 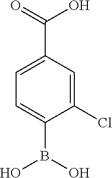 [SMILES: O=C(O)c1ccc(B(O)O)c(Cl)c1] | 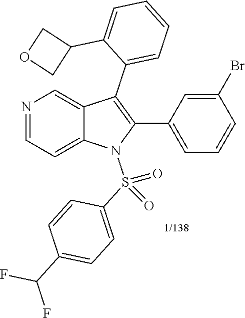 [SMILES: O=S(=O)(c1ccc(C(F)F)cc1)n1c(-c2cccc(Br)c2)c(-c2ccccc2C2COC2)c2cnccc21] | 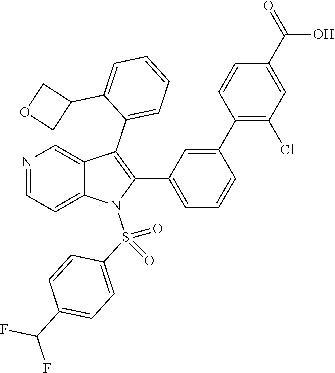 [SMILES: O=C(O)c1ccc(-c2cccc(-c3c(-c4ccccc4C4COC4)c4cnccc4n3S(=O)(=O)c3ccc(C(F)F)cc3)c2)c(Cl)c1] | ¹H-NMR (500 MHz, CD₃OD): δ: 8.60 (d, J = 5.9 Hz, 1H), 8.43 (d, J = 6.0 Hz, 1H), 8.37 (s, 1H), 8.06 (s, 1H), 7.99- 7.97 (m, 1H), 7.76-7.08 (m, 13H), 6.74 (t, J = 55.5 Hz, 1H), 4.65-4.56 (m, 1H), 4.53-4.49 (m, 1H), 4.41-4.37 (m, 1H), 4.30-4.26 (m, 1H), 3.9-3.78 (m, 1H); MS: 671.1 (M + 1)⁺. |
| 8/7 | 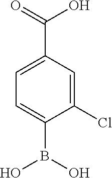 [SMILES: O=C(O)c1ccc(B(O)O)c(Cl)c1] | 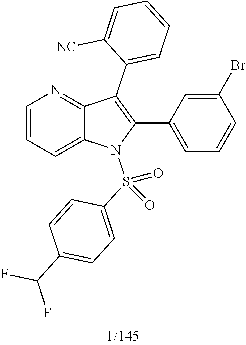 [SMILES: N#Cc1ccccc1-c1c(-c2cccc(Br)c2)n(S(=O)(=O)c2ccc(C(F)F)cc2)c2cccnc12] | 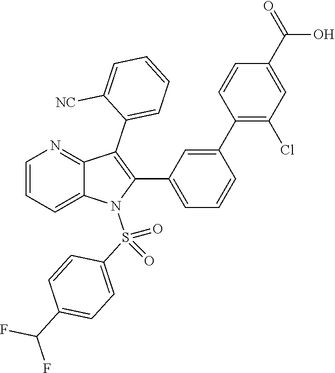 [SMILES: N#Cc1ccccc1-c1c(-c2cccc(-c3ccc(C(=O)O)cc3Cl)c2)n(S(=O)(=O)c2ccc(C(F)F)cc2)c2cccnc12] | ¹H-NMR (500 MHz, CD₃OD) δ: 8.81 (dd, J = 8.5, 1.5 Hz, 1H), 8.56 (dd, J = 4.8, 1.3 Hz, 1H), 8.06 (d, J = 1.5 Hz, 1H), 7.98 (d, J = 8.0 Hz, 1H), 7.77 (d, J = 7.5 Hz, 1H), 7.65-7.04 (m, 13H), 6.70 (t, J = 55.5 Hz, 1H); MS: 640.2 (M + 1)⁺. |
| 8/8 | 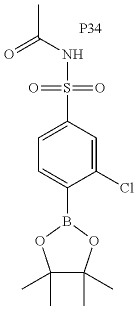 | 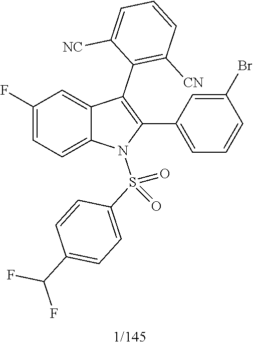 [SMILES: N#Cc1cccc(C#N)c1-c1c(-c2cccc(Br)c2)n(S(=O)(=O)c2ccc(C(F)F)cc2)c2ccc(F)cc12] | 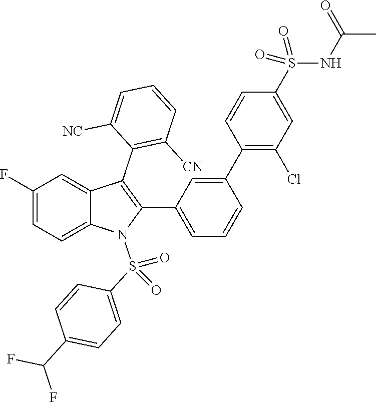 [SMILES: CC(=O)NS(=O)(=O)c1ccc(-c2cccc(-c3c(-c4c(C#N)cccc4C#N)c4cc(F)ccc4n3S(=O)(=O)c3ccc(C(F)F)cc3)c2)c(Cl)c1] | ¹H-NMR (400 MHz, CD₃OD) δ: 8.42 (dd, J = 9.4, 4.2 Hz, 1H), 8.05- 8.03 (m, 3H), 7.96 (dd, J = 8.2, 1.8 Hz, 1H), 7.70 (dd, J = 8.4, 7.6 Hz, 1H), 7.51-7.44 (m, 8H), 7.32 (td, J = 9.2, 2.4 Hz, 1H), 6.97 (s, 1H), 6.94 (dd, J = 8.4, 2.4 Hz, 1H), 6.62 (t, J = 55.4 Hz, 1H), 2.00 (s, 3H); MS: 757.0 (M − 1)⁻. |
| | | 1/139 | | |
| | | Pd(dppf)Cl₂ | | |
| | | K₂CO₃, 95° C., 8 h | | |
| | | in dioxane/water | | |
| | | (17:1) | | |

### Example Compounds Set 10

Example compounds are shown below.

| # | starting material | structure | analytical data |
|---|---|---|---|
| 10 | | 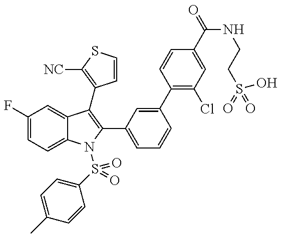 [SMILES: Cc1ccc(S(=O)(=O)n2c(-c3cccc(-c4ccc(C(=O)NCCS(=O)(=O)O)cc4Cl)c3)c(-c3ccsc3C#N)c3cc(F)ccc32)cc1] | |
| 10/1 | 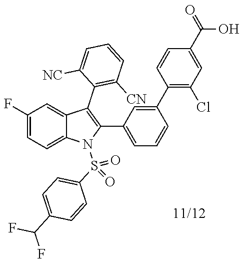 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12] | 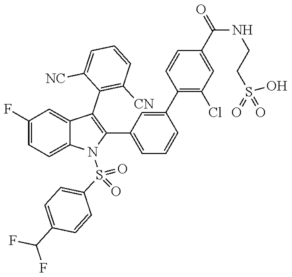 [SMILES: N#Cc1cccc(C#N)c1-c1c(-c2cccc(-c3ccc(C(=O)NCCS(=O)(=O)O)cc3Cl)c2)n(S(=O)(=O)c2ccc(C(F)F)cc2)c2ccc(F)cc12] | ¹H-NMR (500 MHz, CD₃OD) δ: 8.45-8.43 (m, 1H), 8.07 (br s, 2H), 7.94 (d, J = 2.0 Hz, 1H), 7.82 (dd, J = 8.0, 1.5 Hz, 1H), 7.78 (t, J = 7.8 Hz, 1H), 7.55-7.43 (m, 7H), 7.36-7.28 (m, 2H), 7.04 (s, 1H), 6.95 (dd, J = 8.5, 2.5 Hz, 1H), 6.68 (t, J = 55.5 Hz, 1H), 3.84 (t, J = 6.5 Hz, 2H), 3.13 (t, J = 6.5 Hz, 2H); MS: 786.9 (M − 1)⁻. |
| 10/2 | 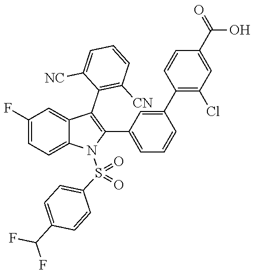 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12] | 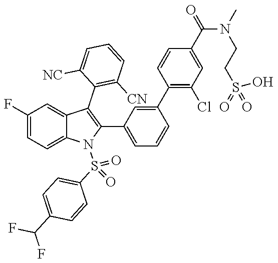 [SMILES: CN(CCS(=O)(=O)O)C(=O)c1ccc(-c2cccc(-c3c(-c4c(C#N)cccc4C#N)c4cc(F)ccc4n3S(=O)(=O)c3ccc(C(F)F)cc3)c2)c(Cl)c1] | ¹H-NMR (500 MHz, CD₃OD) δ: 8.44 (dd, J = 9.3, 4.3 Hz, 1H), 8.06 (br s, 2H), 7.72 (t, J = 7.8 Hz, 1H), 7.59-7.47 (m, 9H), 7.35-7.32 (m, 2H), 7.02-6.58 (m, 3H), 3.97- 3.77 (m, 2H), 3.24-3.10 (m, 5H); MS: 801.0 (M − 1)⁻. |
| 10/3 | 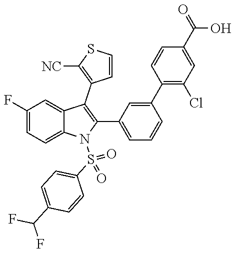 [SMILES: N#Cc1sccc1-c1c(-c2cccc(-c3ccc(C(=O)O)cc3Cl)c2)n(S(=O)(=O)c2ccc(C(F)F)cc2)c2ccc(F)cc12] | 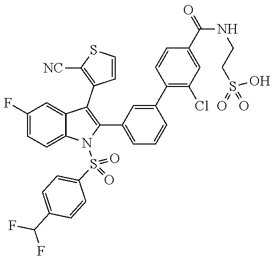 [SMILES: N#Cc1sccc1-c1c(-c2cccc(-c3ccc(C(=O)NCCS(=O)(=O)O)cc3Cl)c2)n(S(=O)(=O)c2ccc(C(F)F)cc2)c2ccc(F)cc12] | ¹H-NMR (500 MHz, CD₃OD) δ: 8.42 (dd, J = 9.0, 4.5 Hz, 1H), 7.97 (d, J = 1.5 Hz, 1H), 7.85-7.81 (m, 2H), 7.56-7.49 (m, 6H), 7.44 (d, J = 7.0 Hz, 1H), 7.39 (d, J = 8.0 Hz, 1H), 7.31 (td, J = 9.0, 2.5 Hz, 1H), 7.09-7.06 (m, 2H), 6.98 (d, J = 5.0 Hz, 1H), 6.71 (t, J = 55.5 Hz, 1H), 3.84 (t, J = 6.8 Hz, 2H), 3.13 (t, J = 6.8 Hz, 2H); MS: 768.0 (M − 1)⁻. |
| 10/4 | 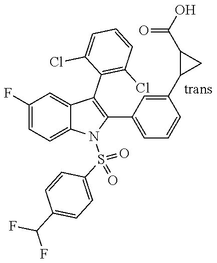 [SMILES: O=C(O)C1CC1c1cccc(-c2c(-c3c(Cl)cccc3Cl)c3cc(F)ccc3n2S(=O)(=O)c2ccc(C(F)F)cc2)c1] | 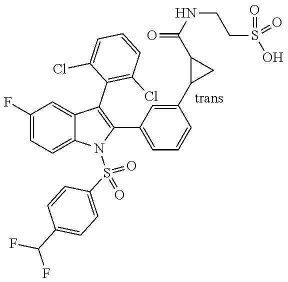 [SMILES: O=C(NCCS(=O)(=O)O)C1CC1c1cccc(-c2c(-c3c(Cl)cccc3Cl)c3cc(F)ccc3n2S(=O)(=O)c2ccc(C(F)F)cc2)c1] | ¹H-NMR (400 MHz, CD₃OD) δ: 8.37 (dd, J = 9.2, 4.4 Hz, 1H), 7.59 (d, J = 8.0 Hz, 2H), 7.47 (d, J = 8.0 Hz, 2H), 7.40-7.35 (m, 3H), 7.26-7.14 (m, 4H), 6.95-6.64 (m, 3H), 3.69-3.61 (m, 2H), 3.00 (t, J = 6.6 Hz, 2H), 2.33-2.28 (m, 1H), 1.64 (br s, 1H), 1.47-1.42 (m, 1H), 1.06 (br s, 1H); MS: 735.0 and 737.0 (M − 1)⁻. |

### Example Compounds Set 11

Example compounds are shown below.

| # | starting material | structure | analytical data |
|---|---|---|---|
| 11 | | 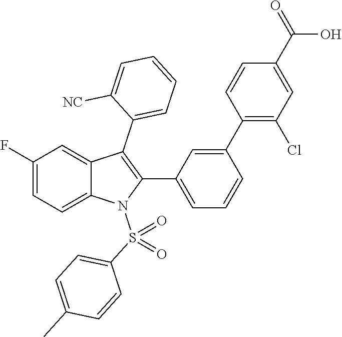 [SMILES: Cc1ccc(S(=O)(=O)n2c(-c3cccc(-c4ccc(C(=O)O)cc4Cl)c3)c(-c3ccccc3C#N)c3cc(F)ccc32)cc1] | |
| 11/1 | 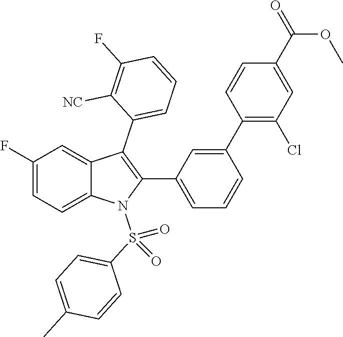 | 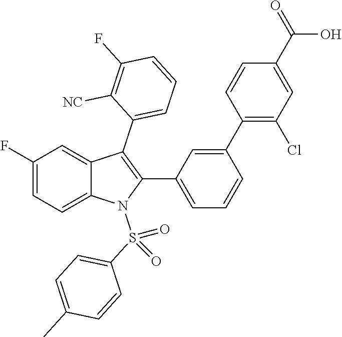 [SMILES: Cc1ccc(S(=O)(=O)n2c(-c3cccc(-c4ccc(C(=O)O)cc4Cl)c3)c(-c3cccc(F)c3C#N)c3cc(F)ccc32)cc1] | ¹H-NMR (500 MHz, CD₃OD) δ: 8.41 (dd, J = 9.3, 4.3 Hz, 1H), 8.08 (d, J = 1.0 Hz, 1H), 8.02 (dd, J = 1.8, 8.3 Hz, 1H), 7.69-7.64 (m, 1H), 7.49-7.26 (m, 8H), 7.18- 1.15 (m, 3H), 7.02 (s, 1H), 6.97 (dd, J = 2.5, 8.5 Hz, 1H), 2.25 (s, 3H); MS: 637.0 (M − 1)⁻. |
| | 1/33 | | |
| 11/2 | 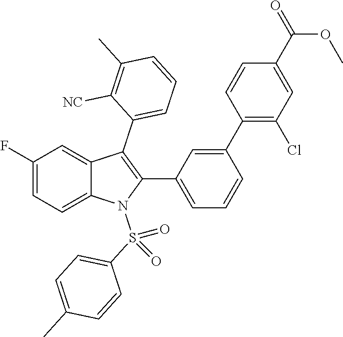 | 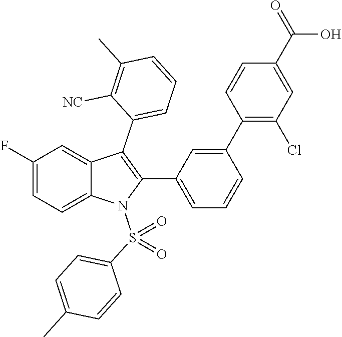 [SMILES: Cc1ccc(S(=O)(=O)n2c(-c3cccc(-c4ccc(C(=O)O)cc4Cl)c3)c(-c3cccc(C)c3C#N)c3cc(F)ccc32)cc1] | ¹H-NMR (500 MHz, CD₃OD) δ: 8.39 (dd, J = 9.3, 4.3 Hz, 1H), 8.05 (d, J = 1.5 Hz, 1H), 7.98 (dd, J = 8.0, 1.5 Hz, 1H), 7.51-7.43 (m, 4H), 7.39 (d, J = 8.0 Hz, 1H), 7.31 (d, J = 8.0 Hz, 1H), 7.28- 6.98 (m, 7H), 6.88 (dd, J = 8.5, 2.5 Hz, 1H), 2.51 (s, 3H), 2.24 (s, 3H); MS: 633.0 (M − 1)⁻. |
| | 1/34 | | |
| 11/3 | 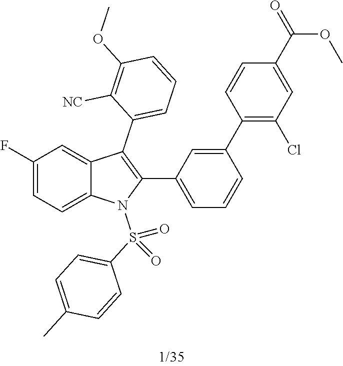 | 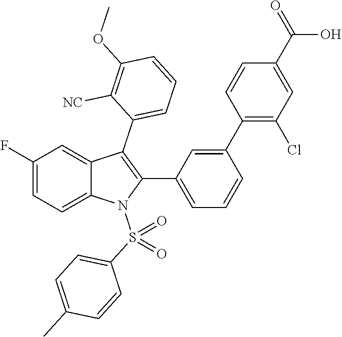 [SMILES: COc1cccc(-c2c(-c3cccc(-c4ccc(C(=O)O)cc4Cl)c3)n(S(=O)(=O)c3ccc(C)cc3)c3ccc(F)cc23)c1C#N] | ¹H-NMR (500 MHz, CD₃OD) δ: 8.38 (dd, J = 9.0, 4.5 Hz, 1H), 8.07 (d, J = 1.5 Hz, 1H), 8.00 (dd, J = 8.0, 1.5 Hz, 1H), 7.57-7.54 (m, 1H), 7.48-7.44 (m, 3H), 7.37 (d, J = 7.5 Hz, 1H), 7.27-7.14 (m, 6H), 7.03 (s, 1H), 6.88 (dd, J = 8.3, 2.8 Hz, 1H), 6.83 (br s, 1H), 3.95 (s, 3H), 2.24 (s, 3H); MS: 649.0 (M − 1)⁻. |
| 11/4 | 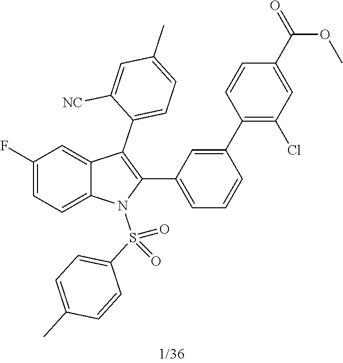 | 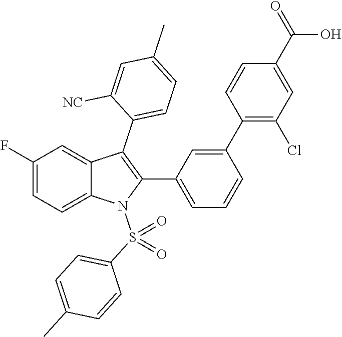 [SMILES: Cc1ccc(S(=O)(=O)n2c(-c3cccc(-c4ccc(C(=O)O)cc4Cl)c3)c(-c3ccc(C)cc3C#N)c3cc(F)ccc32)cc1] | ¹H-NMR (500 MHz, CD₃OD) δ: 8.38 (dd, J = 9.3, 4.3 Hz, 1H), 8.07 (d, J = 1.5 Hz, 1H), 7.99 (dd, J = 8.0, 1.5 Hz, 1H), 7.63-7.54 (m, 1H), 7.45-7.42 (m, 4H), 7.36 (d, J = 8.0 Hz, 1H), 7.30-7.14 (m, 6H), 7.02 (s, 1H), 6.88 (dd, J = 8.5, 2.5 Hz, 1H), 2.39 (s, 3H), 2.24 (s, 3H); MS: 633.0 (M − 1)⁻. |
| 11/5 | 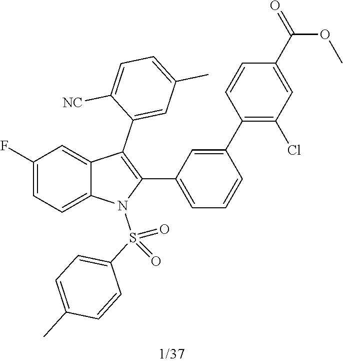 | 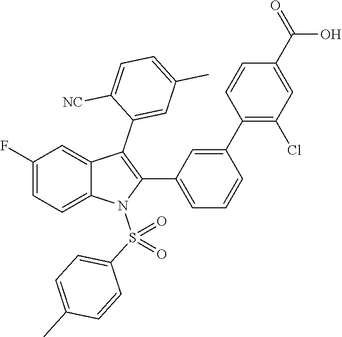 [SMILES: Cc1ccc(S(=O)(=O)n2c(-c3cccc(-c4ccc(C(=O)O)cc4Cl)c3)c(-c3cc(C)ccc3C#N)c3cc(F)ccc32)cc1] | ¹H-NMR (500 MHz, CD₃OD) δ: 8.39 (dd, J = 9.0, 4.5 Hz, 1H), 8.07 (d, J = 2.0 Hz, 1H), 8.00 (dd, J = 8.0, 1.5 Hz, 1H), 7.62 (d, J = 8.0 Hz, 1H), 7.46-7.44 (m, 3H), 7.36 (d, J = 8.5 Hz, 1H), 7.33-7.14 (m, 7H), 7.03 (s, 1H), 6.89 (dd, J = 8.3, 2.8 Hz, 1H), 2.35 (s, 3H), 2.24 (s, 3H); MS: 633.0 (M − 1)⁻. |
| 11/6 | 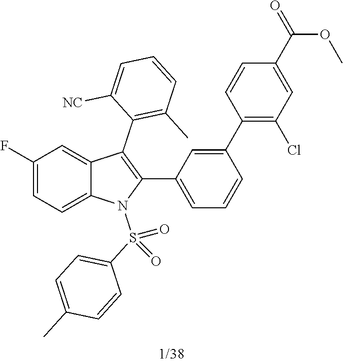 | 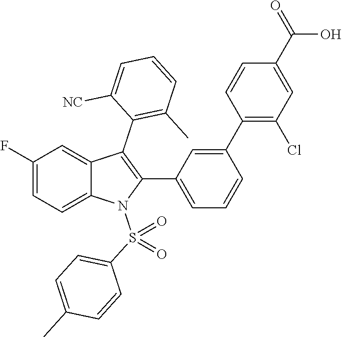 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1] | ¹H-NMR (500 MHz, CD₃OD) δ: 8.40 (dd, J = 9.3, 4.3 Hz, 1H), 8.08 (s, 1H), 8.00 (dd, J = 8.0, 1.5 Hz, 1H), 7.58-7.15 (m, 13H), 6.71 (dd, J = 8.0, 2.5 Hz, 1H), 2.25 (s, 3H), 1.90 (s, 3H); MS: 633.0 (M − 1)⁻. |
| 11/7 | 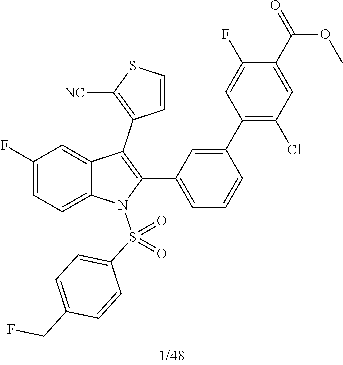 | 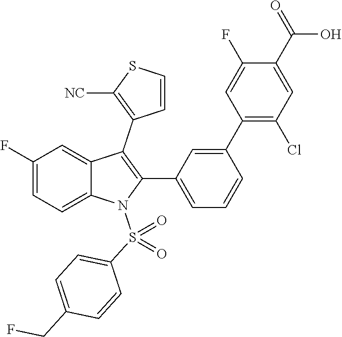 [SMILES: N#Cc1sccc1-c1c(-c2cccc(-c3cc(F)c(C(=O)O)cc3Cl)c2)n(S(=O)(=O)c2ccc(CF)cc2)c2ccc(F)cc12] | ¹H-NMR (500 MHz, CD₃OD) δ: 8.43 (dd, J = 9.0, 4.5 Hz, 1H), 8.03 (d, J = 7.0 Hz, 1H), 7.85 (d, J = 5.5 Hz, 1H), 7.57-7.44 (m, 5H), 7.38 (d, J = 8.5 Hz, 2H), 7.30 (dt, J = 2.7, 9.2 Hz, 1H), 7.21 (d, J = 10.5 Hz, 1H), 7.15 (s, 1H), 7.07 (dd, J = 8.5, 2.5 Hz, 1H), 6.99 (d, J = 5.5 Hz, 1H), 5.35 (d, J = 47.0 Hz, 2H); MS: 660.9 (M − 1)⁻. |
| 11/8 | 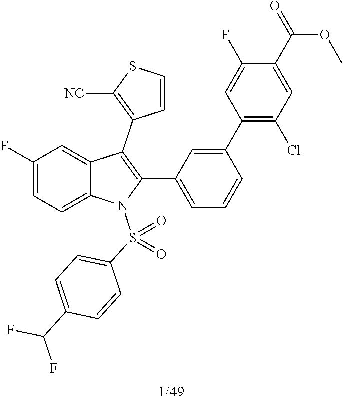 | 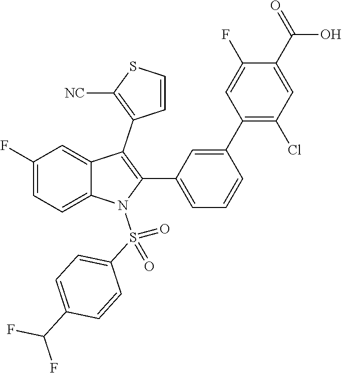 [SMILES: N#Cc1sccc1-c1c(-c2cccc(-c3cc(F)c(C(=O)O)cc3Cl)c2)n(S(=O)(=O)c2ccc(C(F)F)cc2)c2ccc(F)cc12] | ¹H-NMR (500 MHz, CD₃OD) δ: 8.43 (dd, J = 9.3, 4.3 Hz, 1H), 8.03 (d, J = 6.5 Hz, 1H), 7.86 (d, J = 5.0 Hz, 1H), 7.58-7.49 (m, 6H), 7.43 (d, J = 7.5 Hz, 1H), 7.31 (td, J = 9.0, 2.5 Hz, 1H), 7.22 (d, J = 10.5 Hz, 1H), 7.16 (s, 1H), 7.09 (dd, J = 2.5, 8.5 Hz, 1H), 7.01 (d, J = 5.0 Hz, 1H), 6.73 (t, J = 55.5 Hz, 1H); MS: 678.9 (M − 1)⁻. |
| 11/9 | 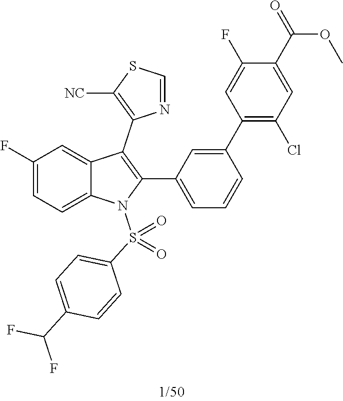 | 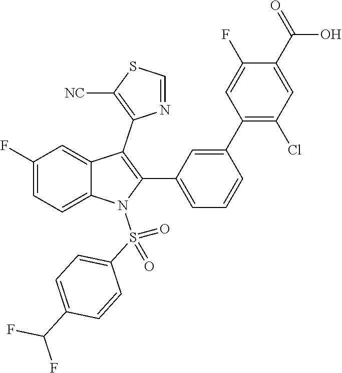 [SMILES: N#Cc1scnc1-c1c(-c2cccc(-c3cc(F)c(C(=O)O)cc3Cl)c2)n(S(=O)(=O)c2ccc(C(F)F)cc2)c2ccc(F)cc12] | ¹H-NMR (500 MHz, CD₃OD) δ: 9.33 (s, 1H), 8.46 (dd, J = 9.3, 4.3 Hz, 1H), 8.04 (d, J = 6.5 Hz, 1H), 7.61-7.50 (m, 6H), 7.41 (d, J = 8.0 Hz, 1H), 7.33 (d, J = 2.5 Hz, 1H), 7.27 (d, J = 11.0 Hz, 1H), 7.21-7.18 (m, 2H), 6.73 (t, J = 55.5 Hz, 1H); MS: 679.9 (M − 1)⁻. |
| 11/10 | 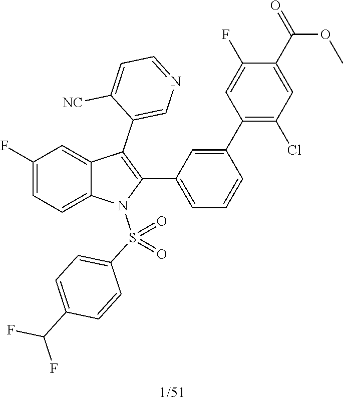 | 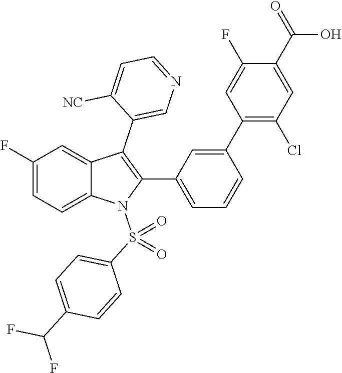 [SMILES: N#Cc1ccncc1-c1c(-c2cccc(-c3cc(F)c(C(=O)O)cc3Cl)c2)n(S(=O)(=O)c2ccc(C(F)F)cc2)c2ccc(F)cc12] | ¹H-NMR (500 MHz, CD₃OD) δ: 8.72 (d, J = 4.5 Hz, 1H), 8.55 (br s, 1H), 8.44 (dd, J = 4.3, 9.3 Hz, 1H), 8.01 (d, J = 6.5 Hz, 1H), 7.78 (d, J = 5.0 Hz, 1H), 7.60-7.04 (m, 11H), 6.73 (t, J = 55.5 Hz, 1H); MS: 673.9 (M − 1)⁻. |
| 11/11 | 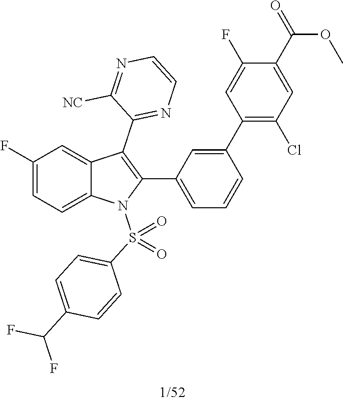 | 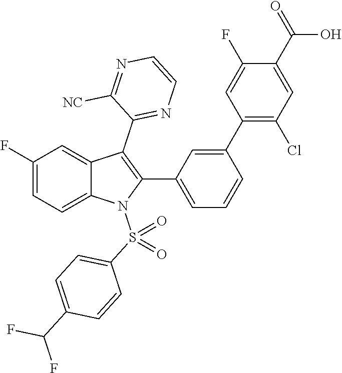 [SMILES: N#Cc1nccnc1-c1c(-c2cccc(-c3cc(F)c(C(=O)O)cc3Cl)c2)n(S(=O)(=O)c2ccc(C(F)F)cc2)c2ccc(F)cc12] | ¹H-NMR (500 MHz, CD₃OD) δ: 8.90 (d, J = 2.5 Hz, 1H), 8.71 (d, J = 2.5 Hz, 1H), 8.47 (dd, J = 9.5, 4.5 Hz, 1H), 8.01 (d, J = 5.0 Hz, 1H), 7.78-7.18 (m, 11H), 6.73 (t, J = 55.5 Hz, 1H); MS: 674.9 (M − 1)⁻. |
| 11/12 | 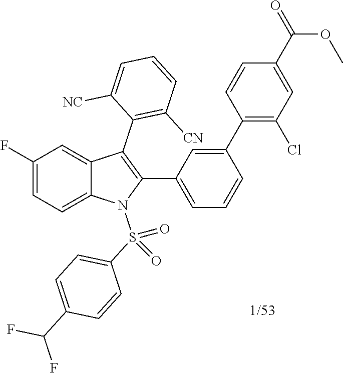 | 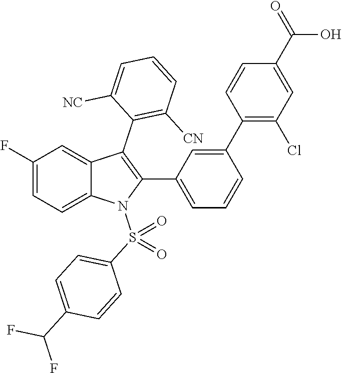 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, CD₃OD) δ: 8.45 (dd, J = 4.3, 9.3 Hz, 1H), 8.08-8.00 (m, 4H), 7.72 (t, J = 7.8 Hz, 1H), 7.55- 7.32 (m, 9H), 7.08 (s, 1H), 6.95 (dd, J = 2.5, 8.5 Hz, 1H), 6.68 (t, J = 55.5 Hz, 1H); MS: 679.9 (M − 1)⁻. |
| 11/13 | 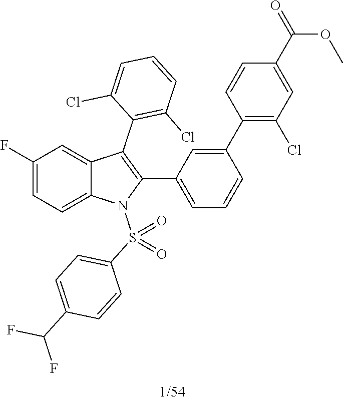 | 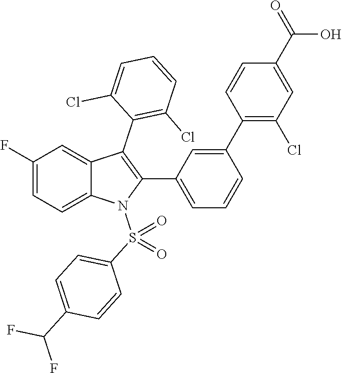 [SMILES: O=C(O)c1ccc(-c2cccc(-c3c(-c4c(Cl)cccc4Cl)c4cc(F)ccc4n3S(=O)(=O)c3ccc(C(F)F)cc3)c2)c(Cl)c1] | ¹H-NMR (500 MHz, CD₃OD) δ: 8.39 (dd, J = 4.8, 9.3 Hz, 1H), 8.08 (d, J = 1.5 Hz, 1H), 7.98 (dd, J = 1.5, 8.0 Hz, 1H), 7.56-7.25 (m, 12H), 7.21 (s, 1H), 6.73 (t, J = 55.3 Hz, 1H), 6.68 (dd, J = 2.5, 8.5 Hz, 1H); MS: 697.9 (M − 1)⁻. |
| 11/14 | 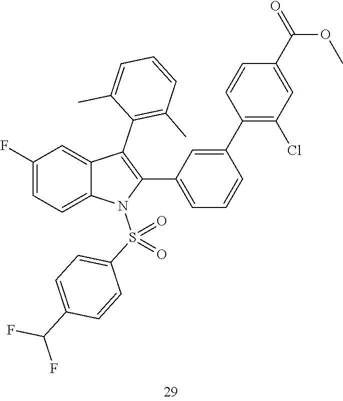 | 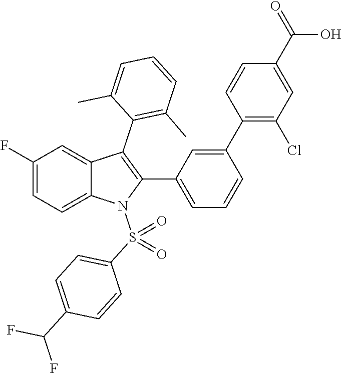 [SMILES: Cc1cccc(C)c1-c1c(-c2cccc(-c3ccc(C(=O)O)cc3Cl)c2)n(S(=O)(=O)c2ccc(C(F)F)cc2)c2ccc(F)cc12] | ¹H-NMR (400 MHz, CD₃OD) δ: 8.41 (dd, J = 4.2, 9.0 Hz, 1H), 8.07 (d, J = 1.6 Hz, 1H), 7.94 (dd, J = 1.6, 8.0 Hz, 1H), 7.57-7.40 (m, 7H), 7.29-7.15 (m, 4H), 7.04 (d, J = 8.4 Hz, 2H), 6.75 (t, J = 55.6 Hz, 1H), 6.57 (d, J = 2.6, 8.2 Hz, 1H), 1.63 (s, 6H); MS: 658.0 (M − 1)⁻. |
| 11/15 | 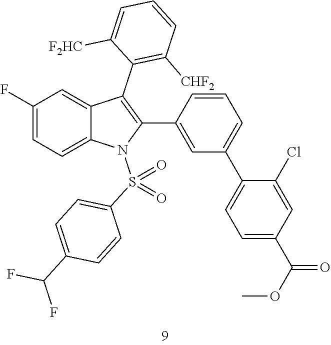 | 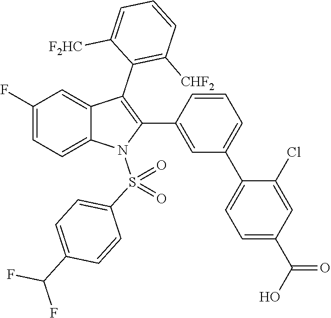 [SMILES: O=C(O)c1ccc(-c2cccc(-c3c(-c4c(C(F)F)cccc4C(F)F)c4cc(F)ccc4n3S(=O)(=O)c3ccc(C(F)F)cc3)c2)c(Cl)c1] | ¹H-NMR (400 MHz, CD₃OD) δ: 8.44 (dd, J = 4.4, 9.2 Hz, 1H), 8.08 (d, J = 1.6 Hz, 1H), 7.97 (dd, J = 1.4, 3.8 Hz, 1H), 7.83 (d, J = 7.6 Hz, 2H), 7.73 (t, J = 7.8 Hz, 1H), 7.61-7.55 (m, 4H), 7.47-7.23 (m, 5H), 6.91 (s, 1H), 6.77-6.63 (m, 2H), 6.01 (t, J = 55.0 Hz, 2H); MS: 729.9 (M − 1)⁻. |
| 11/16 | 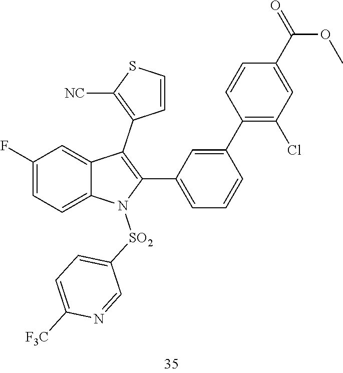 | 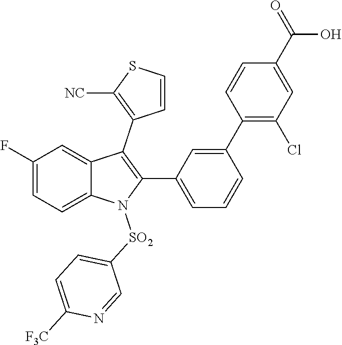 [SMILES: N#Cc1sccc1-c1c(-c2cccc(-c3ccc(C(=O)O)cc3Cl)c2)n(S(=O)(=O)c2ccc(C(F)(F)F)nc2)c2ccc(F)cc12] | ¹H-NMR (500 MHz, DMSO-d₆) δ: 13.39 (s, 1H), 8.78 (d, J = 2.5 Hz, 1H), 8.34 (dd, J = 9.0, 4.5 Hz, 1H), 8.12-7.93 (m, 5H), 7.57-7.55 (m, 2H), 7.50-7.41 (m, 3H), 7.23- 7.21 (m, 2H), 7.04- 7.02 (m, 1H); MS: 679.6 (M − 1)⁻. |
| 11/17 | 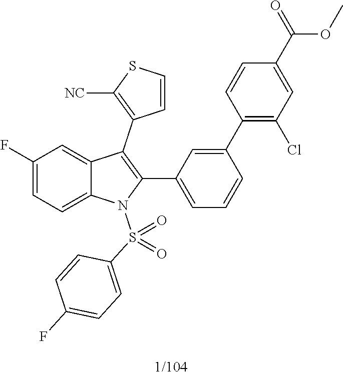 | 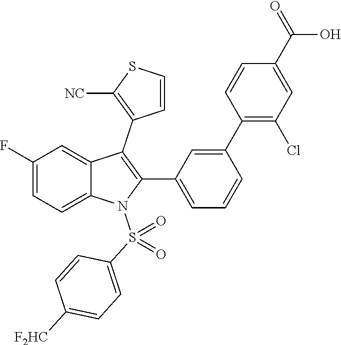 [SMILES: N#Cc1sccc1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, DMSO-d₆) δ: 8.30 (dd, J = 9.2, 4.4 Hz, 1H), 8.04 (d, J = 5.1 Hz, 1H), 7.99-7.88 (m, 2H), 7.70-7.49 (m, 6H), 7.47-7.35 (m, 3H), 7.20 (dd, J = 8.6, 2.6 Hz, 1H), 7.12- 7.10 (m, 1H), 7.05- 7.03 (m, 1H), 7.01 (t, J = 55.0 Hz, 1H); MS: 660.9 (M − 1)⁻. |
| 11/18 | 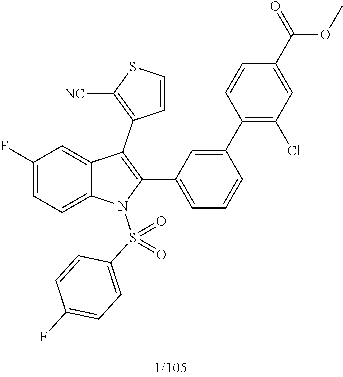 | 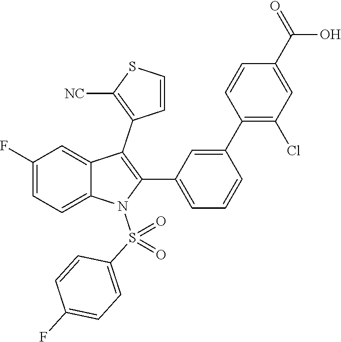 [SMILES: N#Cc1sccc1-c1c(-c2cccc(-c3ccc(C(=O)O)cc3Cl)c2)n(S(=O)(=O)c2ccc(F)cc2)c2ccc(F)cc12] | ¹H-NMR (500 MHz, DMSO-d₆) δ: 8.30 (dd, J = 9.2, 4.4 Hz, 1H), 8.04 (d, J = 5.1 Hz, 1H), 7.99- 7.88 (m, 2H), 7.58- 7.45 (m, 4H), 7.45-7.35 (m, 3H), 7.31 (t, J = 8.8 Hz, 2H), 7.19 (dd, J = 8.6, 2.6 Hz, 1H), 7.14 (s, 1H), 7.03 (d, J = 4.9 Hz, 1H); MS: 628.9 (M − 1)⁻. |
| 11/19 | 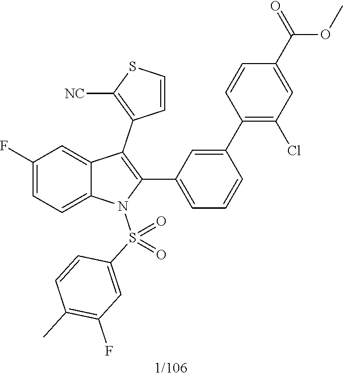 | 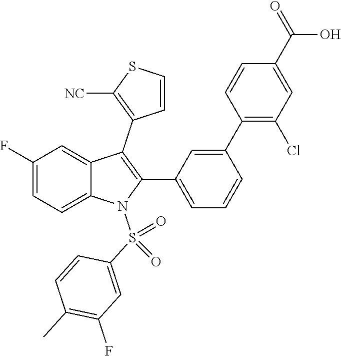 [SMILES: Cc1ccc(S(=O)(=O)n2c(-c3cccc(-c4ccc(C(=O)O)cc4Cl)c3)c(-c3ccsc3C#N)c3cc(F)ccc32)cc1F] | ¹H-NMR (500 MHz, DMSO-d₆) δ: 13.42 (s, 1H), 8.31 (dd, J = 9.2 4.5 Hz, 1H), 8.05- 7.93 (m, 3H), 7.55-7.53 (m, 2H), 7.48-7.36 (m, 4H), 7.24-7.11 (m, 4H), 7.05 (d, J = 4.8 Hz, 1H), 2.15 (s, 3H); MS: 642.9 (M − 1)⁻. |
| 11/20 | 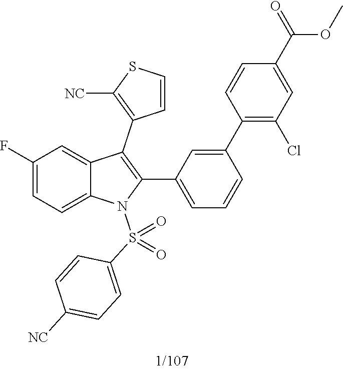 | 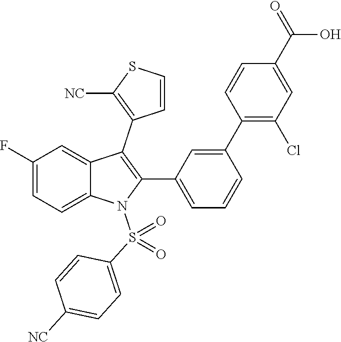 [SMILES: N#Cc1ccc(S(=O)(=O)n2c(-c3cccc(-c4ccc(C(=O)O)cc4Cl)c3)c(-c3ccsc3C#N)c3cc(F)ccc32)cc1] | ¹H-NMR (500 MHz, DMSO-d₆) δ: 13.46 (s, 1H), 8.31- 8.28 (m, 1H), 8.05 (d, J = 5.1 Hz, 1H), 8.02-7.92 (m, 4H), 7.59-7.50 (m, 4H), 7.48-7.37 (m, 3H), 7.22-7.20 (m, 1H), 7.12 (s, 1H), 7.04 (d, J = 5.0 Hz, 1H); MS: 635.9 (M − 1)⁻. |
| 11/21 | 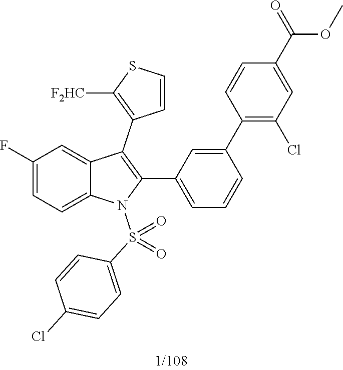 | 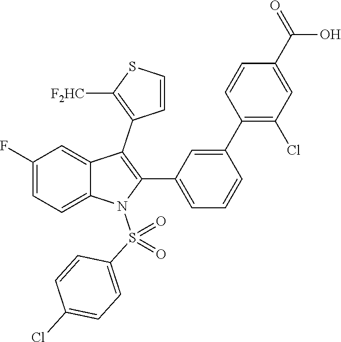 [SMILES: O=C(O)c1ccc(-c2cccc(-c3c(-c4ccsc4C(F)F)c4cc(F)ccc4n3S(=O)(=O)c3ccc(Cl)cc3)c2)c(Cl)c1] | ¹H-NMR (400 MHz, CD₃OD) δ: 8.36 (dd, J = 9.2, 4.4 Hz, 1H), 8.07 (d, J = 1.6 Hz, 1H), 7.97 (dd, J = 8.0, 1.6 Hz, 1H), 7.64 (d, J = 5.0 Hz, 1H), 7.52- 7.33 (m, 7H), 7.31-7.22 (m, 2H), 7.12 (s, 1H), 6.91 (dd, J = 8.5, 2.5 Hz, 1H), 6.82 (br s, 1H), 6.34 (t, J = 54.8 Hz, 1H); MS: 669.8 (M − 1)⁻. |
| 11/22 | 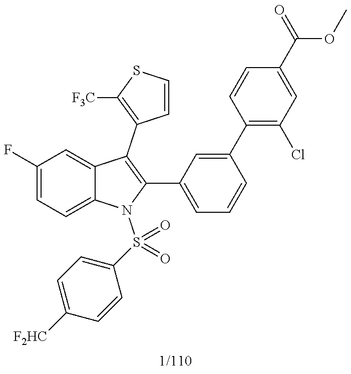 | 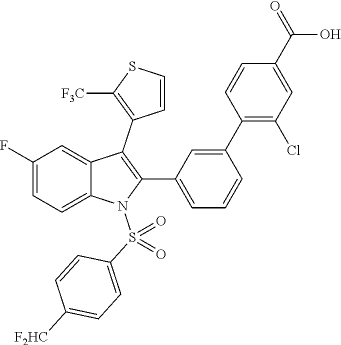 [SMILES: O=C(O)c1ccc(-c2cccc(-c3c(-c4ccsc4C(F)(F)F)c4cc(F)ccc4n3S(=O)(=O)c3ccc(C(F)F)cc3)c2)c(Cl)c1] | ¹H-NMR (500 MHz, CD₃OD) δ: 8.38 (dd, J = 9.2, 4.3 Hz, 1H), 8.11 (d, J = 1.3 Hz, 1H), 8.01 (d, J = 6.9 Hz, 1H), 7.70 (d, J = 4.0 Hz, 1H), 7.57-7.24 (m, 10H), 6.85-6.63 (m, 3H); MS: 703.9 (M − 1)⁻. |
| 11/23 | 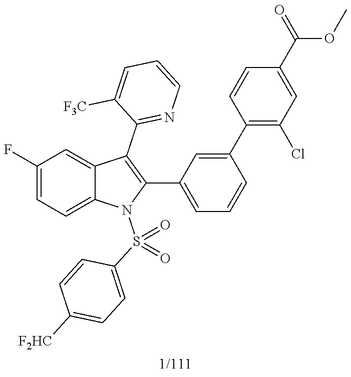 | 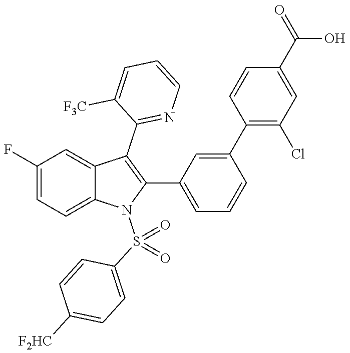 [SMILES: O=C(O)c1ccc(-c2cccc(-c3c(-c4ncccc4C(F)(F)F)c4cc(F)ccc4n3S(=O)(=O)c3ccc(C(F)F)cc3)c2)c(Cl)c1] | ¹H-NMR (500 MHz, CD₃OD) δ: 8.78 (d, J = 4.6 Hz, 1H), 8.42 (dd, J = 9.2, 4.3 Hz, 1H), 8.21 (d, J = 7.5 Hz, 1H), 8.09 (d, J = 1.5 Hz, 1H), 8.01 (dd, J = 8.0, 1.5 Hz, 1H), 7.70- 7.06 (m, 11H), 6.79- 6.77 (m, 1H), 6.76 (t, J = 56.0, 1H); MS: 699.0 (M − 1)⁻. |
| 11/24 | 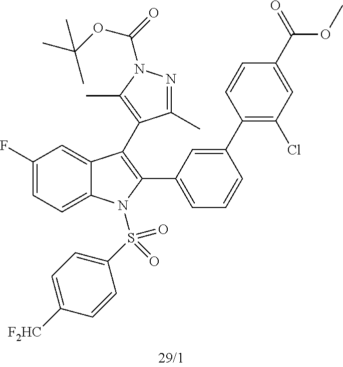 | 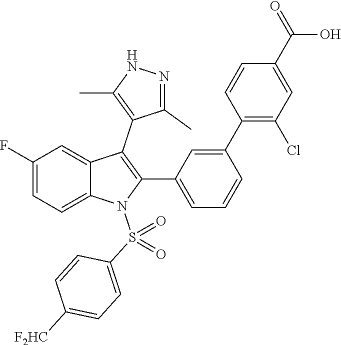 [SMILES: Cc1n[nH]c(C)c1-c1c(-c2cccc(-c3ccc(C(=O)O)cc3Cl)c2)n(S(=O)(=O)c2ccc(C(F)F)cc2)c2ccc(F)cc12] | ¹H-NMR (400 MHz, CD₃OD) δ: 8.36 (dd, J = 9.1, 4.4 Hz, 1H), 8.07 (d, J = 1.5 Hz, 1H), 7.97 (dd, J = 7.9, 1.6 Hz, 1H), 7.56-7.43 (m, 7H), 7.33 (d, J = 8.0 Hz, 1H), 7.25 (dt, J = 4.4, 8.8 Hz, 1H), 7.15 (s, 1H), 6.82- 6.79 (m, 1H), 6.73 (t, J = 55.2 Hz, 1H), 1.70 (s, 6H); MS: 650.1 (M + 1)⁺. |
| 11/25 | 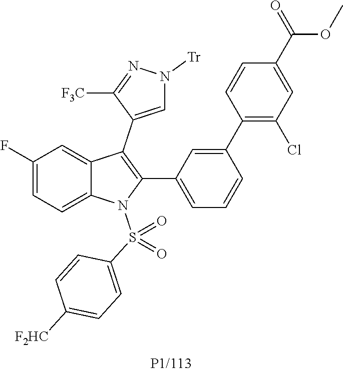 | 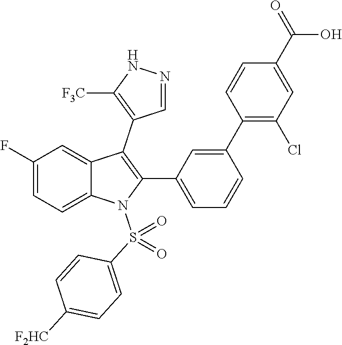 [SMILES: O=C(O)c1ccc(-c2cccc(-c3c(-c4cn[nH]c4C(F)(F)F)c4cc(F)ccc4n3S(=O)(=O)c3ccc(C(F)F)cc3)c2)c(Cl)c1] | ¹H-NMR (500 MHz, CD₃OD) δ: 8.37 (dd, J = 9.1, 4.3 Hz, 1H), 8.09 (d, J = 1.4 Hz, 1H), 7.99 (dd, J = 8.0, 1.5 Hz, 1H), 7.68-6.89 (m, 11H), 6.88- 6.86 (m, 1H), 6.73 (t, J = 55.0 Hz, 1H); MS: 688.0 (M − 1)⁻. |
| 11/26 | 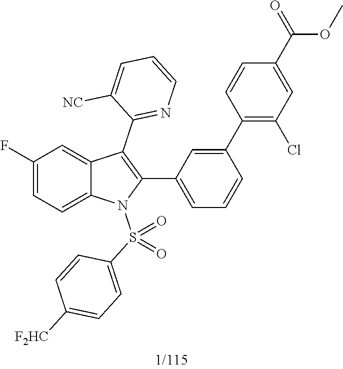 | 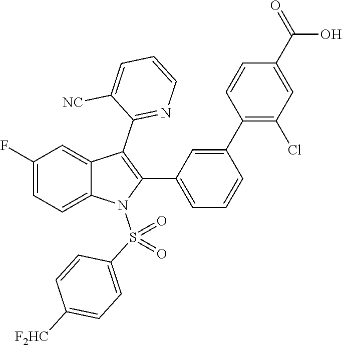 [SMILES: N#Cc1cccnc1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, CD₃OD) δ: 8.84 (dd, J = 5.0, 1.6 Hz, 1H), 8.45 (dd, J = 9.2, 4.3 Hz, 1H), 8.16 (dd, J = 8.0, 1.5 Hz, 1H), 8.07 (s, 1H), 8.00 (d, J = 8.0 Hz, 1H), 7.82-7.10 (m, 11H), 7.05 (dd, J = 8.4, 2.5 Hz, 1H), 6.71 (t, J = 55.6 Hz, 1H); MS: 655.9 (M − 1)⁻. |
| 11/27 | 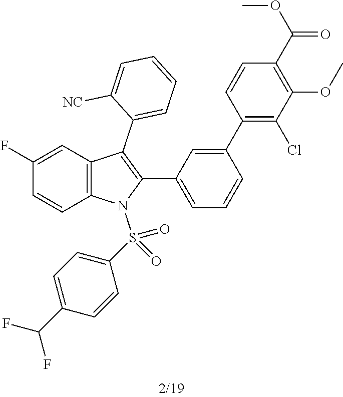 | 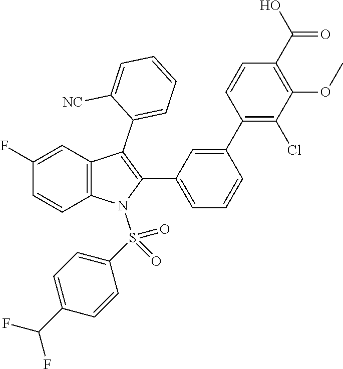 [SMILES: COc1c(C(=O)O)ccc(-c2cccc(-c3c(-c4ccccc4C#N)c4cc(F)ccc4n3S(=O)(=O)c3ccc(C(F)F)cc3)c2)c1Cl] | ¹H-NMR (500 MHz, DMSO-d₆) δ: 8.28 (dd, J = 9.0, 4.5 Hz, 1H), 7.88 (d, J = 6.0 Hz, 1H), 7.76-7.36 (m, 12H), 7.12-6.90 (m, 4H), 3.82 (s, 3H); MS: 684.9 (M − 1)⁻. |
| 11/28 | 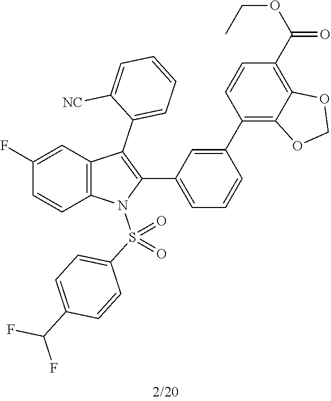 | 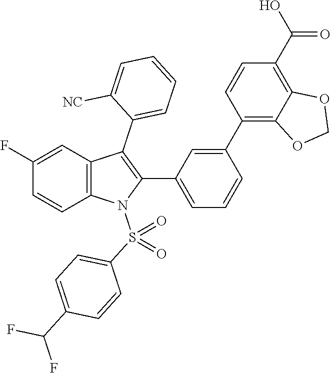 [SMILES: N#Cc1ccccc1-c1c(-c2cccc(-c3ccc(C(=O)O)c4c3OCO4)c2)n(S(=O)(=O)c2ccc(C(F)F)cc2)c2ccc(F)cc12] | ¹H-NMR (500 MHz, CD₃OD) δ: 8.41 (dd, J = 9.0, 4.0 Hz, 1H), 7.78 (d, J = 7.5 Hz, 2H), 7.61- 7.39 (m, 10H), 7.30-7.26 (m, 2H), 6.95 (d, J = 8.5 Hz, 1H), 6.90- 6.87 (m, 1H), 6.70 (t, J = 55.5 Hz, 1H), 6.08 (d, J = 4.5 Hz, 2H); MS: 665.0 (M − 1)⁻. |
| 11/29 | 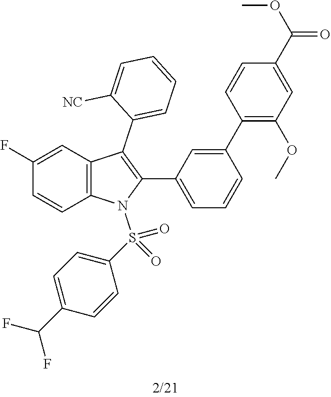 | 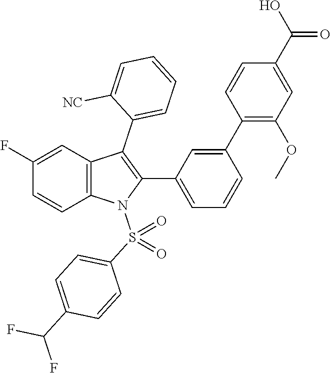 [SMILES: COc1cc(C(=O)O)ccc1-c1cccc(-c2c(-c3ccccc3C#N)c3cc(F)ccc3n2S(=O)(=O)c2ccc(C(F)F)cc2)c1] | ¹H-NMR (400 MHz, CD₃OD) δ: 8.40 (dd, J = 9.2, 4.4 Hz, 1H), 7.79-7.50 (m, 10H), 7.35-7.17 (m, 6H), 6.88 (dd, J = 8.4, 2.4 Hz, 1H), 6.69 (t, J = 55.6 Hz, 1H), 3.83 (s, 3H); MS: 651.0 (M − 1)⁻. |
| 11/30 | 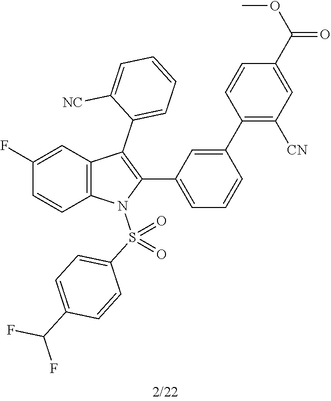 | 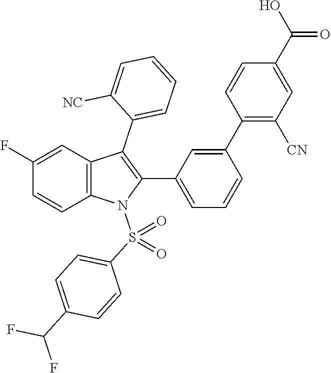 [SMILES: N#Cc1cc(C(=O)O)ccc1-c1cccc(-c2c(-c3ccccc3C#N)c3cc(F)ccc3n2S(=O)(=O)c2ccc(C(F)F)cc2)c1] | ¹H-NMR (400 MHz, CD₃OD) δ: 8.43-8.40 (m, 2H), 8.29 (dd, J = 8.2, 1.4 Hz, 1H), 7.77-7.75 (m, 1H), 7.67-7.26 (m, 13H), 6.91 (dd, J = 2.8, 8.4 Hz, 1H), 6.74 (t, J = 55.6 Hz, 1H); MS: 646.0 (M − 1)⁻. |
| 11/31 | 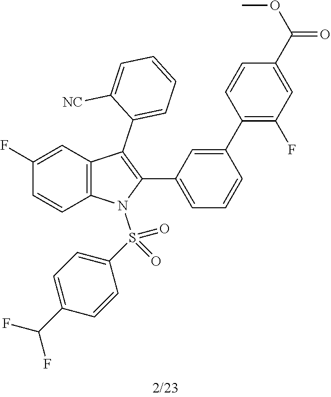 | 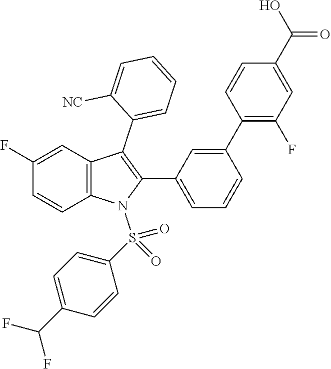 [SMILES: N#Cc1ccccc1-c1c(-c2cccc(-c3ccc(C(=O)O)cc3F)c2)n(S(=O)(=O)c2ccc(C(F)F)cc2)c2ccc(F)cc12] | ¹H-NMR (400 MHz, CD₃OD) δ: 8.41 (d, J = 9.2, 4.4 Hz, 1H), 7.87 (dd, J = 1.6, 8.0 Hz, 1H), 7.76 (dd, J = 1.4, 11.0 Hz, Hz, 1H), 7.65-7.26 (m, 13H), 6.89 (dd, J = 2.4, 8.4 Hz, 1H), 6.72 (t, J = 55.6 Hz, 1H); MS: 639.0 (M − 1)⁻. |
| 11/32 | 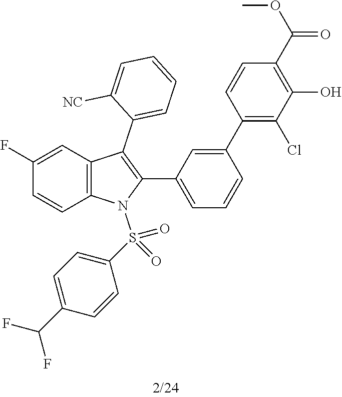 | 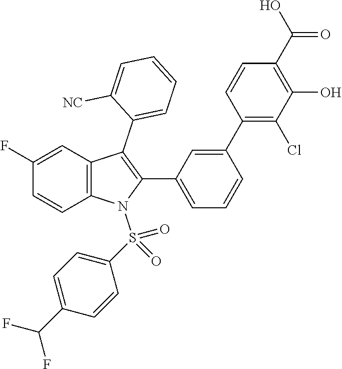 [SMILES: N#Cc1ccccc1-c1c(-c2cccc(-c3ccc(C(=O)O)c(O)c3Cl)c2)n(S(=O)(=O)c2ccc(C(F)F)cc2)c2ccc(F)cc12] | ¹H-NMR (400 MHz, CD₃OD) δ: 8.41 (dd, J = 9.2, 4.4 Hz, 1H), 7.83 (d, J = 8.4 Hz, 1H), 7.78- 6.55 (m, 16H); MS: 670.9 (M − 1)⁻ |
| 11/33 | 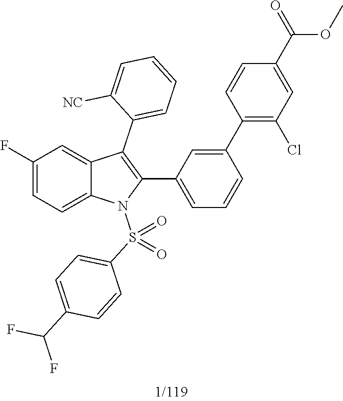 [SMILES: COC(=O)c1ccc(-c2cccc(-c3c(-c4ccccc4C#N)c4cc(F)ccc4n3S(=O)(=O)c3ccc(C(F)F)cc3)c2)c(Cl)c1] | 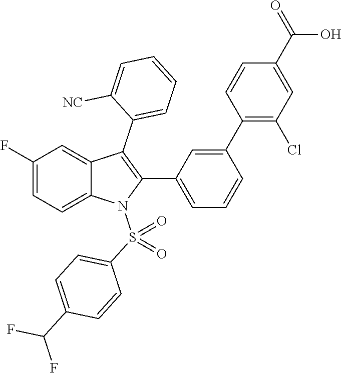 [SMILES: N#Cc1ccccc1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, MeOD) δ: 8.40 (dd, J = 9.3, 4.3 Hz, 1H), 8.07 (d, J = 2.0 Hz, 1H), 7.99 (dd, J = 7.8, 1.8 Hz, 1H), 7.77 (d, J = 7.5 Hz, 1H), 7.64 (t, J = 7.5 Hz, 1H), 7.56-7.44 (m, 8H), 7.35-7.26 (m, 3H), 7.09 (br s, 1H), 6.90 (dd, J = 8.5, 2.5 Hz, 1H), 6.70 (t, J = 55.5 Hz, 1H); MS: 670.9 (M − 1)⁻. |
| 11/34 | 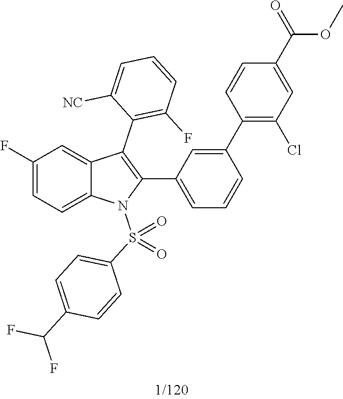 [SMILES: COC(=O)c1ccc(-c2cccc(-c3c(-c4c(F)cccc4C#N)c4cc(F)ccc4n3S(=O)(=O)c3ccc(C(F)F)cc3)c2)c(Cl)c1] | 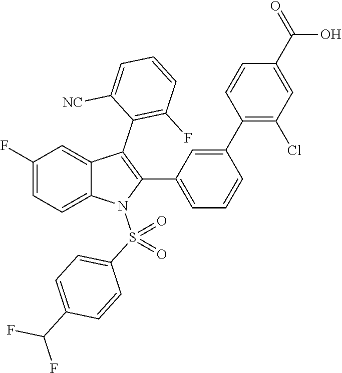 [SMILES: N#Cc1cccc(F)c1-c1c(-c2cccc(-c3ccc(C(=O)O)cc3Cl)c2)n(S(=O)(=O)c2ccc(C(F)F)cc2)c2ccc(F)cc12] | ¹H-NMR (400 MHz, MeOD) δ: 8.42 (dd, J = 9.4, 4.2 Hz, 1H), 8.06 (d, J = 1.6 Hz, 1H), 7.98 (dd, J = 7.8, 1.4 Hz, 1H), 7.64-7.41 (m, 10H), 7.34-7.28 (m, 2H), 7.05 (br s, 1H), 6.90 (dd, J = 8.2, 2.6 Hz, 1H), 6.70 (t, J = 55.4 Hz, 1H); MS: 670.9 (M − 1)⁻. |
| 11/35 | 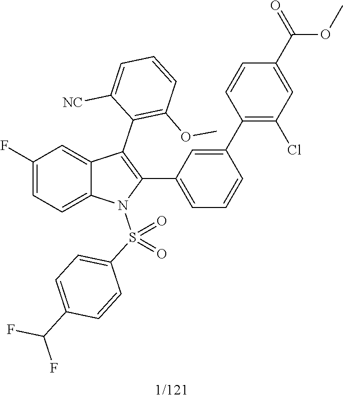 | 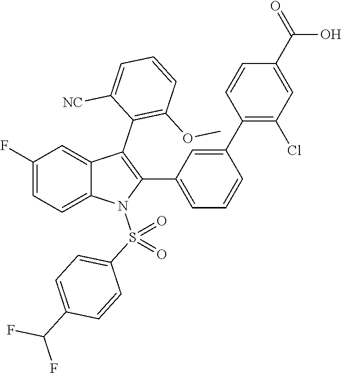 | ¹H-NMR (500 MHz, MeOD) δ: 8.37 (dd, J = 9.3, 4.3 Hz, 1H), 8.08 (d, J = 1.0 Hz, 1H), 7.99 (dd, J = 7.5, 1.5 Hz, 1H), 7.56-7.23 (m, 12H), 7.10 (br s, 1H), 6.76 (dd, J = 1.8, 8.3 Hz, 1H), 6.72 (t, J = 55.5 Hz, 1H), 3.59 (s, 3H); MS: 670.9 (M − 1)⁻. |
| 11/36 | 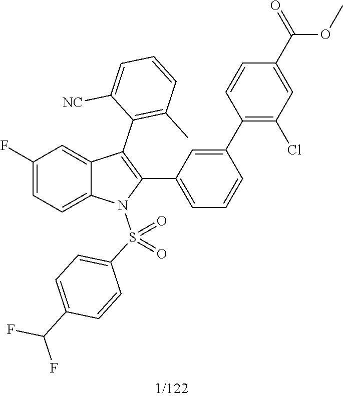 | 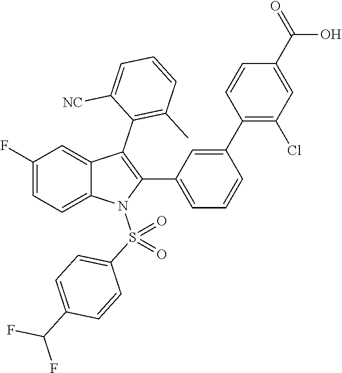 [SMILES: Cc1cccc(C#N)c1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, MeOD) δ: 8.41 (dd, J = 9.3, 4.3 Hz, 1H), 8.08 (d, J = 1.5 Hz, 1H), 8.00 (dd, J = 8.0, 1.5 Hz, 1H), 7.60-7.15( m, 13H), 6.82-6.60 (m, 2H), 1.88 (s, 3H); MS: 670.9 (M − 1)⁻. |
| 11/37 | 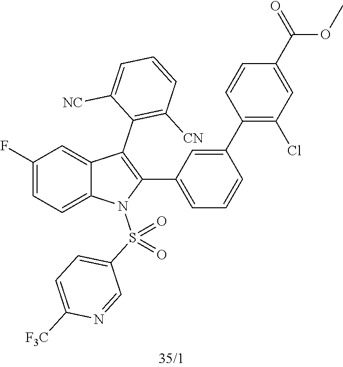 | 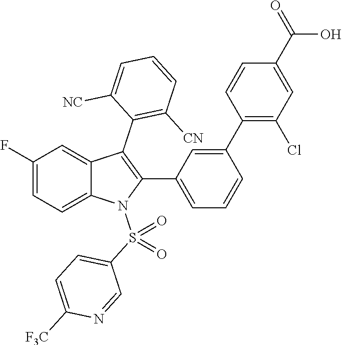 [SMILES: N#Cc1cccc(C#N)c1-c1c(-c2cccc(-c3ccc(C(=O)O)cc3Cl)c2)n(S(=O)(=O)c2ccc(C(F)(F)F)nc2)c2ccc(F)cc12] | ¹H-NMR (400 MHz, MeOD) δ: 8.68 (d, J = 2.0 Hz, 1H), 8.50 (dd, J = 9.2, 4.0 Hz, 1H), 8.09- 7.98 (m, 5H), 7.83 (d, J = 8.4 Hz, 1H), 7.74 (t, J = 7.8 Hz, 1H), 7.55-7.38 (m, 5H), 7.20 (s, 1H), 6.97 (dd, J = 8.2, 2.6 Hz, 1H); MS: 698.9 (M − 1)⁻. |
| 11/38 | 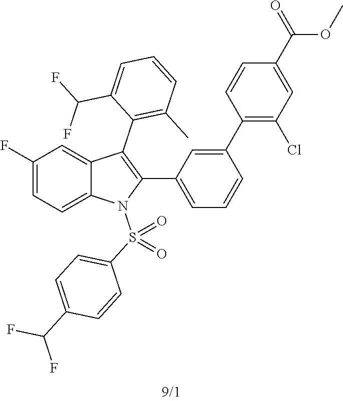 | 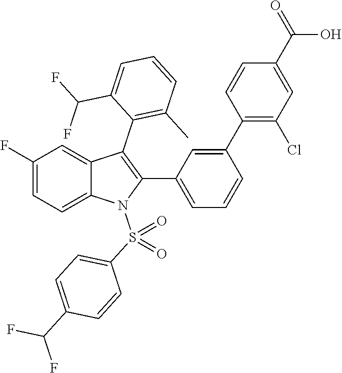 [SMILES: Cc1cccc(C(F)F)c1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, MeOD) δ: 8.42 (dd, J = 9.0, 4.5 Hz, 1H), 8.09 (d, J = 2.0 Hz, 1H), 7.98 (dd, J = 8.0, 1.5 Hz, 1H), 7.59-7.25 (m, 12H), 7.21 (br s, 1H), 6.77 (t, J = 55.5 Hz, 1H), 6.59 (dd, J = 8.0, 2.5 Hz, 1H), 5.90 (t, J = 55.5 Hz, 1H), 1.70 (s, 3H); MS: 694.0 (M − 1)⁻. |
| C11/ 39 | 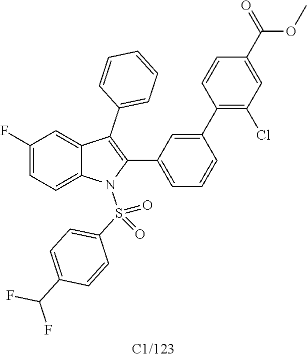 | 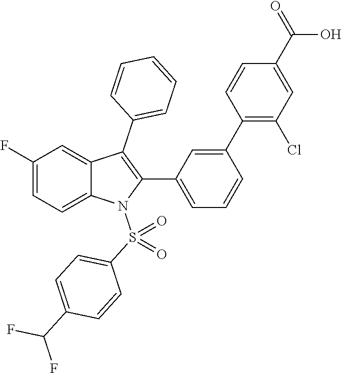 [SMILES: O=C(O)c1ccc(-c2cccc(-c3c(-c4ccccc4)c4cc(F)ccc4n3S(=O)(=O)c3ccc(C(F)F)cc3)c2)c(Cl)c1] | ¹H-NMR (400 MHz, CD₃OD) δ: 8.40 (dd, J = 9.2, 4.4 Hz, 1H), 8.07 (d, J = 1.6 Hz, 1H), 7.97 (dd, J = 8.0, 1.6 Hz, 1H), 7.56-7.45 (m, 7H), 7.34-7.21 (m, 5H), 7.14-7.11 (m, 4H), 6.73 (t, J = 55.6 Hz, 1H); MS: 630.0 (M − 1)⁻. |
| 11/40 | 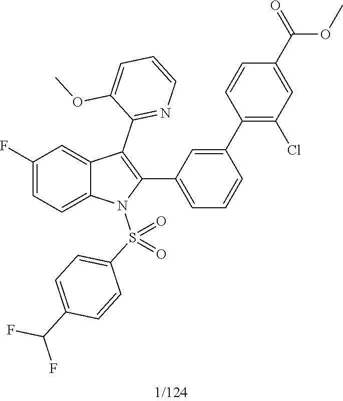 | 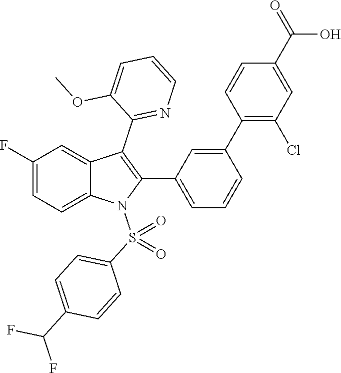 [SMILES: COc1cccnc1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, CD₃OD) δ: 8.41 (dd, J = 9.0, 4.5 Hz, 1H), 8.19 (dd, J = 1.0, 4.5 Hz, 1H), 8.08 (d, J = 1.0 Hz, 1H), 8.01 (dd, J = 7.8, 1.8 Hz, 1H), 7.56-7.46 (m, 9H), 7.39 (d, J = 8.0 Hz, 1H), 7.29-7.25 (m, 1H), 7.14 (br s, 1H), 7.00 (dd, J = 9.0, 2.5 Hz, 1H), 6.74 (t, J = 55.5 Hz, 1H), 3.50 (s, 3H); MS: 663.0 (M + 1)⁺. |
| 11/41 | 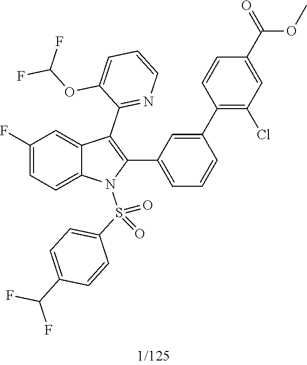 | 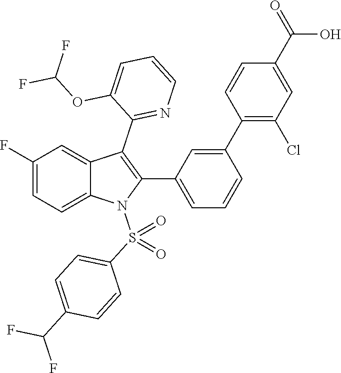 [SMILES: O=C(O)c1ccc(-c2cccc(-c3c(-c4ncccc4OC(F)F)c4cc(F)ccc4n3S(=O)(=O)c3ccc(C(F)F)cc3)c2)c(Cl)c1] | ¹H-NMR (400 MHz, CD₃OD) δ: 8.44-8.40 (m, 2H), 8.07 (d, J = 1.6 Hz, 1H), 8.00 (dd, J = 8.0, 1.2 Hz, 1H), 7.67 (d, J = 8.4 Hz, 1H), 7.57-7.25 (m, 10H), 7.14 (br s, 1H), 6.98 (dd, J = 8.6, 2.6 Hz, 1H), 6.73 (t, J = 55.6 Hz, 1H), 6.51 (t, J = 72.6 Hz, 1H); MS: 699.0 (M + 1)⁺. |
| 11/42 | 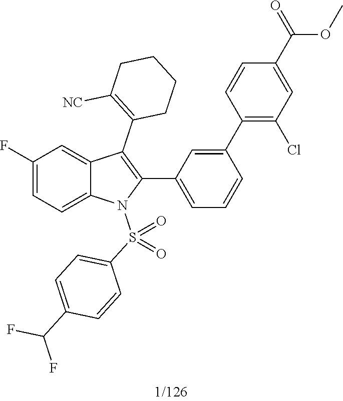 | 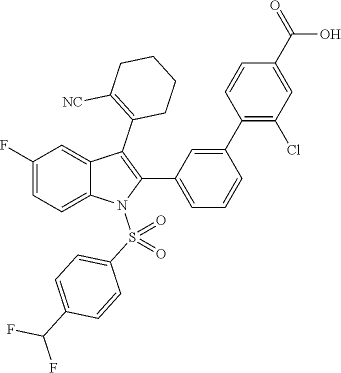 [SMILES: N#CC1=C(c2c(-c3cccc(-c4ccc(C(=O)O)cc4Cl)c3)n(S(=O)(=O)c3ccc(C(F)F)cc3)c3ccc(F)cc23)CCCC1] | ¹H-NMR (400 MHz, CD₃OD) δ: 8.32 (dd, J = 9.0, 4.6 Hz, 1H), 8.16 (d, J = 1.6 Hz, 1H), 8.07 (dd, J = 7.8, 1.4 Hz, 1H), 7.62-7.22 (m, 10H), 7.15 (dd, J = 8.2, 2.6 Hz, 1H), 6.70 (t, J = 55.6 Hz, 1H), 2.43-2.16 (m, 3H), 1.92-1.43 (m, 5H); MS: 659.0 (M − 1)⁻. |
| 11/43 | 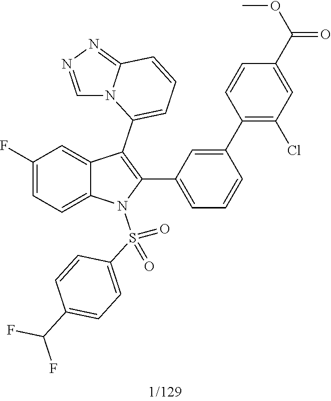 | 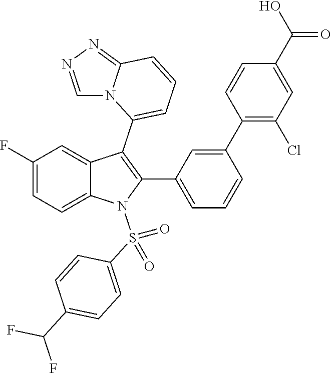 [SMILES: O=C(O)c1ccc(-c2cccc(-c3c(-c4cccc5nncn45)c4cc(F)ccc4n3S(=O)(=O)c3ccc(C(F)F)cc3)c2)c(Cl)c1] | ¹H-NMR (500 MHz, CD₃OD) δ: 8.67 (br s, 1H), 8.51-8.48 (m, 1H), 8.00-7.93 (m, 2H), 7.75 (d, J = 9.5 Hz, 1H), 7.63-7.00 (m, 13H), 6.73 (t, J = 55.3 Hz, 1H); MS: 671.0 (M − 1)⁻. |
| 11/44 | 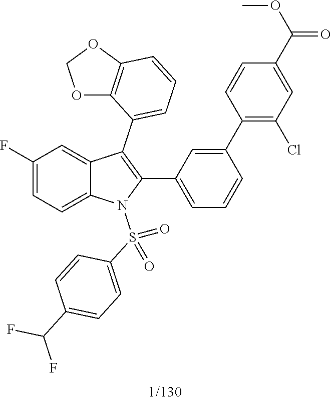 | 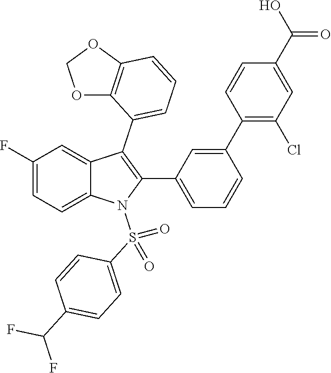 [SMILES: O=C(O)c1ccc(-c2cccc(-c3c(-c4cccc5c4OCO5)c4cc(F)ccc4n3S(=O)(=O)c3ccc(C(F)F)cc3)c2)c(Cl)c1] | ¹H-NMR (500 MHz, CD₃OD) δ: 8.38 (dd, J = 9.1, 4.3 Hz, 1H), 8.08 (d, J = 1.5 Hz, 1H), 7.99 (dd, J = 7.8, 1.4 Hz, 1H), 7.56-7.415 (m, 7H), 7.35 (d, J = 8.0 Hz, 1H), 7.27-7.22 (m, 1H), 7.16 (s, 1H), 7.07 (dd, J = 8.5, 2.5 Hz, 1H), 6.84-6.60 (m, 4H), 5.65 (s, 2H); MS: 674.0 (M − 1)⁻. |
| 11/45 | 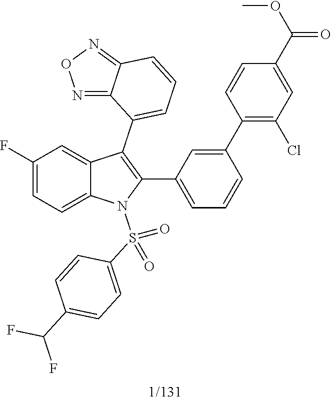 | 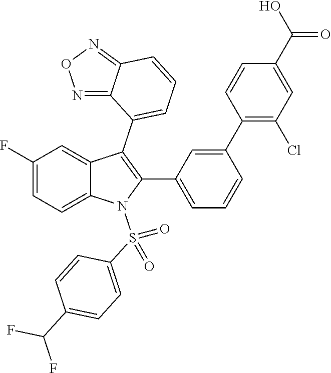 [SMILES: O=C(O)c1ccc(-c2cccc(-c3c(-c4cccc5nonc45)c4cc(F)ccc4n3S(=O)(=O)c3ccc(C(F)F)cc3)c2)c(Cl)c1] | ¹H-NMR (500 MHz, CD₃OD) δ: 8.45 (dd, J = 9.3, 4.3 Hz, 1H), 8.01 (d, J = 1.5 Hz, 1H), 7.98 (dd, J = 8.0, 1.5 Hz, 1H), 7.85 (d, J = 9.0 Hz, 1H), 7.59-7.55 (m, 4H), 7.49-7.28 (m, 7H), 7.07-7.05 (m, 2H), 6.72 (t, J = 55.5 Hz, 1H); MS: 617.9 (M − 1)⁻. |
| 11/46 | 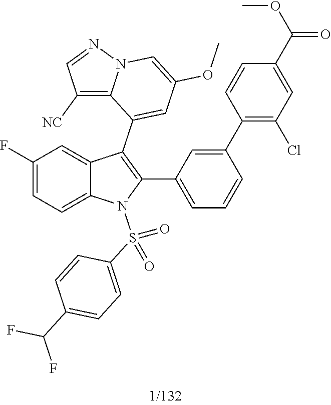 | 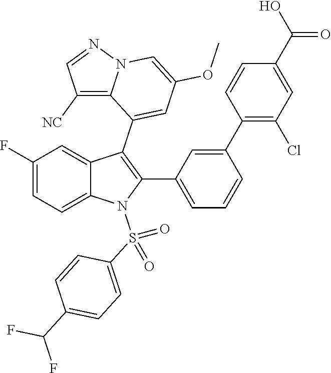 [SMILES: COc1cc(-c2c(-c3cccc(-c4ccc(C(=O)O)cc4Cl)c3)n(S(=O)(=O)c3ccc(C(F)F)cc3)c3ccc(F)cc23)c2c(C#N)cnn2c1] | ¹H-NMR (500 MHz, CD₃OD) δ: 8.43-8.40 (m, 2H), 8.22-7.97 (m, 3H), 7.54-6.72 (m, 13H), 3.86 (s, 3H); MS: 725.0 (M − 1)⁻. |
| 11/47 | 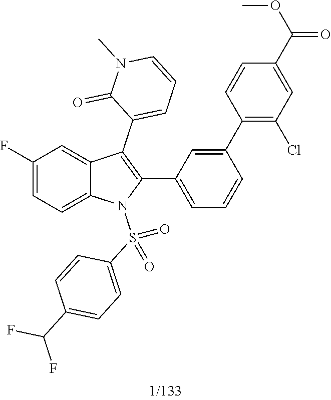 | 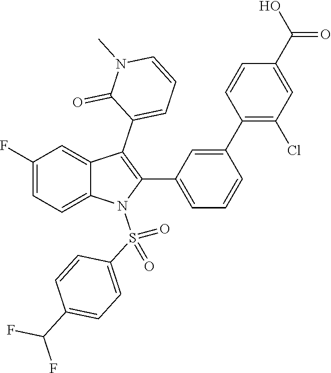 [SMILES: Cn1cccc(-c2c(-c3cccc(-c4ccc(C(=O)O)cc4Cl)c3)n(S(=O)(=O)c3ccc(C(F)F)cc3)c3ccc(F)cc23)c1=O] | ¹H-NMR (500 MHz, CD₃OD) δ: 8.34 (dd, J = 9.3, 4.3 Hz, 1H), 8.08 (s, 1H), 8.01 (d, J = 8.0 Hz, 1H), 7.64 (dd, J = 2.0, 6.5 Hz, 1H), 7.59-7.42 (m, 8H), 7.23-7.18 (m, 3H), 7.00 (dd, J = 2.3, 8.8 Hz, 1H), 6.72 (t, J = 55.5 Hz, 1H), 6.27-6.25 (m, 1H), 3.57 (s, 3H); MS: 661.0 (M − 1)⁻. |
| C11/ 48 | 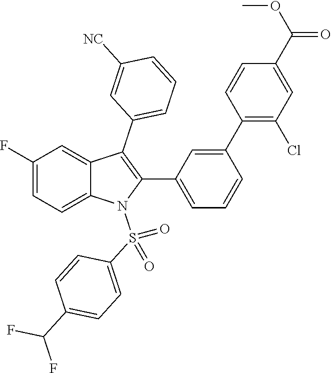 | 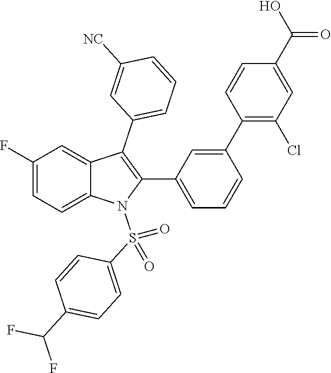 [SMILES: N#Cc1cccc(-c2c(-c3cccc(-c4ccc(C(=O)O)cc4Cl)c3)n(S(=O)(=O)c3ccc(C(F)F)cc3)c3ccc(F)cc23)c1] | ¹H-NMR (500 MHz, CD₃OD) δ: 8.42 (dd, J = 9.0, 4.5 Hz, 1H), 8.06 (d, J = 1.5 Hz, 1H), 7.98 (dd, J = 8.0, 1.5 Hz, 1H), 7.64-7.46 (m, 11H), 7.36 (d, J = 7.5 Hz, 1H), 7.31-7.26 (m, 1H), 7.18 (dd, J = 8.8, 2.8 Hz, 1H), 7.10 (s, 1H), 6.73 (t, J = 55.8 Hz, 1H); MS: 654.9 (M − 1)⁻. |
| | C1/134 | | |
| 11/49 | 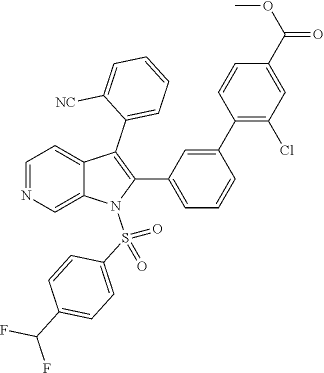 | 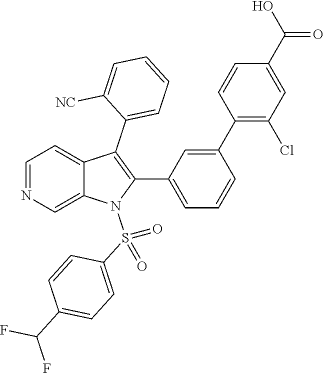 [SMILES: N#Cc1ccccc1-c1c(-c2cccc(-c3ccc(C(=O)O)cc3Cl)c2)n(S(=O)(=O)c2ccc(C(F)F)cc2)c2cnccc12] | ¹H-NMR (400 MHz, DMSO-d₆) δ: 9.52 (s, 1H), 8.52 (d, J = 5.2 Hz, 1H), 7.94-7.88 (m, 3H), 7.71-7.32 (m, 12H), 7.14-6.86 (m, 2H); MS: 638.0 (M − 1)⁻. |
| | 1/135 | | |
| 11/50 | 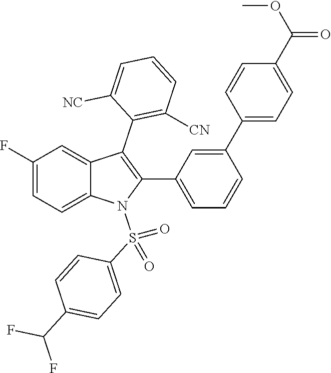 | 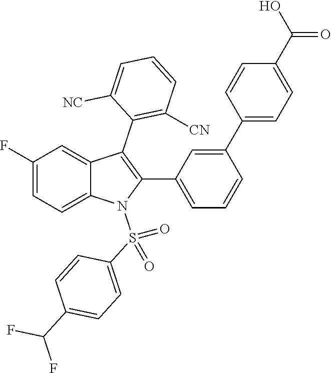 [SMILES: N#Cc1cccc(C#N)c1-c1c(-c2cccc(-c3ccc(C(=O)O)cc3)c2)n(S(=O)(=O)c2ccc(C(F)F)cc2)c2ccc(F)cc12] | ¹H-NMR (500 MHz, DMSO-d₆) δ: 8.36 (dd, J = 9.5, 4.0 Hz, 1H), 8.25 (d, J = 9.0 Hz, 2H), 8.03 (d, J = 8.5 Hz, 2H), 7.79 (t, J = 7.8 Hz, 2H), 7.68 (d, J = 8.0 Hz, 2H), 7.60-7.56 (m, 4H), 7.50-7.40 (m, 3H), 7.26 (dd, J = 2.5, 8.5 Hz, 1H), 7.19 (d, J = 7.5 Hz, 1H), 7.04 (t, J = 55.3 Hz, 1H); MS: 646.0 (M − 1)⁻. |
| | 2/25 | | |
| 11/51 | 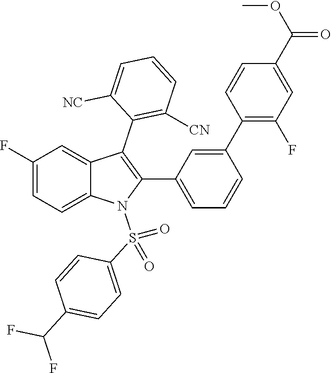 | 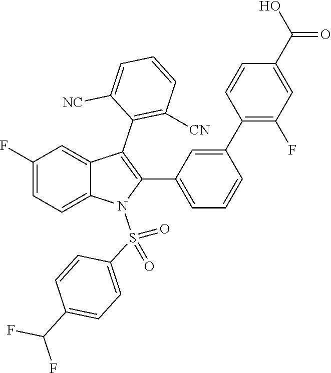 [SMILES: N#Cc1cccc(C#N)c1-c1c(-c2cccc(-c3ccc(C(=O)O)cc3F)c2)n(S(=O)(=O)c2ccc(C(F)F)cc2)c2ccc(F)cc12] | ¹H-NMR (500 MHz, CD₃OD) δ: 8.45 (dd, J = 9.5, 4.0 Hz, 1H), 8.06 (br s, 2H), 7.89 (dd, J = 8.0, 1.5 Hz, 1H), 7.78- 7.63 (m, 3H), 7.56-7.29 (m, 9H), 6.95 (dd, J = 8.5, 2.5 Hz, 1H), 6.69 (t, J = 55.8 Hz, 1H); MS: 664.0 (M − 1)⁻. |
| | 2/26 | | |
| 11/52 | 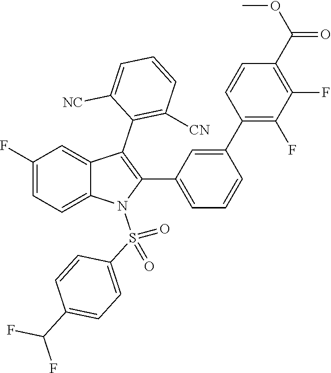 | 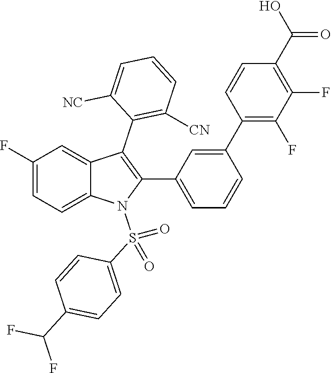 [SMILES: N#Cc1cccc(C#N)c1-c1c(-c2cccc(-c3ccc(C(=O)O)c(F)c3F)c2)n(S(=O)(=O)c2ccc(C(F)F)cc2)c2ccc(F)cc12] | ¹H-NMR (500 MHz, CD₃OD) δ: 8.45 (dd, J = 9.0, 4.5 Hz, 1H), 8.07 (br s, 2H), 7.77-7.64 (m, 3H), 7.57-7.48 (m, 5H), 7.41-7.29 (m, 3H), 7.21-7.18 (m, 1H), 6.96 (dd, J = 8.5, 2.5 Hz, 1H), 6.71 (t, J = 55.8 Hz, 1H); MS: 682.0 (M − 1)⁻. |
| | 2/27 | | |
| 11/53 | 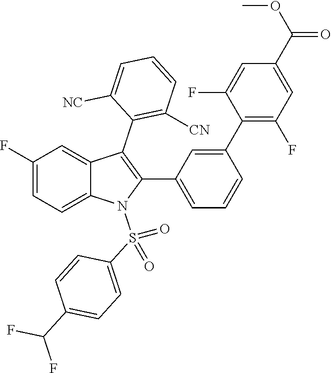 | 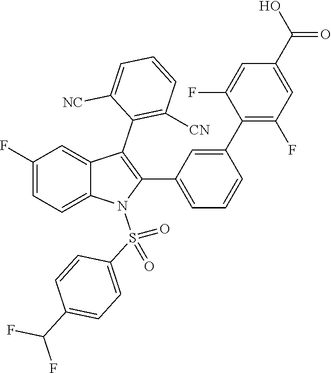 [SMILES: N#Cc1cccc(C#N)c1-c1c(-c2cccc(-c3c(F)cc(C(=O)O)cc3F)c2)n(S(=O)(=O)c2ccc(C(F)F)cc2)c2ccc(F)cc12] | ¹H-NMR (500 MHz, CD₃OD) δ: 8.44 (dd, J = 9.0, 4.0 Hz, 1H), 8.07-8.03 (m, 2H), 7.72 (t, J = 8.3 Hz, 1H), 7.66-7.50 (m, 9H), 7.33 (td, J = 9.0, 2.5 Hz, 1H), 7.00 (s, 1H), 6.95 (dd, J = 8.0, 2.5 Hz, 1H), 6.68 (t, J = 55.5 Hz, 1H); MS: 682.0 (M − 1)⁻. |
| | 2/28 | | |
| 11/54 | 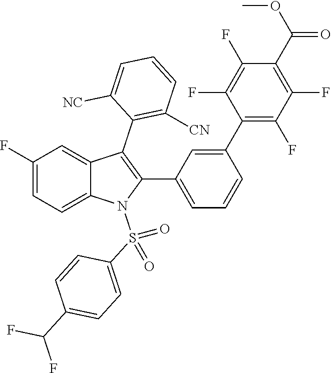 | 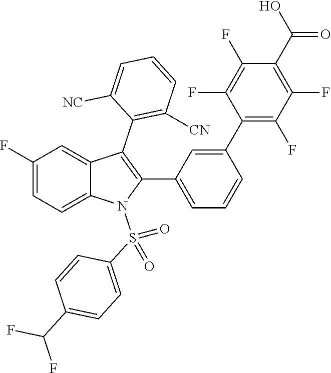 [SMILES: N#Cc1cccc(C#N)c1-c1c(-c2cccc(-c3c(F)c(F)c(C(=O)O)c(F)c3F)c2)n(S(=O)(=O)c2ccc(C(F)F)cc2)c2ccc(F)cc12] | ¹H-NMR (500 MHz, CD₃OD) δ: 8.44 (dd, J = 9.3, 4.8 Hz, 1H), 8.12-8.02 (m, 2H), 7.77-7.72 (m, 1H), 7.62-7.51 (m, 7H), 7.35 (td, J = 9.3, 2.8 Hz, 1H), 7.06 (s, 1H), 6.97 (dd, J = 8.0, 2.5 Hz, 1H), 6.71 (t, J = 55.5 Hz, 1H); MS: 737.1 (M + 18)⁺. |
| | 2/29 | | |
| 11/55 | 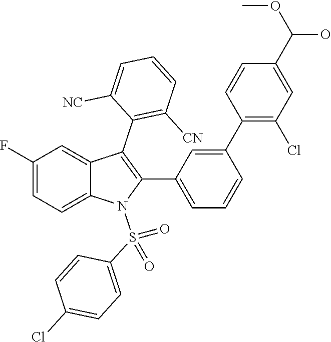 [SMILES: COC(O)c1ccc(-c2cccc(-c3c(-c4c(C#N)cccc4C#N)c4cc(F)ccc4n3S(=O)(=O)c3ccc(Cl)cc3)c2)c(Cl)c1] | 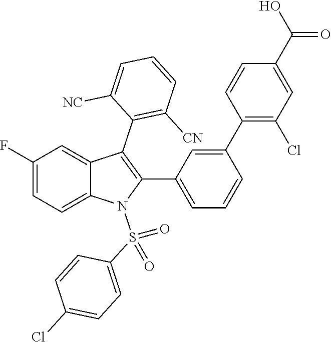 [SMILES: N#Cc1cccc(C#N)c1-c1c(-c2cccc(-c3ccc(C(=O)O)cc3Cl)c2)n(S(=O)(=O)c2ccc(Cl)cc2)c2ccc(F)cc12] | ¹H-NMR (500 MHz, CD₃OD) δ: 8.42 (dd, J = 9.5, 4.0 Hz, 1H), 8.09-8.04 (m, 3H), 8.00 (dd, J = 1.5, 8.0 Hz, 1H), 7.73 (t, J = 7.8 Hz, 1H), 7.49-7.44 (m, 3H), 7.38- 7.29 (m, 6H), 7.05 (d, J = 1.0 Hz, 1H), 6.95 (dd, J = 8.5, 2.5 Hz, 1H); MS: 663.9/666.0 (M − 1)⁻. |
| | 1/136 | | |
| 11/56 | 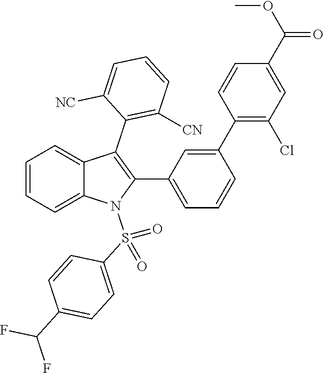 | 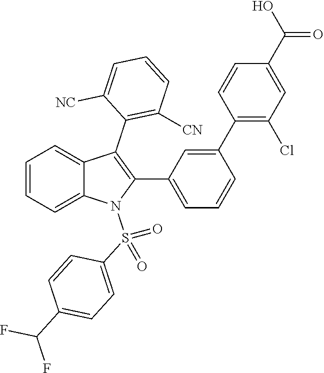 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccccc12] | ¹H-NMR (500 MHz, CD₃OD) δ: 8.43 (d, J = 8.5 Hz, 1H), 8.09- 8.04 (m, 3H), 7.98 (dd, J = 7.5, 1.5 Hz, 1H), 7.72 (t, J = 8.0 Hz, 1H), 7.58-7.47 (m, 8H), 7.42 (t, J = 7.8 Hz, 1H), 7.34 (d, J = 8.0 Hz, 1H), 7.19 (d, J = 7.5 Hz, 1H), 7.07 (s, 1H), 6.66 (t, J = 55.5 Hz, 1H); MS: 661.9 (M − 1)⁻. |
| | 1/137 | | |
| 11/57 | 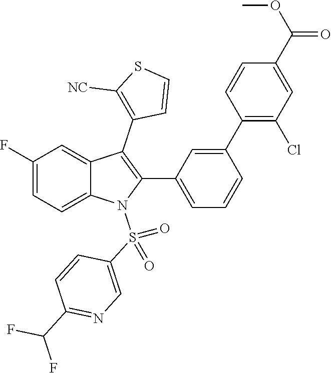 | 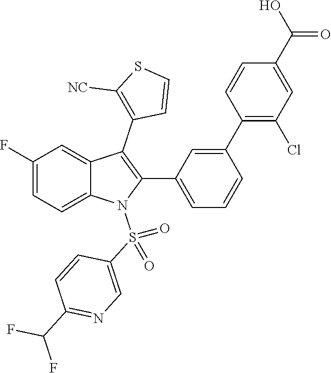 [SMILES: N#Cc1sccc1-c1c(-c2cccc(-c3ccc(C(=O)O)cc3Cl)c2)n(S(=O)(=O)c2ccc(C(F)F)nc2)c2ccc(F)cc12] | ¹H-NMR (400 MHz, CD₃OD) δ: 8.60 (d, J = 2.0 Hz, 1H), 8.44 (dd, J = 4.4, 9.2 Hz, 1H), 8.10 (s, 1H), 8.03-7.31 (m, 9H), 7.21 (s, 1H), 7.09 (dd, J = 2.8, 8.4 Hz, 1H), 7.00 (d, J = 4.8 Hz, 1H), 6.65 (t, J = 54.6 Hz, 1H); MS: 663.8 (M + 1)⁺. |
| | 35/2 | | |
| 11/58 | 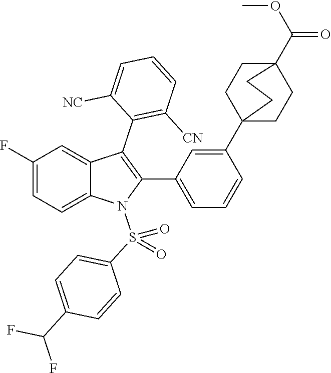 | 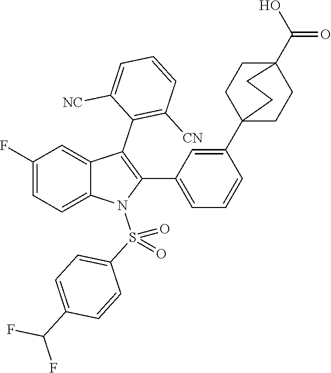 [SMILES: N#Cc1cccc(C#N)c1-c1c(-c2cccc(C34CCC(C(=O)O)(CC3)CC4)c2)n(S(=O)(=O)c2ccc(C(F)F)cc2)c2ccc(F)cc12] | ¹H-NMR (500 MHz, CD₃OD) δ: 8.45 (dd, J = 9.3, 4.3 Hz, 1H), 8.07-7.98 (m, 2H), 7.69 (t, J = 8.0 Hz, 1H), 7.58 (d, J = 8.5 Hz, 2H), 7.43 (d, J = 8.0 Hz, 2H), 7.37- 7.31 (m, 2H), 7.24 (t, J = 7.8 Hz, 1H), 7.11 (d, J = 8.0 Hz, 1H), 6.97-6.93 (m, 2H), 6.79 (t, J = 55.8 Hz, 1H), 1.90- 1.87 (m, 6H), 1.70-1.67 (m, 6H); MS: 678.0 (M − 1)⁻. |
| | 1/138 | | |
| 11/59 | 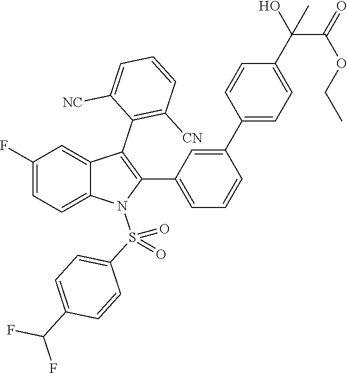 | 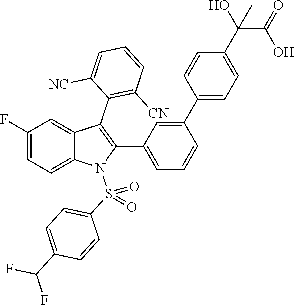 [SMILES: CC(O)(C(=O)O)c1ccc(-c2cccc(-c3c(-c4c(C#N)cccc4C#N)c4cc(F)ccc4n3S(=O)(=O)c3ccc(C(F)F)cc3)c2)cc1] | ¹H-NMR (500 MHz, CD₃OD) δ: 8.46 (dd, J = 9.2, 4.3 Hz, 1H), 8.05 (d, J = 7.9 Hz, 2H), 7.75- 7.62 (m, 4H), 7.52 (q, J = 8.6 Hz, 4H), 7.47-7.28 (m, 5H), 7.21 (d, J = 7.8 Hz, 1H), 6.96 (dd, J = 8.3, 2.5 Hz, 1H), 6.67 (t, J = 55.6 Hz, 1H), 1.79 (s, 3H); MS: 690.0 (M − 1)⁻. |
| | 2/30 | | |
| 11/60 | 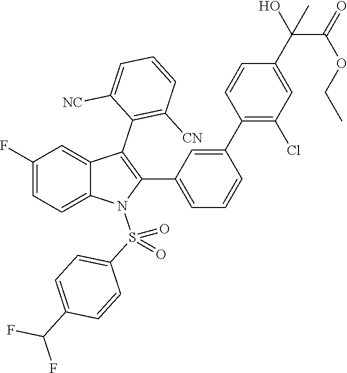 | 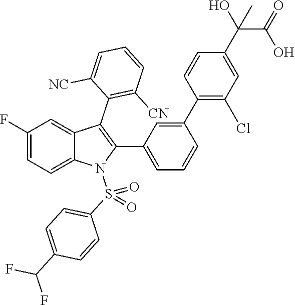 [SMILES: CC(O)(C(=O)O)c1ccc(-c2cccc(-c3c(-c4c(C#N)cccc4C#N)c4cc(F)ccc4n3S(=O)(=O)c3ccc(C(F)F)cc3)c2)c(Cl)c1] | ¹H-NMR (500 MHz, CD₃OD) δ: 8.44 (dd, J = 9.5, 4.3 Hz, 1H), 8.06 (br s, 2H), 7.75-7.70 (m, 2H), 7.62-7.59 (m, 1H), 7.52-7.48 (m, 4H), 7.45- 7.43 (m, 2H), 7.35- 7.31 (m, 1H), 7.21 (d, J = 7.5 Hz, 1H), 6.98-6.93 (m, 2H), 6.64 (t, J = 55.6 Hz, 1H), 1.77 (s, 3H); MS: 724.0 (M − 1)⁻. |
| | 2/31 | | |
| 11/61 | 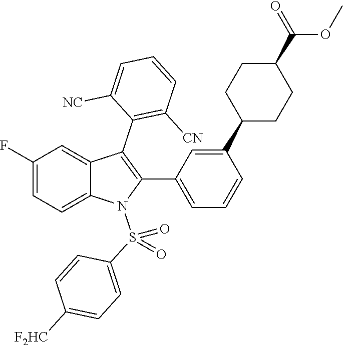 | 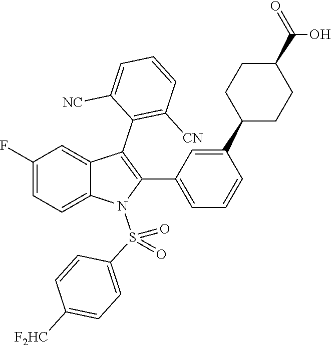 [SMILES: N#Cc1cccc(C#N)c1-c1c(-c2cccc([C@H]3CC[C@@H](C(=O)O)CC3)c2)n(S(=O)(=O)c2ccc(C(F)F)cc2)c2ccc(F)cc12] | ¹H-NMR (500 MHz, CD₃OD) δ: 8.45-8.42 (m, 1H), 8.02 (br s, 2H), 7.68 (t, J = 7.9 Hz, 1H), 7.57 (d, J = 8.3 Hz, 2H), 7.45 (d, J = 8.3 Hz, 2H), 7.37- 7.13 (m, 4H), 6.98-6.60 (m, 3H), 2.65 (s, 1H), 2.50-2.45 (m, 1H), 2.17-2.14 (m, 2H), 1.71-1.11 (m, 6H); MS: 652.0 (M − 1)⁻. |
| | 43/1 | | |
| 11/62 | 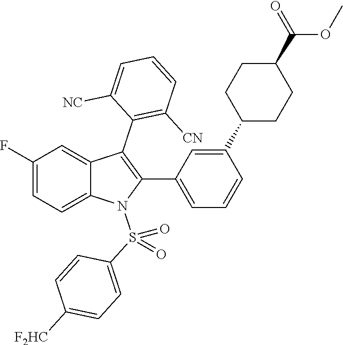 | 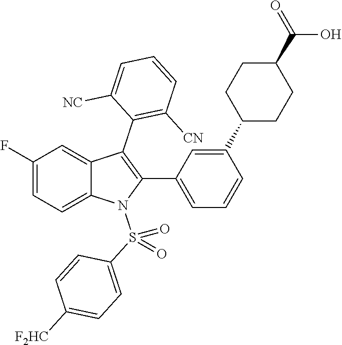 [SMILES: N#Cc1cccc(C#N)c1-c1c(-c2cccc([C@H]3CC[C@H](C(=O)O)CC3)c2)n(S(=O)(=O)c2ccc(C(F)F)cc2)c2ccc(F)cc12] | ¹H-NMR (500 MHz, CD₃OD) δ: 8.44 (dd, J = 9.2, 4.3 Hz, 1H), 8.04- 8.00 (m, 2H), 7.69 (t, J = 7.9 Hz, 1H), 7.58 (d, J = 8.3 Hz, 2H), 7.47 (d, J = 8.3 Hz, 2H), 7.32 (td, J = 9.1, 2.5 Hz, 1H), 7.29-7.16 (m, 2H), 7.09-7.07 (m, 1H), 7.01-6.61 (m, 3H), 2.46-2.41 (m, 1H), 2.31-2.25 (m, 1H), 2.08 (d, J = 11.8 Hz, 2H), 1.79 (br s, 2H), 1.55 (qd, J = 13.0, 3.3 Hz, 2H), 1.32 (br s, 2H); MS: 652.0 (M − 1)⁻. |
| | 43/2 | | |
| 11/63 | 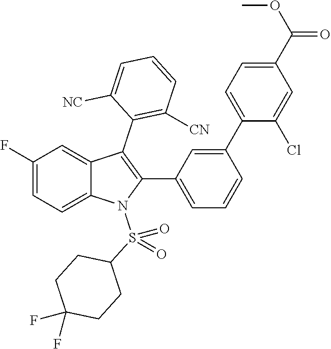 | 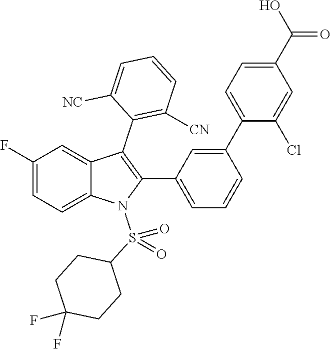 [SMILES: N#Cc1cccc(C#N)c1-c1c(-c2cccc(-c3ccc(C(=O)O)cc3Cl)c2)n(S(=O)(=O)C2CCC(F)(F)CC2)c2ccc(F)cc12] | ¹H-NMR (500 MHz, CD₃OD) δ: 8.28 (dd, J = 4.3, 9.3 Hz, 1H), 8.12 (d, J = 7.5 Hz, 2H), 9.09 (d, J = 2.0 Hz, 1H), 7.99 (dd, J = 1.5, 8.0 Hz, 1H), 7.77 (t, J = 7.8 Hz, 1H), 7.53- 7.33 (m, 6H), 7.04 (dd, J = 2.5, 9.0 Hz, 1H), 3.48-3.43 (m, 1H), 2.04 (br s, 2H), 1.80-1.67 (m, 6H); MS: 672.0 (M − 1)⁻. |
| | 1/142 | | |
| 11/64 | 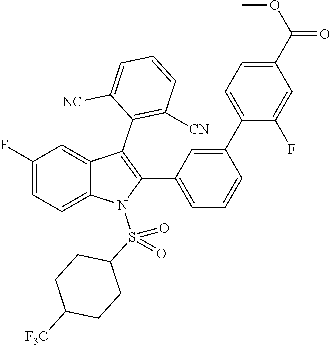 | 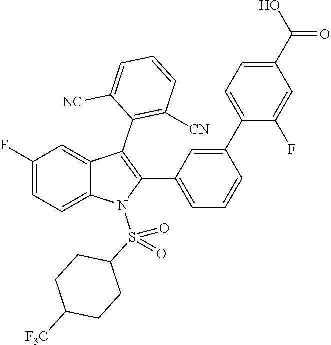 [SMILES: N#Cc1cccc(C#N)c1-c1c(-c2cccc(-c3ccc(C(=O)O)cc3F)c2)n(S(=O)(=O)C2CCC(C(F)(F)F)CC2)c2ccc(F)cc12] | ¹H-NMR (500 MHz, DMSO-d₆) δ: 8.29 (d, J = 7.5 Hz, 2H), 8.16 (dd, J = 4.3, 9.3 Hz, 1H), 7.84- 7.81 (m, 2H), 7.71 (dd, J = 11.3, 1.3 Hz, 1H), 7.62 (d, J = 7.0 Hz, 1H), 7.51-7.43 (m, 4H), 7.38 (d, J = 8.0 Hz, 1H), 7.30 (dd, J = 2.5, 8.5 Hz, 1H), 3.56-3.51 (m, 1H), 2.30-2.22 (m, 1H), 1.79-1.67 (m, 4H), 1.44 (q, J = 11.8 Hz, 2H), 1.22 (q, J = 11.8 Hz, 2H); MS: 688.0 (M − 1)⁻. |
| | 1/143 | | |
| 11/65 | 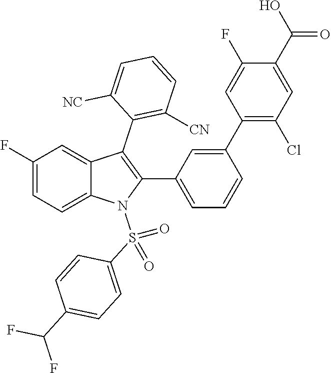 [SMILES: N#Cc1cccc(C#N)c1-c1c(-c2cccc(-c3cc(F)c(C(=O)O)cc3Cl)c2)n(S(=O)(=O)c2ccc(C(F)F)cc2)c2ccc(F)cc12] | 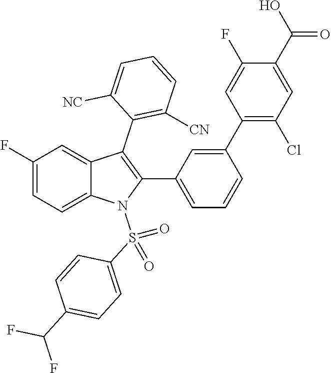 [SMILES: N#Cc1cccc(C#N)c1-c1c(-c2cccc(-c3cc(F)c(C(=O)O)cc3Cl)c2)n(S(=O)(=O)c2ccc(C(F)F)cc2)c2ccc(F)cc12] | ¹H-NMR (500 MHz, DMSO-d₆) δ: 8.44 (dd, J = 4.3, 9.3 Hz, 1H), 8.07 (d, J = 7.0 Hz, 2H), 8.01 (d, J = 6.5 Hz, 1H), 7.73 (t, J = 8.0 Hz, 1H), 7.56- 7.32 (m, 8H), 7.19 (d, J = 11.0 Hz, 1H), 7.11 (s, 1H), 6.96 (dd, J = 2.5, 8.0 Hz, 1H), 6.70 (t, J = 55.8 Hz, 1H); MS: 697.9 (M −1)⁻. |
| | 1/144 | | |
| 11/66 | 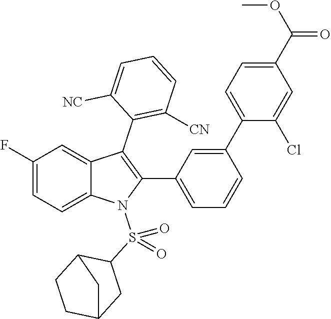 | 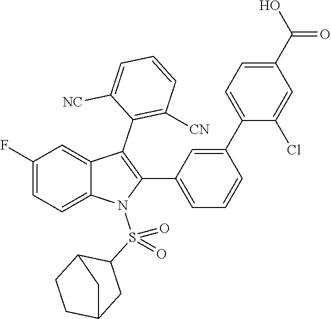 [SMILES: N#Cc1cccc(C#N)c1-c1c(-c2cccc(-c3ccc(C(=O)O)cc3Cl)c2)n(S(=O)(=O)C2CC3CCC2C3)c2ccc(F)cc12] | ¹H-NMR (500 MHz, CD₃OD) δ: 8.29 (dd, J = 9.0, 4.0 Hz, 1H), 8.13-8.08 (m, 3H), 7.99 (dd, J = 8.5, 1.5 Hz, 1H), 7.78-7.74 (m, 1H), 7.57-7.29 (m, 6H), 7.03-6.99 (m, 1H), 3.18 (dd, J = 8.5, 6.0 Hz, 1H), 2.38-2.25 (m, 2H), 1.85-1.05 (m, 8H); MS: 648.0 (M − 1)⁻. |
| | 1/146 | | |
| 11/67 | 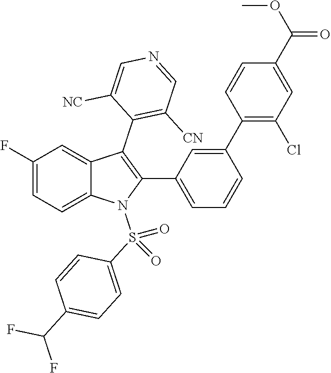 | 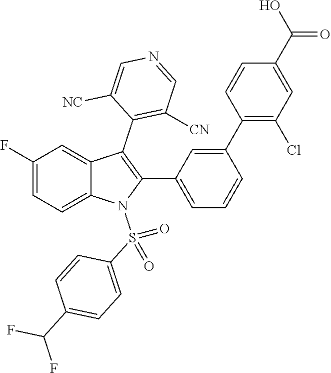 [SMILES: N#Cc1cncc(C#N)c1-c1c(-c2cccc(-c3ccc(C(=O)O)cc3Cl)c2)n(S(=O)(=O)c2ccc(C(F)F)cc2)c2ccc(F)cc12] | ¹H-NMR (500 MHz, CD₃OD) δ: 9.15 (br s, 2H), 8.48 (dd, J = 9.3, 4.3 Hz, 1H), 8.08 (d, J = 1.0 Hz, 1H), 8.00 (dd, J = 8.0, 1.5 Hz, 1H), 7.55-7.48 (m, 6H), 7.44-7.38 (m, 3H), 7.13 (dd, J = 8.0, 2.5 Hz, 1H), 7.01 (s, 1H), 6.68 (t, J = 55.8 Hz, 1H); MS: 681.0 (M − 1)⁻. |
| | 1/147 | | |
| 11/68 | 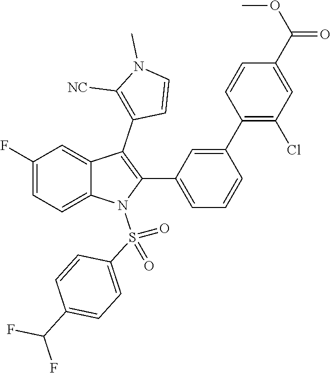 | 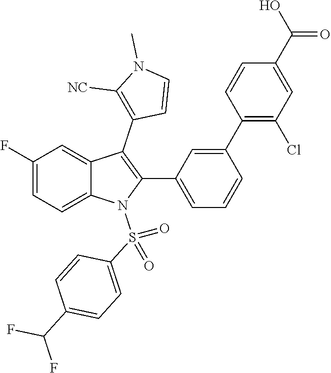 [SMILES: Cn1ccc(-c2c(-c3cccc(-c4ccc(C(=O)O)cc4Cl)c3)n(S(=O)(=O)c3ccc(C(F)F)cc3)c3ccc(F)cc23)c1C#N] | ¹H-NMR (400 MHz, CD₃OD) δ: 8.37 (dd, J = 9.2, 4.4 Hz, 1H), 8.10 (s, 1H), 8.00 (d, J = 7.6 Hz, 1H), 7.55-7.41 (m, 8H), 7.27-7.22 (m, 1H), 7.16 (s, 1H), 7.10 (dd, J = 8.8, 2.4 Hz, 1H), 6.99 (d, J = 2.8 Hz, 1H), 6.70 (t, J = 55.4 Hz, 1H), 6.03 (d, J = 2.8 Hz, 1H), 3.72 (s, 3H); MS: 658.0 (M − 1)⁻. |
| | 1/148 | | |
| 11/69 | 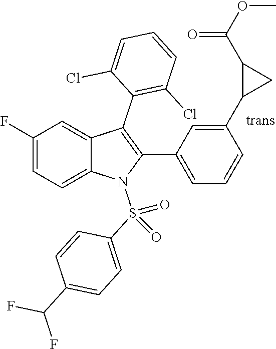 | 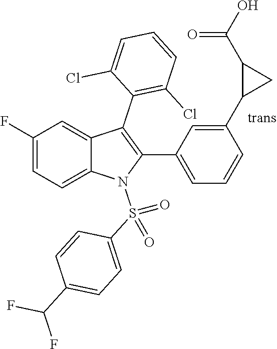 [SMILES: O=C(O)C1CC1c1cccc(-c2c(-c3c(Cl)cccc3Cl)c3cc(F)ccc3n2S(=O)(=O)c2ccc(C(F)F)cc2)c1] | ¹H-NMR (400 MHz, CD₃OD) δ: 8.38 (dd, J = 9.2, 4.4 Hz, 1H), 7.58 (d, J = 8.4 Hz, 2H), 7.45 (d, J = 8.4 Hz, 2H), 7.39-7.16 (m, 7H), 6.94-6.65 (m, 3H), 2.38-2.34 (m, 1H), 1.61-1.55 (m, 1H), 1.50-1.45 (m, 1H), 1.10 (br s, 1H); MS: 628.0 and 630.0 (M − 1)⁻. |
| | 1/149 | | |

### Example Compounds Set 12

Example compounds are shown below.

| # | building block | structure | analytical data |
|---|---|---|---|
| 12 | | 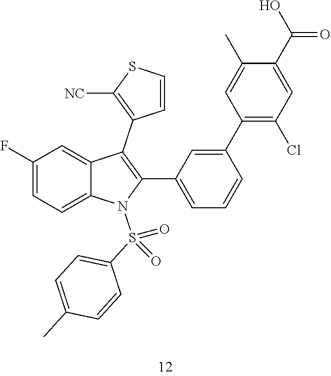 [SMILES: Cc1ccc(S(=O)(=O)n2c(-c3cccc(-c4cc(C)c(C(=O)O)cc4Cl)c3)c(-c3ccsc3C#N)c3cc(F)ccc32)cc1] | |
| 12/1 | 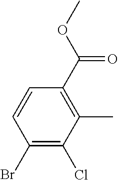 | 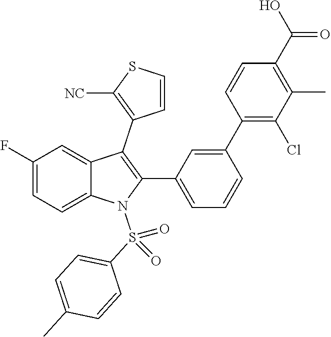 [SMILES: Cc1ccc(S(=O)(=O)n2c(-c3cccc(-c4ccc(C(=O)O)c(C)c4Cl)c3)c(-c3ccsc3C#N)c3cc(F)ccc32)cc1] | ¹H-NMR (500 MHz, DMSO-d₆) δ: 8.29 (dd, J = 9.3, 4.3 Hz, 1H), 8.03 (d, J = 5.0 Hz, 1H), 7.68 (d, J = 8.0 Hz, 1H), 7.53-7.15 (m, 10H), 7.03 (d, J = 5.0 Hz, 1H), 6.98 (s, 1H), 2.55 (s, 3H), 2.23 (s, 3H); MS: 639.0 (M − 1)⁻. |
| 12/2 | 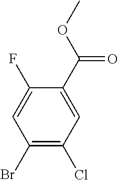 | 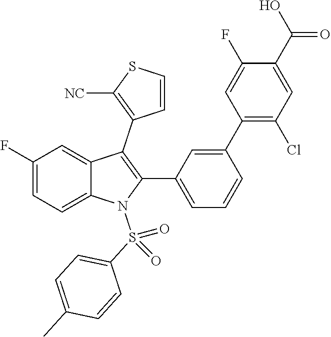 [SMILES: Cc1ccc(S(=O)(=O)n2c(-c3cccc(-c4cc(F)c(C(=O)O)cc4Cl)c3)c(-c3ccsc3C#N)c3cc(F)ccc32)cc1] | ¹H-NMR (500 MHz, DMSO-d₆) δ: 8.42 (dd, J = 9.0, 4.5 Hz, 1H), 7.92 (d, J = 7.5 Hz, 1H), 7.84 (d, J = 5.0 Hz, 1H), 7.53-7.46 (m, 3H), 7.30-7.26 (m, 3H), 7.19-6.97 (m, 6H), 2.28 (s, 3H); MS: 643.0 (M − 1)⁻. |
| 12/3 | 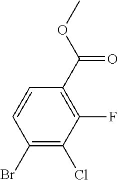 | 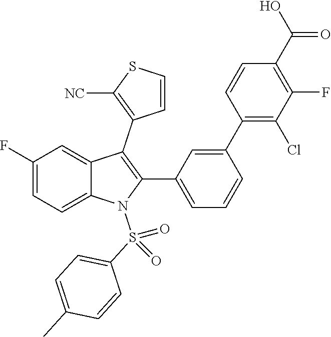 [SMILES: Cc1ccc(S(=O)(=O)n2c(-c3cccc(-c4ccc(C(=O)O)c(F)c4Cl)c3)c(-c3ccsc3C#N)c3cc(F)ccc32)cc1] | ¹H-NMR (500 MHz, CD₃OD) δ: 8.42 (dd, J = 9.3, 4.3 Hz, 1H), 7.85-7.80 (m, 2H), 7.53-7.47 (m, 3H), 7.30-7.25 (m, 3H), 7.17 (d, J = 8.0 Hz, 3H), 7.07 (dd, J = 7.8, 2.8 Hz, 1H), 7.04-6.96 (m, 2H), 2.27 (s, 3H); MS: 642.9 (M − 1)⁻. |
| 12/4 | 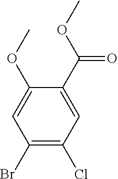 | 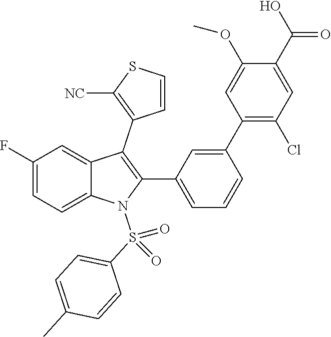 [SMILES: COc1cc(-c2cccc(-c3c(-c4ccsc4C#N)c4cc(F)ccc4n3S(=O)(=O)c3ccc(C)cc3)c2)c(Cl)cc1C(=O)O] | ¹H-NMR (500 MHz, DMSO-d₆) δ: 8.29 (dd, J = 9.3, 4.3 Hz, 1H), 8.03 (d, J = 5.0 Hz, 1H), 7.59-7.50 (m, 3H), 7.43 (d, J = 7.5 Hz, 1H), 7.40-7.34 (m, 3H), 7.24 (d, J = 8.5 Hz, 2H), 7.17 (dd, J = 8.5, 2.5 Hz, 1H), 7.10 (s, 1H), 7.06 (d, J = 5.5 Hz, 1H), 6.94 (s, 1H), 3.82 (s, 3H), 2.22 (s, 3H); MS: 655.0 (M − 1)⁻. |
| 12/5 | 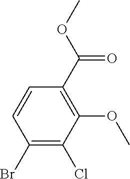 | 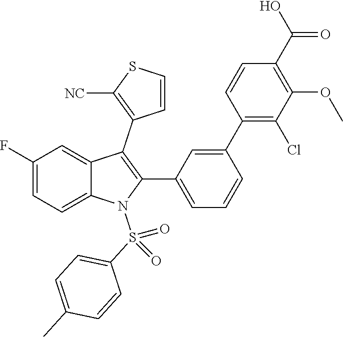 [SMILES: COc1c(C(=O)O)ccc(-c2cccc(-c3c(-c4ccsc4C#N)c4cc(F)ccc4n3S(=O)(=O)c3ccc(C)cc3)c2)c1Cl] | ¹H-NMR (500 MHz, CD₃OD) δ: 8.42 (dd, J = 9.3, 4.3 Hz, 1H), 7.84 (d, J = 5.0 Hz, 1H), 7.69 (d, J = 7.5 Hz, 1H), 7.51-7.45 (m, 3H), 7.29-7.24 (m, 3H), 7.16 (d, J = 8.0 Hz, 2H), 7.09-7.05 (m, 2H), 6.97-6.95 (m, 2H), 3.95 (s, 3H), 2.27 (s, 3H); MS: 655.0 (M − 1)⁻. |
| 12/6 | 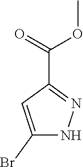 | 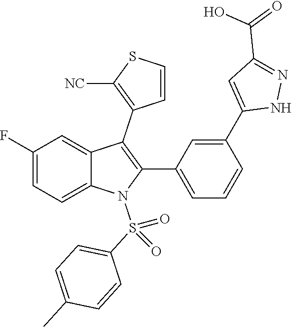 [SMILES: Cc1ccc(S(=O)(=O)n2c(-c3cccc(-c4cc(C(=O)O)n[nH]4)c3)c(-c3ccsc3C#N)c3cc(F)ccc32)cc1] | ¹H-NMR (500 MHz, DMSO-d₆) δ: 8.30 (dd, J = 9.2, 4.4 Hz, 1H), 7.98 (d, J = 5.1 Hz, 1H), 7.88 (d, J = 7.8 Hz, 1H), 7.61 (s, 1H), 7.48-7.33 (m, 4H), 7.29 (d, J = 8.3 Hz, 2H), 7.25-7.13 (m, 2H), 6.99 (d, J = 5.0 Hz, 1H), 6.93 (br s, 1H), 2.28 (s, 3H); MS: 581.0 (M − 1)⁻. |
| 12/7 | 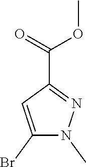 | 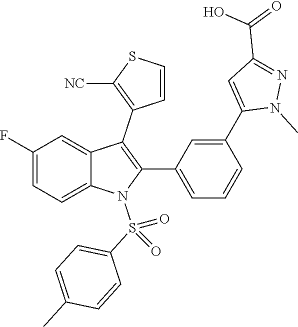 [SMILES: Cc1ccc(S(=O)(=O)n2c(-c3cccc(-c4cc(C(=O)O)nn4C)c3)c(-c3ccsc3C#N)c3cc(F)ccc32)cc1] | ¹H-NMR (500 MHz, DMSO-d₆) δ: 8.30 (dd, J = 9.2, 4.4 Hz, 1H), 7.98 (d, J = 5.1 Hz, 1H), 7.61 (br s, 1H), 7.52 (br s, 1H), 7.48-7.28 (m, 7H), 7.20-7.17 (m, 1H), 7.06 (d, J = 4.5 Hz, 1H), 6.64 (br s, 1H), 3.76 (br s, 3H), 2.28 (s, 3H); MS: 597.2 (M + H)⁺. |
| 12/8 | 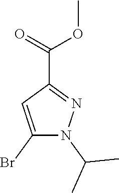 | 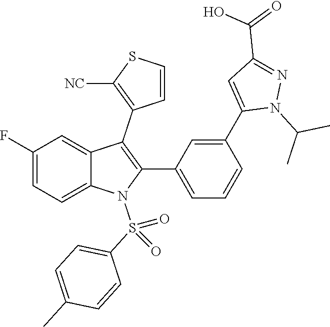 [SMILES: Cc1ccc(S(=O)(=O)n2c(-c3cccc(-c4cc(C(=O)O)nn4C(C)C)c3)c(-c3ccsc3C#N)c3cc(F)ccc32)cc1] | ¹H-NMR (500 MHz, DMSO-d₆) δ: 8.29 (dd, J = 9.2, 4.4 Hz, 1H), 8.07 (d, J = 5.1 Hz, 1H), 7.59-7.49 (m, 2H), 7.45- 7.31 (m, 4H), 7.28 (d, J = 8.3 Hz, 2H), 7.20 (dd, J = 8.6, 2.6 Hz, 1H), 7.10-7.00 (m, 2H), 6.61 (s, 1H), 4.25-4.22 (m, 1H), 2.26 (s, 3H), 1.30 (d, J = 6.5 Hz, 6H); MS: 623.0 (M − 1)⁻. |
| 12/9 | 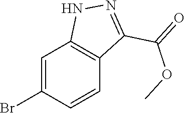 | 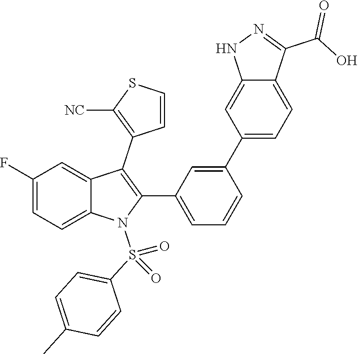 [SMILES: Cc1ccc(S(=O)(=O)n2c(-c3cccc(-c4ccc5c(C(=O)O)n[nH]c5c4)c3)c(-c3ccsc3C#N)c3cc(F)ccc32)cc1] | ¹H-NMR (500 MHz, DMSO-d₆) δ: 8.31 (dd, J = 9.2, 4.4 Hz, 1H), 8.17 (d, J = 8.2 Hz, 1H), 8.04 (d, J = 5.1 Hz, 1H), 7.80 (d, J = 7.6 Hz, 1H), 7.68 (s, 1H), 7.55-7.44 (m, 2H), 7.40-7.36 (m, 4H), 7.28-7.24 (m, 3H), 7.19 (dd, J = 8.5, 2.5 Hz, 1H), 7.09 (d, J = 5.1 Hz, 1H), 2.22 (s, 3H); MS: 631.0 (M − 1)⁻. |
| 12/10 | 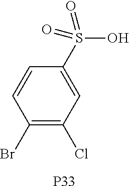 [SMILES: "P33" ; O=S(=O)(O)c1ccc(Br)c(Cl)c1] | 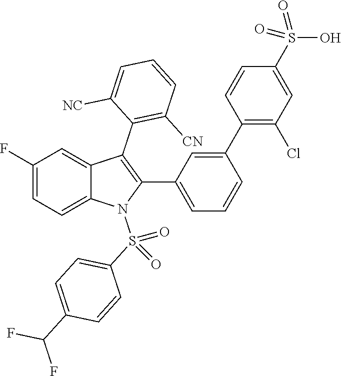 [SMILES: N#Cc1cccc(C#N)c1-c1c(-c2cccc(-c3ccc(S(=O)(=O)O)cc3Cl)c2)n(S(=O)(=O)c2ccc(C(F)F)cc2)c2ccc(F)cc12] | ¹H-NMR (500 MHz, CD₃OD) δ: 8.44 (dd, J = 9.5, 4.5 Hz, 1H), 8.06 (br s, 2H), 7.89 (d, J = 1.5 Hz, 1H), 7.80 (dd, J = 1.3, 7.8 Hz, 1H), 7.72 (t, J = 8.0 Hz, 1H), 7.55-7.48 (m, 7H), 7.36-7.31 (m, 2H), 7.02 (s, 1H), 6.95 (dd, J = 2.3, 8.3 Hz, 1H), 6.68 (t, J = 55.8 Hz, 1H); MS: 715.9 (M − 1)⁻. |

### Example Compounds Set 13

Example compounds are shown below.

| # | starting material | structure | analytical data |
|---|---|---|---|
| 13 | | 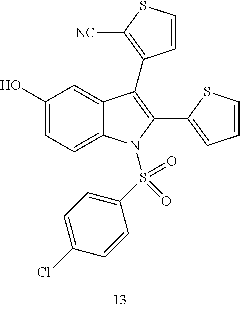 [SMILES: N#Cc1sccc1-c1c(-c2cccs2)n(S(=O)(=O)c2ccc(Cl)cc2)c2ccc(O)cc12] | |
| 13/1 | 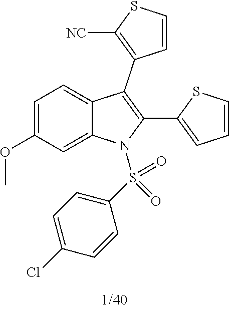 | 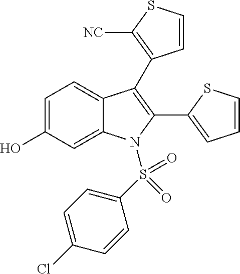 [SMILES: N#Cc1sccc1-c1c(-c2cccs2)n(S(=O)(=O)c2ccc(Cl)cc2)c2cc(O)ccc12] | ¹H-NMR (500 MHz, DMSO-d₆) δ: 9.99 (s, 1H), 8.01 (d, J = 5.0 Hz, 1H), 7.70-7.68 (m, 2H), 7.63 (d, J = 9.0 Hz, 2H), 7.50 (d, J = 8.5 Hz, 2H), 7.16- 7.15 (m, 2H), 7.09-7.08 (m, 1H), 7.00 (d, J = 5.0 Hz, 1H), 6.87 (dd, J = 2.0, 8.5 Hz, 1H); MS: 496.7 (M + 1)⁺. |

### Example Compounds Set 15

Example compounds are shown below.

| # | building block(s) | structure | analytical data |
|---|---|---|---|
| 15 | | 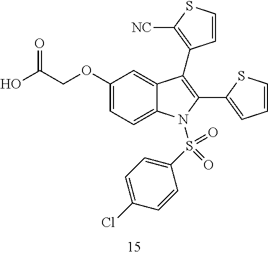 [SMILES: N#Cc1sccc1-c1c(-c2cccs2)n(S(=O)(=O)c2ccc(Cl)cc2)c2ccc(OCC(=O)O)cc12] | |
| 15/1 | 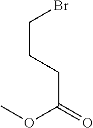 | 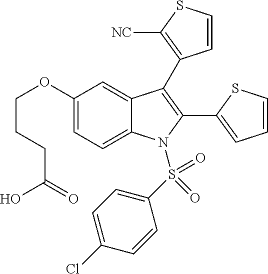 [SMILES: N#Cc1sccc1-c1c(-c2cccs2)n(S(=O)(=O)c2ccc(Cl)cc2)c2ccc(OCCCC(=O)O)cc12] | ¹H-NMR (500 MHz, DMSO-d₆) δ: 12.12 (s, 1H), 8.14 (d, J = 9.0 Hz, 1H), 8.03 (d, J = 5.0 Hz, 1H), 7.73 (d, J = 4.5 Hz, 1H), 7.59 (d, J = 9.0 Hz, 2H), 7.48 (d, J = 9.0 Hz, 2H), 7.20 (d, J = 2.5 Hz, 1H), 7.15-7.11 (m, 2H), 7.02 (d, J = 5.0 Hz, 1H), 6.77 (d, J = 3.0 Hz, 1H), 3.97 (t, J = 6.5 Hz, 2H), 2.37 (t, J = 7.3 Hz, 2H), 1.93 (t, J = 6.8 Hz, 2H); MS: 582.7 (M + 1)⁺. |
| 15/2 | 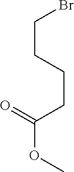 | 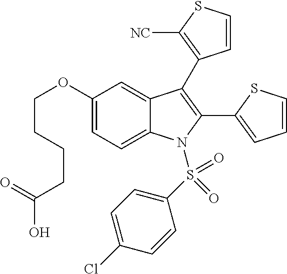 [SMILES: N#Cc1sccc1-c1c(-c2cccs2)n(S(=O)(=O)c2ccc(Cl)cc2)c2ccc(OCCCCC(=O)O)cc12] | ¹H-NMR (500 MHz, DMSO-d₆) δ: 12.03 (br s, 1H), 8.14 (d, J = 9.5 Hz, 1H), 8.04 (d, J = 5.0 Hz, 1H), 7.74 (d, J = 5.0 Hz, 1H), 7.59 (d, J = 8.0 Hz, 2H), 7.48 (d, J = 9.0 Hz, 2H), 7.21-7.20 (m, 1H), 7.15-7.11 (m, 2H), 7.02 (d, J = 4.5 Hz, 1H), 6.77 (d, J = 2.5 Hz, 1H), 3.96 (t, J = 6.0 Hz, 2H), 2.27 (t, J = 7.3 Hz, 2H), 1.75-1.62 (m, 4H); MS: 596.9 (M + 1)⁺. |
| 15/3 | 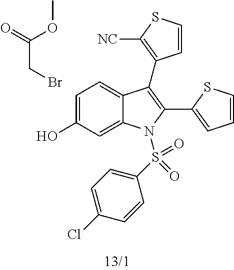 | 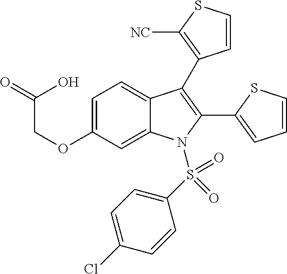 [SMILES: N#Cc1sccc1-c1c(-c2cccs2)n(S(=O)(=O)c2ccc(Cl)cc2)c2cc(OCC(=O)O)ccc12] | ¹H-NMR (500 MHz, DMSO-d₆) δ: 13.20 (s, 1H), 8.03 (d, J = 5.0 Hz, 1H), 7.72 (d, J = 5.0 Hz, 2H), 7.60 (d, J = 8.5 Hz, 2H), 7.54 (d, J = 8.5 Hz, 2H), 7.26 (d, J = 9.0 Hz, 1H), 7.19 (d, J = 3.5 Hz, 1H), 7.12-7.10 (m, 1H), 7.05 (dd, J = 2.0, 9.0 Hz, 1H), 7.01 (d, J = 5.0 Hz, 1H), 4.86 (s, 2H); MS: 554.6 (M + 1)⁺. |
| 15/4 | 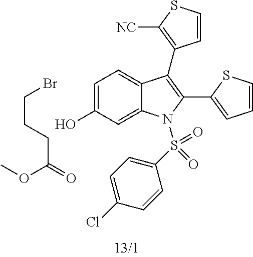 | 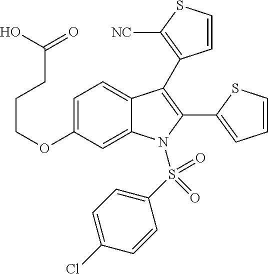 [SMILES: N#Cc1sccc1-c1c(-c2cccs2)n(S(=O)(=O)c2ccc(Cl)cc2)c2cc(OCCCC(=O)O)ccc12] | ¹H-NMR (500 MHz, DMSO-d₆) δ: 8.02 (d, J = 5.0 Hz, 1H), 7.74 (d, J = 2.0 Hz, 1H), 7.71 (d, J = 5.5 Hz, 1H), 7.61 (d, J = 8.5 Hz, 2H), 7.54 (d, J = 8.0 Hz, 2H), 7.25 (d, J = 9.0 Hz, 1H), 7.18 (d, J = 3.0 Hz, 1H), 7.11-7.00 (m, 3H), 4.13 (t, J = 6.5 Hz, 2H), 2.38 (t, J = 7.3 Hz, 2H), 2.03-1.97 (m, 2H); MS: 583.0 (M + 1)⁺. |
| 15/5 | 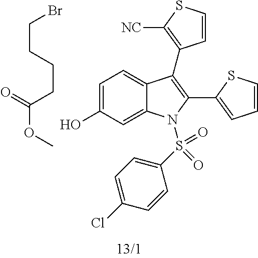 | 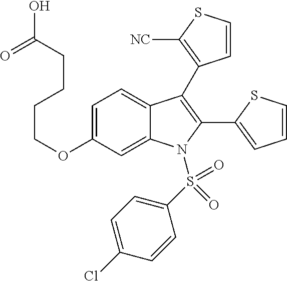 [SMILES: N#Cc1sccc1-c1c(-c2cccs2)n(S(=O)(=O)c2ccc(Cl)cc2)c2cc(OCCCCC(=O)O)ccc12] | ¹H-NMR (500 MHz, DMSO-d₆) δ: 8.03 (d, J = 5.0 Hz, 1H), 7.74 (d, J = 1.5 Hz, 1H), 7.71 (d, J = 5.5 Hz, 1H), 7.61 (d, J = 8.5 Hz, 2H), 7.54 (d, J = 8.5 Hz, 2H), 7.25 (d, J = 9.0 Hz, 1H), 7.18 (d, J = 4.0 Hz, 1H), 7.10 (t, J = 4.3 Hz, 1H), 7.05-7.03 (m, 1H), 7.01 (d, J = 5.0 Hz, 1H), 4.12 (t, J = 6.0 Hz, 2H), 2.31 (t, J = 7.3 Hz, 2H), 1.83-1.78 (m, 2H), 1.73- 1.68 (m, 2H); MS: 597.0 (M + 1)⁺. |
| 15/6 | 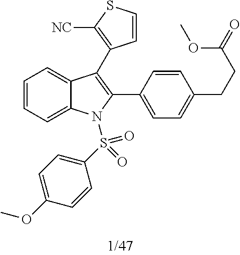 | 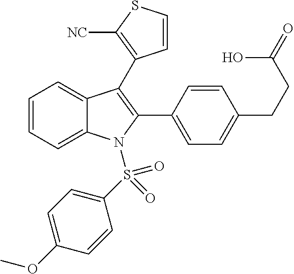 [SMILES: COc1ccc(S(=O)(=O)n2c(-c3ccc(CCC(=O)O)cc3)c(-c3ccsc3C#N)c3ccccc32)cc1] | ¹H-NMR (500 MHz, DMSO-d₆) δ: 12.20 (br s, 1H), 8.26 (d, J = 8.5 Hz, 1H), 7.98 (d, J = 5.0 Hz, 1H), 7.50- 7.47 (m, 1H), 7.40-7.32 (m, 4H), 7.22 (d, J = 8.5 Hz, 2H), 7.16 (d, J = 8.5 Hz, 2H), 6.99 (d, J = 9.0 Hz, 2H), 6.93 (d, J = 5.0 Hz, 1H), 3.78 (s, 3H), 2.87 (t, J = 7.5 Hz, 2H), 2.59 (t, J = 7.5 Hz, 2H); MS: 542.9 (M + 1)⁺. |

### Example Compound Set 17

Example compound is shown below.

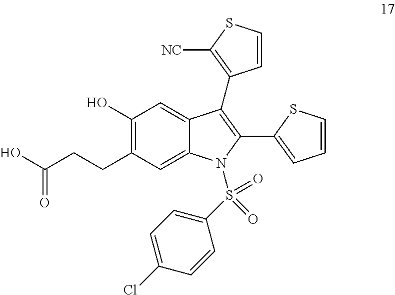 [SMILES: N#Cc1sccc1-c1c(-c2cccs2)n(S(=O)(=O)c2ccc(Cl)cc2)c2cc(CCC(=O)O)c(O)cc12]

### Example Compound Set 19

Example compound is shown below.

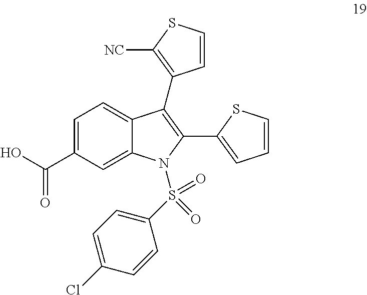 [SMILES: N#Cc1sccc1-c1c(-c2cccs2)n(S(=O)(=O)c2ccc(Cl)cc2)c2cc(C(=O)O)ccc12]

### Example Compounds Set 20

Example compounds are shown below.

| # | building block(s) | structure | analytical data |
|---|---|---|---|
| 20 | | 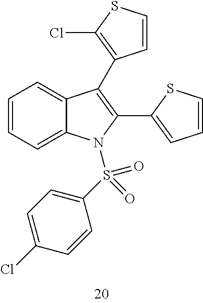 [SMILES: O=S(=O)(c1ccc(Cl)cc1)n1c(-c2cccs2)c(-c2ccsc2Cl)c2ccccc21] | |
| 20/2 | 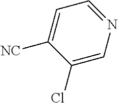 | 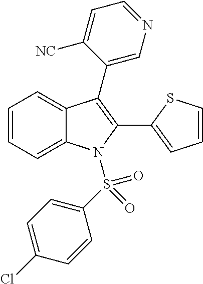 [SMILES: N#Cc1ccncc1-c1c(-c2cccs2)n(S(=O)(=O)c2ccc(Cl)cc2)c2ccccc12] | ¹H-NMR (CDCl₃, 300 MHz) δ: 7.04-7.01 (m, 1H), 7.42-7.19 (m, 8H), 7.48-7.57 (m, 2H), 8.41 (d, J = 8.4 Hz, 1H), 8.55 (s, 1H), 8.70 (d, J = 4.5 Hz, 1H); MS: 476.0 (M + 1)⁺. |
| 20/3 | 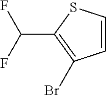 | 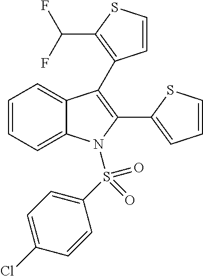 [SMILES: O=S(=O)(c1ccc(Cl)cc1)n1c(-c2cccs2)c(-c2ccsc2C(F)F)c2ccccc21] | ¹H-NMR (CDCl₃, 300 MHz) δ: 6.29 (t, J = 54.9 Hz, 1H), 6.72- 6.73 (m, 1H), 7.03 (dd, J = 3.8, 5.0 Hz, 1H), 7.10 (d, J = 2.7 Hz, 1H), 7.25-7.46 (m, 9H), 8.37 (d, J = 8.4 Hz, 1H); MS: 505.9 (M + 1)⁺. |
| 20/4 | 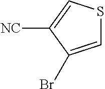 | 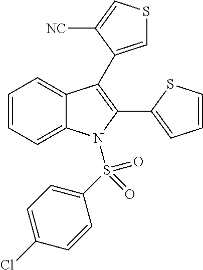 [SMILES: N#Cc1cscc1-c1c(-c2cccs2)n(S(=O)(=O)c2ccc(Cl)cc2)c2ccccc12] | ¹H-NMR (CDCl₃, 300 MHz) δ: 7.04 (dd, J = 3.8, 5.3 Hz, 1H), 7.15 (d, J = 3.3 Hz, 1H), 7.20 (dd, J = 0.9, 3.6 Hz, 1H), 7.26- 7.47 (m, 8H), 7.94 (d, J = 3.3 Hz, 1H), 8.36 (d, J = 8.4 Hz, 1H); MS: 498.0 (M + 18)⁺. |
| 20/5 | 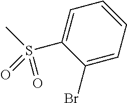 | 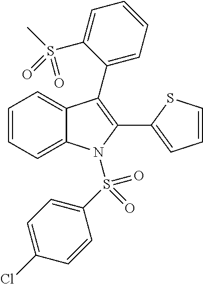 [SMILES: CS(=O)(=O)c1ccccc1-c1c(-c2cccs2)n(S(=O)(=O)c2ccc(Cl)cc2)c2ccccc12] | ¹H-NMR (CDCl₃, 300 MHz) δ: 2.54 (s, 3H), 6.97 (dd, J = 3.8, 8.6 Hz, 1H), 7.02 (s, 1H), 7.07- 7.10 (m, 1H), 7.23-7.57 (m, 10H), 8.14-8.17 (m, 1H), 8.32 (d, J = 8.4 Hz, 1H); MS: 545.0 (M + 18)⁺. |
| 20/6 | 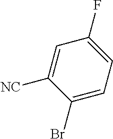 | 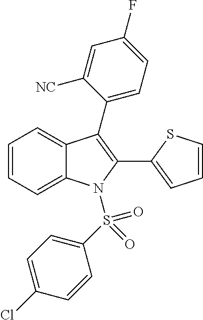 [SMILES: N#Cc1cc(F)ccc1-c1c(-c2cccs2)n(S(=O)(=O)c2ccc(Cl)cc2)c2ccccc12] | ¹H-NMR (CDCl₃, 300 MHz) δ: 6.99-7.02 (m, 1H), 7.16-7.42 (m, 11H), 7.47 (t, J = 8.4 Hz, 1H), 8.36 (d, J = 8.4 Hz, 1H); MS: 493.0 (M + 1)⁺. |
| 20/7 | 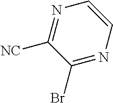 | 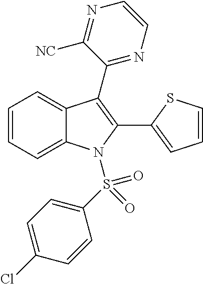 [SMILES: N#Cc1nccnc1-c1c(-c2cccs2)n(S(=O)(=O)c2ccc(Cl)cc2)c2ccccc12] | ¹H-NMR (CDCl₃, 300 MHz) δ: 7.09 (dd, J = 3.9, 5.1 Hz, 1H), 7.26-7.28 (m, 2H), 7.36-7.41 (m, 6H), 7.48-7.52 (m, 1H), 8.43 (d, J = 8.4 Hz, 1H), 8.60 (d, J = 2.4 Hz, 1H), 8.79 (d, J = 2.4 Hz, 1H); MS: 477.0 (M + 1)⁺. |
| 20/11 | 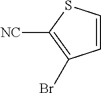 | 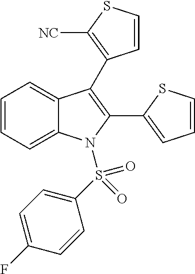 [SMILES: N#Cc1sccc1-c1c(-c2cccs2)n(S(=O)(=O)c2ccc(F)cc2)c2ccccc12] | ¹H-NMR (CDCl₃, 300 MHz) δ: 6.80 (d, J = 5.1 Hz, 1H), 6.98- 7.08 (m, 3H), 7.20-7.22 (m, 1H), 7.33-7.53 (m, 7H), 8.39 (d, J = 8.4 Hz, 1H); MS: 482.0 (M + 18)⁺. |
| | 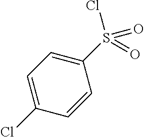 [SMILES: O=S(=O)(Cl)c1ccc(Cl)cc1] | | |
| 20/12 | 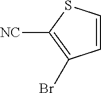 | 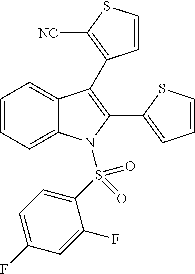 [SMILES: N#Cc1sccc1-c1c(-c2cccs2)n(S(=O)(=O)c2ccc(F)cc2F)c2ccccc12] | ¹H-NMR (CDCl₃, 300 MHz) δ: 6.74-6.84 (m, 2H), 6.89 (d, J = 5.1 Hz, 1H), 7.00-7.03 (m, 1H), 7.20 (d, J = 2.7 Hz, 1H), 7.32- 7.51 (m, 6H), 8.30 (d, J = 9.0 Hz, 1H); MS: 483.0 (M + 1)⁺. |
| | 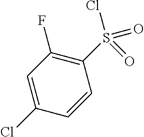 [SMILES: O=S(=O)(Cl)c1ccc(Cl)cc1F] | | |
| 20/13 | 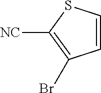 | 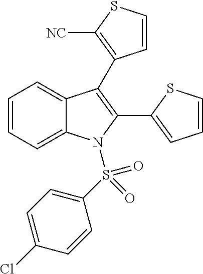 [SMILES: N#Cc1sccc1-c1c(-c2cccs2)n(S(=O)(=O)c2ccc(Cl)cc2)c2ccccc12] | ¹H-NMR (CDCl₃, 300 MHz) δ: 6.81 (d, J = 4.8 Hz, 1H), 7.06 (dd, J = 3.2, 5.0 Hz, 1H), 7.21 (d, J = 2.7 Hz, 1H), 7.29-7.49 (m, 9H), 8.38 (d, J = 8.7 Hz, 1H). |
| | 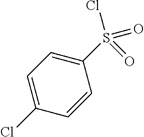 [SMILES: O=S(=O)(Cl)c1ccc(Cl)cc1] | | |
| 20/14 | 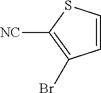 | 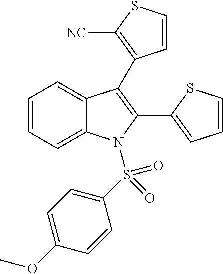 [SMILES: COc1ccc(S(=O)(=O)n2c(-c3cccs3)c(-c3ccsc3C#N)c3ccccc32)cc1] | ¹H-NMR (CDCl₃, 300 MHz) δ: 3.79 (s, 3H), 6.77-6.79 (m, 3H), 7.05 (dd, J = 3.8, 5.0 Hz, 1H), 7.19 (dd, J = 0.9, 3.6 Hz, 1H), 7.31-7.49 (m, 7H), 8.41 (d, J = 8.4 Hz, 1H); MS: 477.0 (M + 1)⁺. |
| | 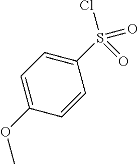 | | |
| 20/15 | 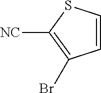 | 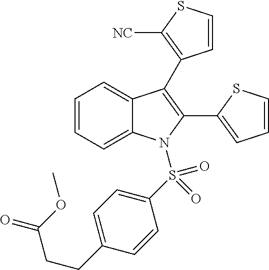 [SMILES: COC(=O)CCc1ccc(S(=O)(=O)n2c(-c3cccs3)c(-c3ccsc3C#N)c3ccccc32)cc1] | ¹H-NMR (CDCl₃, 300 MHz) δ: 2.55-2.60 (m, 2H), 2.93 (t, J = 7.5 Hz, 2H), 3.63 (s, 3H), 6.79 (d, J = 5.1 Hz, 1H), 7.04 (dd, J = 3.9, 5.1 Hz, 1H), 7.15-7.18 (m, 3H), 7.33-7.50 (m, 7H), 8.40 (d, J = 8.4 Hz, 1H); MS: 550.0 (M + 18)⁺. |
| | 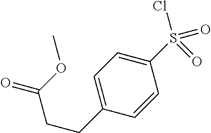 [SMILES: COC(=O)CCc1ccc(S(=O)(=O)Cl)cc1] | | |
| 20/16 | 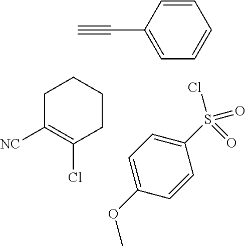 | 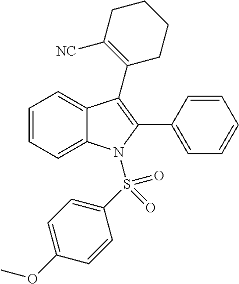 [SMILES: COc1ccc(S(=O)(=O)n2c(-c3ccccc3)c(C3=C(C#N)CCCC3)c3ccccc32)cc1] | ¹H-NMR (DMSO-d₆, 400 MHz) δ: 8.17 (d, J = 8.4 Hz, 1H), 7.48- 7.33 (m, 10H), 6.98-6.94 (m, 2H), 3.75 (s, 3H), 2.29-2.08 (m, 3H), 1.77-1.36 (m, 5H); MS: 486.2 (M + 18)⁺. |
| 20/17 | 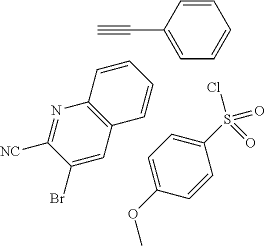 | 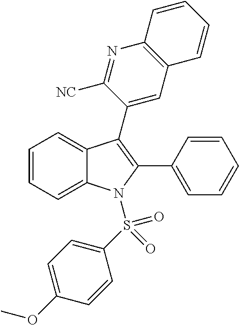 [SMILES: COc1ccc(S(=O)(=O)n2c(-c3ccccc3)c(-c3cc4ccccc4nc3C#N)c3ccccc32)cc1] | ¹H-NMR (DMSO-d₆, 400 MHz) δ: 8.61 (s, 1H), 8.30 (d, J = 8.4 Hz, 1H), 8.12 (d, J = 8.4 Hz, 1H), 8.04 (d, J = 8.0 Hz, 1H), 7.96-7.92 (m, 1H), 7.83-7.79 (m, 1H), 7.55-7.48 (m, 3H), 7.37- 7.29 (m, 7H), 7.05-7.03 (m, 2H), 3.79 (s, 3H); MS: 516.1 (M + 1)⁺. |
| 20/18 | 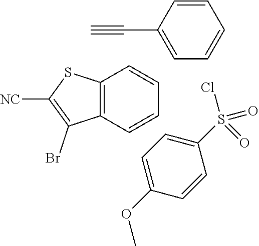 | 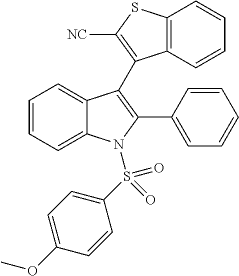 [SMILES: COc1ccc(S(=O)(=O)n2c(-c3ccccc3)c(-c3c(C#N)sc4ccccc34)c3ccccc32)cc1] | ¹H-NMR (DMSO-d₆, 300 MHz) δ: 8.31 (d, J = 8.4 Hz, 1H), 8.16 (d, J = 8.4 Hz, 1H), 7.61-7.50 (m, 2H), 7.43-7.01 (m, 13H), 3.79 (s, 3H); MS: 538.1 (M + 18)⁺. |
| 20/19 | 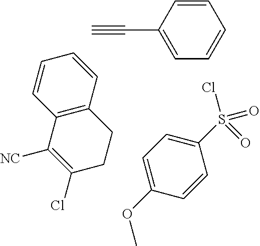 | 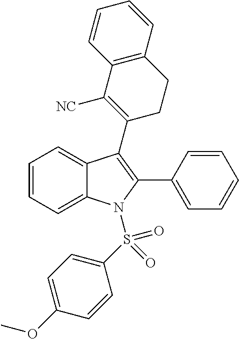 [SMILES: COc1ccc(S(=O)(=O)n2c(-c3ccccc3)c(C3=C(C#N)c4ccccc4CC3)c3ccccc32)cc1] | ¹H-NMR (DMSO-d₆, 400 MHz) δ: 8.23 (d, J = 8.4 Hz, 1H), 7.51- 7.21 (m, 14H), 7.00 (d, J = 8.8 Hz, 2H), 3.77 (s, 3H), 2.86- 2.80 (m, 1H), 2.60-2.49 (m, 2H), 2.15-2.09 (m, 1H); MS: 534.1 (M + 18)⁺. |
| 20/20 | 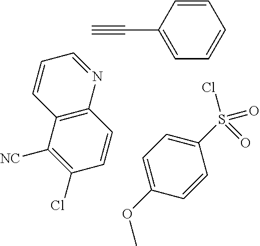 | 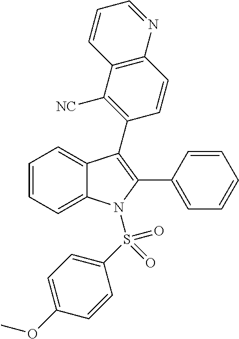 [SMILES: COc1ccc(S(=O)(=O)n2c(-c3ccccc3)c(-c3ccc4ncccc4c3C#N)c3ccccc32)cc1] | ¹H-NMR (DMSO-d₆, 400 MHz) δ: 9.07 (dd, J = 1.2, 4.0 Hz, 1H), 8.44 (d, J = 8.0 Hz, 1H), 8.29 (d, J = 8.4 Hz, 1H), 8.24 (d, J = 8.8 Hz, 1H), 7.79 (dd, J = 4.2, 8.6 Hz, 1H), 7.60 (d, J = 8.8 Hz, 1H), 7.53-7.45 (m, 3H), 7.36- 7.27 (m, 7H), 7.03 (d, J = 9.2 Hz, 2H), 3.79 (s, 3H); MS: 516.1 (M + 1)⁺. |
| 20/21 | 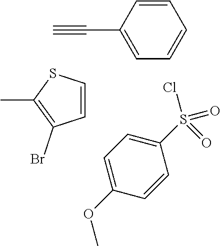 | 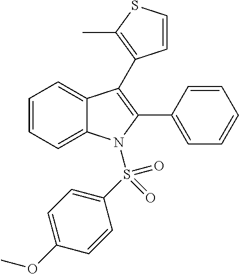 [SMILES: COc1ccc(S(=O)(=O)n2c(-c3ccccc3)c(-c3ccsc3C)c3ccccc32)cc1] | ¹H-NMR (DMSO-d₆, 400 MHz) δ: 8.21 (d, J = 8.4 Hz, 1H), 7.47- 7.25 (m, 10H), 7.14 (d, J = 8.0 Hz, 1H), 7.01-6.97 (m, 2H), 6.65 (d, J = 4.8 Hz, 1H), 3.76 (s, 3H), 1.85 (s, 3H); MS: 460.1 (M + 1)⁺. |
| 20/22 | 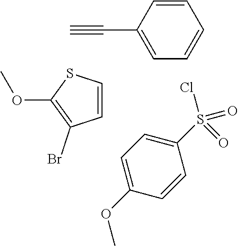 | 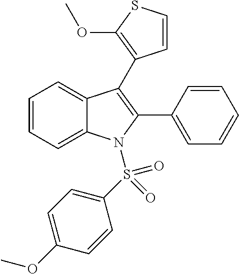 [SMILES: COc1ccc(S(=O)(=O)n2c(-c3ccccc3)c(-c3ccsc3OC)c3ccccc32)cc1] | ¹H-NMR (DMSO-d₆, 400 MHz) δ: 8.20 (d, J = 8.4 Hz, 1H), 7.44- 7.29 (m, 10H), 7.02-6.98 (m, 2H), 6.75 (d, J = 6.0 Hz, 1H), 6.41 (d, J = 5.6 Hz, 1H), 3.77 (s, 3H), 3.54 (s, 3H); MS: 476.1 (M + 1)⁺. |
| 20/23 | 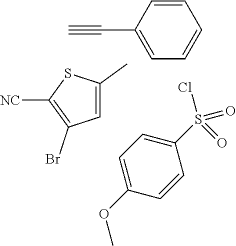 | 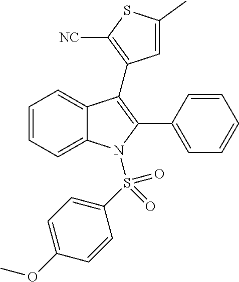 [SMILES: COc1ccc(S(=O)(=O)n2c(-c3ccccc3)c(-c3cc(C)sc3C#N)c3ccccc32)cc1] | ¹H-NMR (DMSO-d₆, 400 MHz) δ: 8.26 (d, J = 8.0 Hz, 1H), 7.52- 7.28 (m, 10H), 7.01 (d, J = 9.2 Hz, 2H), 6.77 (s, 1H), 3.77 (s, 3H), 2.43 (s, 3H); MS: 502.1 (M + 18)⁺. |

### Example Compounds Set 21

Example compounds are shown below.

| # | starting material | structure | analytical data |
|---|---|---|---|
| 21 | | 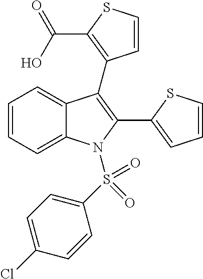 [SMILES: O=C(O)c1sccc1-c1c(-c2cccs2)n(S(=O)(=O)c2ccc(Cl)cc2)c2ccccc12] | |
| | | 21 | |
| 21/1 | 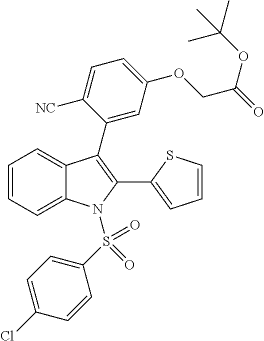 | 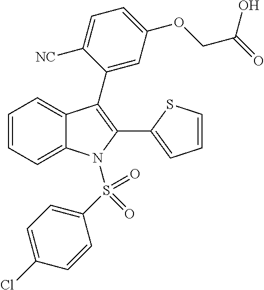 [SMILES: N#Cc1ccc(OCC(=O)O)cc1-c1c(-c2cccs2)n(S(=O)(=O)c2ccc(Cl)cc2)c2ccccc12] | ¹H-NMR (CDCl₃, 400 MHz) δ: 8.34 (d, J = 6.3 Hz, 1H), 7.58- 7.13 (m, 10H), 6.97-6.87 (m, 2H), 6.66 (s, 1H), 4.49 (br s, 2H); MS: 547.0 (M − 1)⁻. |
| | 20/8 | | |
| 21/2 | 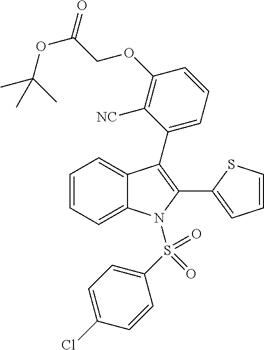 | 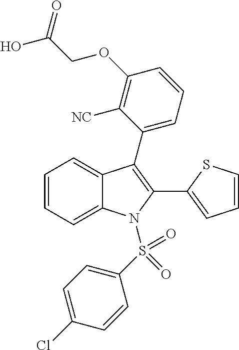 [SMILES: N#Cc1c(OCC(=O)O)cccc1-c1c(-c2cccs2)n(S(=O)(=O)c2ccc(Cl)cc2)c2ccccc12] | ¹H-NMR (DMSO-d₆, 300 MHz) δ: 8.21 (d, J = 8.1 Hz, 1H), 7.65 (d, J = 4.8 Hz, 1H), 7.58-7.48 (m, 6H), 7.37-7.32 (m, 1H), 7.16-7.04 (m, 4H), 6.87 (d, J = 7.8 Hz, 1H), 4.83 (s, 2H); MS: 546.9 (M − 1)⁻. |
| | 20/9 | | |
| 21/3 | 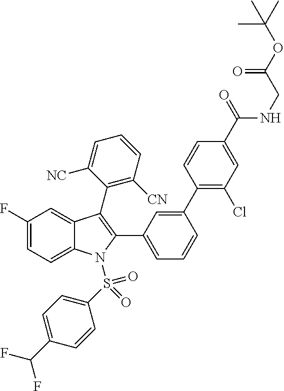 | 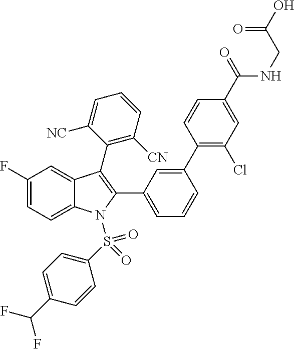 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12] | ¹H-NMR (CD₃OD, 400 MHz) δ: 8.43 (dd, J = 9.4, 4.2 Hz, 1H), 8.05 (d, J = 8.0 Hz, 2H), 7.98 (d, J = 2.0 Hz, 1H), 7.86 (dd, J = 8.0, 2.0 Hz, 1H), 7.71 (t, J = 8.0 Hz, 1H), 7.54-7.30 (m, 9H), 7.09 (s, 1H), 6.94 (dd, J = 8.4, 2.8 Hz, 1H), 6.67 (t, J = 55.6 Hz, 1H), 4.12 (s, 2H); MS: 737.0 M − 1)⁻. |
| | 6/3 | | |

### Example Compound Set 22

Example compound is shown below.

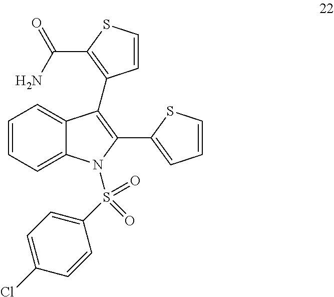 [SMILES: NC(=O)c1sccc1-c1c(-c2cccs2)n(S(=O)(=O)c2ccc(Cl)cc2)c2ccccc12]

### Example Compounds Set 23

Example compounds are shown below.

| # | starting material | structure | analytical data |
|---|---|---|---|
| 23 | | 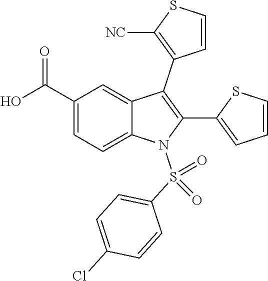 [SMILES: N#Cc1sccc1-c1c(-c2cccs2)n(S(=O)(=O)c2ccc(Cl)cc2)c2ccc(C(=O)O)cc12] | |
| | | 23 | |
| 23/1 | 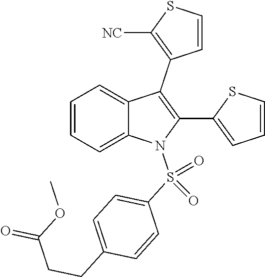 | 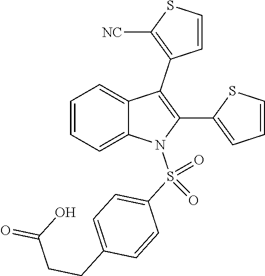 [SMILES: N#Cc1sccc1-c1c(-c2cccs2)n(S(=O)(=O)c2ccc(CCC(=O)O)cc2)c2ccccc12] | ¹H-NMR (CDCl₃, 400 MHz) δ: 8.37 (d, J = 8.7 Hz, 1H), 7.77 (d, J = 5.1, 1H), 7.52-7.45 (m, 2H), 7.39-7.25 (m, 6H), 7.15 (d, J = 3.6 Hz, 1H), 7.07-7.04 (m, 1H), 6.91 (d, J = 5.4 Hz, 1H), 2.92-2.86 (m, 2H), 2.56 (t, J = 7.4 Hz, 2H); MS: 519.0 (M + 1)⁺, 536.1 (M + 18)⁺. |
| | 20/15 | | |
| 23/2 | 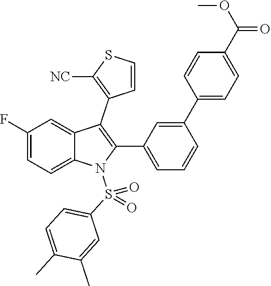 [SMILES: COC(=O)c1ccc(-c2cccc(-c3c(-c4ccsc4C#N)c4cc(F)ccc4n3S(=O)(=O)c3ccc(C)c(C)c3)c2)cc1] | 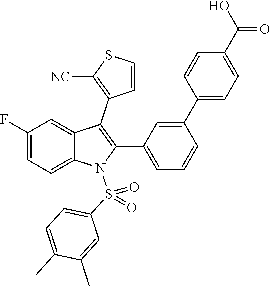 [SMILES: Cc1ccc(S(=O)(=O)n2c(-c3cccc(-c4ccc(C(=O)O)cc4)c3)c(-c3ccsc3C#N)c3cc(F)ccc32)cc1C] | |
| | 8/5 | | |
| 23/3 | 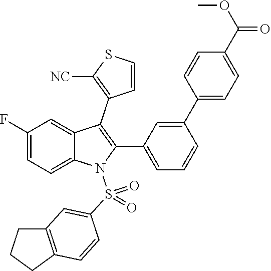 [SMILES: COC(=O)c1ccc(-c2cccc(-c3c(-c4ccsc4C#N)c4cc(F)ccc4n3S(=O)(=O)c3ccc4c(c3)CCC4)c2)cc1] | 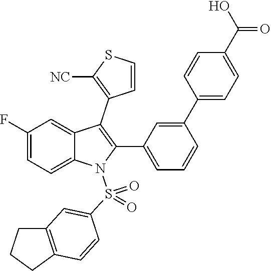 [SMILES: N#Cc1sccc1-c1c(-c2cccc(-c3ccc(C(=O)O)cc3)c2)n(S(=O)(=O)c2ccc3c(c2)CCC3)c2ccc(F)cc12] | |
| | 8/6 | | |

### Example Compound Set 24

Example compound is shown below.

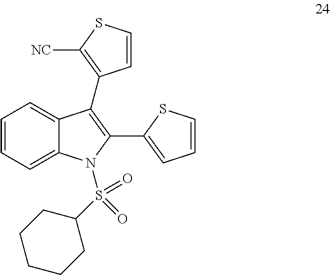 [SMILES: N#Cc1sccc1-c1c(-c2cccs2)n(S(=O)(=O)C2CCCCC2)c2ccccc12]

### Example Compound Set 25

Example compound is shown below.

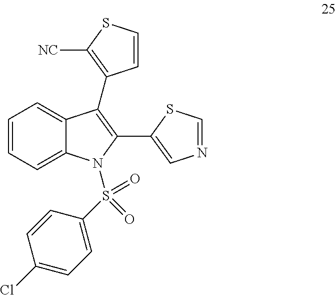 [SMILES: N#Cc1sccc1-c1c(-c2cncs2)n(S(=O)(=O)c2ccc(Cl)cc2)c2ccccc12]

### Example Compounds Set 26

Example compounds are shown below.

| # | starting material | structure | analytical data |
|---|---|---|---|
| 26 | | 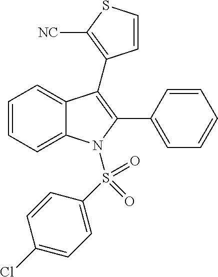 [SMILES: N#Cc1sccc1-c1c(-c2ccccc2)n(S(=O)(=O)c2ccc(Cl)cc2)c2ccccc12] | |
| | | 26 | |
| 26/1 | 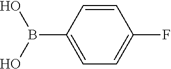 | 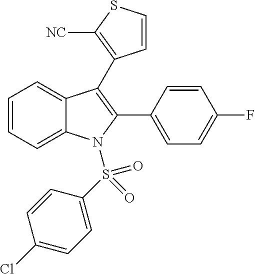 [SMILES: N#Cc1sccc1-c1c(-c2ccc(F)cc2)n(S(=O)(=O)c2ccc(Cl)cc2)c2ccccc12] | ¹H-NMR (DMSO-d₆, 400 MHz) δ: 8.25 (d, J = 8.4 Hz, 1H), 8.03 (d, J = 5.6 Hz, 1H), 7.61-7.58 (m, 2H), 7.55-7.48 (m, 3H), 7.43-7.32 (m, 4H), 7.25-7.21 (m, 2H), 7.00 (d, J = 4.8 Hz, 1H); MS: 509.6 (M + 18)⁺. |

### Example Compounds Set 27

Example compounds are shown below.

| # | starting material | structure | analytical data |
|---|---|---|---|
| 27 | | 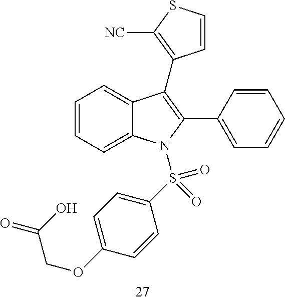 [SMILES: N#Cc1sccc1-c1c(-c2ccccc2)n(S(=O)(=O)c2ccc(OCC(=O)O)cc2)c2ccccc12] | |
| 27/1 | 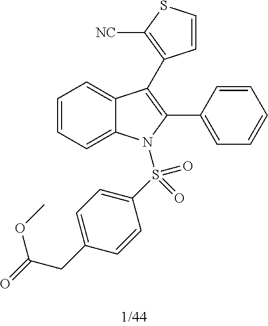 | 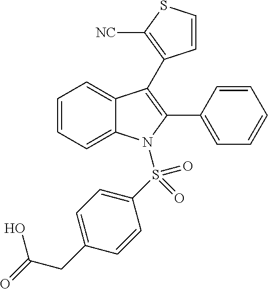 [SMILES: N#Cc1sccc1-c1c(-c2ccccc2)n(S(=O)(=O)c2ccc(CC(=O)O)cc2)c2ccccc12] | ¹H-NMR (500 MHz, DMSO-d₆) δ: 12.55 (br s, 1H), 8.26 (d, J = 9.0 Hz, 1H), 8.00 (d, J = 5.0 Hz, 1H), 7.55- 7.34 (m, 10H), 7.28 (d, J = 7.0 Hz, 2H), 6.98 (d, J = 5.0 Hz, 1H), 3.61 (s, 2H); MS: 499.1 (M + 1)⁺. |
| 27/2 | 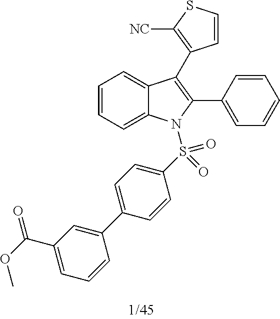 | 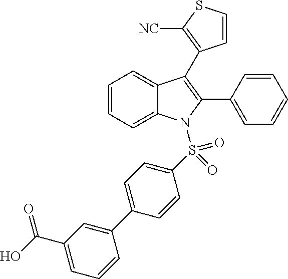 [SMILES: N#Cc1sccc1-c1c(-c2ccccc2)n(S(=O)(=O)c2ccc(-c3cccc(C(=O)O)c3)cc2)c2ccccc12] | ¹H-NMR (500 MHz, DMSO-d₆) δ: 13.18 (br s, 1H), 8.32 (d, J = 8.5 Hz, 1H), 8.17 (s, 1H), 8.00 (d, J = 5.0 Hz, 2H), 7.93 (d, J = 8.0 Hz, 1H), 7.86 (d, J = 8.5 Hz, 2H), 7.63-7.52 (m, 4H), 7.45-7.32 (m, 7H), 6.97 (d, J = 5.0 Hz, 1H); MS: 561.2 (M + 1)⁺. |
| 27/3 | 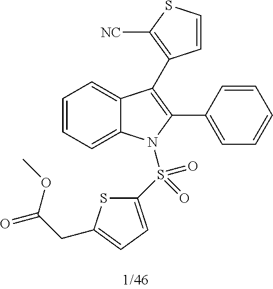 | 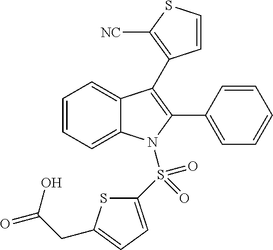 [SMILES: N#Cc1sccc1-c1c(-c2ccccc2)n(S(=O)(=O)c2ccc(CC(=O)O)s2)c2ccccc12] | ¹H-NMR (500 MHz, DMSO-d₆) δ: 12.85 (br s, 1H), 8.21 (d, J = 8.0 Hz, 1H), 8.01 (d, J = 5.5 Hz, 1H), 7.54- 7.51 (m, 1H), 7.44-7.32 (m, 8H), 7.00 (d, J = 5.0 Hz, 1H), 6.95 (d, J = 3.5 Hz, 1H), 3.91 (s, 2H); MS: 505.0 (M + 1)⁺. |

### Example Compound Set 28

Example compound is shown below.

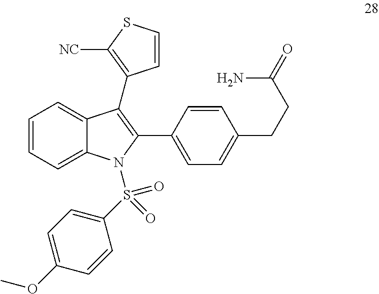 [SMILES: COc1ccc(S(=O)(=O)n2c(-c3ccc(CCC(N)=O)cc3)c(-c3ccsc3C#N)c3ccccc32)cc1]

### Example Compounds Set 30

Example compounds are shown below.

| # | starting material | structure | analytical data |
|---|---|---|---|
| 30 | | 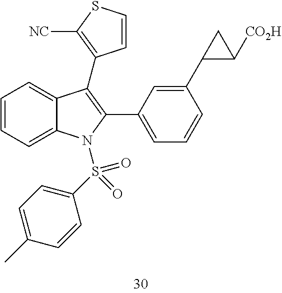 [SMILES: Cc1ccc(S(=O)(=O)n2c(-c3cccc(C4CC4C(=O)O)c3)c(-c3ccsc3C#N)c3ccccc32)cc1] | |
| 30/1 | 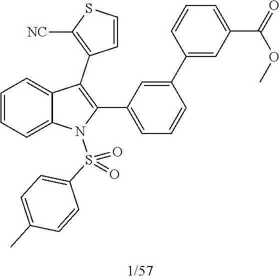 | 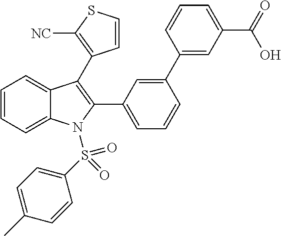 [SMILES: Cc1ccc(S(=O)(=O)n2c(-c3cccc(-c4cccc(C(=O)O)c4)c3)c(-c3ccsc3C#N)c3ccccc32)cc1] | ¹H-NMR (500 MHz, DMSO-d₆) δ: 13.12 (s, 1H), 8.31 (d, J = 8.5 Hz, 1H), 8.07 (s, 1H), 8.01 (d, J = 5.0 Hz, 1H), 7.96 (d, J = 8.0 Hz, 1H), 7.81 (d, J = 7.5 Hz, 1H), 7.76 (d, J = 8.0 Hz, 1H), 7.63-7.60 (m, 1H), 7.55-7.47 (m, 2H), 7.42-7.37 (m, 4H), 7.31-7.25 (m, 3H), 7.07 (d, J = 5.0 Hz, 1H), 2.24 (s, 3H); MS: 574.8 (M + 1)⁺. |
| 30/2 | 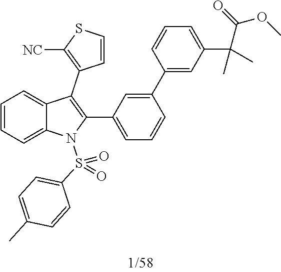 | 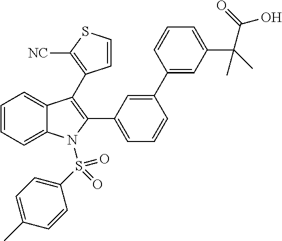 [SMILES: Cc1ccc(S(=O)(=O)n2c(-c3cccc(-c4cccc(C(C)(C)C(=O)O)c4)c3)c(-c3ccsc3C#N)c3ccccc32)cc1] | ¹H-NMR (500 MHz, CD₃OD) δ: 8.43 (d, J = 8.5 Hz, 1H), 7.83 (d, J = 5.0 Hz, 1H), 7.65 (d, J = 8.0 Hz, 1H), 7.51-7.32 (m, 9H), 7.27 (d, J = 8.5 Hz, 2H), 7.22 (s, 1H), 7.12 (d, J = 8.5 Hz, 2H), 6.98 (d, J = 5.0 Hz, 1H) , 2.22 (s, 3H), 1.62 (s, 6H); MS: 615.0 (M + 1)⁺. |
| 30/3 | 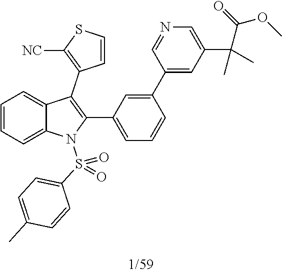 | 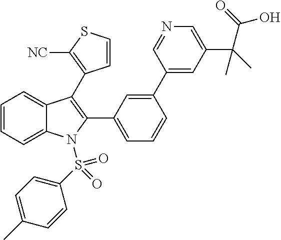 [SMILES: Cc1ccc(S(=O)(=O)n2c(-c3cccc(-c4cncc(C(C)(C)C(=O)O)c4)c3)c(-c3ccsc3C#N)c3ccccc32)cc1] | ¹H-NMR (500 MHz, DMSO-d₆) δ: 12.75 (s, 1H), 8.75 (s, 1H), 8.66 (d, J = 2.0 Hz, 1H), 8.28 (d, J = 8.5 Hz, 1H), 8.05-8.02 (m, 2H), 7.84 (d, J = 7.5 Hz, 1H), 7.62 (s, 1H), 7.54-7.25 (m, 9H), 7.11 (d, J = 5.0 Hz, 1H), 2.23 (s, 3H), 1.61 (s, 6H); MS: 618.1 (M + 1)⁺. |
| 30/4 | 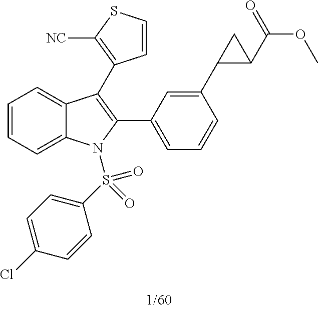 | 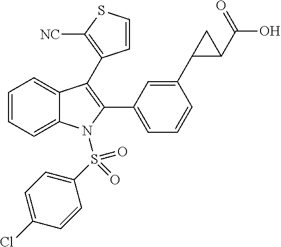 [SMILES: N#Cc1sccc1-c1c(-c2cccc(C3CC3C(=O)O)c2)n(S(=O)(=O)c2ccc(Cl)cc2)c2ccccc12] | ¹H-NMR (500 MHz, DMSO-d₆) δ: 12.35 (s, 1H), 8.26 (d, J = 9.0 Hz, 1H), 8.02 (d, J = 5.0 Hz, 1H), 7.61-7.44 (m, 5H), 7.42-7.08 (m, 4H), 7.07-7.06 (m, 1H), 7.00-6.94 (m, 2H), 2.39-2.34 (m, 1H), 1.72 (s, 1H), 1.44-1.40 (m, 1H), 1.25- 1.23 (m, 1H); MS: 540.8 (M + 1)⁺. |
| 30/5 | 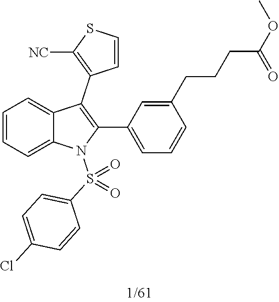 | 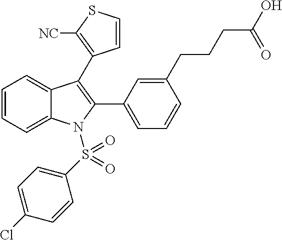 | ¹H-NMR (500 MHz, DMSO-d₆) δ: 12.05 (s, 1H), 8.27 (d, J = 8.5 Hz, 1H), 7.98 (d, J = 5.0 Hz, 1H), 7.60-7.46 (m, 5H), 7.40-7.23 (m, 4H), 7.13 (d, J = 7.5 Hz, 1H), 7.03 (s, 1H), 6.96 (d, J = 5.0 Hz, 1H), 2.56 (t, J = 8.0 Hz, 2H), 2.15 (t, J = 7.0 Hz, 2H), 1.74- 1.70 (m, 2H); MS: 560.8 (M + 1)⁺. |
| 30/6 | 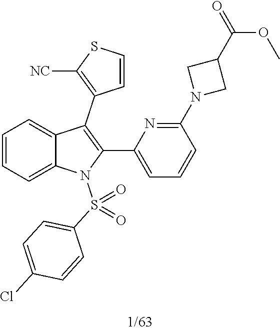 | 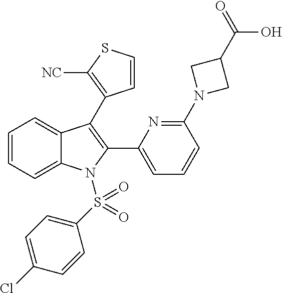 [SMILES: N#Cc1sccc1-c1c(-c2cccc(N3CC(C(=O)O)C3)n2)n(S(=O)(=O)c2ccc(Cl)cc2)c2ccccc12] | ¹H-NMR (500 MHz, CD₃OD) δ: 8.34 (d, J = 8.5 Hz, 1H), 7.96 (d, J = 5.0 Hz, 1H), 7.83-7.79 (m, 1H), 7.73 (d, J = 9.0 Hz, 2H), 7.62- 7.58 (m, 1H), 7.53-7.42 (m, 4H), 7.23 (d, J = 5.0 Hz, 1H), 6.87 (d, J = 8.5 Hz, 1H), 6.77 (d, J = 7.0 Hz, 1H), 4.54-4.43 (m, 4H), 3.77-3.73 (m, 1H); MS: 574.7 (M + 1)⁺. |
| 30/7 | 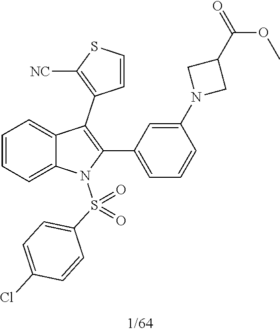 | 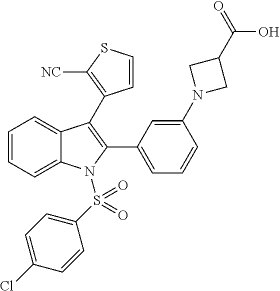 [SMILES: N#Cc1sccc1-c1c(-c2cccc(N3CC(C(=O)O)C3)c2)n(S(=O)(=O)c2ccc(Cl)cc2)c2ccccc12] | ¹H-NMR (500 MHz, CD₃OD) δ: 8.38 (d, J = 8.0 Hz, 1H), 7.77 (d, J = 5.0 Hz, 1H), 7.51-7.34 (m, 7H), 7.16 (t, J = 7.5 Hz, 1H), 6.88 (d, J = 5.0 Hz, 1H), 6.65 (d, J = 7.5 Hz, 1H), 6.55-6.53 (m, 1H), 6.23 (s, 1H), 4.01-3.97 (m, 2H), 3.91-3.88 (m, 2H), 3.54-3.50 (m, 1H); MS: 574.1 (M + 1)⁺. |
| 30/8 | 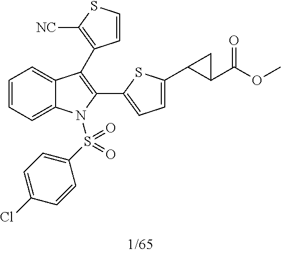 | 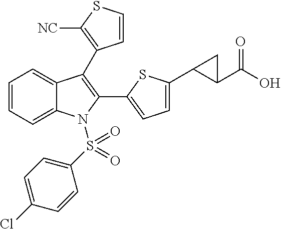 [SMILES: N#Cc1sccc1-c1c(-c2ccc(C3CC3C(=O)O)s2)n(S(=O)(=O)c2ccc(Cl)cc2)c2ccccc12] | ¹H-NMR (400 MHz, DMSO-d₆) δ: 8.26 (d, J = 8.8 Hz, 1H), 8.06 (d, J = 5.5 Hz, 1H), 7.64-7.51 (m, 5H), 7.42-7.36 (m, 2H), 7.05 (d, J = 5.0 Hz, 1H), 7.02 (d, J = 5.0 Hz, 1H), 6.88 (d, J = 3.5 Hz, 1H), 2.52-2.45 (m, 1H), 1.78- 1.75 (m, 1H), 1.48-1.42 (m, 1H), 1.24-1.18 (m, 1H); MS: 582.1 (M + 18)⁺. |
| 30/9 | 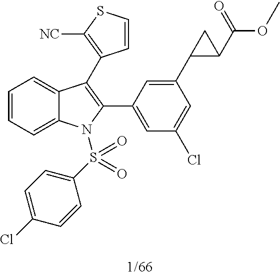 | 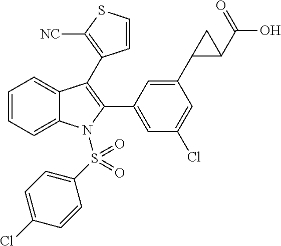 [SMILES: N#Cc1sccc1-c1c(-c2cc(Cl)cc(C3CC3C(=O)O)c2)n(S(=O)(=O)c2ccc(Cl)cc2)c2ccccc12] | ¹H-NMR (500 MHz, DMSO-d₆) δ: 8.25 (d, J = 8.5 Hz, 1H), 8.05 (d, J = 5.0 Hz, 1H), 7.60 (d, J = 8.5 Hz, 2H), 7.55-7.47 (m, 3H), 7.42-7.36 (m, 3H), 7.10 (s, 1H), 7.05 (d, J = 5.0 Hz, 1H), 6.90 (s, 1H), 2.39-2.35 (m, 1H), 1.79-1.72 (m, 1H), 1.43-1.38 (m, 1H), 1.25-1.20 (m, 1H); MS: 610.0 (M + 18)⁺. |
| 30/10 | 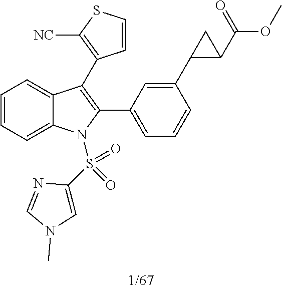 | 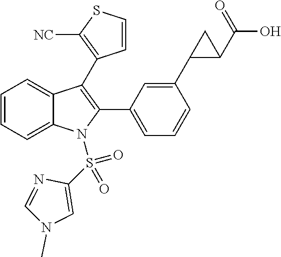 [SMILES: Cn1cnc(S(=O)(=O)n2c(-c3cccc(C4CC4C(=O)O)c3)c(-c3ccsc3C#N)c3ccccc32)c1] | ¹H-NMR (500 MHz, DMSO-d₆) δ: 12.29 (s, 1H), 8.14 (d, J = 8.5 Hz, 1H), 8.00 (d, J = 5.5 Hz, 1H), 7.89 (s, 1H), 7.82 (s, 1H), 7.46- 7.34 (m, 3H), 7.25-7.19 (m, 2H), 7.14-7.10 (m, 2H), 6.99 (d, J = 5.0 Hz, 1H), 3.65 (s, 3H), 2.37- 2.35 (m, 1H), 1.82-1.75 (m, 1H), 1.45-1.38 (m, 1H), 1.28-1.22 (m, 1H); MS: 529.2 (M + 1)⁺. |
| 30/11 | 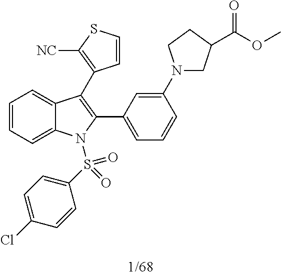 | 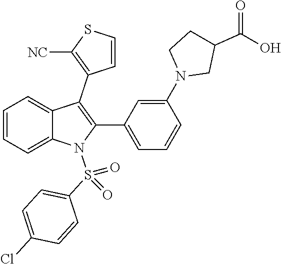 [SMILES: N#Cc1sccc1-c1c(-c2cccc(N3CCC(C(=O)O)C3)c2)n(S(=O)(=O)c2ccc(Cl)cc2)c2ccccc12] | ¹H-NMR (500 MHz, DMSO-d₆) δ: 12.49 (s, 1H), 8.26 (d, J = 8.5 Hz, 1H), 7.99 (d, J = 5.0 Hz, 1H), 7.58-7.40 (m, 5H), 7.40-7.32 (m, 2H), 7.16-7.13 (m, 1H), 6.98 (d, J = 5.0 Hz, 1H), 6.57 (d, J = 8.0 Hz, 1H), 6.52 (d, J = 7.5 Hz, 1H), 6.27 (s, 1H), 3.33- 3.28 (m, 2H), 3.21-3.14 (m, 3H), 2.21-2.13 (m, 2H); MS: 587.8 (M + 1)⁺. |
| 30/12 | 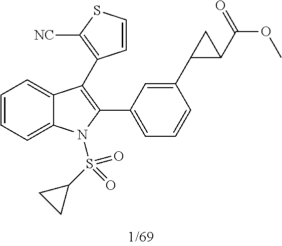 | 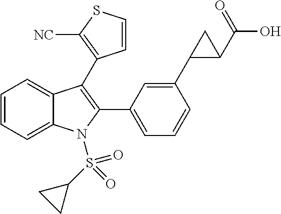 [SMILES: N#Cc1sccc1-c1c(-c2cccc(C3CC3C(=O)O)c2)n(S(=O)(=O)C2CC2)c2ccccc12] | ¹H-NMR (500 MHz, DMSO-d₆) δ: 12.35 (br s, 1H), 8.13 (d, J = 8.0 Hz, 1H), 8.04 (s, 1H), 7.52- 7.39 (m, 3H), 7.27-7.13 (m, 4H), 7.04 (d, J = 5.0 Hz, 1H), 2.88-2.83 (m, 1H), 2.41-2.36 (m, 1H), 1.75-1.72 (m, 1H), 1.43-1.39 (m, 1H), 1.28-1.25 (m, 1H), 0.99-0.94 (m, 4H); MS: 471.0 (M − 18 + H)⁺. |
| 30/13 | 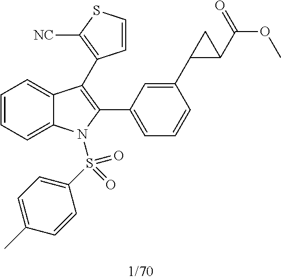 | 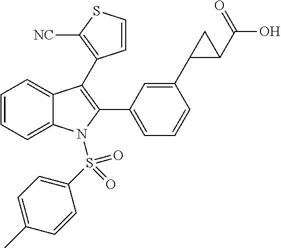 | ¹H-NMR (500 MHz, CD₃OD) δ: 8.37 (d, J = 8.0 Hz, 1H), 7.46- 7.42 (m, 1H), 7.33-7.30 (m, 1H), 7.27-7.17 (m, 9H), 6.74 (s, 1H), 6.60 (d, J = 5.5 Hz, 1H), 2.40-2.36 (m, 1H), 2.35 (s, 3H), 1.76-1.72 (m, 1H), 1.51-1.48 (m, 1H), 1.17-1.13 (m, 1H); MS: 548.0 (M + 1)⁺. |
| 30/14 | 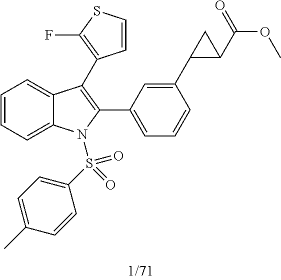 | 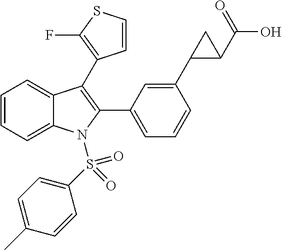 [SMILES: Cc1ccc(S(=O)(=O)n2c(-c3cccc(C4CC4C(=O)O)c3)c(-c3ccsc3F)c3ccccc32)cc1] | ¹H-NMR (500 MHz, DMSO-d₆) δ: 12.38 (s, 1H), 8.25 (d, J = 8.0 Hz, 1H), 7.49-7.45 (m, 1H), 7.36-7.13 (m, 8H), 7.15 (d, J = 7.0 Hz, 1H), 6.96-6.92 (m, 2H), 6.48-6.46 (m, 1H), 2.39-2.34 (m, 1H), 2.31 (s, 3H), 1.73-1.69 (m, 1H), 1.45- 1.40 (m, 1H), 1.27-1.23 (m, 1H); MS: 530.0 (M − 1)⁻. |
| 30/15 | 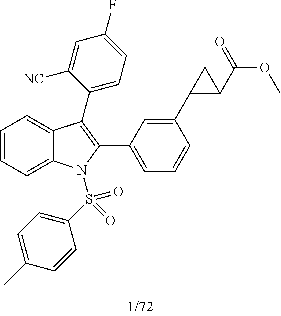 | 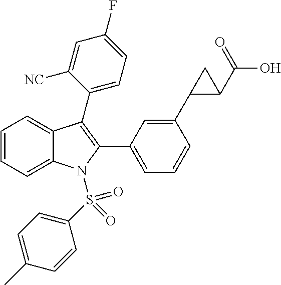 [SMILES: Cc1ccc(S(=O)(=O)n2c(-c3cccc(C4CC4C(=O)O)c3)c(-c3ccc(F)cc3C#N)c3ccccc32)cc1] | ¹H-NMR (500 MHz, DMSO-d₆) δ: 12.35 (br s, 1H), 8.25 (d, J = 8.5 Hz, 1H), 7.88-7.85 (m, 1H), 7.57-7.34 (m, 8H), 7.32-7.18 (m, 3H), 7.06 (s, 1H), 6.84 (s, 1H), 2.32-2.29 (m, 4H), 1.64-1.61 (m, 1H), 1.43-1.39 (m, 1H), 1.19-1.16 (m, 1H); MS: 549.0 (M − 1)⁻. |
| 30/16 | 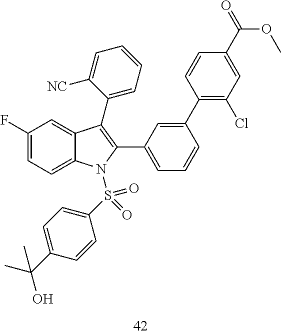 | 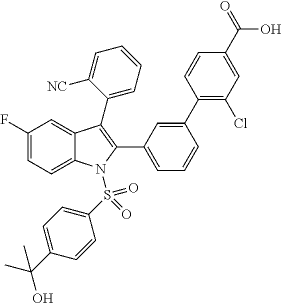 [SMILES: CC(C)(O)c1ccc(S(=O)(=O)n2c(-c3cccc(-c4ccc(C(=O)O)cc4Cl)c3)c(-c3ccccc3C#N)c3cc(F)ccc32)cc1] | ¹H-NMR (400 MHz, DMSO-d₆) δ: 8.32-8.28 (m, 1H), 7.94-7.88 (m, 3H), 7.65-7.24 (m, 12H), 7.04- 6.85 (m, 2H), 5.19 (br s, 1H), 1.23 (s, 6H); MS: 663.0 (M − 1)⁻ |

### Example Compound Set 31

Example compound is shown below.

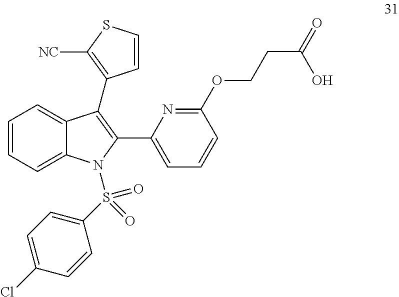 [SMILES: N#Cc1sccc1-c1c(-c2cccc(OCCC(=O)O)n2)n(S(=O)(=O)c2ccc(Cl)cc2)c2ccccc12]

### Example Compounds Set 32

Example compounds are shown below.

| # | starting material | structure | analytical data |
|---|---|---|---|
| 32 | | 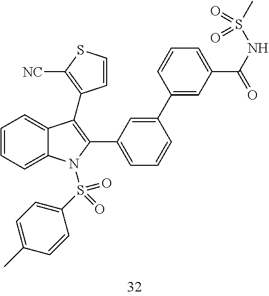 [SMILES: Cc1ccc(S(=O)(=O)n2c(-c3cccc(-c4cccc(C(=O)NS(C)(=O)=O)c4)c3)c(-c3ccsc3C#N)c3ccccc32)cc1] | |
| 32/1 | 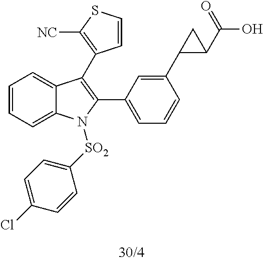 [SMILES: N#Cc1sccc1-c1c(-c2cccc(C3CC3C(=O)O)c2)n(S(=O)(=O)c2ccc(Cl)cc2)c2ccccc12] | 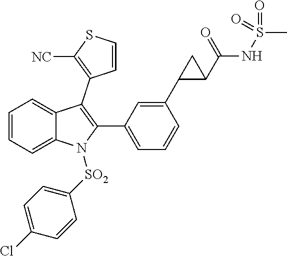 [SMILES: CS(=O)(=O)NC(=O)C1CC1c1cccc(-c2c(-c3ccsc3C#N)c3ccccc3n2S(=O)(=O)c2ccc(Cl)cc2)c1] | ¹H-NMR (500 MHz, DMSO-d₆) δ: 12.09 (s, 1H), 8.25 (d, J = 8.5 Hz, 1H), 8.01 (d, J = 5.0 Hz, 1H), 7.62-7.59 (m, 2H), 7.54- 7.23 (m, 7H), 7.10-6.96 (m, 3H), 3.28 (s, 1H), 2.46-2.42 (m, 1H), 2.09-2.06 (m, 1H), 1.51-1.46 (m, 1H), 1.35-1.32 (m, 1H); MS: 658.0 (M + Na)⁺. |
| 32/2 | 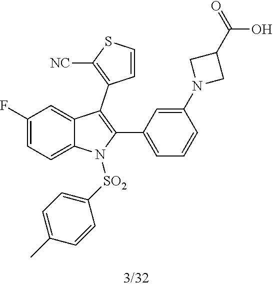 | 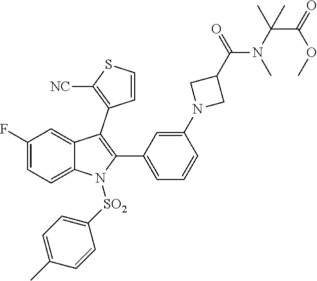 [SMILES: COC(=O)C(C)(C)N(C)C(=O)C1CN(c2cccc(-c3c(-c4ccsc4C#N)c4cc(F)ccc4n3S(=O)(=O)c3ccc(C)cc3)c2)C1] | ¹H-NMR (500 MHz, DMSO-d₆) δ: 8.27 (dd, J = 9.1, 4.4 Hz, 1H), 7.98 (d, J = 5.1 Hz, 1H), 7.46- 7.28 (m, 5H), 7.20-7.07 (m, 2H), 6.94 (d, J = 5.1 Hz, 1H), 6.57- 6.48 (m, 2H), 6.24 (s, 1H), 3.95 (t, J = 7.5 Hz, 2H), 3.89-3.80 (m, 1H), 3.72 (t, J = 6.2 Hz, 2H), 3.52 (s, 3H), 2.87 (s, 3H), 2.33 (s, 3H), 1.34 (s, 6H); MS: 685.0 (M + 1)⁺. |
| 32/3 | 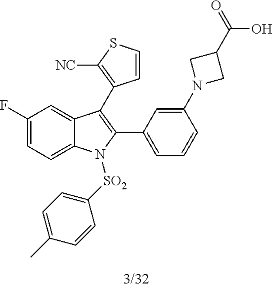 [SMILES: Cc1ccc(S(=O)(=O)n2c(-c3cccc(N4CC(C(=O)O)C4)c3)c(-c3ccsc3C#N)c3cc(F)ccc32)cc1] | 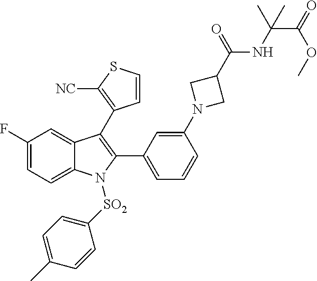 [SMILES: COC(=O)C(C)(C)NC(=O)C1CN(c2cccc(-c3c(-c4ccsc4C#N)c4cc(F)ccc4n3S(=O)(=O)c3ccc(C)cc3)c2)C1] | |
| 32/4 | 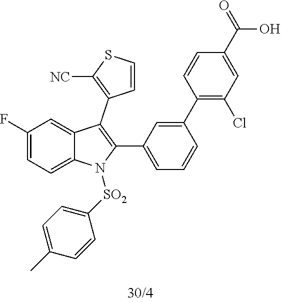 | 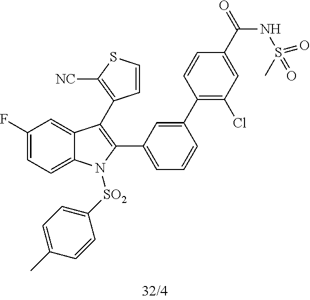 [SMILES: Cc1ccc(S(=O)(=O)n2c(-c3cccc(-c4ccc(C(=O)NS(C)(=O)=O)cc4Cl)c3)c(-c3ccsc3C#N)c3cc(F)ccc32)cc1] | ¹H-NMR (500 MHz, DMSO-d₆) δ: 12.32 (s, 1H), 8.31-8.28 (m, 1H), 8.11 (d, J = 1.5 Hz, 1H), 8.03 (d, J = 5.0 Hz, 1H), 7.99- 7.97 (m, 2H), 7.55-7.35 (m, 4H), 7.41-7.33 (m, 3H), 7.26-7.18 (m, 3H), 7.07-7.03 (m, 2H), 3.38 (s, 3H), 2.22 (s, 3H); MS: 701.9 (M − 1)⁻. |
| 32/5 | 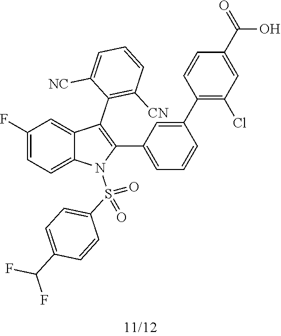 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12] | 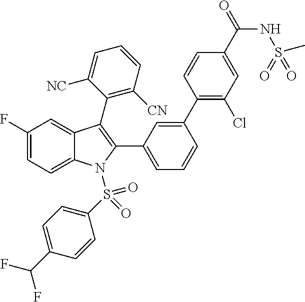 [SMILES: CS(=O)(=O)NC(=O)c1ccc(-c2cccc(-c3c(-c4c(C#N)cccc4C#N)c4cc(F)ccc4n3S(=O)(=O)c3ccc(C(F)F)cc3)c2)c(Cl)c1] | ¹H-NMR (500 MHz, CD₃OD) δ: 8.44 (dd, J = 9.0, 4.0 Hz, 1H), 8.08-8.04 (m, 3H), 7.93 (dd, J = 8.5, 1.5 Hz, 1H), 7.73 (t, J = 8.3 Hz, 1H), 7.55-7.32 (m, 9H), 7.12 (s, 1H), 6.95 (dd, J = 8.0, 2.5 Hz, 1H), 6.69 (t, J = 55.5 Hz, 1H), 3.36 (s, 3H); MS: 756.8 (M − 1)⁻. |

### Example Compounds Set 36

Example compounds are shown below.

| # | starting material | structure | analytical data |
|---|---|---|---|
| 36 | | 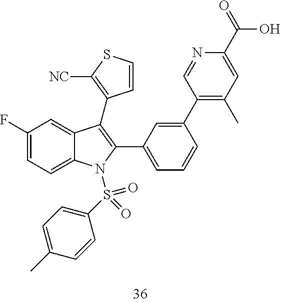 [SMILES: Cc1ccc(S(=O)(=O)n2c(-c3cccc(-c4cnc(C(=O)O)cc4C)c3)c(-c3ccsc3C#N)c3cc(F)ccc32)cc1] | |
| 36/1 | 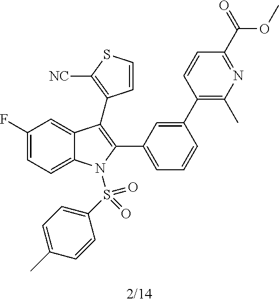 | 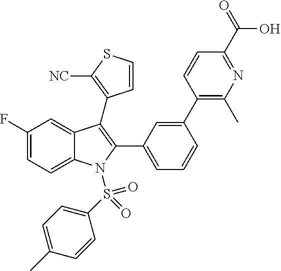 [SMILES: Cc1ccc(S(=O)(=O)n2c(-c3cccc(-c4ccc(C(=O)O)nc4C)c3)c(-c3ccsc3C#N)c3cc(F)ccc32)cc1] | ¹H-NMR (500 MHz, DMSO-d₆) δ: 8.30-8.27 (m, 1H), 8.04 (d, J = 5.0 Hz, 1H), 7.91 (d, J = 7.5 Hz, 1H), 7.65 (d, J = 7.5 Hz, 1H), 7.54- 7.48 (m, 2H), 7.40-7.36 (m, 4H), 7.28-7.26 (m, 2H), 7.19-7.17 (m, 1H), 7.12 (s, 1H), 7.05 (d, J = 5.0 Hz, 1H), 2.30 (s, 3H), 2.25 (s, 3H); MS: 607.8 (M + 1)⁺. |

### Example Compounds Set 37

Example compounds are shown below.

| # | starting material | structure | analytical data |
|---|---|---|---|
| 37 | | 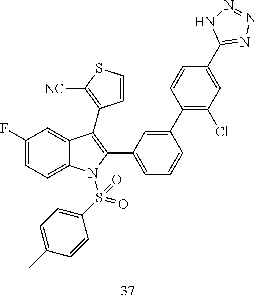 [SMILES: Cc1ccc(S(=O)(=O)n2c(-c3cccc(-c4ccc(-c5nnn[nH]5)cc4Cl)c3)c(-c3ccsc3C#N)c3cc(F)ccc32)cc1] | |
| 37/1 | 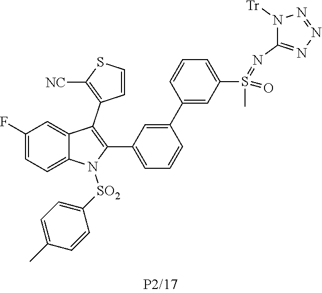 | 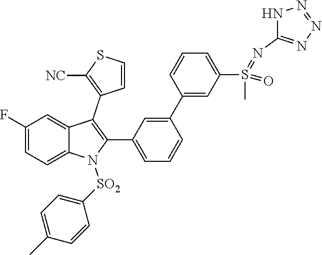 [SMILES: Cc1ccc(S(=O)(=O)n2c(-c3cccc(-c4cccc(S(C)(=O)=Nc5nnn[nH]5)c4)c3)c(-c3ccsc3C#N)c3cc(F)ccc32)cc1] | ¹H-NMR (500 MHz, DMSO-d₆) δ: 8.33- 8.30 (m, 1H), 8.16 (s, 1H), 8.02-7.76 (m, 5H), 7.61 (s, 1H), 7.51 (t, J = 7.5 Hz, 1H), 7.41-7.37 (m, 3H), 7.30-7.26 (m, 3H), 7.20 (dd, J = 8.5, 2.5 Hz, 1H), 7.07 (d, J = 5.0 Hz, 1H), 3.71 (s, 3H), 2.24 (s, 3H); MS: 694.0 (M + 1)⁺. |

### Example Compounds 41/1 and 41/2

Example compounds are shown below.

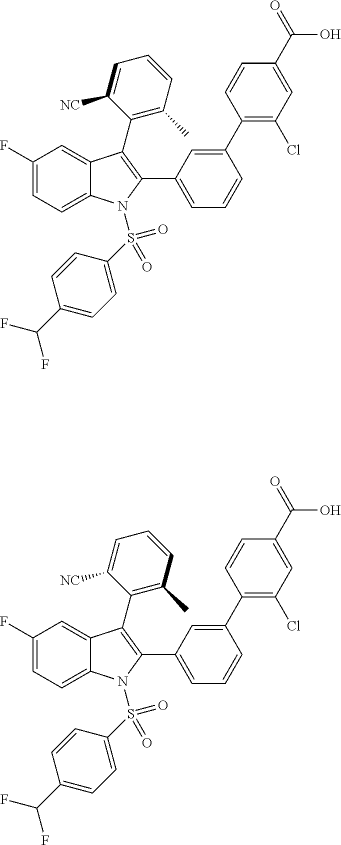

### Separated Atropisomers of 2-chloro-3′-(3-(2-cyano-6-methylphenyl)-1-((4-(difluoro-methyl)phenyl)sulfonyl)-5-fluoro-1H-indol-2-yl)-[1,1′-biphenyl]-4-carboxylic acid (41/1 and 41/2)

### Example Compound Set 44

Example compound is shown below.

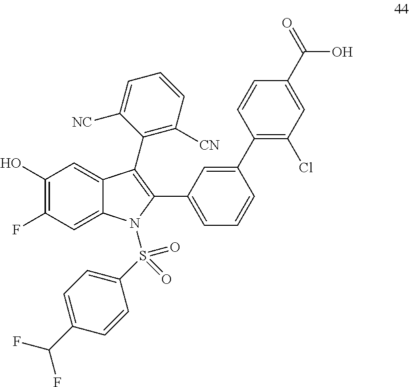 [SMILES: N#Cc1cccc(C#N)c1-c1c(-c2cccc(-c3ccc(C(=O)O)cc3Cl)c2)n(S(=O)(=O)c2ccc(C(F)F)cc2)c2cc(F)c(O)cc12]

### Biological Examples

### Compound Stock Solutions

The tested compounds were usually dissolved, tested and stored as 20 mM stock solutions in DMSO.

Since sulfonyl acetic acid derivatives tend to decarboxylate under these conditions, these stock solutions were prepared, tested and stored as 20 mM DMSO stock solutions containing 100 mM trifluoroacetic acid (5 equivalents). Sulfonyl acetic acid derivatives are shelf stable as solid at rt for long time as reported by Griesbrecht et al. (Synlett 2010:374) or Faucher et al. (J. Med. Chem. 2004; 47:18).

### TR-FRETβ Activity Assay

Recombinant GST-LXRβ ligand-binding domain (LBD; amino acids 156-461; NP009052; SEQ ID NO:4) was expressed in E. coli and purified via gluthatione-sepharose affinity chromatography. N-terminally biotinylated NCoA3 coactivator peptide (SEQ ID NO:7) was chemically synthesized (Eurogentec). Assays were done in 384 well format (final assay volume of 25 μL/well) in a Tris/HCl buffer (pH 6.8) containing KCl, bovine serum albumin, Triton-X-100 and 1 μM 24(S)-25-epoxycholesterol as LXR-prestimulating agonist. Assay buffer was provided and test articles (potential LXR inverse agonists) were titrated to yield final assay concentrations of 50 μM, 16.7 μM, 5.6 μM, 1.9 μM, 0.6 μM, 0.2 μM, 0.07 μM, 0.02 μM, 0.007 μM, 0.002 μM with one vehicle control. Finally, a detection mix was added containing anti GST-Tb cryptate (CisBio; 610SAXLB) and Streptavidin-XL665 (CisBio; 610SAXLB) as fluorescent donor and acceptor, respectively, as well as the coactivator peptide and LXRβ-LBD protein (SEQ ID NO:4). The reaction was mixed thoroughly, equilibrated for 1 h at 4° C. and vicinity of LXRβ and coactivator peptide was detected by measurement of fluorescence in a VictorX4 multiplate reader (PerkinElmer Life Science) using 340 nm as excitation and 615 and 665 nm as emission wavelengths. Assays were performed in triplicates.

### Final Assay Concentrations of Components:

240 mM KCl, 1 μg/μL BSA, 0.002% Triton-X-100, 125 μg/μL anti GST-Tb cryptate, 2.5 ng/μL Streptavidin-XL665, coactivator peptide (400 nM), LXRβ protein (530 μg/mL, i.e. 76 nM).

### LXR Gal4 Reporter Transient Transfection Assays

LXRα and LXRβ activity status was determined via detection of interaction with coactivator and corepressor proteins in mammalian two-hybrid experiments (M2H). For this, via transient transfection the full length (FL) proteins of LXRα (amino acids 1-447; NP005684; SEQ ID NO:1) or LXRβ-(amino acids 1-461; NP009052; SEQ ID NO:2) or the ligand-binding domains (LBD) of LXRα (amino acids 155-447 SEQ ID NO:3) or LXRβ (amino acids 156-461; SEQ ID NO:4) were expressed from pCMV-AD (Stratagene) as fusions to the transcriptional activation domain of NFkB. As cofactors, domains of either the steroid receptor coactivator 1 (SRC1; amino acids 552-887; SEQ ID NO:5) or of the corepressor NCoR (amino acids 1906-2312; NP006302; SEQ ID NO:6) were expressed as fusions to the DNA binding domain of the yeast transcription factor GAL4 (from pCMV-BD; Stratagene). Interaction was monitored via activation of a coexpressed Firefly Luciferase Reporter gene under control of a promoter containing repetitive GAL4 response elements (vector pFRLuc; Stratagene). Transfection efficiency was controlled via cotransfection of constitutively active pRL-CMV Renilla reniformis luciferase reporter (Promega). HEK293 cells were grown in minimum essential medium (MEM) with 2 mM L-glutamine and Earle's balanced salt solution supplemented with 8.3% fetal bovine serum, 0.1 mM non-essential amino acids, 1 mM sodium pyruvate, at 37° C. in 5% CO₂. 3.5×10⁴ cells/well were plated in 96-well cell culture plates in growth medium supplemented with 8.3% fetal bovine serum for 16-20 h to ˜90% confluency. For transfection, medium was taken off and LXR and cofactor expressing plasmids as well as the reporter plasmids are added in 30 μL OPTIMEM/well including polyethylene-imine (PEI) as vehicle. Typical amounts of plasmids transfected/well: pCMV-AD-LXR (5 ng), pCMV-BD-cofactor (5 ng), pFR-Luc (100 ng), pRL-CMV (0.5 ng). Compound stocks were prepared in DMSO, prediluted in MEM to a total volume of 120 μL, and added 4 h after addition of the transfection mixture (final vehicle concentration not exceeding 0.2%). Cells were incubated for additional 16 h, lysed for 10 min in 1×Passive Lysis Buffer (Promega) and Firefly and Renilla luciferase activities were measured sequentially in the same cell extract using buffers containing D-luciferine and coelenterazine, respectively. Measurements of luminescence were done in a BMG-luminometer.

| Materials | Company | Cat. No. |
|---|---|---|
| HEK293 cells | DSMZ | ACC305 |
| MEM | Sigma-Aldrich | M2279 |
| OPTIMEM | LifeTechnologies | 11058-021 |
| FCS | Sigma-Aldrich | F7542 |
| Glutamax | Invitrogen | 35050038 |
| Pen/Strep | Sigma Aldrich | P4333 |
| Sodium Pyruvate | Sigma Aldrich | S8636 |
| Non Essential Amino Acids | Sigma Aldrich | M7145 |
| Trypsin | Sigma-Aldrich | T3924 |
| PBS | Sigma Aldrich | D8537 |
| PEI | Sigma Aldrich | 40.872-7 |
| Passive Lysis Buffer (5x) | Promega | E1941 |
| D-Luciferine | PJK | 260150 |
| Coelentrazine | PJK | 26035 |

TABLE 1
LXR activity data

| | | LBD-M2H | LBD-M2H | FL-M2H | FL-M2H |
|---|---|---|---|---|---|
| Ex. # | FRETβ | Gal4α | Gal4β | Gal4α | Gal4β |
| 1/23 | C | D | D | | |
| 1/26 | B | C | D | | |
| 1/27 | B | C | D | | |
| 1/28 | B | C | D | | |
| 1/39 | A | B | — | | |
| 1/40 | B | C | D | | |
| 1/41 | B | C | D | | |
| 1/42 | B | D | D | | |
| 1/122 | C | | | D | D |
| 1/139 | C | | | D | D |
| 2 | C | | | D | D |
| 2/1 | C | | | C | C |
| 2/2 | C | | | C | C |
| 2/16 | D | | | D | D |
| 2/18 | C | | | C | D |
| 3 | D | | | D | D |
| 3/1 | C | | | C | C |
| 3/2 | B | | | C | D |
| 3/3 | B | | | C | C |
| 3/4 | C | | | C | D |
| 3/5 | C | | | C | C |
| 3/6 | B | | | C | C |
| 3/7 | B | | | — | D |
| 3/8 | B | C | D | | |
| 3/9 | B | C | D | | |
| 3/10 | C | | | C | D |
| 3/11 | B | | | C | — |
| 3/12 | C | | | D | D |
| 3/13 | B | | | D | D |
| 3/14 | B | | | C | C |
| 3/15 | B | | | — | — |
| 3/16 | B | C | C | | |
| 3/17 | B | C | D | | |
| 3/18 | B | C | D | | |
| 3/19 | B | C | C | | |
| 3/20 | B | C | D | | |
| 3/21 | C | | | D | D |
| 3/22 | D | | | D | D |
| 3/23 | C | | | D | D |
| 3/24 | | D | D | D | D |
| 3/25 | B | | | C | D |
| 3/26 | B | | | C | D |
| 3/27 | B | | | C | C |
| 3/28 | A | | | — | B |
| 3/29 | B | | | — | — |
| 3/30 | C | | | D | D |
| 3/31 | C | | | C | C |
| 3/32 | C | | | C | D |
| 3/33 | B | | | C | D |
| 3/34 | C | | | C | D |
| 3/35 | C | | | C | D |
| 3/36 | B | | | C | C |
| 3/37 | C | | | D | D |
| 3/38 | C | | | D | D |
| 3/39 | C | | | C | D |
| 3/40 | B | | | C | D |
| 3/41 | B | | | D | D |
| 3/42 | B | C | D | C | D |
| 3/43 | B | C | C | C | D |
| 3/44 | B | D | D | D | D |
| 3/45 | B | | | C | C |
| 3/46 | C | D | D | D | D |
| 3/47 | C | | | D | D |
| 3/48 | C | | | D | D |
| 3/49 | C | | | D | D |
| 3/50 | C | | | D | D |
| 3/51 | B | C | C | | |
| 3/52 | C | | | C | C |
| 3/53 | D | | | D | D |
| 3/54 | C | | | D | D |
| 3/55 | C | | | D | D |
| 3/56 | D | | | D | D |
| 3/57 | C | | | D | D |
| 3/58 | B | | | D | D |
| 3/59 | D | | | D | D |
| 3/60 | C | | | D | D |
| 3/61 | C | | | C | C |
| 3/62 | C | | | C | D |
| 3/63 | D | | | D | D |
| 3/64 | C | | | D | D |
| 3/65 | C | | | D | D |
| 3/66 | B | | | C | C |
| 3/67 | C | | | C | C |
| 3/68 | C | | | D | D |
| 3/69 | C | | | C | C |
| 3/70 | C | | | C | C |
| 3/71 | D | | | D | D |
| 3/72 | D | | | D | D |
| 3/73 | D | | | D | D |
| 4 | B | | | D | C |
| 4/1 | B | | | C | D |
| 4/2 | B | | | C | C |
| 4/3 | B | | | D | D |
| 5 | C | | | B | B |
| 5/1 | C | | | B | C |
| 5/2 | C | | | C | C |
| 5/3 | C | | | | D |
| 5/4 | B | | | C | D |
| 5/5 | C | | | C | C |
| 5/6 | C | | | D | D |
| 5/7 | C | | | C | D |
| 5/8 | C | | | C | C |
| 5/9 | C | | | C | D |
| 5/10 | B | | | C | C |
| 6 | C | | | D | D |
| 6/1 | C | | | D | D |
| 6/2 | C | | | D | D |
| 6/3 | D | | | D | D |
| 8 | C | | | C | D |
| 8/1 | C | | | D | D |
| 8/3 | C | | | C | D |
| 8/7 | C | | | C | D |
| 8/8 | D | | | D | D |
| 10 | C | | | D | C |
| 10/1 | D | | | D | D |
| 10/2 | D | | | D | D |
| 10/3 | D | | | D | D |
| 10/4 | D | | | C | D |
| 11 | D | | | D | D |
| 11/1 | D | | | D | D |
| 11/2 | D | | | D | D |
| 11/3 | D | | | D | D |
| 11/4 | C | | | D | D |
| 11/5 | D | | | D | D |
| 11/6 | D | | | D | D |
| 11/7 | D | | | D | D |
| 11/8 | D | | | D | D |
| 11/9 | D | | | D | D |
| 11/10 | D | | | D | D |
| 11/11 | D | | | D | D |
| 11/12 | D | | | D | D |
| 11/13 | D | | | D | D |
| 11/14 | D | | | D | D |
| 11/15 | D | | | D | D |
| 11/16 | C | | | D | D |
| 11/17 | D | | | D | D |
| 11/18 | C | | | D | D |
| 11/19 | C | | | D | D |
| 11/20 | C | | | D | D |
| 11/21 | D | | | D | D |
| 11/22 | D | | | D | D |
| 11/23 | D | | | D | D |
| 11/24 | C | | | C | C |
| 11/25 | C | | | C | D |
| 11/26 | D | | | D | D |
| 11/27 | D | | | D | D |
| 11/28 | D | | | D | D |
| 11/29 | D | | | D | D |
| 11/30 | D | | | D | D |
| 11/31 | D | | | D | D |
| 11/32 | D | | | D | D |
| 11/33 | D | | | D | D |
| 11/34 | D | | | D | D |
| 11/35 | D | | | D | D |
| 11/36 | D | | | D | D |
| 11/37 | D | | | D | D |
| 11/38 | D | | | D | D |
| C11/39 | C | | | D | D |
| 11/40 | C | | | D | D |
| 11/41 | D | | | D | D |
| 11/42 | A | | | C | C |
| 11/43 | C | | | C | C |
| 11/44 | D | | | D | D |
| 11/45 | D | | | D | D |
| 11/46 | D | | | D | D |
| 11/47 | C | | | C | C |
| C11/48 | D | | | D | D |
| 11/49 | D | | | D | D |
| 11/50 | D | | | D | D |
| 11/51 | D | | | D | D |
| 11/52 | D | | | D | D |
| 11/53 | D | | | D | D |
| 11/54 | D | | | D | D |
| 11/55 | D | | | D | D |
| 11/56 | D | | | D | D |
| 11/57 | D | | | D | D |
| 11/58 | D | | | D | D |
| 11/59 | D | | | D | D |
| 11/60 | | | | D | D |
| 11/61 | C | | | D | D |
| 11/62 | C | | | D | D |
| 11/63 | C | | | D | D |
| 11/64 | C | | | D | D |
| 11/65 | D | | | D | D |
| 11/66 | C | | | D | D |
| 11/67 | C | | | D | D |
| 11/68 | D | | | D | D |
| 11/69 | D | | | D | D |
| 12 | C | | | D | D |
| 12/1 | C | | | D | D |
| 12/2 | C | | | D | D |
| 12/3 | C | | | D | D |
| 12/4 | C | | | D | D |
| 12/5 | D | | | D | D |
| 12/6 | B | | | C | B |
| 12/7 | C | | | C | C |
| 12/8 | B | | | C | C |
| 12/9 | C | | | C | C |
| 12/10 | D | | | D | D |
| 13 | C | D | D | | |
| 13/1 | B | D | D | | |
| 15 | A | B | A | | |
| 15/1 | B | C | C | | |
| 15/2 | A | C | C | | |
| 15/3 | A | — | — | | |
| 15/4 | B | C | D | | |
| 15/5 | B | D | D | | |
| 15/6 | B | — | C | | |
| 17 | A | — | B | | |
| 19 | A | — | — | | |
| 20 | C | D | D | | |
| 20/2 | B | C | D | | |
| 20/3 | C | D | D | | |
| 20/4 | B | D | D | | |
| 20/5 | B | C | C | | |
| 20/6 | C | C | D | | |
| 20/7 | B | C | D | | |
| 20/11 | C | D | D | | |
| 20/12 | B | D | D | | |
| 20/13 | C | D | D | | |
| 20/14 | C | D | D | | |
| 20/15 | C | — | C | | |
| 20/16 | B | C | C | | |
| 20/17 | — | — | C | | |
| 20/18 | — | C | D | | |
| 20/19 | A | C | C | | |
| 20/20 | B | B | C | | |
| 20/21 | B | C | D | | |
| 20/22 | C | C | C | | |
| 20/23 | B | C | C | | |
| 21 | A | — | B | | |
| 21/1 | B | B | B | | |
| 21/2 | B | B | B | | |
| 21/3 | D | | | D | D |
| 22 | B | C | C | | |
| 23 | A | C | C | | |
| 23/1 | B | — | D | | |
| 23/2 | C | | | D | D |
| 23/3 | C | | | D | D |
| 24 | B | C | D | | |
| 25 | B | C | D | | |
| 26 | C | D | D | | |
| 26/1 | B | C | D | | |
| 27 | B | — | C | | |
| 27/1 | B | — | — | | |
| 27/2 | B | — | — | | |
| 27/3 | A | — | — | | |
| 28 | B | B | C | | |
| 30 | C | D | D | | |
| 30/1 | C | C | D | | |
| 30/2 | B | C | C | | |
| 30/3 | | C | C | | |
| 30/4 | B | C | D | | |
| 30/5 | B | C | D | | |
| 30/6 | B | C | C | | |
| 30/7 | C | D | D | | |
| 30/8 | B | C | C | | |
| 30/9 | B | C | D | | |
| 30/10 | A | — | — | | |
| 30/11 | C | C | D | | |
| 30/12 | B | B | C | | |
| 30/13 | C | C | D | | |
| 30/14 | B | C | C | | |
| 30/15 | B | C | C | | |
| 30/16 | B | | | C | C |
| 31 | B | C | C | | |
| 32 | C | C | C | | |
| 32/1 | C | C | D | | |
| 32/2 | C | | | C | C |
| 32/4 | D | | | D | D |
| 32/5 | D | | | D | D |
| 36 | B | | | C | C |
| 36/1 | C | | | D | D |
| 37 | C | | | D | D |
| 37/1 | C | | | C | C |
| 41/1 | D | | | D | D |
| 41/2 | D | | | D | D |
| 44 | C | | | D | D |

Ranges (EC₅₀): —: no activity measured; A: >10 μM, B: 1 μM to <10 μM, C: 100 nM to <1 μM, D: <100 nM; italic numbers indicate that efficacy (compared to GW2033) is below 40%.

### Pharmacokinetics

The pharmacokinetics of the compounds was assessed in mice after single dosing and oral administrations. Blood/plasma and liver exposure was measured via LC-MS.

The study design was as follows:
Animals: C57/bl6/J (Janvier) males
Diet: standard rodent chow
Dose: 20 mg/kg
Animal handling: animals were withdrawn from food at least 12 h before administration
Design: single dose oral administration, n=3 animals per group
Sacrifice: at stated time point (4, 12 or 24 h) after administration
Bioanalytics: LC-MS of liver and blood/plasma samples

TABLE 2
Study results

| | | | | liver/ |
|---|---|---|---|---|
| | time | blood/plasma | liver | blood |
| Example # | point (h) | exposure | exposure | ratio |
| GSK2033 | 4 | below LLOQ | below LLOQ | — |
| (comparative example) | | (14.4 ng/mL) | (9.6 ng/mL) | |
| SR9238 | 4 | below LLOQ | below LLOQ | — |
| (comparative example) | | | | |
| 3/24 | 4 | D | C | D |
| 3/48 | 12 | below LLOQ | A | — |
| | | (1.2 ng/mL) | | |
| 5/3 | 4 | C | C | C |
| 8 | 4 | B | D | B |
| 23/2 | 4 | B | D | C |
| 30/4 | 4 | C | C | C |
| 30/7 | 4 | D | B | D |

Ranges:
blood/plasma exposure:
A: >1 μM,
B: 300 nM to ≤1 μM,
C: 100 nM to <300 nM,
D: <100 nM;
liver exposure:
A: <300 nM,
B: 300 nM to ≤1 μM,
C: 1 μM to ≤3 μM,
D: >3 μM;
liver/plasma ratio:
A: <3,
B: 3 to ≤10,
C: 10 to ≤30,
D: >30;

We confirmed that structurally unrelated LXR inverse agonists GSK2033 and SR9238 are not orally bioavailable. We found, that compounds from the present disclosure are orally bioavailable and the target tissue liver was effectively reached by such compounds and a systemic exposure, which is not desired, could be minimized.

### Short Term HFD Mouse Model:

The in vivo transcriptional regulation of several LXR target genes by LXR modulators was assessed in mice.

For this, C57BL/6J were purchased from Elevage Janvier (Rennes, France) at the age of 8 weeks. After an acclimation period of two weeks, animals were prefed on a high fat diet (HFD) (Ssniff Spezialdiäten GmbH, Germany, Surwit EF D12330 mod, Cat. No. E15771-34), with 60 kcal % from fat plus 1% (w/w) extra cholesterol (Sigma-Aldrich, St. Louis, MO) for 5 days. Animals were maintained on this diet during treatment with LXR modulators. The test compounds were formulated in 0.5% hydroxypropylmethylcellulose (HPMC) and administered in three doses (from 1.5 to 20 mg/kg each) by oral gavage according to the following schedule: on day one, animals received treatment in the morning and the evening (ca. 17:00), on day two animals received the final treatment in the morning after a 4 h fast and were sacrificed 4 h thereafter. Animal work was conducted according to the national guidelines for animal care in Germany.

Upon termination, liver was collected, dipped in ice cold PBS for 30 seconds and cut into appropriate pieces. Pieces were snap frozen in liquid nitrogen and stored at −80° C. For the clinical chemistry analysis from plasma, alanine aminotransferase (ALT, IU/mL), cholesterol (CHOL, mg/dL) and triglycerides (TG, mg/dL) were determined using a fully-automated bench top analyzer (Respons®910, DiaSys Greiner GmbH, Flacht, Germany) with system kits provided by the manufacturer.

Analysis of gene expression in liver tissue. To obtain total RNA from frozen liver tissue, samples (25 mg liver tissue) were first homogenized with RLA buffer (4M guanidin thiocyanate, 10 mM Tris, 0.97% w:v β-mercapto-ethanol). RNA was prepared using a SV 96 total RNA Isolation system (Promega, Madison, Wisconsin, USA) following the manufacturer's instructions. cDNAs were synthesized from 0.8-1 μg of total RNA using All-in-One cDNA Supermix reverse transcriptase (Absource Diagnostics, Munich, Germany). Quantitative PCR was performed and analyzed using Prime time Gene expression master mix (Integrated DNA Technologies, Coralville, Iowa, USA) and a 384-format ABI 7900HT Sequence Detection System (Applied Biosystems, Foster City, USA). The expression of the following genes was analysed: Stearoyl-CoA desaturase1 (Scd1), fatty acid synthase (Fas) and sterol regulatory element-binding protein1 (Srebp1). Specific primer and probe sequences (commercially available) are listed in Table 3. qPCR was conducted at 95° C. for 3 min, followed by 40 cycles of 95° C. for 15 s and 60° C. for 30 s. All samples were run in duplicates from the same RT-reaction. Gene expression was expressed in arbitrary units and normalized relative to the mRNA of the housekeeping gene TATA box binding protein (Tbp) using the comparative Ct method.

TABLE 3
Primers used for quantitative PCR

| | Forward | Reverse | Sequence |
|---|---|---|---|
| Gene | Primer | Primer | Probe |
| Fasn | CCCCTCT | TTGTGGA | CAGGCTC |
| | GTTAATT | AGTGCAG | AGGGTGT |
| | GGCTCC | GTTAGG | CCCATGT |
| | (SEQ ID | (SEQ ID | T |
| | NO: 8) | NO: 9) | (SEQ ID |
| | | | NO: 10) |
| Scd1 | CTGACCT | AGAAGGT | TGTTTAC |
| | GAAAGCC | GCTAACG | AAAAGTC |
| | GAGAAG | AACAGG | TCGCCCC |
| | (SEQ ID | (SEQ ID | AGCA |
| | NO: 11) | NO: 12) | (SEQ ID |
| | | | NO: 13) |
| Srebp1c | CCATCGA | GCCCTCC | TCTCCTG |
| | CTACATC | ATAGACA | CTTGAGC |
| | CGCTTC | CATCTG | TTCTGGT |
| | (SEQ ID | (SEQ ID | TGC |
| | NO: 14) | NO: 15) | (SEQ ID |
| | | | NO: 16) |
| Tbp | CACCAAT | CAAGTTT | ACTCCTG |
| | GACTCCT | ACAGCCA | CCACACC |
| | ATGACCC | AGATTCA | AGCCTC |
| | (SEQ ID | CG | (SEQ ID |
| | NO: 17) | (SEQ ID | NO: 19) |
| | | NO: 18) | |

TABLE 4
Study results

| | | | | liver/ |
|---|---|---|---|---|
| | | plasma | liver | plasma |
| | dose | exposure | exposure | ratio |
| Ex. # | [mg/kg] | 4 h | 4 h | 4 h |
| 3/48 | 20 | D | C | C |
| 3/59 | 20 | C | C | B |
| 3/64 | 20 | C | D | D |
| 3/73 | 10 | D | B | C |
| 5/3 | 20 | D | C | B |
| 8/8 | 10 | C | B | B |
| 10/1 | 10 | D | B | C |
| 10/2 | 10 | D | D | B |
| 11/11 | 20 | D | D | D |
| 11/12 | 20 | D | C | C |
| 11/13 | 20 | A | C | A |
| 11/16 | 20 | C | C | B |
| 11/17 | 20 | C | D | C |
| 11/23 | 20 | C | C | B |
| 11/26 | 10 | C | C | C |
| 11/27 | 20 | D | C | C |
| 11/33 | 20 | C | C | C |
| 11/37 | 10 | D | C | C |
| 11/49 | 20 | D | C | D |
| 11/51 | 10 | D | C | C |
| 11/53 | 10 | D | C | C |
| 11/62 | 10 | C | C | B |
| 11/63 | 10 | D | C | B |
| 11/65 | 10 | D | C | C |
| 21/3 | 10 | D | C | D |
| 23/2 | 20 | C | A | A |
| 32/5 | 10 | D | B | B |

| | | Fasn suppression | Srebp1c suppr. | Scd1 suppresion |
|---|---|---|---|---|
| | | compared to | compared to | compared to |
| Ex. # | | vehicle | vehicle | vehicle |
| 3/48 | 20 | D | D | D |
| 3/59 | 20 | C | D | C |
| 3/64 | 20 | B | B | D |
| 3/73 | 10 | A | D | D |
| 5/3 | 20 | D | C | C |
| 8/8 | 10 | D | D | D |
| 10/1 | 10 | D | D | D |
| 10/2 | 10 | C | C | C |
| 11/11 | 20 | C | D | D |
| 11/12 | 20 | C | D | D |
| 11/13 | 20 | C | D | D |
| 11/16 | 20 | A | B | C |
| 11/17 | 20 | C | D | D |
| 11/23 | 20 | C | D | D |
| 11/26 | 10 | D | D | C |
| 11/27 | 20 | C | A | D |
| 11/33 | 20 | B | D | D |
| 11/37 | 10 | D | D | D |
| 11/49 | 20 | C | C | D |
| 11/51 | 10 | D | D | D |
| 11/53 | 10 | D | D | D |
| 11/62 | 10 | D | D | D |
| 11/63 | 10 | C | D | C |
| 11/65 | 10 | D | D | D |
| 21/3 | 10 | C | D | D |
| 23/2 | 20 | C | D | D |
| 32/5 | 10 | C | C | C |

Ranges:
plasma exposure:
A: >1 μM,
B: 300 nM to ≤1 μM,
C: 100 nM to <300 nM,
D: <100 nM;
liver exposure:
A: <300 nM,
B: 300 nM to ≤1 μM,
C: 1 μM to ≤10 μM,
D: >10 μM;
liver/plasma ratio:
A: <5,
B: 5 to ≤30,
C: 30 to ≤100,
D: >100;
gene suppression:
A: >0.9,
B: 0.6 to ≤0.9,
C: 0.3 to ≤0.6,
D: <0.3;

Triple oral dosing over two days (day one morning and evening, day two morning) of compounds from the present disclosure in mice lead to a high liver exposure with a favourable liver-to-plasma ratio. Hepatic LXR target genes were effectively suppressed. These genes are involved in the transcriptional regulation of hepatic de-novo lipogenesis (Wang et al., Nat. Rev. Mol. Cell Biol. 2015; 16:678). A suppression of these genes will reduce liver fat (liver triglycerides).

### Comparative Examples

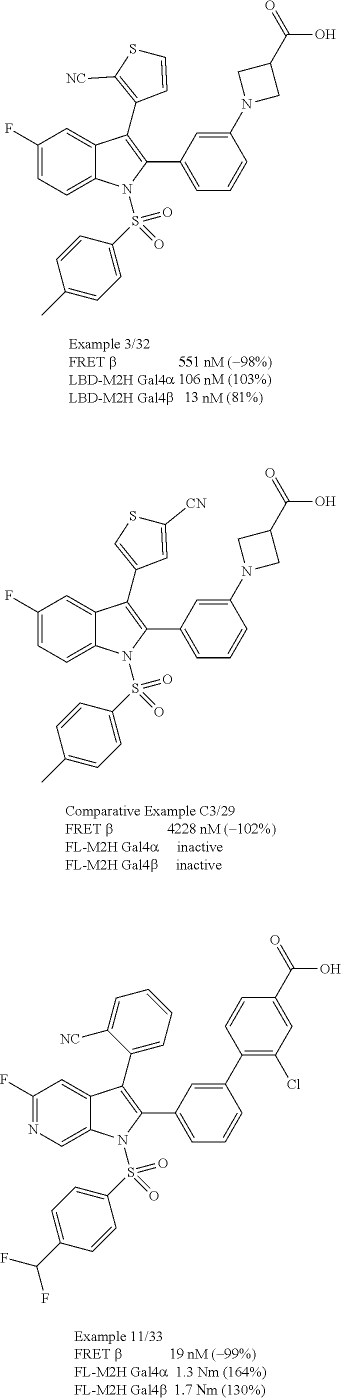 [SMILES: Cc1ccc(S(=O)(=O)n2c(-c3cccc(N4CC(C(=O)O)C4)c3)c(-c3ccsc3C#N)c3cc(F)ccc32)cc1 ; Cc1ccc(S(=O)(=O)n2c(-c3cccc(N4CC(C(=O)O)C4)c3)c(-c3csc(C#N)c3)c3cc(F)ccc32)cc1 ; N#Cc1ccccc1-c1c(-c2cccc(-c3ccc(C(=O)O)cc3Cl)c2)n(S(=O)(=O)c2ccc(C(F)F)cc2)c2cnc(F)cc12]

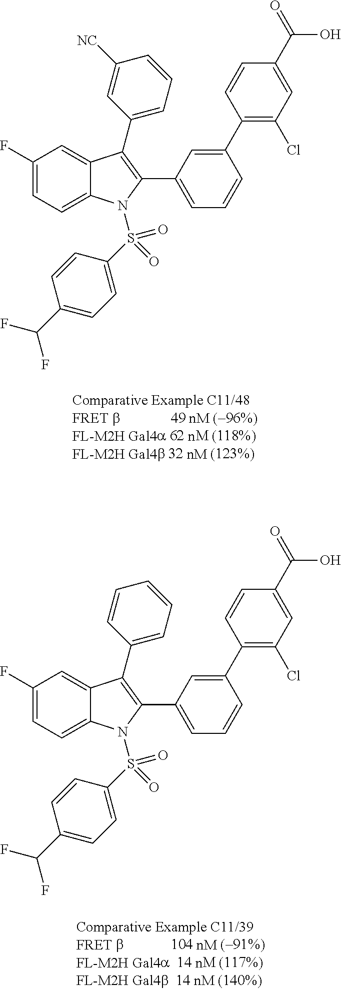 [SMILES: N#Cc1cccc(-c2c(-c3cccc(-c4ccc(C(=O)O)cc4Cl)c3)n(S(=O)(=O)c3ccc(C(F)F)cc3)c3ccc(F)cc23)c1 ; O=C(O)c1ccc(-c2cccc(-c3c(-c4ccccc4)c4cc(F)ccc4n3S(=O)(=O)c3ccc(C(F)F)cc3)c2)c(Cl)c1]

The Comparative Examples illustrate that it can be advantageous, when the cyclic moiety in 3-position of the indole (or analog) has at least one substituent in 1,2-orientation (ortho-substituent).

### Nonclinical Pharmacology

### Primary Pharmacodynamics

The activity and efficacy of Compound 21/3 and Compound pre-6/3, a close structural analog of Compound 21/3, were evaluated across a range of in vitro and in vivo pharmacology studies.

As shown herein, compound 21/3 and compound pre-6/3 are shown below.

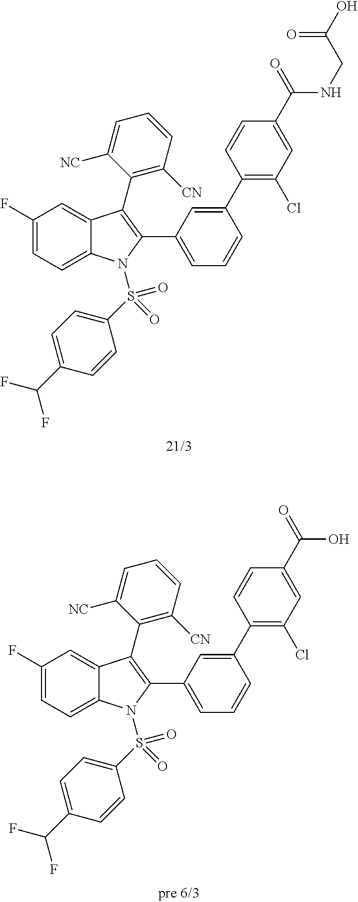 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12 ; N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]

### In Vitro Studies

Compound 21/3 and Compound pre-6/3 are Potent Inhibitors of LXRα and LXRβ

The potencies of Compound 21/3 and its analog, Compound pre-6/3, against LXRα and LXRβ were evaluated in biochemical binding assays, cellular mammalian two-hybrid (M2H) interaction assays evaluating nuclear receptor co-repressor (NCOR) recruitment, and cellular reporter assays evaluating transcriptional activity (Table 5). Compound 21/3 demonstrated comparable binding to, and inhibition of, LXRα and LXRβ activity with a half-maximal effective concentration (EC₅₀) of 7 to 15 nM across all assays. Compound pre-6/3 had a similar profile to Compound 21/3 but was more potent in the cellular M2H and reporter assays.

TABLE 5
Biochemical and Cellular Potency of
Compound 21/3 and Compound pre-6/3

| | compound | compound pre- |
|---|---|---|
| | 21/3 | 6/3 |
| Assay | (nM) | (nM) |
| LXα binding EC₅₀ | 7 ± 16 | 14 ± 5 |
| LXRβ binding EC₅₀ | 15 ± 3 | 17 ± 7 |
| HEK293 LXRα/NCOR recruitment | 8 ± 7 | 0.2 ± 0.1 |
| (M2H) EC₅₀ | | |
| HEK293 LXRβ/NCOR recruitment | 11 ± 19 | 0.4 ± 0.2 |
| (M2H) EC₅₀ | | |
| HT-29 ABCA1 reporter IC₅₀ | 15 ± 4 | 0.7 ± 0.1 |
| HepG2 SREBP1c reporter IC₅₀ | 7 ± 5 | 0.2 ± 0.1 |

ABCA1 = ATP-binding cassette subfamily A member 1,
ABCG1 = ATP binding cassette subfamily G member 1,
IC₅₀ = half maximal inhibitory concentration
Data are mean ± SD. N = 5-38 experiments per assay.

### Compound 21/3 Reduces Lipid Accumulation in Primary Human Hepatocytes and Liver Organoids

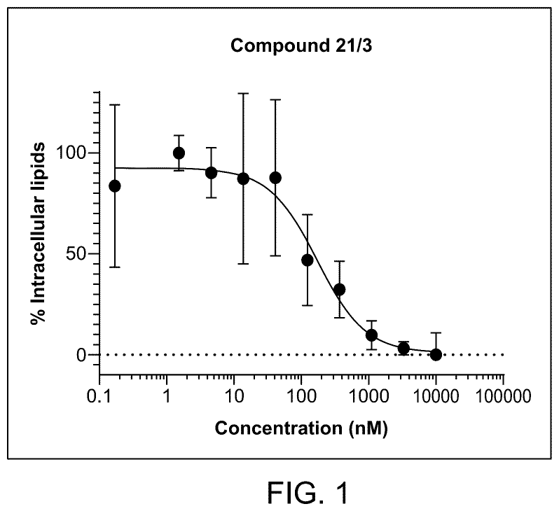

Inhibition of LXR activity in hepatocytes is hypothesized to reduce intracellular TG accumulation via inhibition of DNL. The effects of Compound 21/3 on lipid accumulation in human primary Upcyte® hepatocytes were evaluated. Briefly, human primary Upcyte hepatocytes were cultivated in media containing high concentrations of glucose (25 mM) and palmitate (100 nM) for 5 days in the presence or absence of Compound 21/3, and intracellular lipids were measured by Bodipy™ staining. Compound 21/3 dose-dependently decreased lipid accumulation with an EC₅₀ of 289±34 nM (FIG. 1). FIG. 1 shows a representative dose-response curve for relative fluorescence intensity, normalized to dimethylsulfoxide (DMSO) control, in Upcyte hepatocytes treated with a dose range of Compound 21/3. Data are shown as mean±SD. n=2 wells per treatment; N=8 experiments. FIG. 1 shows that Compound 21/3 lowers lipid droplet accumulation in human primary Upcyte® hepatocytes.

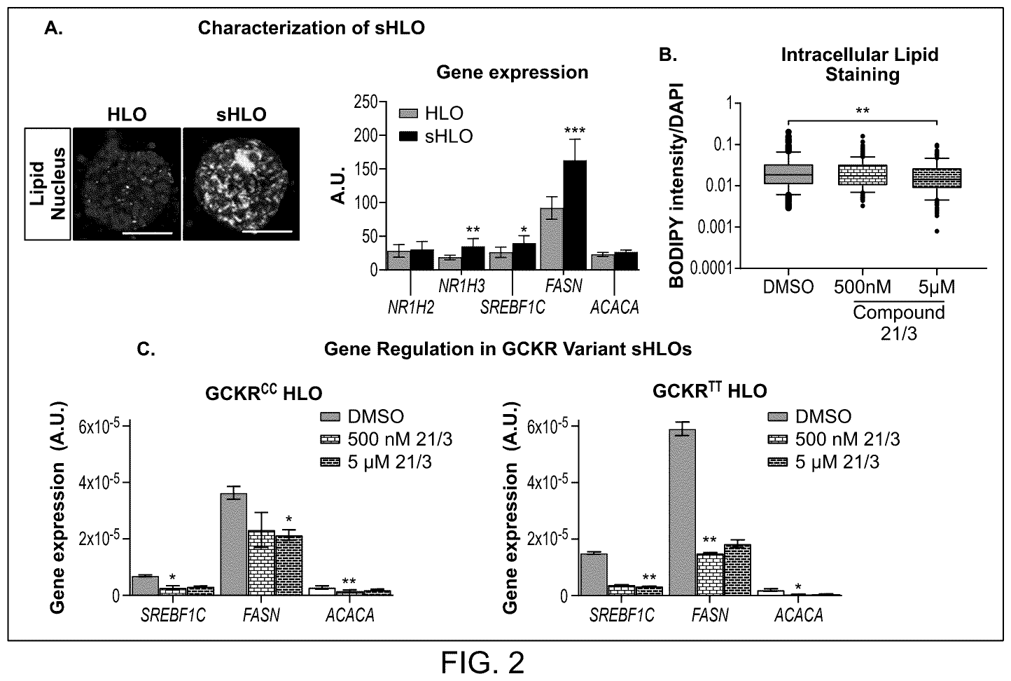

Activity of Compound 21/3 was also evaluated in an induced pluripotent stem cell (iPSC)-derived human liver organoid (HLO) model established from human donors with different genetic backgrounds, including those with known genetic risk variants for NASH (Ouchi, Togo et al. 2019). The HLOs were exposed to high concentrations of lipid to induce a reproducible steatotic NASH phenotype (sHLO) with increased expression of DNL genes, including NR1H3, the gene that encodes LXRα (FIG. 2A) (Ouchi, Togo et al. 2019, Minniti, Pedrelli et al. 2020). Compound 21/3 dose-dependently reduced intracellular lipid content in patient-derived sHLOs (FIG. 2B). Mutations in GCKR have been associated with increased circulating lipids, hepatic DNL, and diseases including diabetes and NASH (Fernandes Silva, Vangipurapu et al. 2019). Compound 21/3 similarly suppressed the expression of DNL-associated genes in sHLOs derived from homozygous carriers of the wild-type (CC) or mutant (TT) alleles of GCKR, supporting the potential therapeutic benefit of Compound 21/3 in patients with enhanced hepatic DNL (FIG. 2C). FIG. 2A-2C shows that Compound 21/3 lowers lipid droplet accumulation and lipogenic gene expression in steatotic human liver organoids.

### In Vivo Studies

Compound 21/3 and Compound pre-6/3 have Beneficial Effects on Systemic and Hepatic Lipid Homeostasis, Insulin Sensitivity, and Hepatic Fibrosis in Nonclinical Rodent Models

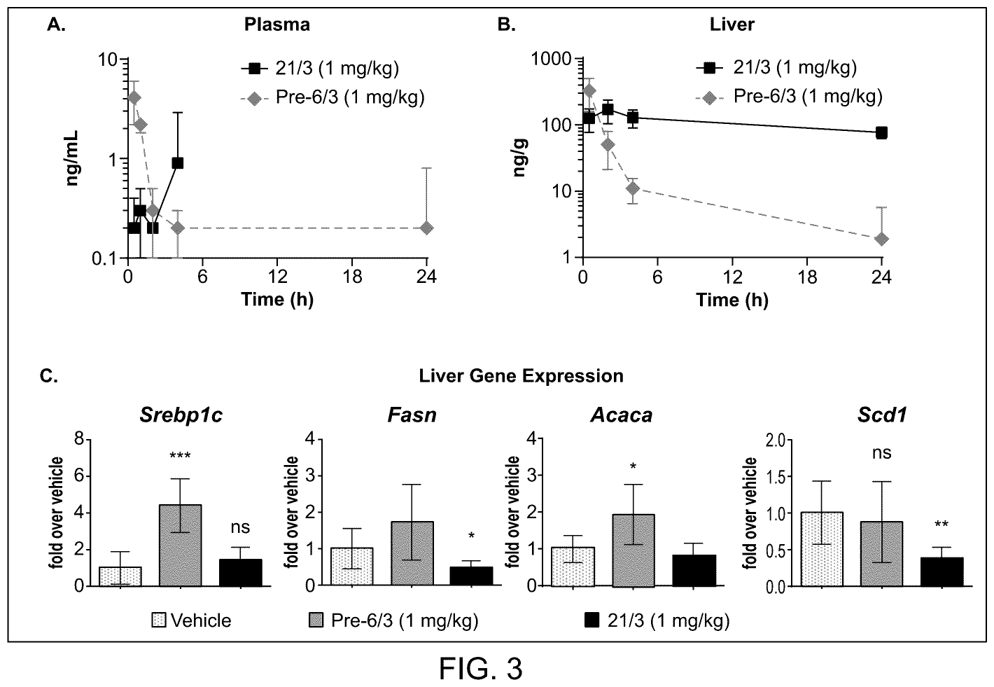

The efficacy of Compound 21/3 and Compound pre-6/3 were evaluated in diet-induced obese (DIO) mouse and rat models. Despite comparable in vitro potency, Compound 21/3 and Compound pre-6/3 demonstrate different pharmacology in vivo with Compound 21/3 having lower plasma exposures, and higher and sustained liver exposures than Compound pre-6/3 (FIG. 3A-B). As a result of greater liver loading, mice treated with a single dose of Compound 21/3, but not Compound pre-6/3, demonstrated sustained target engagement (assessed by expression of LXR target genes) in the liver 24 hours post-dose (FIG. 3C). FIG. 3A-C shows that Compound 21/3 and Compound pre-6/3 demonstrate different pharmacology in vivo.

### Compound 21/3 Improves Hepatic and Systemic Lipid Homeostasis in Dysmetabolic Rodents

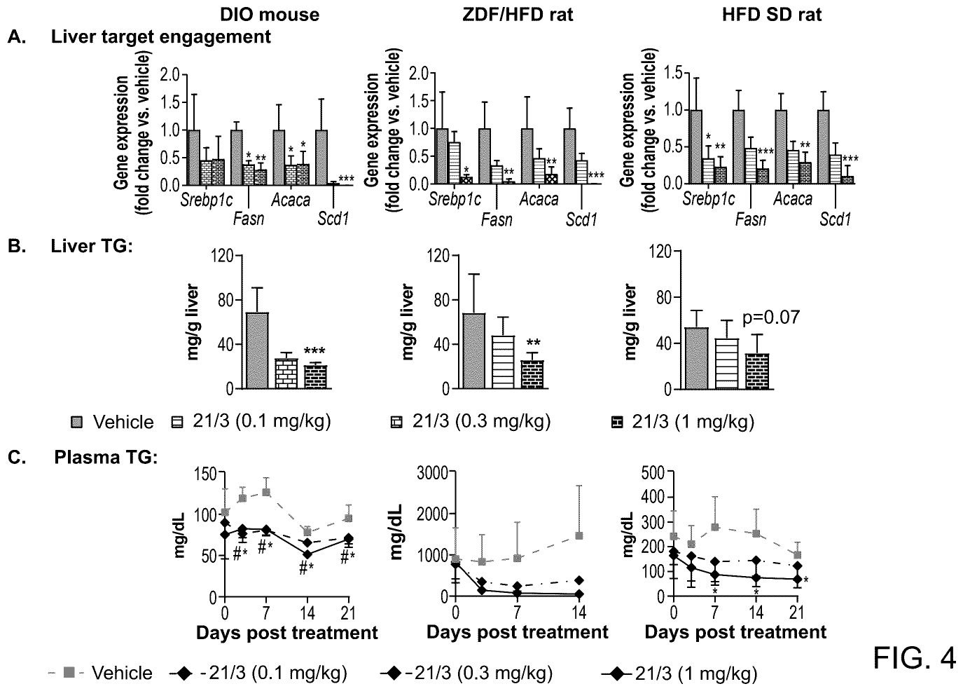

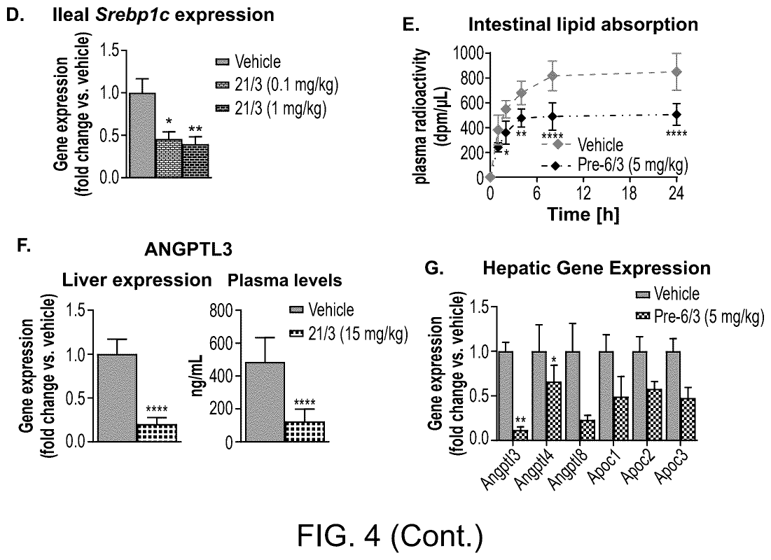

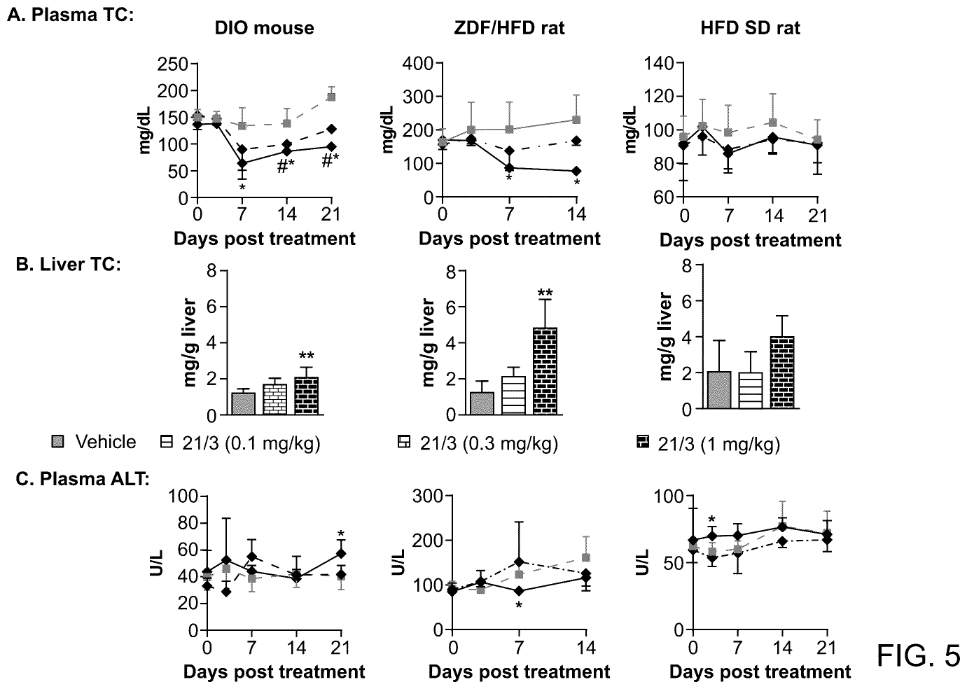

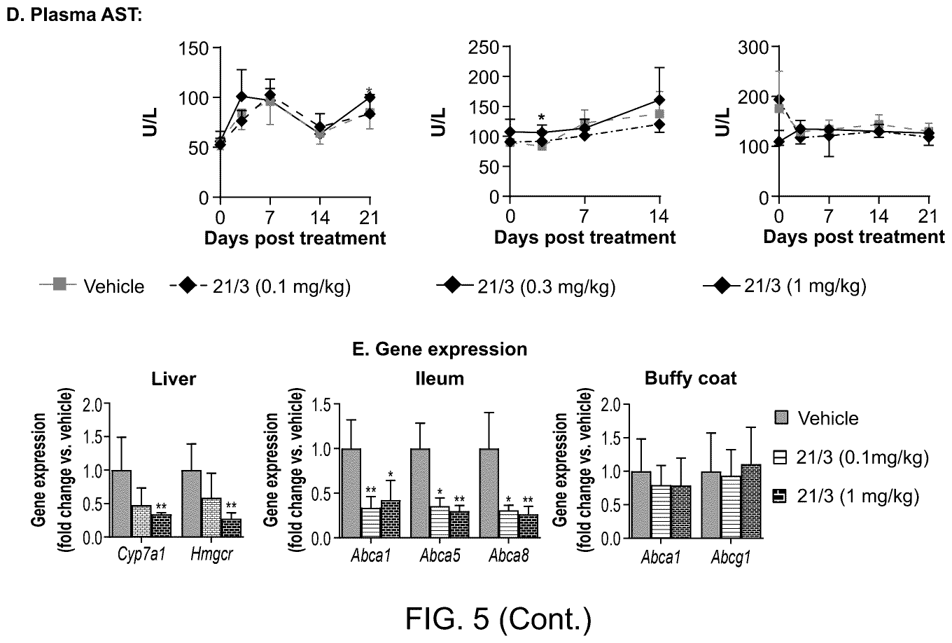

The efficacy of Compound 21/3 was evaluated in three dysmetabolic rodent models including DIO mice, HFD-fed ZDF rats, and HFD-fed SD rats (FIG. 4 and FIG. 5). Briefly, DIO mice were fed a HFD (60% kcal from fat) for 14 weeks and treated with Compound 21/3 (0.3 and 1 mg/kg once daily by oral gavage) for three weeks, while ZDF and SD rats were fed HFD for two weeks prior to treatment with Compound 21/3 (0.1 and 1 mg/kg once daily by oral gavage) for two or three weeks, respectively. FIG. 4 shows that Compound 21/3 reduces liver and plasma tg in multiple dysmetabolic rodent models. FIG. 5 shows that Compound 21/3 lowers plasma total cholesterol and alters expression of hepatic and intestinal, but not peripheral, cholesterol-related genes.

Compound 21/3 dose-dependently reduced the expression of DNL genes including Srebp1c, Fasn, Acaca (ACC1), and Scd1 in all 3 models (FIG. 4A). Compound 21/3 dose-dependently reduced liver TG with maximal reductions of 65% (p≤0.001), 49% (p≤0.01), and 42% (p=0.07) versus vehicle in DIO mice, HFD-fed ZDF rats, and HFD-fed SD rats, respectively (FIG. 4B). Compound 21/3 also reduced plasma TG levels in all three models (FIG. 4C). In HFD-fed ZDF and SD rats, in which diet-induced hypertriglyceridemia was pronounced, the highest dose of Compound 21/3 (1 mg/kg) reduced plasma TG by 96% and 59%, respectively, when compared with vehicle (p≤0.05).

In addition to the effects of Compound 21/3 on hepatic DNL, the observed reductions in plasma TG in these models may be mediated by reduced intestinal TG absorption, as well as increased clearance of TG-containing lipoproteins from the circulation. Indeed, Compound 21/3 dose-dependently reduced intestinal Srebp1c expression in the DIO mouse (FIG. 4D) and in HFD-fed ZDF and SD rats (data not shown). Further, LXR inhibition with Compound pre-6/3 reduced intestinal lipid absorption as measured by reduced uptake of ³H-labelled triolein tracer in HFD-fed mice (FIG. 4E). Additionally, Compound 21/3 reduced hepatic expression and plasma levels of ANGPTL3, an inhibitor of LPL, the predominant enzyme involved in the clearance of TG-rich lipoproteins from the circulation (FIG. 4F). Loss-of-function variants in ANGPTL3 are associated with reduced serum lipids and a lower risk of CAD (Dewey, Gusarova et al. 2017). Similarly, hepatic expression of Angptl3, Angptl4, and Angptl8, as well as ApoC1, ApoC2, and ApoC3, another inhibitor of LPL, were suppressed in HFD-fed ZDF rats treated with Compound pre-6/3 (FIG. 4G). As with ANGPTL3, large genetic studies have demonstrated that loss-of-function variants in ApoC3 have approximately 40% reductions in plasma TG and 40% lower incidence of CAD (Crosby, Peloso et al. 2014).

In DIO mice and HFD-fed ZDF rats, in which total plasma cholesterol levels are significantly elevated, Compound 21/3 also dose-dependently reduced plasma total cholesterol (TC) by 49% and 67%, respectively, at the end of treatment when compared with vehicle levels (p≤0.05) (FIG. 5A). However, Compound 21/3 caused a statistically significant increase in liver TC, particularly in the higher dose groups (FIG. 5B). Importantly, these changes in hepatic cholesterol were not associated with substantial elevations in liver biochemistry tests, including plasma ALT or aspartate aminotransferase (AST), compared with vehicle treatment (FIG. 5C-D). Moreover, the cholesterol content in the diets of these rodents is very high (up to 1%), likely accentuating this pharmacology.

Compound 21/3 dose-dependently reduced the expression of cholesterol synthetic (Hmgcr) and metabolizing (Cyp7a1) enzymes in the liver, and cholesterol efflux transporters (Abca1, Abcg5, and Abcg8) in the intestine (FIG. 5E). However, Compound 21/3 did not alter expression of the cholesterol efflux transporters Abca1 and Abcg1 in the buffy coat, the fraction of collected blood that primarily contains white blood cells. These data indicate that Compound 21/3 acts primarily in the liver and intestine and does not inhibit LXR activity in peripheral tissues, which are responsible for reverse cholesterol transport and associated with atherogenic risk (Rader and Ikewaki 1996).

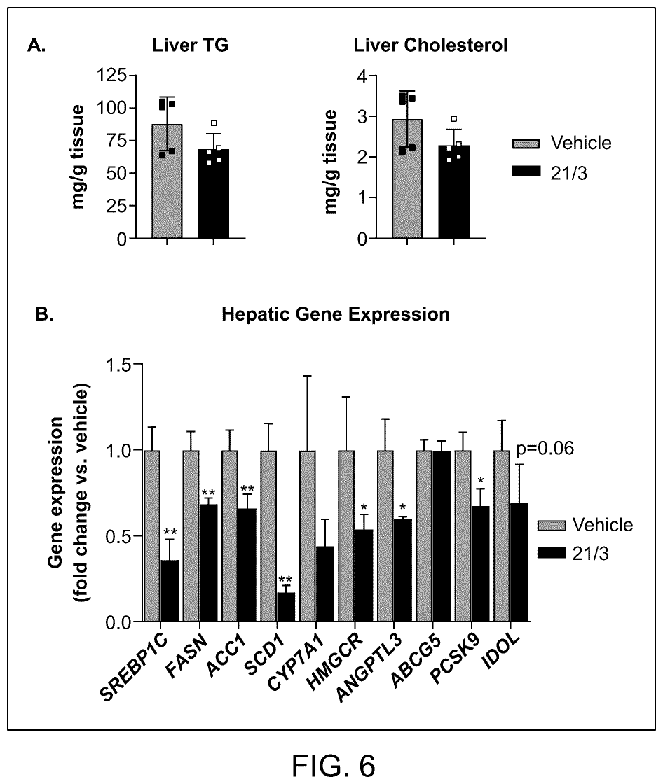

Since features of cholesterol metabolism differ between rodents and humans, the humanized liver chimeric PXB® mouse model was utilized to further explore the effects of Compound 21/3 on lipid metabolism in a more relevant nonclinical model (Tateno, Yoshizane et al. 2004). Briefly, PXB mice were treated with either vehicle or Compound 21/3 (1 mg/kg once daily by oral gavage) for 8 days. The pharmacokinetics (PK) data from this study are presented herein. Compound 21/3 trended to reduce both hepatic TG and TC content, despite the short duration (8 days) of dosing (FIG. 6A). Moreover, the hepatic expression of genes involved in the metabolism of cholesterol and TG were significantly reduced in Compound 21/3-treated mice relative to vehicle (FIG. 6B). Specifically, Compound 21/3 reduced the expression of the cholesterol synthesis gene, HMGCR, as well as in genes that reduce the hepatic uptake of LDL-C (PCSK9, IDOL) and conversion of cholesterol to bile acids (CYP7A1). As expected, marked reductions in ANGPTL3 and DNL-associated genes (SREBP1C, ACACA, FASN, SCD1) were also observed. FIG. 6 shows that Compound 21/3 demonstrates target engagement in humanized liver chimeric mice.

These data provide strong support for the therapeutic potential of Compound 21/3 to regulate lipid homeostasis in human livers.

In summary, Compound 21/3 improves hepatic and systemic lipid homeostasis in multiple dysmetabolic rodent models, resulting in significant reductions in liver TG content and circulating TG and cholesterol levels. Due to its preferential hepatic uptake following oral administration, Compound 21/3 activity is primarily restricted to the liver and intestine and avoids inhibition of reverse cholesterol transport. Thus, these data suggest that liver-selective LXR inhibition using Compound 21/3 may have both cardio- and hepato-protective properties.

### LXR Inverse Agonism has Beneficial Effects on Whole-body Insulin Sensitivity

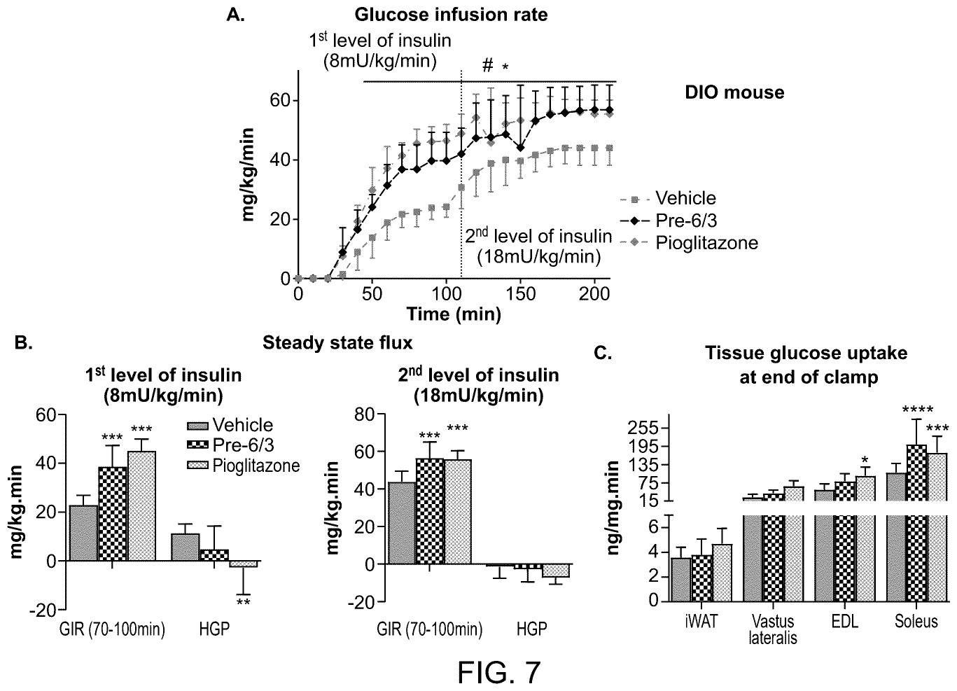

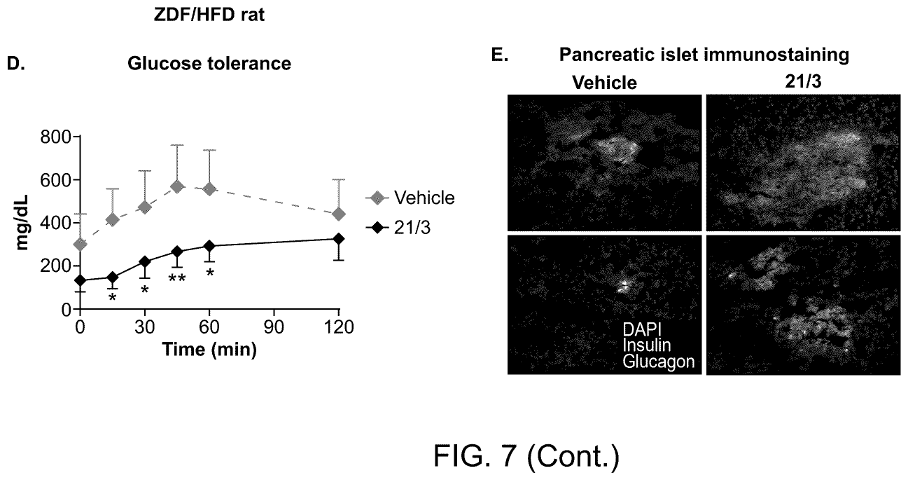

The effects of chronic dosing of Compound 21/3 and Compound pre-6/3 were evaluated in DIO mice and HFD-fed ZDF rats, respectively. Briefly, C57BL/6 mice were fed a HFD for 16 weeks prior to treatment with Compound pre-6/3 (5 mg/kg) or pioglitazone (30 mg/kg; positive control) once daily by oral gavage. After four weeks of treatment, a two-step hyperinsulinemic-euglycemic clamp along with the infusion of radiotracers to monitor glucose disposal and hepatic glucose production was performed. Glucose infusion rate, the rate at which glucose is infused in order to maintain euglycemia, was significantly higher in mice treated with Compound pre-6/3 versus vehicle at both stages of insulin infusion (8 and 18 mU/kg/min), and non-inferior to pioglitazone, indicative of enhanced systemic insulin sensitivity (FIG. 7A-B). These changes were associated with a reduction in hepatic glucose production in the Compound pre-6/3-treated mice, more evident during the 8 mU/kg/min insulin infusion (FIG. 7B). Glucose utilization by muscle, measured at the end of the clamp, was also significantly higher with Compound pre-6/3 than vehicle (FIG. 7C).

Five weeks of treatment with Compound 21/3 (15 mg/kg once daily by oral gavage) also improved glucose homeostasis in HFD-fed ZDF rats. Glucose tolerance, measured by an oral glucose tolerance test after 4 weeks of dosing, was significantly improved with Compound 21/3 at all points assayed (FIG. 7D). Furthermore, immunostaining of pancreatic sections of animals treated with Compound 21/3 demonstrated more prominent staining for insulin relative to vehicle-treated mice, suggesting that Compound 21/3 may have protective effects on the pancreatic β-cell (FIG. 7E).

These data demonstrate that LXR inverse agonism with Compound 21/3 (or its analog Compound pre-6/3) improves insulin action in metabolic tissues such as the liver and muscle and may prevent pancreatic β-cell decline. In addition to the beneficial effects on the liver and circulating lipids, improvements in insulin sensitivity with Compound 21/3 may offer additional cardioprotective benefits. FIG. 7 shows that LXR inverse agonism improves insulin sensitivity and preserves pancreatic β-cell mass.

LXR Inverse Agonism has Beneficial Effects on Fibrosis Progression, Fibrogenesis, and Hepatic Steatosis in a Rodent Model of NASH with Advanced Fibrosis

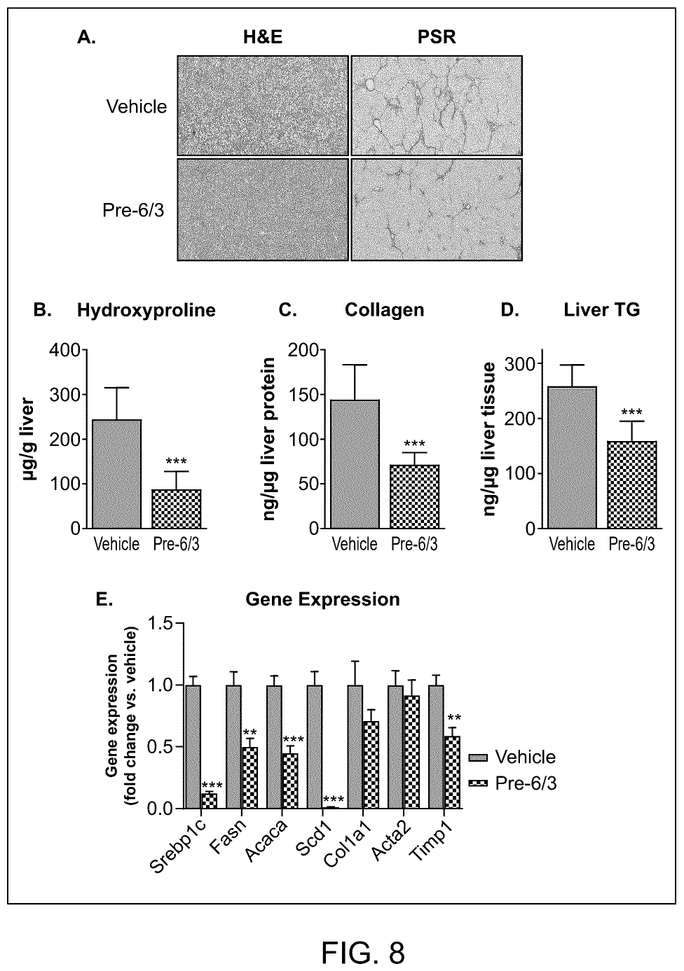

The CDHFD model of NASH is characterized by reduced phosphatidylcholine synthesis in the liver and impaired TG secretion in VLDL particles, which results in hepatic lipid accumulation, inflammation, oxidative stress, and severe fibrosis. Liver injury in CDHFD-fed rats can be exacerbated by further inducing hypoxia using sodium nitrate (Takayama, Egashira et al. 2009). Wistar Han rats were treated with CDHFD/sodium nitrate for a total of 12 weeks and dosed with Compound pre-6/3 (5 mg/kg once daily by oral gavage) from Weeks 6 to 12. Compound pre-6/3 significantly decreased fibrosis, assessed histologically with picrosirus red (PSR) staining which identifies collagen fibrils deposited in the liver (FIG. 8A). Moreover, compared with vehicle treatment, Compound pre-6/3 decreased hepatic hydroxyproline and collagen content by 64% (p≤0.001) and 50% (p≤0.001), respectively (FIG. 8B-C). Compound pre-6/3 also decreased liver TG content (FIG. 8D) and the hepatic expression of genes involved in DNL (Srebp1c, Scd1, Fasn), and fibrogenesis and hepatic stellate cell activation (Timp1, Col1a1, Acta2) (FIG. 8E). FIG. 8 shows that Compound pre-6/3 reduces steatosis and progression of liver fibrosis in the rat CDHFD model.

The totality of the nonclinical primary pharmacology studies demonstrates that liver-targeted LXR inhibition with Compound 21/3 or its analog, Compound pre-6/3, has beneficial therapeutic effects on plasma TG and cholesterol concentrations, hepatic TG content, insulin sensitivity, and liver fibrosis across multiple rodent models. Limited systemic activity of Compound 21/3 due to its preferential distribution to the liver and intestine avoids the risk of inhibiting reverse cholesterol transport, and potential atherogenic effects, which is a risk of systemic LXR inhibition. These data provide a strong rationale for the evaluation of Compound 21/3 to treat subjects with severe dyslipidemia and NASH and suggest that Compound 21/3 may be cardioprotective in these subjects due to its benefits on lipid and glucose metabolism.

### A Study Evaluating the Safety, Tolerability, Pharmacokinetics, and Pharmacodynamics of Single and Multiple Ascending Doses of Compound 21/3 in Healthy Subjects

### Objectives

The primary objectives are to evaluate the safety and tolerability of escalating single and multiple doses of Compound 21/3 and to characterize the single- and multiple-dose pharmacokinetics (PK) of Compound 21/3 and its metabolite(s). The secondary objectives are to evaluate the pharmacodynamics (PD) of liver X receptor (LXR) inverse agonism by Compound 21/3 as measured by metabolic parameters including fasting lipids and serum biomarkers of de novo lipogenesis (DNL); to characterize Compound 21/3 dose and/or exposure-response relationships for PD markers; to evaluate exploratory biomarkers; and to evaluate the effects of fasted vs. fed dosing on the PK profile of Compound 21/3.

### Study Design

A double-blind, placebo-controlled, single- and multiple-ascending dose FIH Phase 1 study to evaluate the safety, tolerability, PK, and PD of Compound 21/3 in healthy adult subjects was performed. A total of up to 150 subjects were enrolled, including 100 subjects in Parts A and B, and up to 50 subjects in adaptive cohorts in Part C. Within each cohort, 10 subjects were randomized, including 8 subjects randomized to Compound 21/3 and 2 subjects to PTM (placebo-to-match). In single-dose cohorts in Parts A and in any Part C cohort evaluating a higher total daily dose than previously evaluated, safety and tolerability data collected through Day 2 from two sentinel subjects, one randomized to Compound 21/3 and one to PTM, were evaluated prior to dosing of the remaining subjects in each cohort.

An approximately even distribution of healthy male and non-pregnant, non-lactating female subjects 18 to 55 years of age, inclusive, were to be enrolled in the study. There were no significant medical conditions by history, physical examination, or laboratory evaluation. Subjects were not allowed to participate in more than one cohort of the study. Within each MAD cohort, attempts were made to enrich for >3 subjects with elevated TG and/or LDL-C. Following the completion of Screening and admission assessments, eligible subjects were to be enrolled on Day −1. Safety and tolerability assessments, including adverse event (AE) monitoring, laboratory tests, physical examinations, and ECG evaluations were performed throughout the study.

The duration of dosing were to be 1 day for the SAD cohorts in Part A and C, and 14 days for the MAD cohorts in Parts B and C. In all subjects in the MAD cohorts, subjects also received PTM on Day −1.

The overall study duration, post-screening, was up to 18 days for Part A, and up to 32 days for Parts B and C.

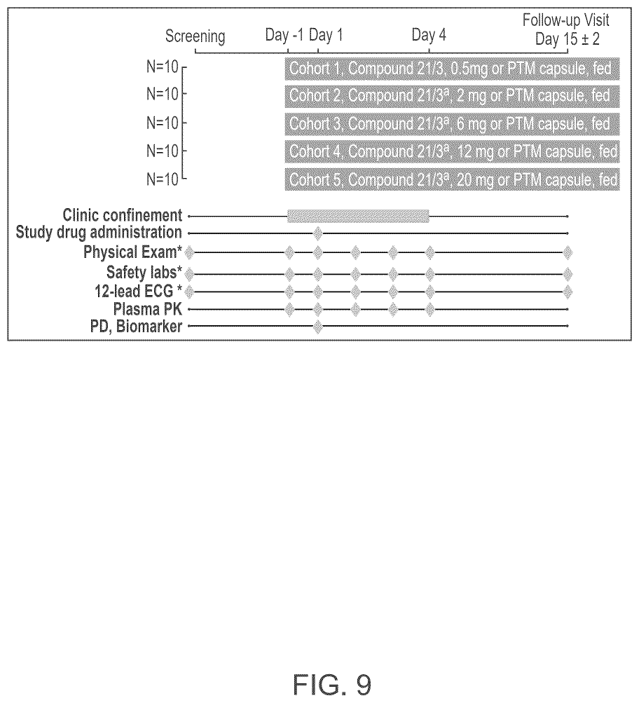

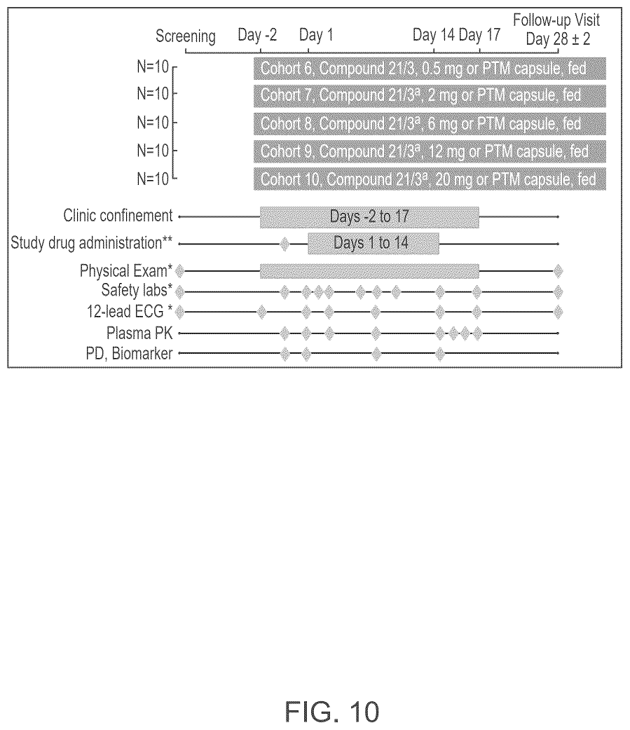

This study proceeded in 3 parts, with progression within and between parts governed by reviews of safety and any available and relevant PK and/or PD data, and the application of stopping rules. The sponsor was allowed to choose not to initiate any or all adaptive cohorts in Part C if deemed unnecessary based on observations in Parts A and B. The overall study design and the design of each part are described herein, and the study schemas are presented in FIG. 9 for SAD cohorts in Parts A and C, and FIG. 10 for MAD cohorts in Parts B and C.

Intensive PK sampling over 72 h postdose on Day 1 (SAD and MAD) and Day 14 (MAD) was performed. Compound 21/3 plasma concentrations were determined using validated liquid chromatography-tandem mass spectrometry assay. PK parameters were estimated via noncompartmental methods using Pheonix® WinNonlin® 6.2.1 and 8.3.4 (Certara, LP, Princeton, NJ).

Lipid parameters, including apolipoprotein B (apoB), were evaluated by NMR LipoProfile® (Labcorp, Burlington, NC. Plasma ApoC3 and ANGPTL3 were evaluated by ELISA (360biolabs, Melbourne, Australia). Peripheral RCT (reverse cholesterol transport) were evaluated by assessing changes in expression of ABCA1/ABCG1 in PBMCs, predose and 4 hours postdose on Days 1 and 14 (Gnomix, Bedford Park, Australia).

The table below shows subject enrollment and demographics for SAD cohorts.

| | 0.5 mg; | 2 mg; | 6 mg; | 12 mg; | 20 mg; | placebo; |
|---|---|---|---|---|---|---|
| | n = 8 | n = 8 | n = 8 | n = 8 | n = 8 | n = 10 |
| Age, y | 38 | 30 | 33 | 33 | 31 | 40 |
| | (18, 48) | (18, 48) | (24, 47) | (21, 48) | (20, 53) | (21, 50) |
| male | 3 | 1 | 3 | 2 | 4 | 4 |
| | (38) | (13) | (38) | (25) | (50) | (40) |
| BMI, | 25.1 | 24.7 | 28.0 | 30.2 | 24.8 | 25.8 |
| kg/m² | (19.5, | (21.5, | (20.2, | (22.4, | (19.9, | (21.7, |
| | 32.2) | 28.3) | 34.0) | 34.5) | 32.2) | 35.0) |

Data are median (min, max or n (%). BMI, body mass index.

The table below shows subject enrollment and demographics for MAD cohorts.

| | 0.5 mg; | 2 mg; | 6 mg; | 6 mg (fasted); | 12 mg; | Placebo; |
|---|---|---|---|---|---|---|
| | n = 8 | n = 8 | n = 8 | n = 8 | n = 8 | n = 10 |
| Age, y | 28 (21, 36) | 26 (19, 30) | 33 (26, 54) | 28 (20, 53) | 29 (19, 49) | 26 (20, 43) |
| Male | 4 (50) | 6 (75) | 7(88) | 8 (100) | 6 (75) | 8 (80) |
| BMI, kg/m² | 25.4 (19.2, 34.5) | 23.7 (19.9, 26.6) | 24.2 (19.5, 31.4) | 21.9 (19.8, 25.8) | 23.2 (20.3, 34.6) | 24.5 (19.8, 30.1) |
| TG, mmol/L | 0.8 (0.5, 1.2) | 1.05 (0.5, 3.0) | 1.45 (0.6, 2.3) | 0.95 (0.8, 1.5) | 0.85 (0.5, 1.3) | 1.1 (0.5, 1.8) |
| Total cholesterol, | 4.35 (2.7, 5.2) | 4.65 (3.7, 5.4) | 4.8 (3.4, 5.9) | 4.55 (3.3, 5.5) | 4.05 (3.3, 5.1) | 4.5 (3.2, 5.7) |
| mmol/L | | | | | | |
| LDL-C, mmol/L | 2.4 (1.2, 3.4) | 2.8 (2.1, 3.7) | 2.75 (1.8, 4.2) | 2.45 (1.5, 3.9) | 2.2 (1.6, 3.3) | 2.75 (1.6, 4.0) |
| HDL-C, mmol/L | 1.32 (0.79, 2.16) | 1.30 (0.83, 1.54) | 1.29 (0.79, 1.57) | 1.63 (0.97, 2.17) | 1.44 (0.88, 2.00) | 1.30 (0.93, 1.95) |

Data are median (min, max or n (%). Lipid parameters measured by chemistry predose on Day 1.

### Part A: Single-Ascending Dose (SAD) (Cohorts 1-5)

Part A evaluated the safety, tolerability, PK, and PD of single-ascending doses of oral Compound 21/3 or placebo under fed conditions. Part A proceeded in up to 5 dose-escalation cohorts and governed by a review of safety, tolerability, and any available and relevant PK and/or PD data, and study-specific stopping criteria. Planned study treatments within each cohort are provided in Table 6 below.

TABLE 6
Treatments in Cohorts 1-5

| Cohort | Single Ascending Doses (Day 1) |
|---|---|
| 1 | 0.5 mg Compound 21/3 (1 × 0.5 mg) or PTM capsule SD, fed |
| 2 | 2 mg Compound 21/3^{a} (1 × 2 mg) or PTM capsule SD, fed |
| 3 | 6 mg Compound 21/3^{a} (3 × 2 mg) or PTM capsule SD, fed |
| 4 | 12 mg Compound 21/3^{a} (2 × 5 mg; 1 × 2 mg) |
| | or PTM capsule SD, fed |
| 5 | 20 mg Compound 21/3^{a} (4 × 5 mg) or PTM capsule SD, fed |

PTM = placebo-to-match;
SD = single dose
^{a}The planned dose may be modified based on observed safety, tolerability, and any relevant and available PK and/or PD data. Any modified dose would be within 3-fold of a dose previously tested.

Within each cohort, 10 unique subjects were randomized 4:1 to receive either blinded Compound 21/3 (N=8) or PTM (N=2). All study drugs in Part A were administered in the morning following a standard meal.

At the initiation of each cohort in Part A, prior to randomization of the entire cohort, two sentinel subjects will be randomized, one to Compound 21/3 at the dose to be evaluated in the planned cohort, and one to corresponding PTM. Enrollment and randomization of the remaining eight subjects in each cohort were determined upon evaluation of all safety and tolerability data through Day 2 for these two sentinel subjects.

For cohorts 2-5, initiation of single-dose administration was permitted after evaluation of cumulative blinded safety data, and any relevant and available PK and/or PD data up to and including Day 4, following single-dose administration of the previous cohort.

A schematic of the activities within Part A cohorts is shown in FIG. 9. Adaptive SAD cohorts in Part C follow the same study schema. In FIG. 9, for each cohort, subjects will be randomized to 8 active and 2 placebo-to-match. For Part C (Cohorts 11-15): Adaptive SAD Cohorts will follow the same schema as in Part A and Adaptive MAD Cohorts will follow the same schema as Part B. In FIG. 9:
“a” notes that: The planned doses may be modified based on the observed safety, tolerability, and any relevant and available PK and/or PD data. Any modified dose will be within 3-fold of a dose previously tested.
“*” notes that: Complete PE at Screening, Day-1, Day 4, and Follow-up visit at Day 15±2; symptom-driven exam performed at other time points indicated.
“ø” notes that: Safety labs on Day 1 to occur 12 hours after study drug dosing; 12-lead ECG on Day 1 to occur 2 hours after study drug dosing.

### Part B: Multiple-Ascending Dose (MAD) (Cohorts 6-10)

Part B evaluated the safety, tolerability, PK, and PD of multiple-ascending doses of oral Compound 21/3 or placebo under fed conditions. Part B will proceed in up to 5 dose-escalation cohorts and will be governed by a review of safety, tolerability, and any available and relevant PK and/or PD data, and study-specific stopping criteria. Planned study treatments within each cohort are provided in Table 7 below.

TABLE 7
Treatments in Cohorts 6-10

| Cohort | Day 1 | Multiple Ascending Doses (Days 1-14) |
|---|---|---|
| 6 | PTM capsule SD | 0.5 mg Compound 21/3 (1 × 0.5 mg) or PTM capsule, fed |
| 7 | PTM capsule SD | 2 mg Compound 21/3^{a} (1 × 2 mg) or PTM capsule, fed |
| 8 | PTM capsules SD | 6 mg Compound 21/3^{a} (3 × 2 mg) or PTM capsules, fed |
| 9 | PTM capsules SD | 12 mg Compound 21/3^{a} (2 × 5 mg; 1 × 2 mg) or PTM |
| | | capsules, fed |
| 10 | PTM capsules SD | 20 mg Compound 21/3^{a} (4 × 5 mg) or PTM capsules, fed |

PTM = placebo-to-match;
SD = single dose
^{a}The planned dose may be modified based on observed safety, tolerability, and any relevant and available PK and/or PD data. Any modified dose would be within 3-fold of a dose previously tested.

An attempt was made to enroll at least 3 subjects in each MAD cohort with Screening TG ≥150 mg/dL and/or low-density lipoprotein cholesterol (LDL-C) ≥130 mg/dL.

Within each cohort, 10 unique subjects were randomized 4:1 to receive either blinded Compound 21/3 (N=8) or PTM (N=2) QD for 14 days.

Part B (Cohort 6) was initiated after the evaluation of cumulative safety, tolerability, and any relevant and available PK and/or PD data from all subjects enrolled in Cohort 3 in Part A (up to and including Day 4).

For Cohorts 7-10, dosing was initiated after evaluation of cumulative safety, tolerability, and any relevant and available PK and/or PD data from all subjects enrolled in the previous cohort in Part B through Day 14.

All study drugs in Part B will be administered in the morning following a standard meal.

Part C: Adaptive SAD and/or MAD (Cohorts 11-15)

Part C included optional, adaptive cohorts to evaluate the safety, tolerability, PK, and PD of single- and/or multiple-ascending doses of oral Compound 21/3 or placebo under fasted or fed conditions. Based on available safety, PK, and/or PD data generated in Part A and Part B, as applicable, doses for Part C (Cohorts 11-15) will be chosen, up to a total daily dose of 50 mg. Planned study treatments within each cohort are provided in Table 8 below.

TABLE 8
Treatments in Cohorts 11-15

| | | Single Dose (Day 1) or |
|---|---|---|
| Cohort | Day 1^{a} | Multiple Doses (Days 1-14) |
| 11-15 | PTM capsule(s) | ≤50 mg total daily dose Compound |
| | QD or BID | 21/3 or PTM, administered |
| | | QD or BID, fasting or fed |

BID = twice daily;
PTM = placebo-to-match;
QD = once daily
^{a}Dosing of PTM on Day 1 performed in multiple-dose adaptive cohorts (as in Part B)

Part C proceeded in up to 5 dose-escalation cohorts and was governed by a review of safety, tolerability, and any available and relevant PK and/or PD data generated from previous Part A, B, and/or C cohorts, as applicable.

An attempt was made to enroll at least 3 subjects in each cohort with Screening TG ≥150 mg/dL and/or low-density lipoprotein cholesterol (LDL-C) ≥130 mg/dL.

Within each cohort, 10 unique subjects were randomized 4:1 to receive up to a total daily dose of 50 mg Compound 21/3 (N=8) or PTM (N=2) for either 1 day (as in Part A) or 14 days (as in Part B).

The frequency of dosing (QD or BID in MAD cohorts) and fasting versus fed status (in SAD or MAD cohorts) may also be modified versus Parts A and B. If dosing is administered in the fed state, standard meal conditions may also be modified at discretion. QD doses were administered in the morning following a standard meal, and BID doses were administered after a standard morning meal and evening snack, with the second dose administered 12 hours (±10 minutes) after the morning dose. Once determined, dose level, duration of dosing, frequency of dosing, and meal conditions remained consistent within a cohort. For any MAD cohort in Part C with BID dosing, both morning and evening doses must be administered on Day 14.

Multiple-dose, adaptive cohorts in Part C were initiated in parallel with previous cohorts if the total daily dose under evaluation is at or below a dose already evaluated in Part B. If the total daily dose in any cohort is greater than has been previously evaluated, then sentinel dosing including one subject randomized to placebo and one subject to Compound 21/3 was included. Randomization of the remaining eight subjects in the cohort will be based on evaluation of all safety and tolerability data through Day 2 for these two sentinel subjects. If doses chosen in 2 or more adaptive cohorts exceed the dose evaluated in a previous cohort in Part B, these cohorts were conducted in a staggered manner (lowest dose first), with the same stopping rules applied.

A schematic of the activities within MAD cohorts in Parts B and C is shown in FIG. 10. In FIG. 10, for each cohort, subjects will be randomized to 8 active and 2 placebo-to-match. For Part C (Cohorts 11-15): Adaptive SAD Cohorts will follow the same schema as in Part A and Adaptive MAD Cohorts will follow the same schema as Part B. In FIG. 10:
“a” notes that: The planned doses may be modified (reduced) based on the observed safety, tolerability, and any relevant and available PK and/or PD data. Any modified dose will be within 3-fold of a dose previously tested.
“*” notes that: Complete PE at Screening, Day-2, Day 17, and Follow-up visit on Day 28±2; symptom-driven PE on all other days of confinement.
“**” notes that: All multiple-dose subjects in Part B and C will be administered PTM on Day −1.
“¥” notes that: Safety labs on Day 1 to occur 12 hours after study drug dosing; 12-lead ECG on Day 1 to occur 2 hours after study drug dosing.

### Dose Escalation Criteria

### SAD Cohorts in Parts A and C

At the initiation of each single-dose cohort in Part A and in any single-dose cohort in Part C evaluating a higher total daily dose of Compound 21/3 than previously evaluated, two sentinel subjects will be randomized, one to Compound 21/3 at the dose to be evaluated in the planned cohort, and one to corresponding PTM. Enrollment and randomization of the remaining eight subjects in each cohort will be determined upon evaluation of all safety and tolerability data through Day 2 for these two sentinel subjects.

Initiation of single-dose administration at a higher dose was determined by evaluation of safety and any relevant and available PK and/or PD data through Day 4 for all subjects enrolled in the previous dosing cohort.

### MAD Cohorts in Part B

Dosing in Cohort 3 of the SAD portion of the study (Part A) was completed prior to proceeding with the MAD portion of the study. Initiation of multiple dosing within Cohort 6 was determined by a review of the cumulative safety, and any relevant and available PK and/or PD data through Day 4 of Cohort 3 in Part A.

Initiation of subsequent multiple-dose cohorts at a higher dose was determined upon evaluation of all safety data, and any relevant and available PK and/or PD data through Day 14 from all subjects enrolled in the previous multiple-dose cohort.

In both Parts A and B, escalation to a dose higher than previously studied may occur only in the absence of dose-limiting toxicity and/or not meeting any prespecified stopping criteria.

### Adaptive MAD Cohorts in Part C

Based on safety and available PK and/or PD data from cohorts in Part A and Part B (if applicable), Compound 21/3 doses for Part C may be chosen up to a total daily dose of 50 mg. If the total daily dose in any cohort in Part C is greater than has been evaluated in any prior cohort, then sentinel dosing including one subject randomized to placebo and one subject randomized to Compound 21/3 would be completed. Randomization of the remaining eight subjects in the cohort was determined based on evaluation of all safety and tolerability data through Day 2 for these two sentinel subjects. Additionally, if doses chosen in two or more adaptive cohorts exceed the dose evaluated in a previous cohort, those cohorts were conducted in a staggered manner (lowest dose first), with the same stopping rules applied. Part C cohorts may be initiated in parallel with cohorts in Part B if the total daily dose under evaluation is at or below a dose already evaluated.

The sponsor may choose not to initiate any or all adaptive cohorts if deemed unnecessary.

### Protocol-Specific Stopping Criteria

### Subject Study Drug Discontinuation Criteria

The severity of AEs was graded using the Common Terminology Criteria for Adverse Events version 5.0 (CTCAE v5.0).

Study drug must be discontinued in subjects who experience any of the following:
A confirmed, treatment-emergent, treatment-related, serious AE (SAE) or ≥Grade 3 AE
Any ≥Grade 3 clinically significant laboratory abnormality (confirmed by repeat testing) as defined by the Common Terminology Criteria for Adverse Events (CTCAE) v5.0 for Severity of Adverse Events and Laboratory Abnormalities
Symptoms of drug-related hepatotoxicity, and/or ALT or AST>5×ULN, or ALT>3×ULN and total bilirubin >2×ULN or INR>1.5, confirmed by immediate repeat testing

Study drug and/or study procedures may also be discontinued in the following instances:
Intercurrent illness that would, in the judgment of the investigator, affect assessments of clinical status to a significant degree
Unacceptable toxicity, as defined in the toxicity management section of the protocol, or toxicity that, in the judgment of the investigator, compromises the ability to continue study-specific procedures or is considered not to be in the subject's best interest
Subject request to discontinue for any reason
Subject noncompliance
Pregnancy during the study
Investigator discretion
Discontinuation of the study at the request of the researchers, regulatory agency, or an Institutional Review Board (IRB)/Independent Ethics Committee (IEC)

### Study Specific Stopping Criteria

Dose escalation or study drug dosing of a cohort can be suspended and based on a full review of the clinical data by the medical monitor (MM) or designee, and in discussion with the investigator, may be halted when:
1 subject dosed with Compound 21/3 experiences a treatment-emergent SAE deemed possibly related to study drug in the opinion of the investigator
2 subjects dosed with Compound 21/3 within 1 cohort experience elevations in liver biochemistry tests of ALT and/or AST>5×the ULN, or ALT>3×ULN and total bilirubin >2×ULN or INR>1.5, confirmed by immediate repeat testing
2 or more subjects dosed with Compound 21/3 experience the same ≥Grade 3 AE deemed possibly related to the study drug in the opinion of the investigator
2 or more subjects dosed with Compound 21/3 experience a clinically significant ≥Grade 3 laboratory abnormality of similar nature (confirmed on repeat testing) and possibly related to the study drug in the opinion of the investigator
The number and/or severity of AEs justify study discontinuation
The sponsor requests study discontinuation Decisions to reinitiate the study can be made in consultation with the sponsor and pending a comprehensive safety review.

### Duration of Dosing

The duration of dosing for each part was as follows:
Part A (SAD): 1 day
Part B (MAD): 15 days (including PTM dosing for all subjects on Day −1)
Part C (Adaptive SAD and/or MAD): 1 or 15 days (including PTM dosing for all multiple-dose subjects on Day −1)

### Clinical Confinement

Following Screening and admission procedures, eligible subjects were confined to the study center beginning at admission (Day −1 for SAD cohorts; Day −2 for MAD cohorts) until the completion of assessments on Day 4 (SAD cohorts), or Day 17 (MAD cohorts).

Subjects returned 14±2 days after the last dose for an in-clinic follow-up visit (i.e., Day 15 [±2 days] for SAD cohorts; Day 28 [±2 days] for MAD cohorts.

### Pharmacokinetic Assessments

Plasma concentrations of Compound 21/3 (and its metabolites, as applicable) were determined and PK evaluated. PK parameters would be estimated, as appropriate. Plasma concentrations of other metabolites may also be determined and PK explored.

### Pharmacodynamic Assessments

Blood samples and stool samples for assessment of PD biomarkers related to Compound 21/3 and its mechanism of action was collected throughout the study.

### Safety Assessments

Safety assessments were performed throughout the study.

### Results: SAD Cohorts

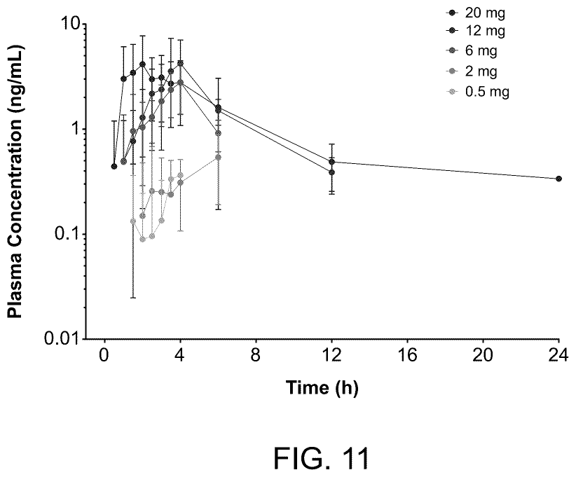

Plasma concentration time profiles on Day 1 in the SAD study are shown in FIG. 11 and the table below.

| | 0.5 mg: | 2 mg: | 6 mg: | 12 mg: | 20 mg: |
|---|---|---|---|---|---|
| | n = 8 | n = 8 | n = 8 | n = 8 | n = 8 |
| AUC_{inf} | 1.2 | 8.5 | 10.0 | 17.1 | 20.6 |
| (h · ng/mL) | (n = 1) | (107.5) | (17.5) | (32.5) | (40.7) |
| | | (n = 3) | (n = 8) | (n = 8) | (n = 8) |
| Cₘₐₓ | 0.37 | 0.54 | 3.33 | 4.69 | 6.79 |
| (ng/mL) | (7.7) | (56.4) | (28.5) | (76.5) | (42.8) |
| | (n = 3) | (n = 8) | (n = 8) | (n = 8) | (n = 8) |
| Tₘₐₓ | 3.5 | 3.75 | 4.0 | 3.0 | 2.5 |
| (h) | (1.5, 4.0) | (2.5, 6.0) | (1.5, 4.1) | (1.0, 4.0) | (1.0, 6.0) |
| | (n = 3) | (n = 8) | (n = 8) | (n = 8) | (n = 8) |
| t_{1/2} | 2.10 | 4.96 | 1.37 | 2.02 | 2.36 |
| (h) | (n = 1) | (1.38, 30.49) | (0.74, 2.76) | (1.45, 4.18) | (1.09, 6.55) |
| | | (n = 3) | (n = 8) | (n = 8) | (n = 8) |

PK parameters are presented as mean (% CV) except t_{1/2} and Tₘₐₓ, which are presented as median (min, max).
Number of subjects with calculable PK parameter indicated in parentheses..

The SAD study shows the following.
Compound 21/3 was rapidly absorbed, with Tₘₐₓ occurring ˜3 to 4 hours postdose.
There were a short t_{1/2} (˜2 to 3 hours) and low maximal plasma concentrations (Cₘₐₓ<7 ng/mL) consistent with rapid hepatic uptake.
There was less than dose-proportional increases in plasma exposure.

### Results: MAD Cohorts

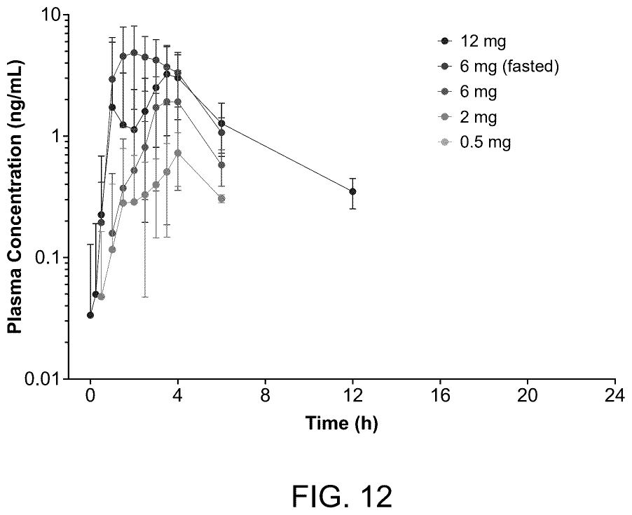

Plasma concentration time profiles on Day 14 in the MAD study are shown in FIG. 12 and the table below.

| | 0.5 mg: | 2 mg: | 6 mg: | 6 mg (fasted): | 12 mg: |
|---|---|---|---|---|---|
| | n = 8 | n = 8 | n = 8 | n = 8 | n = 8 |
| AUCₜₐᵤ | — | 2.7 | 6.05 | 19.0 | 14.6 |
| (h · ng/mL) | | (8.56) | (43.15) | (34.78) | (28.01) |
| | | (n = 4) | (n = 8) | (n = 7) | (n = 8) |
| Cₘₐₓ | — | 0.95 | 2.16 | 6.37 | 4.85 |
| (ng/mL) | | (20.6) | (75.0) | (44.3) | (72.6) |
| | | (n = 6) | (n = 8) | (n = 7) | (n = 8) |
| Tₘₐₓ | — | 4.0 | 3.5 | 2.0 | 3.5 |
| (h) | | (1.0, 4.1) | (1.5, 4.0) | (1.0, 3.5) | (1.0, 6.0) |
| | | (n = 6) | (n = 8) | (n = 7) | (n = 8) |
| t_{1/2} | — | 1.43 | 1.51 | 1.46 | 1.74 |
| (h) | | (1.06, 4.04) | (0.52, 1.83) | (1.09, 2.86) | (0.92, 5.25) |
| | | (n = 4) | (n = 8) | (n = 7) | (n = 8) |

Unless indicated, all doses were administered in fed state.
PK parameters are presented as mean (% CV) except t_{1/2} and Tₘₐₓ, which are presented as median (min, max).
Number of subjects with calculable PK parameter indicated in parentheses. Blank cells indicate parameter could not be calculated due to low systemic exposures of Compound 21/3.

The MAD study shows the following.
Mean Compound 21/3 steady-state t₁/2 ranged from 1.4 to 2.6 h.
Compound 21/3 AUC and Cₘₐₓ increased less than dose proportionally and were about 25-50% lower on Day 14 vs Day 1 with fed dosing.
With fasted dosing, exposures were similar on Day 1 and 14 and −0.3-fold higher than with fed dosing.

### Results: MAD Cohorts-Safety and Tolerability

Safety and tolerability results from the MAD study are shown in the table below.

| | 0.5 mg; | 2 mg; | 6 mg; | 6 mg (fasted); | 12 mg; | Placebo; |
|---|---|---|---|---|---|---|
| | n = 8 | n = 8 | n = 8 | n = 8 | n = 8 | n = 10 |
| Any TEAE | 7 (88) | 3 (38) | 5 (63) | 1 (13) | 3 (38) | 4 (40) |
| Diarrhea | 2 (25) | 0 (0) | 1 (13) | 0 (0) | 2 (25) | 1 (10) |
| Headache | 1 (13) | 0 (0) | 1 (13) | 0 (0) | 2 (25) | 2 (20) |
| Abdominal pain | 1 (13) | 0 (0) | 1 (13) | 1 (13) | 1 (13) | 0 (0) |
| Back pain | 0 (0) | 0 (0) | 2 (25) | 0 (0) | 0 (0) | 1 (10) |
| Pruritis | 4 (50) | 0 (0) | 0 (0) | 0 (0) | 0 (0) | 0 (0) |
| Grade ≥2 TEAE | 0 (0) | 1 (13)* | 0 (0) | 0 (0) | 0 (0) | 0 (0) |
| Treatment-related | 0 (0) | 0 (0) | 1 (13) | 0 (0) | 2 (25) | 1 (10) |
| TEAE | | | | | | |
| TEAE leading to study | 0 (0) | 0 (0) | 0 (0) | 0 (0) | 0 (0) | 0 (0) |
| drug discontuation | | | | | | |
| Serious TEAE | 0 (0) | 0 (0) | 0 (0) | 0 (0) | 0 (0) | 0 (0) |

Unless indicated, all doses were administered in fed state.
TEAE, treatment-emergent AE. TEAEs reported by at least 3 subjects overall are listed by MedDRA Preferred Term.
*Grade 2 thrombophlebitis considered unrelated to study treatment.

The MAD study shows the following.
Compound 21/3 at 0.5, 2, 6, and 12 mg for 14 days were well tolerated.
All TEAEs in the Compound 21/3 cohorts were non-serious, and all but one were mild in severity.
Treatment-related TEAEs, all mild in severity and gastrointestinal in nature, occurred in 3 subjects treated with Compound 21/3 (abdominal discomfort and diarrhea [6 mg fed; n=1]; abdominal pain and/or diarrhea [12 mg; n=2]) and 1 subject with placebo (nausea).
Mild (Grade 1), transient increase in serum ALT observed in one subject in the Compound 21/3-6 mg group and 4 subjects in the 12 mg Compound 21/3 group (both fed).
No other clinically significant laboratory abnormalities or changes in vital signs or ECG parameters.

### Safety, Pharmacokinetics, and Lipid Lowering Effects of the Oral, Liver-Targeted Liver X Receptor (LXR) Inverse Agonist in Healthy Volunteers

Background. The liver X receptors are oxysterol-activated nuclear hormone receptors that regulate cholesterol homeostasis and de novo lipogenesis. Compound 12/3 is an oral, liver-targeted inverse agonist of LXRα/β in development for severe hypertriglyceridemia (SHTG) and NASH.

Methods. In a randomized, double-blind, placebo (PBO)-controlled Phase 1 study, healthy subjects (n=8/2 Compound 21/3 vs PBO/cohort) received single ascending doses (SAD) of Compound 21/3 (0.5, 2, 6, 12, or 20 mg) or multiple ascending doses (MAD) QD for 14 days (0.5, 2, 6, or 12 mg) while fed or 6 mg fasted. Safety, PK, serum lipid parameters by NMR LipoProfile®, and gene expression in PBMCs were evaluated.

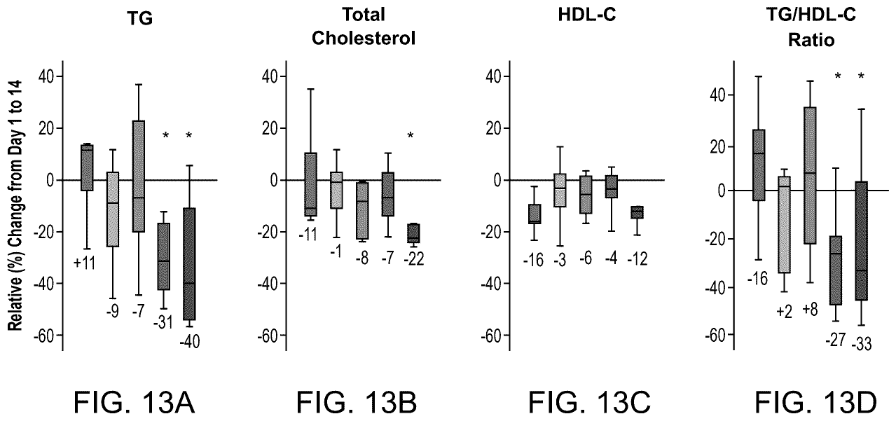

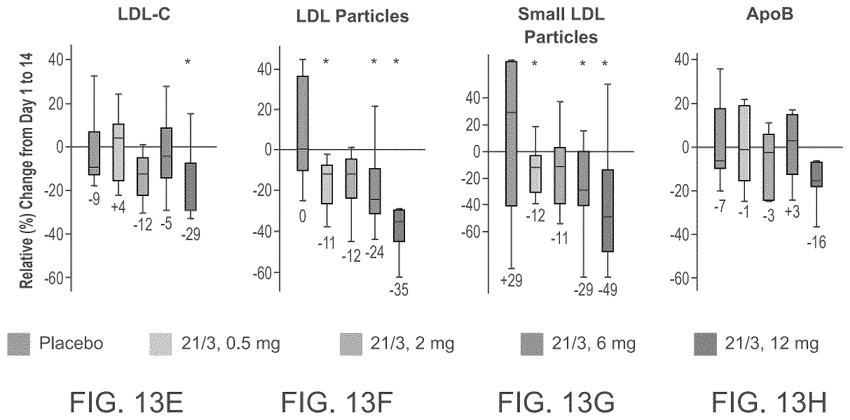

Results. The proportion of subjects with adverse events (AEs) was 60% with PBO and 13-50% with Compound 21/3 in the SAD cohorts (n=50), and 40% and 13-88%, respectively, in the MAD cohorts (n=50). All AEs were mild except an unrelated, Grade 2 thrombophlebitis in a subject on Compound 21/3 2 mg. Compound 21/3 was rapidly absorbed (Tmax 2-4 h) with a short steady-state half-life (T1/2˜1.5-2.5 h) and low maximal plasma concentrations (Cmax <7 ng/mL), consistent with rapid hepatic uptake. With fed dosing, Compound 21/3 AUC and Cmax increased less than dose proportionally and were 25-50% lower on Day 14 vs Day 1. With fasted dosing, exposures were similar on Days 1 and 14 and ˜3-fold higher than with fed dosing. Significant, dose-dependent reductions in serum triglycerides, total and LDL-C, total and small LDL particles, and TG/HDL-C ratio—but not HDL-C—were observed with Compound 21/3 (FIGS. 13A-13H). In FIG. 13A-13H, figures on graphs indicate median relative (%) change in predose NMR Lipoprofile® parameter from Day 1 to Day 14. Also, *p<0.05 for comparison of change from baseline vs. placebo by Mann-Whitney test. Also, Compound 21/3-6 mg fed and fasted groups combined (N=16); Compound 21/3-0.5, 2, and 12 mg groups (N=8 each); pooled placebo group (N=10).

FIG. 13A shows relative (%) change in triglycerides from Day 1 to 14 during the MAD phase of the study. FIG. 13B shows relative (%) change in total cholesterol from Day 1 to 14 during the MAD phase of the study. FIG. 13C shows relative (%) change in HDL-C from Day 1 to 14 during the MAD phase of the study. FIG. 13D shows relative (%) change in TG/HDL-C ratio from Day 1 to 14 during the MAD phase of the study. FIG. 13E shows relative (%) change in LDL-C from Day 1 to 14 during the MAD phase of the study. FIG. 13F shows relative (%) change in LDL particles from Day 1 to 14 during the MAD phase of the study. FIG. 13G shows relative (%) change in small LDL particles from Day 1 to 14 during the MAD phase of the study. FIG. 13H shows relative (%) change in ApoB from Day 1 to 14 during the MAD phase of the study. FIG. 13A-13H indicate median relative change in NMR LipoProfile® parameter from Day 1 to Day 14. “*” notes that: p<0.05 for comparison of change from Day 1 vs. placebo by Mann-Whitney test.

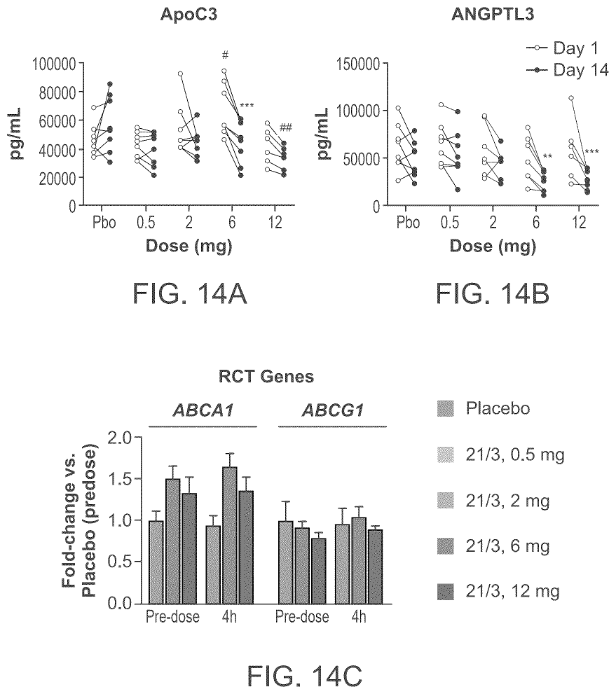

The MAD study shows that Compound 21/3 reduces plasma ApoC3 and ANGPTL3, but does not impact peripheral RCT. FIG. 14A shows levels of ApoC3 according to doses of Compound 21/3 and placebo from Day 1 to Day 14. FIG. 14B shows levels of ANGPTL3 according to doses of Compound 21/3 and placebo from Day 1 to Day 14. FIG. 14C shows levels of RCT genes according to doses of Compound 21/3 and placebo. In FIG. 14A to 14C, ApoC3 and ANGPTL3 measured 12 h postdose. RCT assessed predose and 4 h postdose on Day 14. In FIG. 14A-14C, “*” indicates that p<0.05; “*” indicates that *p<0.01; “***” indicates that p<0.001; “****” indicates that p<0.0001 vs. Day 1; “#” indicates that p<0.05; “##” indicates that p≤0.01 vs. placebo on Day 1 or 14 by two-way ANOVA.

Reductions in ApoC3 and ANGTPL3 are expected to enhance lipoprotein lipase (LPL)-mediated clearance of TG-rich lipoproteins. Compound 21/3 did not impact the expression of LXR target genes in PBMCs, including the RCT genes ABCA1/ABCG1

The MAD study shows the following.
Single and multiple daily doses of the liver-targeted LXR inverse agonist Compound 21/3 up to 20 mg/d for 14 days were safe and well tolerated in healthy subjects.
Liver targeted pharmacology of Compound 21/3 supported by its short half-life (˜1.5-2.5 h), low systemic exposures, and lack of effect on expression of genes involved in peripheral reverse cholesterol transport (i.e., ABCA1, ABCG1).
Compound 21/3 caused significant, dose-dependent improvements in atherogenic lipoproteins, including TG, total and LDL-C, LDL particles, and apoB, particularly in subjects with higher baseline values.
The tolerability, PK profile, and lipid lowering benefits of Compound 21/3 were notable.

Accordingly, the study shows that Compound 21/3 is safe and well tolerated in healthy subjects. The tolerability, PK profile, and lipid lowering benefits of Compound 21/3 were notable. Compound 21/3 can target the liver and has a short half life and low systemic exposure, which results in low toxicity. Administration of Compound 21/3 resulted in significant, dose-dependent improvements in atherogenic lipoproteins, including TG, total and LDL-C, LDL particles, and apoB, particularly in subjects with higher baseline values.

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A method of treating dyslipidemia or a metabolic disorder associated with an impairment in lipid homeostasis in a subject in need thereof, comprising administering to the subject in need thereof an effective amount of a compound represented by Formula (I) 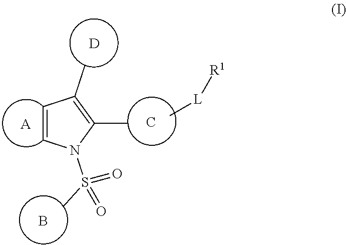
a glycine conjugate, tauro conjugate, enantiomer, diastereomer, tautomer, N-oxide, solvate, prodrug and pharmaceutically acceptable salt thereof,
wherein [symbol]
is an annelated 5- to 6-membered cycle forming a 6-membered aryl or a 5- to 6-membered heteroaryl containing 1 to 3 heteroatoms independently selected from N, O and S, wherein this cycle is unsubstituted or substituted with 1 to 4 substituents independently selected from the group consisting of halogen, CN, SF₅, NO₂, C₁₋₆-alkyl, oxo, C₀₋₆-alkylene-OR¹¹, C₀₋₆-alkylene-(3- to 6-membered cycloalkyl), C₀₋₆-alkylene-(3- to 6-membered heterocycloalkyl), C₀₋₆-alkylene-S(O)ₙR¹¹, C₀₋₆-alkylene-NR¹¹S(O)₂R¹¹, C₀₋₆-alkylene-S(O)₂NR¹¹R¹², C₀₋₆-alkylene-NR¹¹S(O)₂NR¹¹R¹², C₀₋₆-alkylene-CO₂R¹¹, O—C₁₋₆-alkylene-CO₂R¹¹, C₀₋₆-alkylene-O—COR¹¹, C₀₋₆-alkylene-CONR¹¹R¹², C₀₋₆-alkylene-NR¹¹—COR¹¹, C₀₋₆-alkylene-NR¹¹—CONR¹¹R¹², C₀₋₆-alkylene-O—CONR¹¹R¹², C₀₋₆-alkylene-NR¹¹—CO₂R¹¹ and C₀₋₆-alkylene-NR¹¹R¹²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl; and
wherein optionally two adjacent substituents on the aryl or heteroaryl moiety form a 5- to 8-membered partially unsaturated cycle optionally containing 1 to 3 heteroatoms independently selected from O, S or N, and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, O—C₁₋₄-alkyl and O-halo-C₁₋₄-alkyl;
\textcircled{B} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl,
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
\textcircled{C} is selected from the group consisting of 6- or 10-membered aryl and 5- to 10-membered heteroaryl containing 1 to 3 heteroatoms independently selected from N, O and S,
wherein cycloalkyl, heterocycloalkyl, aryl and heteroaryl are unsubstituted or substituted with 1 to 4 substituents independently selected from the group consisting of halogen, CN, SF₅, NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-(6-membered aryl), C₀₋₆-alkylene-(5- to 6-membered heteroaryl), C₀₋₆-alkylene-S(O)ₙR³¹, C₀₋₆-alkylene-NR³¹S(O)₂R³¹, C₀₋₆-alkylene-S(O)₂NR³¹R³², C₀₋₆-alkylene-NR³¹S(O)₂NR³¹R³², C₀₋₆-alkylene-CO₂R³¹, O—C₁₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹ and C₀₋₆-alkylene-NR³¹R³²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
\textcircled{D} is selected from the group consisting of 3- to 10-membered cycloalkyl, 3- to 10-membered heterocycloalkyl containing 1 to 3 heteroatoms independently selected from N, O and S, 6- to 14-membered aryl and 5- to 14-membered heteroaryl containing 1 to 4 heteroatoms independently selected from N, O and S,
wherein cycloalkyl, heterocycloalkyl, aryl and heteroaryl are unsubstituted or substituted with 1 to 6 substituents independently selected from the group consisting of halogen, CN, SF₅, NO₂, oxo, C₁₋₄-alkyl, C₀₋₆-alkylene-OR²¹, C₀₋₆-alkylene-(3- to 6-membered cycloalkyl), C₀₋₆-alkylene-(3- to 6-membered heterocycloalkyl), C₀₋₆-alkylene-S(O)ₙR²¹, C₀₋₆-alkylene-NR²¹S(O)₂R²¹, C₀₋₆-alkylene-S(O)₂NR²¹R²², C₀₋₆-alkylene-NR²¹S(O)₂NR²¹R²², C₀₋₆-alkylene-CR⁴¹(═N—OR⁴¹), C₀₋₆-alkylene-CO₂R²¹, O—C₁₋₆-alkylene-CO₂R²¹, C₀₋₆-alkylene-O—COR²¹, C₀₋₆-alkylene-CONR²¹R²², C₀₋₆-alkylene-NR²¹—COR²¹, C₀₋₆-alkylene-NR²¹—CONR²¹R²², C₀₋₆-alkylene-O—CONR²¹R²², C₀₋₆-alkylene-NR²¹—CO₂R²¹ and C₀₋₆-alkylene-NR²¹R²²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, CO—OC₁₋₄-alkyl, C₁₋₄-alkyl, halo-C₁₋₄-alkyl, O—C₁₋₄-alkyl and O-halo-C₁₋₄-alkyl;
and wherein optionally two adjacent substituents on the aryl or heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the cycloalkyl or heterocycloalkyl moiety form a 5- to 6-membered unsaturated cycle optionally containing 1 to 3 heteroatoms independently selected from O, S or N,
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
wherein \textcircled{D} has a substituent from above in 1,2-orientation regarding to the connection towards 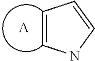
or has an annelated additional cycle in 1,2-orientation;
L is selected from the group consisting of a bond, C₁₋₆-alkylene, C₂₋₆-alkenylene, C₂₋₆-alkinylene, 3- to 10-membered cycloalkylene, 3- to 10-membered heterocycloalkylene containing 1 to 4 heteroatoms independently selected from N, O and S, 6- or 10-membered arylene and 5- to 10-membered heteroarylene containing 1 to 4 heteroatoms independently selected from N, O and S,
wherein alkylene, alkenylene, alkinylene, cycloalkylene, heterocycloalkylene, arylene and heteroarylene are unsubstituted or substituted with 1 to 6 substituents independently selected from the group consisting of halogen, CN, SF₅, NO₂, oxo, C₁₋₄-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(O)ₙR⁴¹, C₀₋₆-alkylene-NR⁴¹S(O)₂R⁴¹, C₀₋₆-alkylene-S(O)₂NR⁴¹R⁴², C₀₋₆-alkylene-NR⁴¹S(O)₂NR⁴¹R⁴², C₀₋₆-alkylene-CO₂R⁴¹, O—C₁₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹ and C₀₋₆-alkylene-NR⁴¹R⁴²,
wherein alkyl, alkylene, cycloalkyl and heterocycloalkyl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the arylene and heteroarylene moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, C₁₋₄-alkyl, halo-C₁₋₄-alkyl, O—C₁₋₄-alkyl and O-halo-C₁₋₄-alkyl;
R¹ is selected from the group consisting of H, halogen, CN, SF₅, NO₂, oxo, C₁₋₄-alkyl, C₀₋₆-alkylene-OR⁴¹, Y—C₀₋₆-alkylene-(3- to 6-membered cycloalkyl), Y—C₀₋₆-alkylene-(3- to 6-membered heterocycloalkyl), Y—C₀₋₆-alkylene-(6-membered aryl), Y—C₀₋₆-alkylene-(5- to 6-membered heteroaryl), C₀₋₆-alkylene-S(═O)(—R⁴¹)═N—R⁷⁵, X—C₁₋₆-alkylene-S(═O)(—R⁴¹)═N—R⁷⁵, C₀₋₆-alkylene-S(O)ₙR⁴¹, X—C₁₋₆-alkylene-S(O)ₙR⁴¹, C₀₋₆-alkylene-S(═NR⁷¹)R⁴¹, X—C₁₋₆-alkylene-S(═NR⁷¹)R⁴¹, C₀₋₆-alkylene-S(O)(═NR⁷¹)R⁴¹, X—C₁₋₆-alkylene-S(O)(═NR⁷¹)R⁴¹, C₀₋₆-alkylene-S(═NR⁷¹)₂R⁴¹, X—C₁₋₆-alkylene-S(═NR⁷¹)₂R⁴¹, C₀₋₆-alkylene-NR⁴¹S(O)₂R⁴¹, X—C₁₋₆-alkylene-NR⁴¹S(O)₂R⁴¹, C₀₋₆-alkylene-S(O)₂NR⁴¹R⁴², X—C₁₋₆-alkylene-S(O)₂NR⁴¹R⁴², C₀₋₆-alkylene-NR⁴¹S(O)₂NR⁴¹R⁴², X—C₁₋₆-alkylene-NR⁴¹S(O)₂NR⁴¹R⁴², C₀₋₆-alkylene-SO₃R⁴¹, X—C₁₋₆-alkylene-SO₃R⁴¹, C₀₋₆-alkylene-CO₂R⁴¹, X—C₁₋₆-alkylene-CO₂R⁴¹, C₀₋₆-alkylene-O—COR⁴¹, X—C₁₋₆-alkylene-O—COR⁴¹, C₀₋₆-alkylene-CONR⁴¹R⁴², X—C₁₋₆-alkylene-CONR⁴¹R⁴², C₀₋₆-alkylene-CONR⁴¹OR⁴¹, X—C₁₋₆-alkylene-CONR⁴¹OR⁴¹, C₀₋₆-alkylene-CONR⁴¹SO₂R⁴¹, X—C₁₋₆-alkylene-CONR⁴¹SO₂R⁴¹, C₀₋₆-alkylene-NR⁴¹—COR⁴¹, X—C₁₋₆-C₀₋₆-alkylene-NR⁴¹—COR⁴¹, C₀₋₆-alkylene-NR⁴¹—CONR⁴¹R⁴², X—C₁₋₆-alkylene-NR⁴¹—CONR⁴¹R⁴², C₀₋₆-alkylene-O—CONR⁴¹R⁴², X—C₁₋₆-alkylene-O—CONR⁴¹R⁴², C₀₋₆-alkylene-NR⁴¹—CO₂R⁴¹, X—C₁₋₆-alkylene-NR⁴¹—CO₂R⁴¹, C₀₋₆-alkylene-NR⁴¹R⁴², X—C₁₋₆-alkylene-NR⁴¹R⁴²,
wherein alkyl, alkylene, cycloalkyl, heterocycloalkyl, aryl and heteroaryl is unsubstituted or substituted with 1 to 6 substituents independently selected from halogen, CN, oxo, hydroxy, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
and wherein optionally two adjacent substituents on the aryl and heteroaryl moiety form a 5- to 8-membered partially unsaturated cycle optionally containing 1 to 3 heteroatoms independently selected from O, S or N, and
wherein this additional cycle is unsubstituted or substituted with 1 to 4 substituents independently selected from halogen, CN, oxo, OH, CO₂H, CO₂—C₁₋₄-alkyl, CONHCH₂CO₂H, CONH(CH₂)₂SO₃H, C₁₋₄-alkyl, halo-C₁₋₄-alkyl, O—C₁₋₄-alkyl and O-halo-C₁₋₄-alkyl;
R¹¹, R¹², R²¹, R²², R³¹, R³², R⁴¹, R⁴², R⁵¹ are independently selected from H and C₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
or R¹¹ and R¹², R²¹ and R²², R³¹ and R³², R⁴¹ and R⁴², respectively, when taken together with the nitrogen to which they are attached complete a 3- to 6-membered ring containing carbon atoms and optionally containing 1 or 2 heteroatoms independently selected from O, S or N; and
wherein the new formed cycle is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
R⁷¹ is independently selected from H, CN; NO₂, C₁₋₄-alkyl and C(O)—OC₁₋₄-alkyl,
wherein alkyl is unsubstituted or substituted with 1 to 3 substituents independently selected from halogen, CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), OH, oxo, CO₂H, CO₂—C₁₋₄-alkyl, CONHCH₂CO₂H, CONH(CH₂)₂SO₃H, SO₃H, O—C₁₋₄-alkyl and O-halo-C₁₋₄-alkyl;
R⁷⁵ is independently selected from C₁₋₄-alkyl, 3- to 6-membered cycloalkyl, 3- to 6-membered heterocycloalkyl, 6-membered aryl and 5- to 6-membered heteroaryl,
wherein alkyl, cycloalkyl, heterocycloalkyl, aryl and heteroaryl is unsubstituted or substituted with 1 to 3 substituents independently selected from halogen, CN, Me, Et, CHF₂, CF₃, OH, oxo, CO₂H, CONHCH₂CO₂H, CONH(CH₂)₂SO₃H, SO₃H, OMe, OEt, OCHF₂, and OCF₃;
X is independently selected from O, NR⁵¹, S(O)ₙ, S(═NR⁷¹), S(O)(═NR⁷¹) and S(═NR⁷¹)₂;
Y is independently selected from a bond, O, NR⁵¹, S(O)ₙ, S(═NR⁷¹), S(O)(═NR⁷¹) and S(═NR⁷¹)₂;
n is independently selected from 0 to 2;
and with the proviso, that the following structures are excluded:

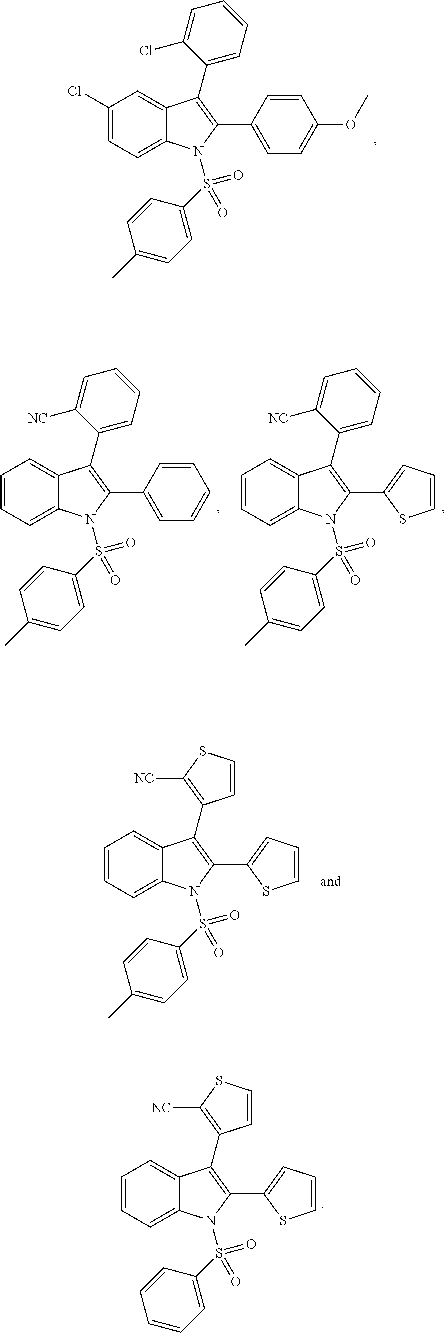

2-5. (canceled)

6. The method according to claim 1, wherein 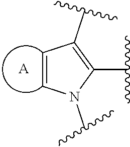
is selected from 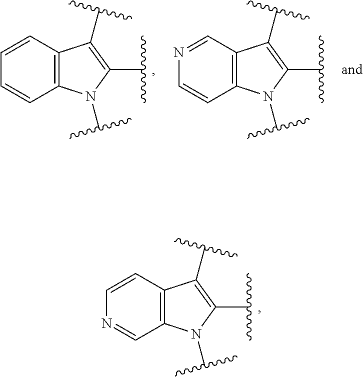
wherein [symbol] is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, NH₂, NHC₁₋₄-alkyl, N(C₁₋₄-alkyl)₂, SO₂—C₁₋₄-alkyl and SO₂-halo-C₁₋₄-alkyl.

7. The method according to claim 1, wherein
\textcircled{B} is selected from the group consisting of phenyl, naphthyl, pyridyl, pyrimidinyl, thiophenyl, thiazolyl, cyclopentyl, cyclohexyl, bicyclo[1.1.1]pentyl, bicyclo[2.2.2]octyl, bicyclo[2.2.1]heptyl, pentacyclo[4.2.0.0^{2,5}.0^{3,8}.0^{4,7}]octyl and piperidinyl,
wherein the cycle is unsubstituted or substituted with 1 to 3 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH and halo-C₁₋₄-alkyl-OH; and wherein optionally two adjacent substituents on the phenyl ring form together a —(CH₂)₃—, —(CH₂)₄—, —OCF₂O— and —OCH₂O— group.

8. The method according to claim 1, wherein
\textcircled{C} is selected from phenyl, pyridyl and thiophenyl; wherein phenyl, pyridyl and thiophenyl is unsubstituted or substituted with 1 to 3 substituents independently selected from the group consisting of F, Cl, CN, OH, oxo, C₁₋₄-alkyl, halo-C₁₋₄-alkyl, O—C₁₋₄-alkyl and O-halo-C₁₋₄-alkyl; and wherein residue -L-R¹ is linked in 1,3-orientation regarding the connection towards 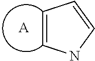
and L is not a bond.

9. The method according to claim 1, wherein
-L-R¹ is selected from 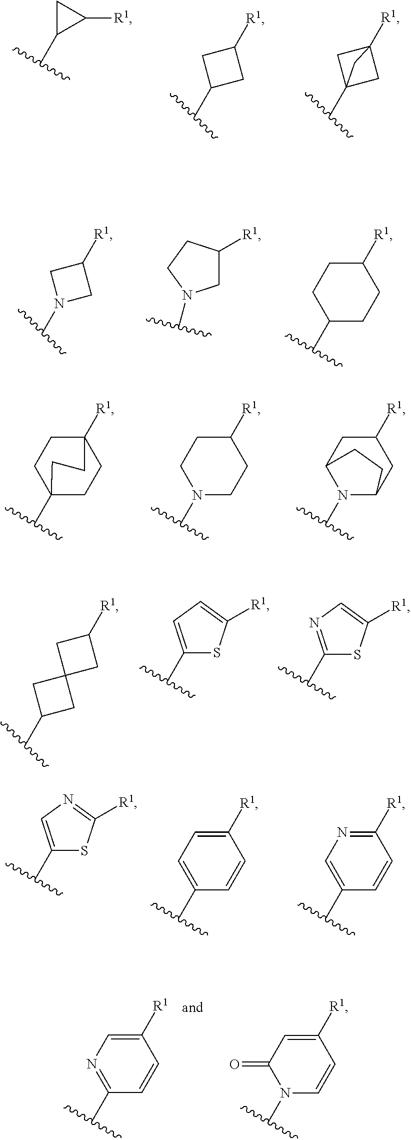
wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

10. The method according to claim 1, wherein
R¹ is selected from CO₂H, tetrazole, CH₂CO₂H, OCH₂CO₂H, SO₂CH₂CO₂H, CHMeCO₂H, CMe₂CO₂H, C(OH)MeCO₂H, CONHSO₂Me and CONH(OH); and optionally the glycine and tauro conjugate thereof.

11. The method according to claim 1, wherein
\textcircled{D} is selected from the group consisting 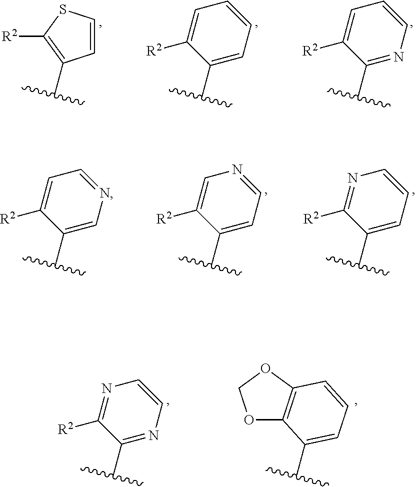
wherein
R² is selected from Me, F, Cl, CN, Me, CHO, CHF₂, CF₃, SO₂Me, 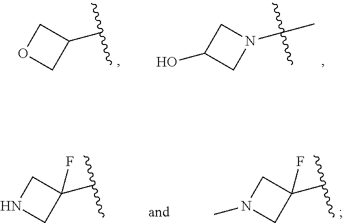
and
wherein \textcircled{D} is optionally further substituted with 1 to 2 substituents selected from the group consisting F, Cl, CN, Me, OMe, CHO, CHF₂ and CF₃.

12. The method according to claim 1, wherein
\textcircled{D} is selected from the group consisting of

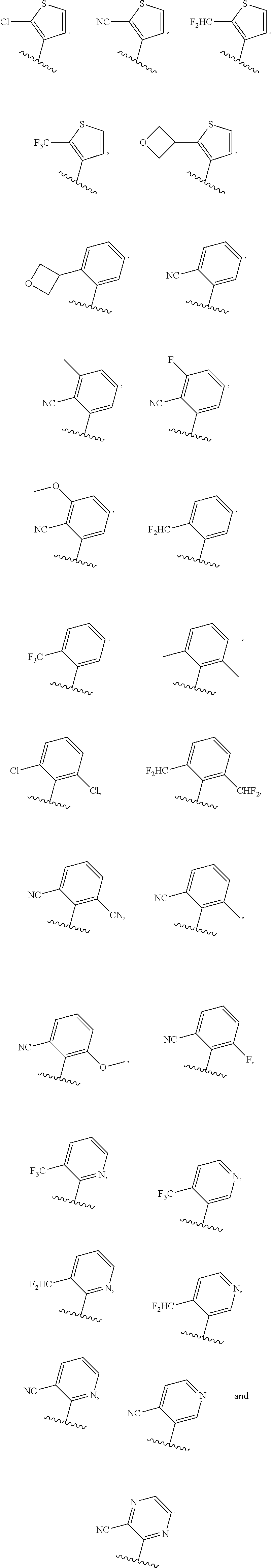

13. The method according to claim 1, wherein Formula (I) contains a substituent selected from the group consisting of CO₂H, tetrazole, CONHSO₂Me and CONH(OH); and optionally the glycine and tauro conjugate thereof.

14. The method according to claim 1, wherein
L-R¹ is 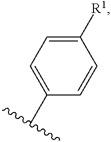
wherein the cycle is unsubstituted or further substituted with 1 to 4 substituents independently selected from the group consisting of F, Cl, Br, CN, OH, oxo, C₁₋₄-alkyl, halo-C₁₋₄-alkyl, O—C₁₋₄-alkyl, O-halo-C₁₋₄-alkyl, C₁₋₄-alkyl-OH, halo-C₁₋₄-alkyl-OH, SO₂—C₁₋₄-alkyl and SO₂-halo-C₁₋₄-alkyl; and wherein optionally two adjacent substituents on the phenyl ring form together a —(CH₂)₃—, —(CH₂)₄—, —OCF₂O— and —OCH₂O— group.

15-20. (canceled)

21. The method according to claim 1, wherein the compound is 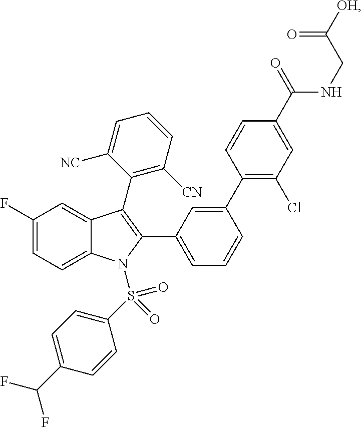 [SMILES: N#Cc1cccc(C#N)c1-c1c(-c2cccc(-c3ccc(C(=O)NCC(=O)O)cc3Cl)c2)n(S(=O)(=O)c2ccc(C(F)F)cc2)c2ccc(F)cc12]
or a pharmaceutically acceptable salt thereof.

22. (canceled)

23. The method according to claim 1, wherein the compound is 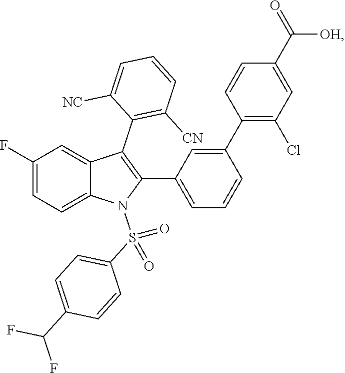 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12]
or a glycine conjugate thereof.

24-25. (canceled)

26. The method according to claim 1, wherein the method is for treating dyslipidemia.

27. The method according to claim 1, wherein the dyslipidemia is hypertriglyceridemia (HTG), severe hypertriglyceridemia (SHTG), familial hypercholesterolemia, heterozygous familial hypercholesterolemia, homozygous familial hypercholesterolemia, familial chylomicronemia syndrome, mixed disorder chylomicronemia, hypercholesterolemia, familial combined hyperlipidemia, familial dysbetalipoproteinemia, or mixed dyslipidemia.

28. The method according to claim 1, wherein the dyslipidemia is severe hypertriglyceridemia (SHTG).

29. The method according to claim 1, wherein the dyslipidemia is characterized by abnormal concentrations of one or more lipids and/or apolipoproteins.

30. The method according to claim 1, wherein the dyslipidemia is characterized by an elevated concentration of total cholesterol, LDL cholesterol, triglycerides (TG), or any combination of the foregoing.

31. The method according to claim 1, wherein the dyslipidemia is characterized by a decreased concentration of HDL cholesterol.

32. The method according to claim 1, wherein the method decreases a risk of pancreatitis in the subject.

33. The method according to claim 1, wherein the method is for treating a metabolic disorder associated with an impairment in lipid homeostasis.

34. The method according to claim 1, wherein the method comprises treating a metabolic disorder associated with an impairment in de novo lipogenesis.

35. The method according to claim 1, wherein the method comprises treating non-alcoholic fatty liver disease (NAFLD) or nonalcoholic steatohepatitis (NASH) in a subject in need thereof, the subject having a glucokinase regulatory protein (GCKR) phenotype.

36. The method according to claim 34, wherein de novo lipogenesis is elevated in the subject.

37. The method according to claim 34, wherein expression of lipogenic genes in the subject and/or lipid accumulation in the subject is reduced.

38. The method, according to claim 1, wherein the subject is administered 0.1 to 25 mg per day of the compound of formula (I), or a glycine conjugate, tauro conjugate, enantiomer, diastereomer, tautomer, N-oxide, solvate, prodrug and pharmaceutically acceptable salt thereof.

39. The method of claim 38, wherein 5 to 15 mg of the compound is administered.
